(19) United States
(12) Patent Application Publication
(10) Pub. No.: US 2026/0325302 A1
(43) Pub. Date: Oct. 22, 2026

(54) HEART VALVE REPAIR DEVICES AND DELIVERY DEVICES THEREFOR

(71) Applicant: Edwards Lifesciences Corporation, Irvine, CA (US)

(72) Inventors: Itay Avinathan, Tel-Aviv (IL); Aviv Galon, Tel Aviv (IL); Natan Anatoly Bukhdruker, Tirat Carmel (IL); Ido Halabi, Givatayim (IL); Meir Abucasis, Hadera (IL); Ofry Efraim Yossef, Tel-Aviv (IL); Sergio Delgado, Irvine, CA (US); David Maimon, Atlit (IL); Radhika Vaid, Rancho Mission Viejo, CA (US); John Washington Campbell, Irvine, CA (US); Gil Hamerman, Kfar Yona (IL); Itai Pelled, Ramat-Hasharon (IL); Lauren R. Freschauf, Mission Viejo, CA (US); Grant Matthew Stearns, Costa Mesa, CA (US); Steven M. Ford, Laguna Beach, CA (US); Mauricio Guerrero, Hawthorne, CA (US); Tejler Javier Gasga, Mission Viejo, CA (US); Christina Marie Santini, Huntington Beach, CA (US); Waina Michelle Chu, Tustin, CA (US); Hao-Chung Yang, Tustin, CA (US); Eva Adriana de la Cruz Romito, Irvine, CA (US); William Diederich Meador, Irvine, CA (US); Yevgeniy Davidovich Kaufman, Denver, CO (US); Jian Lin Phan, Fountain Valley, CA (US); Kim Huong Thi Huynh, Garden Grove, CA (US); Sarah Kazmi, Orange, CA (US)

(21) Appl. No.: 19/565,454

(22) Filed: Mar. 12, 2026

Related U.S. Application Data
(63) Continuation of application No. PCT/US2024/046357, filed on Sep. 12, 2024.
(60) Provisional application No. 63/582,497, filed on Sep. 13, 2023.
(60) Provisional application No. 63/598,923, filed on Nov. 14, 2023.
(60) Provisional application No. 63/557,814, filed on Feb. 26, 2024.
(60) Provisional application No. 63/653,682, filed on May 30, 2024.

(51) Int. Cl. A61F 2/24

(52) U.S. Cl. CPC A61F 2/246; A61F 2/2466; A61F 2220/0008; A61F 2220/0091; A61F 2230/0093

(57) ABSTRACT

Devices are configured to be positioned within a native heart valve to impede regurgitation through the native heart valve. The devices can include an expandable coaptation element, a coaptation element that can be manually adjusted in size and/or can include paddles and a coaptation element made from a single piece of sheet material.

### RELATED APPLICATIONS

The present application is a continuation of Patent Cooperation Treaty Application No. PCT/US2024/046357, filed on Sep. 12, 2024, which claims the benefit of U.S. Provisional Patent Application Nos. 63/582,497, filed on Sep. 13, 2023, 63/598,923, filed on Nov. 14, 2023, 63/557,814, filed on Feb. 26, 2024, and 63/653,682 filed on May 30, 2024, which are all incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to limit the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices and systems for repairing and/or treating a native valve of a patient are disclosed. The devices can be valve repair devices, implantable devices, valve treatment devices, implants, etc. While the devices may be described and/or depicted as implantable devices in some examples herein, similar configurations can be used on other devices, e.g., valve repair devices, treatment devices, etc., that are not necessarily implanted and may be removed after treatment.

In some implementations, there is provided a device (e.g., a treatment device, a repair device, an implantable device, an implant, etc.) that is configured to be positioned within a native heart valve to allow the native heart valve to form a more effective seal.

In some implementations, an implantable device or implant includes an anchor portion. In some implementations, the anchor portion includes one or more anchors. In some implementations, each anchor includes one or more paddles that are each moveable between an open position and a closed position.

In some implementations, a valve repair device includes a first body portion having a first longitudinal axis and a first anchor portion configured to grasp a first leaflet of a native heart valve. In some implementations, the first anchor portion includes a first link having a first portion attached to the first body portion and a second portion opposite the first portion and a second link having a second portion attached to the second portion of the first link and the second link has first portion extending through the first body portion along the first longitudinal axis. In some implementations, axial movement of the second link causes the first link to pivot relative to the first body portion between a first open condition and a first closed condition.

In some implementations, the first link is pivotably attached to the first body portion at a first distal end of the first body portion. In some implementations, the first link is a rigid. In some implementations, the first link is configured to bend at the first proximal portion. In some implementations, the first distal portion is pivotably attached to the second distal portion.

In some implementations, axial movement of the second link from a first retracted position relative to the first body portion to a first extended position relative to the first body portion moves the first link from the first closed condition to the first open condition. In some implementations, the second link includes a flexible first intermediate portion connecting the second portion to the first portion.

In some implementations, the first link has a first capture ready condition between the first open condition and the first closed condition. In the first capture ready condition, an angle between the second portion of the second link and the first portion of the second link is greater in the first capture ready condition than in either the first open condition or the first closed condition.

In some implementations, the angle between the second portion of the second link and the first portion of the second link increases when moving from the first open condition to the first capture ready condition. In some implementations, the angle between the second portion of the second link and the first portion of the second link decreases when moving from the first capture ready condition to the first closed condition.

In some implementations, the device includes first gripping member attached to the first link. In some implementations, the first gripping member includes first moveable arm and a first fixed arm/In some implementations, the first fixed arm is attached to the first link. In some implementations, a first actuation line attached to the first moveable arm and configured to open the first gripping member.

In some implementations, the valve repair device includes a second body portion having a second longitudinal axis and a second anchor portion configured to grasp a second leaflet of the native heart valve.

In some implementations, the second anchor portion includes a third link and a fourth link. In some implementations, the third link has a third proximal portion attached to the second body portion and a third distal portion opposite the third proximal portion. In some implementations, the fourth link has a fourth distal portion attached to the third distal portion and a fourth proximal portion extend through the second body portion along the second longitudinal axis. In some implementations, axial movement of the fourth link causes the third link to pivot relative to the second body portion between a second open condition and a second closed condition.

In some implementations, the third link is pivotably attached to the second body portion at a second distal end of the second body portion. In some implementations, the third link is a rigid. In some implementations, the third link is configured to bend at the third proximal portion. In some implementations, the third distal portion is pivotably attached to the fourth distal portion.

In some implementations, axial movement of the fourth link from a second retracted position relative to the second body portion to a second extended position relative to the second body portion moves the third link from the second closed condition to the second open condition. In some implementations, fourth link further comprises a flexible second intermediate portion connecting the fourth distal portion to the fourth proximal portion.

In some implementations, the third link has a second capture ready condition between the second open condition and the second closed condition. In some implementations, an angle between the fourth distal portion and the fourth proximal portion is greater in the second capture ready condition than in either the second open condition or the second closed condition. In some implementations, the angle between the fourth distal portion and the fourth proximal portion increases when moving from the second open condition to the second capture ready condition. In some implementations, the angle between the fourth distal portion and the fourth proximal portion decreases when moving from the second capture ready condition to the second closed condition.

In some implementations, movement of the first link between the first open condition and the first closed condition is independent of movement of the third link between the second open condition and the second closed condition.

In some implementations, the first body portion is attached to the second body portion. In some implementations, the first body portion has a first inner face and a first outer face, and the second body portion has a second inner face and a second outer face. In some implementations, the first body portion is attached to the second body portion such that the first inner face faces in an opposite direction to the second inner face. In some implementations, the first outer face is directly attached to the second outer face.

In some implementations, the first outer face and the second outer face are attached to a coaptation element at spaced apart locations. In some implementations, the first outer face and the second outer face are attached to opposite sides of the coaptation element.

In some implementations, the coaptation element is expandable. In some implementations, the coaptation element is expandable independent of movement of the second link or the fourth link.

In some implementations, the valve repair device includes a second gripping member attached to the third link. In some implementations, the second gripping member comprises a second moveable arm and a second fixed arm, wherein the second fixed arm is attached to the third link. In some implementations, a second actuation line is attached to the second moveable arm and configured to open the second gripping member.

In some implementations, a method of repairing a native heart valve includes positioning a valve repair device in an open condition at the native heart valve, where the valve repair device has a first body portion, a first link, and a second link. In some implementations, the method includes moving the second link relative to the first body portion proximally along a first longitudinal axis of the first body portion to pivot the first link to a capture ready condition.

In some implementations, the method includes closing a first gripping member attached to the first link to grasp a first valve leaflet of the native heart valve in the first gripping member.

In some implementations, the method includes moving the second link relative to the first body portion further proximally along the first longitudinal axis of the first body portion to pivot the first link from the capture ready position to a closed condition.

In some implementations, closing the first gripping member includes releasing tension in a first actuation line attached to the first gripping member. In some implementations, closing the first gripping member includes engaging the first valve leaflet with one or more friction-enhancing elements on the first gripping member.

In some implementations, the second link has a first portion and a second portion. In some implementations, moving the second link relative to the first body portion to pivot the first link to the capture ready condition includes increasing an angle between the second portion of the first link and the first portion of the first link.

In some implementations, increasing the angle between the second portion of the first link and the first portion of the first link includes flexing a first intermediate portion of the second link. In some implementations, moving the second link relative to the first body portion to pivot the first link from the capture ready condition to the open condition includes decreasing the angle between the second portion of the first link and the first portion of the first link.

In some implementations, the valve repair device includes a second body portion, a third link, and a fourth link. In some implementations, the method includes moving the fourth link relative to the second body portion proximally along a second longitudinal axis of the second body portion to pivot the third link to a capture ready condition.

In some implementations, the method includes closing a second gripping member attached to the third link to grasp a second valve leaflet of the native heart valve in the second gripping member.

In some implementations, the method includes moving the fourth link relative to the second body portion further proximally along the second longitudinal axis of the second body portion to pivot the third link from the capture ready position to a closed condition.

In some implementations, closing the second gripping member includes releasing tension in a second actuation line attached to the second gripping member.

In some implementations, the fourth link has a second distal portion and a second proximal portion. In some implementations, moving the fourth link relative to the second body portion to pivot the third link to the capture ready condition includes increasing an angle between the second distal portion and the second proximal portion. In some implementations, increasing the angle between the second distal portion and the second proximal portion further comprises flexing a second intermediate portion of the fourth link. In some implementations, moving the fourth link relative to the second body portion to pivot the third link from the capture ready condition to the open condition includes decreasing the angle between the second distal portion and the second proximal portion.

In some implementations, moving the second link relative to the first body portion is independent of moving the fourth link relative to the second body portion. In some implementations, the first body portion is attached to the second body portion.

In some implementations, the first body portion is attached to a first side of a coaptation element, and the second body portion is attached to a second side of the coaptation element.

In some implementations, the method further includes radially expanding the coaptation element. In some implementations, radially expanding the coaptation element is independent of moving the second link.

In some implementations, a valve repair device includes a coaptation element, a first anchor connected to a first side of the coaptation element, and a second anchor connected to a second side of the coaptation element. In some implementations, the coaptation element is movable between an expanded condition and a collapsed position.

In some implementations, the first anchor is configured to grasp a first leaflet of a native heart valve and move between an open condition and a closed condition. In some implementations, the second anchor configured to grasp a second leaflet of the native heart valve and move between the open condition and the closed condition.

In some implementations, the coaptation element is movable between an expanded condition and a collapsed position independent of the first anchor and the second anchor moving between the open condition and the closed condition.

In some implementations, the valve repair device includes a paddle and a gripping member. In some implementations, the paddle has a proximal portion, a distal portion opposite the proximal portion, and a joint portion connecting the proximal portion to the distal portion and configured to allow the distal portion to pivot relative to the proximal portion.

In some implementations, the gripping member has a fixed arm and a moveable arm pivotably attached to the fixed arm and is moveable with the distal portion of the paddle.

In some implementations, the coaptation element an expandable frame member and an expandable mechanism mounted within the expandable frame member. In some implementations, the expandable mechanism an expandable member connected to the expandable frame member and an actuation mechanism mounted inside the expandable member.

In some implementations, the actuation mechanism is configured to move the expandable member and the expandable frame member between the expanded configuration and a collapsed configuration.

In some implementations, the expandable frame member includes a plurality of interconnected struts. In some implementations, the proximal portion of the paddle is connected to the expandable frame member. In some implementations, the frame member includes a plurality of fixed height posts interconnected by the plurality of struts and the proximal portion of the paddle is connected to a post.

In some implementations, in the open condition, the distal portion of the paddle extends distally from a distal end of the frame member. In some implementations, the paddle is biased to the closed condition. In some implementations, the paddle includes a shape memory alloy shape set to the closed position. In some implementations, the fixed arm of the gripping member is attached to the distal portion of the paddle. In some implementations, the paddle includes a mounting arm extending proximally from a distal end of the paddle, and wherein the fixed arm of the gripping member is attached to the mounting arm.

In some implementations, the joint portion includes a plurality of torsional spring segments arranged in a repeating pattern and separated by cutouts. In some implementations, the joint portion includes one or more torsional spring coils wound about a coil axis that is traverse to a longitudinal axis of the valve repair device. In some implementations, during movement of the anchor portion from the closed condition to the open condition, the coil axis rotates distally. In some implementations, during movement of the anchor portion from the closed condition to the open condition, the coil axis displaces axially in the distal direction.

In some implementations, the first anchor includes a paddle, a gripping member, and a link connecting the paddle and the gripping member. In some implementations, the paddle has a first proximal portion, a first distal portion opposite the first proximal portion, and a joint portion connecting the first proximal portion to the first distal portion. In some implementations, the joint portion is configured to allow the first distal portion to pivot relative to the proximal end portion. In some implementations, the gripping member is moveable with the distal portion of the paddle, the gripping member having a fixed arm and a moveable arm pivotably attached to the fixed arm.

In some implementations, the link includes a second proximal portion, a second distal portion, and an intermediate portion between the second proximal portion and the second distal portion. In some implementations, the first distal portion of the paddle is attached to the second distal portion of the link. In some implementations, the second proximal portion of the link is attached to the coaptation element.

In some implementations link is S-shaped having a first curved portion connecting the second proximal portion to the intermediate portion and a second curved portion connecting the intermediate portion to the second distal portion. In some implementations, the first curved portion is configured to flex open when the first anchor moves from the closed condition to the open condition. In some implementations the fixed arm of the gripping member is attached to the intermediate portion of the link. In some implementations, the link is biased to the closed condition.

In some implementations, the paddle is biased to the closed position. In some implementations, the joint portion comprises a plurality of torsional spring segments arranged in a repeating pattern and separated by cutouts. In some implementations, the joint portion includes one or more torsional spring coils wound about a coil axis that is traverse to a longitudinal axis of the valve repair device.

In some implementations, the valve repair device includes a connector having a distal end portion connected to the second distal portion of the link and a proximal end portion connected to an anchor actuation element. In some implementations, the paddle is attached to the distal end portion of the connector. In some implementations, the anchor actuation element includes an actuation cap. In some implementations, the distal end portion of the connector is moveable radially relative to the actuation cap. In some implementations, the actuation cap is received in a slot in the distal end portion of the connector.

In some implementations, a method of repairing a native heart valve includes positioning a valve repair device at the native heart valve where the valve repair device includes a coaptation element movable between an expanded condition and a collapsed position, a first anchor connected to a first side of the coaptation element and moveable between an open condition and a closed condition, and a second anchor connected to a second side of the coaptation element and moveable between the open condition and the closed condition.

In some implementations, the method includes moving the valve repair device to a capture ready condition. In some implementations, the method includes positioning the first anchor proximate a first leaflet of the native heart valve and grasping the first leaflet with the first anchor.

In some implementations, the method includes expanding the coaptation element to assist in positioning the second anchor proximate a second leaflet of the native heart valve and grasping the second leaflet with the second anchor. In some implementations, the method includes moving the valve repair device to a closed condition.

In some implementations, grasping the first leaflet with the first anchor includes pinching the first leaflet between a first fixed arm and a first movable arm of a first gripping member. In some implementations, pinching the first leaflet between the first fixed arm and the first movable arm includes releasing tension in a first actuation line attached to the first moveable arm.

In some implementations, expanding the coaptation element includes expanding a frame member of the coaptation element with an actuation mechanism that the frame member is mounted around. In some implementations, expanding the coaptation element occurs after the first anchor has grasped the first leaflet and before the second anchor has grasped the second leaflet. In some implementations, expanding the coaptation element further comprising moving the second anchor radially away from the first anchor.

In some implementations, grasping the second leaflet with the second anchor includes pinching the second leaflet between a second fixed arm and a second movable arm of a second gripping member. In some implementations, pinching the second leaflet between the second fixed arm and the second movable arm includes releasing tension in a second actuation line attached to the second moveable arm.

In some implementations, moving the valve repair device to a closed condition includes pivoting a first distal portion of a first paddle toward the coaptation element. In some implementations, moving the valve repair device to a closed condition further comprises pivoting a second distal portion of a second paddle toward the coaptation element.

In some implementations, pivoting the first distal portion of the first paddle toward the toward the coaptation element includes biasing the first distal portion toward the toward the coaptation element with a torsional spring joint portion positioned between the first distal portion and the first proximal portion. In some implementations, pivoting a first distal portion of a first paddle toward the coaptation element pivots the first gripping member toward the coaptation element.

In some implementations, any of the valve repair devices disclosed herein can be coupled to a delivery system having a catheter and a control handle to deliver the valve repair device to a native heart valve.

In some implementations, a method of repairing and replacing a native valve includes securing a valve repair device to leaflets of the native valve. A cover of the valve repair device is modified such that blood flows through the valve repair device. The valve repair device is expanded. A replacement valve is expanded inside the valve repair device.

In some implementations, the valve repair device is modified by one or more of cutting the cover, ablating the cover, piercing the cover, and removing a portion of the cover.

In some implementations, a coaptation portion of the valve repair device is expanded. In some implementations, the valve repair device is expanded with a balloon.

In some implementations, the valve repair device is expanded by expansion of the replacement valve.

In some implementations, a system for repairing and replacing a native heart valve includes a valve repair device, a tool for modifying a cover, a device for expanding the valve repair device, and a replacement valve. In some implementations, the valve repair device is configured to be secured to leaflets of the native valve.

In some implementations, the tool for modifying the cover of the valve repair device modifies the cover such that blood can flow through the valve repair device.

In some implementations, the device configured to expand the valve repair device can expand the valve repair device while the valve repair device is secured to the leaflets of the native valve.

In some implementations, the replacement valve is configured to expand into contact with and inside surface of the valve repair device.

In some implementations, the tool for modifying the cover is configured to modify the cover by one or more of cutting the cover, ablating the cover, piercing the cover, and removing a portion of the cover.

In some implementations, the system includes a balloon configured to expand the valve repair device.

In some implementations, the replacement valve is configured to expand the valve repair device.

In some implementations, the stretchable cover is treated to decrease the permeability of the cover.

The stretchable cover can be coated with a polymer to decrease the permeability of the cover.

In some implementations, one or more polymer strands made from one or more of TPU, silicone, polyolefin and elastic yarn are woven into the stretchable cover to decrease the permeability of the cover.

In some implementations, the expandable coaptation element is part of a system (e.g., valve repair system, valve treatment system, etc.) that includes a delivery system including a catheter and a control handle and a device (e.g., valve repair device, valve treatment device, implant, etc.) coupled to the delivery system.

In some implementations, the device includes an anchor portion configured to attach to leaflets of a native heart valve. In some implementations, the expandable coaptation element is attached to the anchor portion.

In some implementations, a method of inhibiting regurgitation between native heart valve leaflets includes positioning a device (e.g., valve repair device, valve treatment device, implant, etc.) between the valve leaflets, attaching an anchor portion of the device to the valve leaflets, and expanding a coaptation element attached to the anchor portion.

In some implementations, expanding the coaptation element includes expanding an expandable member positioned within a frame member to move the frame member from a collapsed position to an expanded configuration.

In some implementations, the frame member has a height that remains the same in both the collapsed position and the expanded configuration.

In some implementations, expanding the expandable member includes bending a plurality of strips on the expandable member.

In some implementations, expanding the expandable member includes axially moving one of a distal member and a proximal member relative to the other of the distal member and a proximal member within a passage of the expandable member.

In some implementations, axially moving one of a distal member and a proximal member relative to the other of the distal member and a proximal member includes rotating the proximal member relative to the distal member.

In some implementations, axially moving one of a distal member and a proximal member relative to the other of the distal member and a proximal member further includes threading the proximal member into a passage of the distal member.

In some implementations, axially moving one of a distal member and a proximal member relative to the other of the distal member and a proximal member includes axially moving the distal member within a passage of the proximal member.

In some implementations, the method includes locking the position of the distal member within the passage of the proximal member. In some implementations, the method includes inhibiting axial movement of the proximal member while allowing rotation movement.

In some implementations, an expandable coaptation element for inhibiting regurgitation between native heart valve leaflets includes an expandable mechanism and an expandable frame or spacer. In some implementations, the expandable mechanism is configured to move between an expanded configuration and a collapsed configuration.

In some implementations, the expandable frame is attached around the expandable mechanism.

In some implementations, the expandable mechanism is configured to expand in only two opposite directions.

In some implementations, the expandable frame has a circular cross-section when the expandable mechanism is in the collapsed configuration and the expandable frame has an oval cross-section when the expandable mechanism is in the expanded configuration.

In some implementations, the expandable mechanism comprises a plurality of struts.

In some implementations, a valve treatment device includes a coaptation element movable between an expanded condition and a collapsed position, a first anchor and a second anchor connected to a first side and a second side, respectively, of the coaptation element, and a polymer-based cover at least partially encapsulating the coaptation element. In some implementations, the polymer-based cover is movable between an expanded condition and a collapsed position with the coaptation element.

In some implementations, the coaptation element further includes an expandable frame member and an expandable mechanism mounted within the expandable frame member.

In some implementations, the expandable mechanism includes an expandable member connected to the expandable frame member, and/or an actuation mechanism mounted inside the expandable member. In some implementations, the polymer-based cover at least partially encapsulates the expandable frame member.

In some implementations, the polymer-based cover comprises and/or is formed from a polymeric material having a Shore A durometer of from about 70 to about 95, as measured in accordance with ASTM D-2240.

In some implementations, the polymer-based cover comprises and/or is formed from a polymeric material selected from the group consisting of thermoplastic polyurethanes, polyester polyurethanes, polycarbonate-based urethanes, fluoropolymers, fluoro-oligomers, polyethylenes, polyacrylates, polysiloxanes, and mixtures thereof. In some implementations, the polymeric material is a thermoplastic polyurethane including fluorine, silicone, or both.

In some implementations, the polymer-based cover can be formed as a distinct layer on the coaptation element or by impregnating the polymeric material into a material of the coaptation element. In some implementations, the polymer-based cover comprises a fiber- or textile-reinforced polymeric material.

In some implementations, a device (e.g., a valve repair device, which can be used with a valve of a heart of a living subject and/or simulation) can include first and second paddle portions and a spacer.

In some implementations, the spacer can include a frame. The frame can have at least first and second frame members.

In some implementations, the spacer can further include at least one pivot connection. In some implementations, the at least one pivot connection allows one of the at least first and second frame members to pivot with respect to the other.

In some implementations, the first and second frame members are pivotable about the at least one pivot connection to move the spacer between a collapsed position and an expanded configuration.

In some implementations, the collapsed position comprises the at least first and second frame members having a first distance between them. In some implementations, the expanded configuration comprises the at least first and second frame members having a second distance between them that is greater than the first distance.

In some implementations, the at least one pivot connection comprises a first pivot connection at a first spacer frame location.

In some implementations, the at least one pivot connection comprises a second pivot connection at a second spacer frame location. In some implementations, the first spacer frame location can be located a first end of the spacer and the second spacer frame location can be located at a second end of the spacer, the second end of the spacer opposite the first end of the spacer.

In some implementations, the at least first and second frame members each comprise first and second cross portions and a middle section therebetween.

In some implementations, the first and second cross portions can cross over each other and/or overlap with each other near at least one of the first pivot connection and/or the second pivot connection.

In some implementations, at least a section of the first and second cross portions can be positioned perpendicular to a longitudinal axis of the device. In some implementations, at least a portion of the middle section of the first and second frame members can be positioned parallel to a longitudinal axis of the device.

In some implementations, the at least one pivot connection comprises first and second pivots defining a rotational axis.

In some implementations, the collapsed position comprises a first spacer volume.

In some implementations, the expanded configuration comprises a second spacer volume greater than the first spacer volume.

In some implementations, a device (e.g., a valve repair device, which can be used with a valve of a heart of a living subject and/or simulation) includes first and second paddle portions and a spacer. The spacer is between the first and second paddle portions.

In some implementations, the first and second paddle portions are movable between an open position and a closed position. In some implementations, moving the first and second paddle portions from the closed position to the open position narrows the spacer.

In some implementations, the heart valve repair device includes a frame. In some implementations, the frame has at least first and second frame members and a cap engagement portion for engaging an actuation cap.

In some implementations, when the actuation cap moves away from the cap engagement portion, the first and second paddle portions move from the closed position toward the open position.

In some implementations, the spacer is shape set in a narrow configuration.

In some implementations, moving the first and second paddle portions from the open position to the closed position widens the spacer.

In some implementations, moving the first and second paddle portions from the closed position to the open position automatically narrows the spacer.

In some implementations, a device (e.g., a valve repair device, which can be used with a valve of a heart of a living subject and/or simulation) includes first and second paddle portions and a spacer. The spacer is between the first and second paddle portions.

In some implementations, the first and second paddle portions are movable between an open position and a closed position. In some implementations, moving the first and second paddle portions from the open position to the closed position widens the spacer.

In some implementations, the spacer includes a frame having at least first and second frame members and a cap engagement portion for engaging an actuation cap. In some implementations, the actuation cap can apply force to the cap engagement portion when the first and second paddle portions move from the open position to the closed position.

In some implementations, the spacer is shape set in a narrow configuration.

In some implementations, moving the first and second paddle portions from the open position to the closed position automatically widens the spacer.

In some implementations, moving the first and second paddle portions from the closed position to the open position automatically narrows the spacer.

In some implementations a device (e.g., a valve repair device, which can be used with a valve of a heart of a living subject and/or simulation) includes first and second paddle portions and a spacer. The spacer is between the first and second paddle portions.

In some implementations, the spacer includes a frame having at least first and second frame members.

In some implementations, each frame member includes first and second flex portions and a middle portion therebetween.

In some implementations, each frame member includes a flanged mounting portion.

In some implementations, the spacer includes a brace portion. In some implementations, the brace portion connects each frame member to another frame member. For instance, the brace portion can connect the first frame member to the second frame member.

In some implementations, opening the first and second paddle portions moves the spacer from an expanded configuration to a collapsed position.

In some implementations, the spacer includes a support bushing coupled to the flanged mounting portion. The support bushing can be positioned inside the frame. For instance, the support bushing can be positioned between the first frame member and the second frame member.

In some implementations, when the spacer is in a collapsed position (e.g., narrow configuration), a gap is formed between the support bushing and the brace. In some implementations, when the spacer is in an expanded configuration, the gap between the support bushing and the brace is closed.

In some implementations, the expanded configuration provides a spacer width that is greater than a width of the spacer in the collapsed position.

In some implementation, the collapsed position provides a spacer length that is greater than a length of the spacer in the expanded configuration.

In some implementations, the expanded configuration comprises at least the first and second flex portions having a bent shape that is different from the bent shape of first and second flex portions in the collapsed position.

In some implementations, at least one of the first and second flex portions abuts a portion of the support bushing when the spacer is in the expanded configuration.

In some implementations, the collapsed position comprises the spacer without an actuating force being applied.

In some implementations, the expanded configuration comprises the spacer having an actuating force applied.

In some implementations, in the first and second flex portions flex outwardly from the device upon application of an actuating force.

In some implementations, the spacer includes a space or a gap allows movement of the brace portion as the spacer changes from the collapsed position to the expanded configuration.

In some implementations, the spacer is shape-set to the collapsed position.

In some implementations, the spacer is connected to a bushing.

In some implementations, the bushing comprises a retainer portion adjacent the spacer flanged mounting portion.

In some implementations, the first and second flex portions and the middle portion comprise a sinuous shape in the expanded configuration.

In some implementations, a device (e.g., a valve repair device, which can be used with a valve of a heart of a living subject and/or simulation) comprises a pair of paddles and a spacer formed from a unitary sheet of material.

In some implementations, the unitary sheet of material has a middle portion between first and second end portions of the sheet material.

In some implementations, first and second spacer portions are at the first and second end portions of the sheet material.

In some implementations, the unitary sheet of material can include first and second paddle portions. The first and second paddle portions can be positioned between the first and second spacer portions. The middle portion can be positioned between the first and second paddle portions.

In some implementations, a plurality of first bend portion are formed between the first and second spacer portions and the first and second paddle portions to form the sheet into the paddles and spacer.

In some implementations, the device includes a plurality of second bend portions between the middle portion and the first and second paddle portions.

In some implementations, the device comprises a plurality of third bend portions disposed within the paddles.

In some implementations, the unitary sheet of material is folded to form the spacer and the pair of paddles. The spacer can be positioned between the pair of paddles when the unitary sheet of material is in a folded configuration.

In some implementations, the first and second spacer portions each comprise at least one spacer member.

In some implementations, the first and second spacer portions each comprise a plurality of struts.

In some implementations, the first and second spacer portions each comprise at least one outer strut and at least one inner strut.

In some implementations, the middle portion comprises an aperture.

In some implementations, the first and second paddle portions comprise one or more apertures.

In some implementations, the unitary sheet of material further comprises a sinuous shape.

In some implementations, the first and second spacer portions each comprise a sinuous middle region.

In some implementations, the first and second spacer portions each comprise at least one spacer frame member having a looped middle region.

In some implementations, a same spacer portion is manually sizable between a wide configuration and a narrow configuration. For instance, the first spacer portion can be manually sizable between a wide configuration and a narrow configuration. In some implementations, the second spacer portion can be manually sizable between a wide configuration and a narrow configuration.

In some implementations, a system and/or an apparatus (which can be used with a valve of a heart, e.g., of a living subject or of a simulation) includes first and second anchors and a coaptation element disposed between the first and second anchors.

In some implementations, the first and second anchors are movable between an open position and a closed position.

In some implementations, the coaptation element includes a fabric material that defines a shape of the coaptation element.

In some implementations, the coaptation element is configured to reduce stress on leaflets of a native heart valve when the leaflets are pressed against the coaptation element by the first and second anchors.

In some implementations, the fabric material comprises a lofted cloth material.

In some implementations, the device further comprises a cover that is positioned over the fabric material.

In some implementations, the cover comprises a woven biocompatible fabric that is configured to promote tissue ingrowth.

In some implementations, the fabric material is disposed around a shaft.

In some implementations, each of the first and second anchors comprises an inner paddle, an outer paddle, and a paddle frame.

In some implementations, the device further comprises a distal cap, and the first and second anchors are connected to the distal cap. In some implementations, movement of the distal cap relative to the coaptation element causes the first and second anchors to move between the opened and closed positions.

In some implementations, a system and/or an apparatus (which can be used with a valve of a heart, e.g., of a living subject or of a simulation) includes first and second anchors and a coaptation element disposed between the first and second anchors.

In some implementations, the first and second anchors are movable between an open position and a closed position.

In some implementations, the coaptation element is movable between an elongated configuration and an expanded configuration.

In some implementations, the coaptation element has a first width when in the elongated configuration and a second width when in the expanded configuration. In some implementations, the second width is greater than the first width.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, the coaptation element is configured to be moved to the elongated configuration by one or more actuation elements.

In some implementations, the coaptation element comprises a first end member, a second end member, and a flexible body portion attached to and extending between the first and second end members.

In some implementations, at least one of the first and second end members is movable to move the flexible body portion such that the coaptation element moves between the elongated configuration and the expanded configuration.

In some implementations, the first end member is disposed on a proximal end of the flexible body portion. In such implementations, the first end member is movable in a proximal direction to move the coaptation element to the elongated configuration and movable in a distal direction to move the coaptation element to the expanded configuration.

In some implementations, the first end member is threadably attached to a shaft such that rotation of the first end member relative to the shaft causes the first end member to move and cause the coaptation element to move between the elongated and expanded configurations.

In some implementations, at least one of the first and second end members is made of a material that has a greater stiffness than the flexible body portion.

In some implementations, the flexible body portion comprises a braided metal fabric.

In some implementations, the flexible body portion comprises a flexible metal fabric material that includes strips extending between the first and second end members.

In some implementations, the coaptation element comprises a cap. In some implementations, the cap is configured to be engaged by an actuation element to move the coaptation element between the elongated and expanded configurations.

In some implementations, the coaptation element comprises a flexible body member, a proximal element attached to a proximal end of the flexible body member, and a distal element attached to a distal end of the flexible body member.

In some implementations, the proximal element is movable between an extended position and a retracted position relative to the distal element. In some implementations, the coaptation element is in the elongated configuration when the proximal element is in the extended position, and the coaptation element is in the expanded configuration when the proximal element is in the retracted position.

In some implementations, the proximal element comprises a cam body member and the distal element comprises a plunger member.

In some implementations, the coaptation element comprises a flexible body member and a fluid actuator attached to the flexible body member. The fluid actuator is movable between an extended position and a shortened position. The coaptation element is in the elongated configuration when the fluid actuator is in the extended position, and the coaptation element is in the expanded configuration when the fluid actuator is in the shortened position.

In some implementations, the coaptation element further comprises a fluid line that is in fluid communication with the fluid actuator.

In some implementations, the coaptation element comprises a coating that causes the coaptation element to have an acoustic impedance that is between about 1.5 MRayl and about 1.8 MRayl.

In some implementations, the coaptation element comprises silicone rubber, and wherein the coating comprises at least one of barium sulfate, titanium dioxide, and zirconium dioxide.

In some implementations, a system and/or an apparatus (which can be used with a valve of a heart, e.g., of a living subject or of a simulation) includes a delivery device and a heart valve repair device.

In some implementations, the delivery device includes an actuation tube.

In some implementations, the heart valve repair device includes first and second anchors and a coaptation element disposed between the first and second anchors.

In some implementations, the first and second anchors are movable between an open position and a closed position.

In some implementations, the coaptation element is movable between an elongated configuration and an expanded configuration. In some implementations, the coaptation element has a first width when in the elongated configuration and a second width when in the expanded configuration. In some implementations, the second width is greater than the first width.

In some implementations, the actuation tube is configured to engage and be extended over the coaptation element to cause the coaptation element to be in the elongated configuration. Disengagement between the actuation tube and the coaptation element causes the coaptation element to be in the expanded configuration.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, a system and/or an apparatus (which can be used with a valve of a heart, e.g., of a living subject or of a simulation) includes a delivery device and a heart valve repair device.

In some implementations, the delivery device includes one or more actuation lines.

In some implementations, the heart valve repair device includes first and second anchors and a coaptation element disposed between the first and second anchors.

In some implementations, the first and second anchors are movable between an open position and a closed position.

In some implementations, the coaptation element is movable between an elongated configuration and an expanded configuration. In some implementations, the coaptation element has a first width when in the elongated configuration and a second width when in the expanded configuration, where the second width is greater than the first width.

In some implementations, when the one or more actuation lines are attached to the coaptation element, providing a force to the one or more actuation lines causes the coaptation element to be in the elongated configuration. In some implementations, removal of the force from the one or more actuation lines causes the coaptation element to be in the expanded configuration.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, a system and/or an apparatus (which can be used with a valve of a heart, e.g., of a living subject or of a simulation) includes a delivery device and a heart valve repair device.

In some implementations, the delivery device includes one or more actuation lines.

In some implementations, the heart valve repair device includes first and second anchors and a coaptation element disposed between the first and second anchors.

In some implementations, the first and second anchors are movable between an open position and a closed position.

In some implementations, the coaptation element is movable between an elongated configuration and an expanded configuration. In some implementations, the coaptation element has a first end member, a second end member, and a flexible body portion attached to the first and second end members.

In some implementations, the first end member is movable relative to the second end member to move the flexible body portion such that the coaptation element moves between the elongated configuration and the expanded configuration.

In some implementations, the coaptation element has a first width when in the elongated configuration and a second width when in the expanded configuration, where the second width is greater than the first width. In some implementations, the actuation element of the delivery device is configured to engage the first end member of the coaptation element to move the first end member relative to the second end member.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, the first end member is disposed on a proximal end of the flexible body portion. The first end member is movable in a proximal direction to move the coaptation element to the elongated configuration and movable in a distal direction to move the coaptation element to the expanded configuration.

In some implementations, the first end member is threadably attached to a shaft, and the actuation element is configured to engage the first end member to cause the first end member to rotate relative to the shaft to cause the first end member to move and cause the coaptation element to move between the elongated and expanded configurations.

In some implementations, at least one of the first and second end members is made of a material that has a greater stiffness than the flexible body portion.

In some implementations, the flexible body portion comprises a braided metal fabric material.

In some implementations, the flexible body portion comprises a flexible metal fabric material that includes strips extending between the first and second end members.

In some implementations, a system and/or an apparatus (which can be used with a valve of a heart, e.g., of a living subject or of a simulation) includes a delivery device and a heart valve repair device.

In some implementations, the delivery device includes one or more actuation lines.

In some implementations, the heart valve repair device includes first and second anchors and a coaptation element disposed between the first and second anchors. The first and second anchors are movable between an open position and a closed position.

In some implementations, the coaptation element is movable between an elongated configuration and an expanded configuration.

In some implementations, the coaptation element has cap.

In some implementations, the coaptation element has a first width when in the elongated configuration and a second width when in the expanded configuration, where the second width is greater than the first width.

In some implementations, the actuation element of the delivery device is configured to engage the cap of the coaptation element to move the cap and cause the coaptation element to be in the elongated configuration. Disengagement between the actuation element and the cap causes the coaptation element to be in the expanded configuration.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, a system and/or an apparatus (which can be used with a valve of a heart, e.g., of a living subject or of a simulation) includes a delivery device and a heart valve repair device.

In some implementations, the delivery device includes one or more actuation lines.

In some implementations, the heart valve repair device includes first and second anchors and a coaptation element disposed between the first and second anchors.

In some implementations, the first and second anchors are movable between an open position and a closed position.

In some implementations, the coaptation element is movable between an elongated configuration and an expanded configuration. In some implementations, the coaptation element includes a flexible body member, a proximal element attached to a proximal end of the flexible body member, and a distal element attached to a distal end of the flexible body member.

In some implementations, the proximal element is movable between an extended position and a retraced position relative to the distal element. In some implementations, the coaptation element is in the elongated configuration when the proximal element is in the extended position, and the coaptation element is in the expanded configuration when the proximal element is in the retracted position.

In some implementations, the coaptation element has a first width when in the elongated configuration and a second width when in the expanded configuration, where the second width is greater than the first width. The actuation element of the delivery device is configured to engage the proximal element of the coaptation element to move the proximal element between the extended position and the retracted position.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, the proximal element comprises a cam body member and the distal element comprises a plunger member.

In some implementations, a system and/or an apparatus (which can be used with a valve of a heart, e.g., of a living subject or of a simulation) includes a delivery device and a heart valve repair device. The delivery device includes one or more actuation lines. The heart valve repair device includes first and second anchors and a coaptation element disposed between the first and second anchors. The first and second anchors are movable between an open position and a closed position. The coaptation element is movable between an elongated configuration and an expanded configuration. The coaptation element includes a fluid actuator that is movable between an extended position and a shortened position. The coaptation element is in the elongated configuration when the fluid actuator is in the extended position, and the coaptation element is in the expanded configuration when the fluid actuator is in the shortened position. The coaptation element has a first width when in the elongated configuration and a second width when in the expanded configuration, where the second width is greater than the first width. The fluid line of the delivery device is configured to move the fluid actuator between the extended position and the shortened position.

In some implementations, a ratio of the second width to the first width is between 10/1 and 4/3, between 8/1 and 3/2, between 5/1 and 2/1, between 3/1 and 2/1 or any subrange of these ranges.

In some implementations, a gap between the first anchor and the coaptation element when the first anchor is in the open position and the coaptation element is in the elongated configuration is larger than when the first anchor is in the open position and the coaptation element is in the expanded configuration.

In some implementations, the coaptation element comprises Nitinol. In some implementations, the coaptation element can comprise one or more of a Nitinol braid and/or one or more Nitinol strips.

In some implementations, the coaptation element is shape set in the expanded configuration.

In some implementations, a system and/or an apparatus (which can be used with a valve of a heart, e.g., of a living subject or of a simulation) includes first and second anchors and a coaptation element disposed between the first and second anchors. The first and second anchors are movable between an open position and a closed position. The coaptation element comprises a coating that causes the coaptation element to have an acoustic impedance that is between about 1.5 MRayl and 1.8 MRayl.

In some implementations, the coaptation element comprises silicone rubber.

In some implementations, the coating comprises at least one of barium sulfate, titanium dioxide, and zirconium dioxide.

In some implementations, each of the first and second anchors comprises an inner paddle, and outer paddle, and a paddle frame.

In some implementations, the device further comprises a distal cap, where the first and second anchors are connected to the distal cap such that movement of the distal cap relative to the coaptation element causes the first and second anchors to move between the opened and closed positions.

In some implementations, the distal cap comprises a coating that causes the distal cap to have an acoustic impedance that is between about 1.5 MRayl and 1.8 MRayl.

In some implementations, the coating of the distal cap comprises at least one of barium sulfate, titanium dioxide, and zirconium dioxide.

Some implementations described herein relate to a heart valve repair device or system (which can be used with a valve of a heart, e.g., of a living subject or of a simulation). The heart valve repair device can include first and second anchors that are movable between an open position, a first closed position, and a second closed position. The heart valve repair device can include a coaptation element between the first and second anchors. In some implementations, the coaptation element is movable between an elongated configuration and an expanded configuration. In some implementations, the coaptation element has a first width when in the elongated configuration and a second width when in the expanded configuration. In some implementations, moving the anchors from the first closed position to the second closed position moves the coaptation element from the elongated condition to the expanded condition.

In some implementations, the second width is greater than the first width.

In some implementations, each of the first and second anchors comprises an inner paddle, an outer paddle, and a paddle frame.

In some implementations, the valve repair device includes a distal cap, wherein the first and second anchors are connected to the distal cap such that movement of the distal cap relative to the coaptation element causes the first and second anchors to move between the opened and closed positions.

In some implementations, the outer paddles comprise paddle extension members.

In some implementations, the paddle extension members are narrower when the anchors are in the first closed position than when the anchors are in the second position.

In some implementations, the paddle extension members are shape set when the anchors are in the first closed position.

In some implementations, the coaptation element and the outer paddles are shape set when the anchors are in the first closed position.

In some implementations, tension applied to the anchors by captured leaflets of the native valve causes the coaptation element to decrease in width.

Some implementations described herein relate to a heart valve repair device or system (which can be used with a valve of a heart, e.g., of a living subject or of a simulation). The heart valve repair device can include first and second anchors that are movable between an open position, a partially open position, and a closed position. In some implementations, each of the first and second anchors comprises an inner paddle, an outer paddle, and a paddle frame. In some implementations, the heart valve repair device includes a cover covering at least one of the inner paddle and the outer paddle of the first and second anchors. In some implementations, the heart valve repair device includes an extension of the cover extending from the anchors when the anchors are in the partially open position so that the width of the anchors including the extension is wider than the width of the anchors without the extension.

In some implementations, the extension is attached to the cover covering the inner paddle.

In some implementations, the extension is attached to the outer paddle.

In some implementations, the extension is attached to the paddle frame.

In some implementations, the extension is attached to the cover covering the outer paddle.

In some implementations, the extension is attached to the inner paddle.

In some implementations, the extension is attached to both the inner paddle and the outer paddle.

In some implementations, the extension is integrally formed with the cover.

In some implementations, the extension comprises and/or is formed from a loop of material attached to the cover.

Some implementations described herein relate to a heart valve repair device or system (which can be used with a valve of a heart, e.g., of a living subject or of a simulation). The heart valve repair device can include a plurality of anchors that are movable between an open position and a closed position. In some implementations, the heart valve repair device can include a gripping member attached to each of the plurality of anchors. In some implementations, the heart valve repair device can include a cover covering an end of each gripping member. In some implementations, the cover can include a tube of material extending from a first end to a second end, wherein first end comprises an opening and the second end comprises a closed portion. In some implementations, the gripping member is inserted into the opening of the cover.

In some implementations, the gripping member comprises a plurality of friction enhancing-elements that protrude through the cover.

In some implementations, a single line of stitching is formed across the first end of the cover to close the opening of the cover.

In some implementations, the closed end of the cover is formed by flattening and fusing the second end of the tube of material.

In some implementations, the second end of the tube of material is folded before being fused.

In some implementations, the closed end of the cover comprises an attachment loop.

In some implementations, the closed end of the cover comprises an attachment hole.

In some implementations, the coaptation element is arranged between the plurality of anchors in the closed position so that leaflets of the native valve are captured between the coaptation element and the plurality of anchors when the anchors are closed; and openings formed between the leaflets during diastole are larger than openings formed between the leaflets when pinched together by the anchors absent the coaptation element.

Some implementations described herein relate to a heart valve repair system (which can be used with a valve of a heart, e.g., of a living subject or of a simulation). The heart valve repair system can include a valve repair device and a delivery system. The delivery system includes a catheter assembly and a control handle. The control handle can include a control knob connected to a drive shaft. The control handle can include a multiplier gearbox that is engaged by the drive shaft. The control handle can include an actuation element that is operatively coupled to an output shaft of the multiplier gearbox. In some implementations, the actuation element extends through the catheter assembly to the valve repair device. In some implementations, rotating the control knob causes the actuation element to rotate via the multiplier gearbox so that a width of the coaptation element of the valve repair device is changed.

In some implementations, the output shaft of the multiplier gearbox is connected to a drive barrel. In some implementations, the drive barrel comprises a central drive slot. In some implementations, the actuation element is attached to a drive nut that slideably engages the central drive slot of the drive barrel.

In some implementations, the drive nut comprises a support ring that is connected to a main body of the drive nut via arms. In some implementations, the drive barrel comprises longitudinal slots connected to the central drive slot for providing clearance for the arms of the drive nut.

In some implementations, the heart valve repair system includes a slider comprising a semi-circular grooved cradled for supporting and engaging the support ring of the drive nut. In some implementations, moving the slider in a proximal and a distal direction engages and moves the drive nut and actuation element in the proximal and the distal direction.

In some implementations, the multiplier gear box is a planetary gear box. In some implementations, the drive shaft is connected to a carrier of the planetary gear box and the output shaft is connected to a sun gear of the planetary gearbox.

In some implementations, the multiplier gear box has a gear ratio between the drive shaft and the output shaft in a range of 1:2 to 1:10, or in a range of 1:3 to 1:8, or in a range of 1:4 to 1:6, or 1:5.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, anthropomorphic ghost, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example device or implant, in various stages of deployment;

FIG. 15 shows an example device that is similar to the device illustrated by FIGS. 8-14, but where the paddles are independently controllable;

FIGS. 16-21 show the example device of FIGS. 8-14 being delivered and deployed within a native valve;

FIG. 22 shows a perspective view of an example device in a closed position;

FIG. 23 shows a perspective view of an example device in a closed position;

FIG. 24 illustrates an example device with paddles in an open position;

FIG. 25A illustrates an example device with paddles in a closed position;

FIG. 25B illustrates a top view of an example device;

FIG. 26 illustrates a perspective view of an example device having paddles of adjustable widths;

FIG. 27 is a cross-section of the example device of FIG. 26 in which the device is bisected;

FIG. 28 is another cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31 illustrates a perspective view of an example expandable coaptation element of a device;

FIG. 32 illustrates a view of an example device having the expandable coaptation element shown in FIG. 31 implanted within a native valve;

FIG. 33 illustrates a perspective view of an example device with an expandable coaptation element having a shell;

FIGS. 34-35 show example constructions of a component of the shell shown in FIG. 33;

FIG. 36 illustrates a front view of the device of FIG. 33;

FIG. 37 illustrates a top view of the device of FIG. 33;

FIG. 38 illustrates a side view of the device of FIG. 33 in an expanded configuration;

FIG. 39 illustrates a side view of the device of FIG. 33 in a narrowed configuration;

FIG. 40 illustrates a perspective view of an example of an expandable sleeve of a coaptation element;

FIG. 41 illustrates a top view of the expandable sleeve of the coaptation element of FIG. 40 in a narrowed configuration;

FIG. 42 illustrates a perspective view of the expandable sleeve of the coaptation element of FIG. 40 when the expandable coaptation element is in an expanded configuration;

FIG. 43 illustrates a top view of the expandable sleeve of the coaptation element of FIG. 42 in an expanded configuration;

FIG. 44 illustrates an expandable coaptation element in a non-tensioned condition;

FIG. 45 illustrates an expandable coaptation element in a tensioned position;

FIG. 46-48 illustrate different types of expansion of expandable coaptation elements from a top view;

FIG. 49 illustrates a top view of an example device having an expandable coaptation element of FIGS. 44 and 45 implanted in a native heart valve with the expandable coaptation element in a non-tensioned condition;

FIG. 50 illustrates a side view of the device and native heart valve of FIG. 49;

FIG. 51 illustrates a top view of an example device having an expandable coaptation element of FIGS. 44 and 45 implanted in a native heart valve with the expandable coaptation element in a tensioned condition;

FIG. 52 illustrates a side view of the device and native heart valve of FIG. 51;

FIG. 53 illustrates a perspective view of an example expandable spacer or coaptation element in an expanded condition;

FIG. 54 illustrates a front view of the expandable spacer or coaptation element of FIG. 53;

FIG. 55 illustrates a perspective cross-section view of the example expandable spacer or coaptation element of FIG. 53 taken along the line 598-598 of FIG. 54;

FIG. 56 illustrates a cross-section view of the example expandable spacer or coaptation element of FIG. 53 taken along the line 598-598 of FIG. 54;

FIG. 57 illustrates a perspective view of an example expandable spacer or coaptation element in an expanded condition;

FIG. 58 illustrates a front view of the expandable spacer or coaptation element of FIG. 57;

FIG. 59 illustrates a perspective cross-section view of the example expandable spacer or coaptation element of FIG. 57 taken along the line 602-602 of FIG. 58;

FIG. 60 illustrates a cross-section view of the example expandable spacer or coaptation element of FIG. 57 taken along the line 602-602 of FIG. 58;

FIG. 61 illustrates a perspective view of an example expandable spacer or coaptation element in an unexpanded condition with a latch tube in an unlatched condition;

FIG. 62 illustrates a front view of the expandable spacer or coaptation element of FIG. 61;

FIG. 63 illustrates a perspective cross-section view of the example expandable spacer or coaptation element of FIG. 61 taken along the line 606-606 of FIG. 62;

FIG. 64 illustrates a cross-section view of the example expandable spacer or coaptation element of FIG. 61 taken along the line 606-606 of FIG. 62;

FIG. 65 illustrates a perspective view of the example expandable spacer or coaptation element of FIG. 61 with the latch tube in a latched condition;

FIG. 66 illustrates a front view of the expandable spacer or coaptation element of FIG. 65;

FIG. 67 illustrates a perspective cross-section view of the example expandable spacer or coaptation element of FIG. 65 taken along the line 610-610 of FIG. 66;

FIG. 68 illustrates a cross-section view of the example expandable spacer or coaptation element of FIG. 65 taken along the line 610-610 of FIG. 66;

FIG. 69 illustrates an enlarged detailed view of the example expandable spacer or coaptation element of FIG. 61 taken in area 607 of FIG. 63;

FIG. 70 illustrates an enlarged detailed view of the example expandable spacer or coaptation element of FIG. 65 taken in area 611 of FIG. 67;

FIG. 71 illustrates an example of a portion of an expandable coaptation element;

FIG. 72 illustrates a cell within a frame of the expandable coaptation element of FIG. 71;

FIG. 73 illustrates the cell within a frame of FIG. 72 in elongated configuration;

FIG. 74 illustrates the cell of FIG. 71 with an example actuation member;

FIG. 75 illustrates an example of an expandable coaptation element for a device;

FIG. 76 illustrates an example of an expandable coaptation element for a device;

FIG. 77 illustrates an example of an expandable coaptation element for a device;

FIG. 78 illustrates a side view of an example of a device attached to leaflets of a heart valve;

FIG. 79 illustrates a top view of the device and heart valve of FIG. 78;

FIG. 80 illustrates a side view of an example of a device in an open configuration and an extension or blocking member for a heart valve;

FIG. 81 illustrates the device and cap of FIG. 80 where the devise is in a closed configuration;

FIG. 82 illustrates a perspective view of an example frame member of an expandable coaptation element;

FIG. 83 illustrates a sectioned perspective view of the frame member of FIG. 82;

FIG. 84 illustrates a top view of the frame member of FIG. 82 with an example expandable/expansion member attached;

FIG. 85 illustrates a side sectional view of the expandable/expansion member and the frame member of FIG. 84;

FIG. 86 illustrates a sectioned perspective view of the expandable/expansion member and the frame member of FIG. 84;

FIG. 87 illustrates a perspective view of the expandable/expansion member of FIG. 84 in an expanded configuration;

FIG. 88 illustrates a perspective view of the expandable/expansion member of FIG. 84 in a collapsed configuration;

FIG. 89 illustrates an exploded perspective view of the expandable/expansion member and an example actuation mechanism for the expandable coaptation element;

FIG. 90 illustrates a sectional view of the expandable/expansion member and the actuation mechanism of FIG. 89 taken along the 90-90 line;

FIG. 91 illustrates a sectional view of the expandable/expansion member and the actuation mechanism of FIG. 89 taken along the 91-91 line;

FIG. 92 illustrates a perspective view of the expandable/expansion member and the actuation mechanism of FIG. 89;

FIG. 93 illustrates a front view of the expandable/expansion member and the actuation mechanism of FIG. 89;

FIG. 94 illustrates a front view of an example expandable/expansion member and an example actuation mechanism for an expandable coaptation element;

FIG. 95 illustrates an exploded view of the actuation mechanism of FIG. 94;

FIG. 96 illustrates a sectional view of the expandable/expansion member and the actuation mechanism of FIG. 94 taken along the 96-96 line;

FIG. 97 illustrates a sectional view of the expandable/expansion member and the actuation mechanism of FIG. 94 taken along the 97-97 line;

FIG. 98 illustrates a perspective view of the sectioned expandable/expansion member and the actuation mechanism of FIG. 97;

FIG. 99 illustrates an example expandable/expansion member and an example actuation mechanism for an expandable coaptation element;

FIG. 100 illustrates an example expandable coaptation element in a collapsed configuration;

FIG. 101 illustrates the expandable coaptation element of FIG. 100 in an expanded configuration;

FIG. 102 illustrates a plan view of a portion of an example cover for an expandable coaptation element with the cover in a first state;

FIG. 103 illustrates a plan view of a portion of a stretchable portion of the cover of FIG. 102 with the cover in a second state;

FIG. 104 illustrates a plan view of a portion of a stretchable portion of the cover of FIG. 102;

FIG. 105 illustrates a plan view of a plain weave of an example cover for an expandable coaptation element;

FIG. 106 illustrates a plan view of a portion of an example cover for an expandable coaptation element, where stretchable portions are in a pre-heated state;

FIG. 107 illustrates a plan view of a portion of an example cover for an expandable coaptation element, where stretchable portions are in a post-heated state;

FIG. 108 illustrates a plan view of an example cover having a stretchable portion that is attached to a paddle frame of a device, where the paddle frame is in an expanded configuration and the cover is in a stretched position;

FIG. 109 illustrates a plan view of an example cover having a stretchable portion that is attached to a paddle frame of a device, where the paddle frame is in a narrowed configuration and the cover is in a normal position;

FIG. 110 shows a schematic view an example cover for an expandable coaptation element;

FIG. 111 illustrates a perspective view of an example device or implant or component thereof in a closed position;

FIG. 112 illustrates a perspective view of an example device or implant or component thereof in a closed position;

FIG. 113 illustrates a side view of the device or implant (or component thereof) of FIG. 112;

FIGS. 114-117 illustrate the device or implant (or component thereof) of FIG. 111 in various stages of deployment;

FIGS. 118-121 illustrate arrangements for using one or more of the devices or implants (or components thereof) of FIG. 111;

FIGS. 122-128 illustrate an example device or implant in various stages of deployment;

FIGS. 129-133 illustrate an example device or implant in various stages of deployment;

FIGS. 134-138 illustrate an example device or implant in various stages of deployment;

FIGS. 139-143 illustrate an example device or implant in various stages of deployment;

FIGS. 144-146 illustrate an example device or implant in various stages of deployment;

FIG. 147 illustrates an exploded perspective view of an example device or implant or component(s) thereof;

FIG. 148 illustrates a perspective view of the example device or implant (or component(s) thereof) of FIG. 147;

FIG. 149 illustrates a perspective view of the example device or implant (or component(s) thereof) of FIG. 147;

FIG. 150 illustrates a perspective view of the example device or implant (or component(s) thereof) of FIG. 147 in a closed position;

FIG. 151 illustrates a perspective view of the example device or implant (or component(s) thereof) of FIG. 147 initiating opening of the device or implant;

FIG. 152 illustrates a perspective view of the example device or implant (or component(s) thereof) of FIG. 147 in a partially open position;

FIG. 153 illustrates a perspective view of the example device or implant (or component(s) thereof) of FIG. 147 in a capture ready position, but with the clasp in a closed position;

FIG. 154 illustrates a perspective view of the example device or implant (or component(s) thereof) of FIG. 147 approaching a bailout position, but with the clasp in a closed position;

FIG. 154 illustrates a perspective view of the example device or implant (or component(s) thereof) of FIG. 147 in a bailout position, but with the clasp in a closed position;

FIG. 156 illustrates a perspective view of an example device or implant;

FIG. 157 illustrates a front view of the example device or implant of FIG. 156;

FIG. 158 illustrates a front view of the example device or implant of FIG. 156 attached to leaflets of a native valve;

FIG. 159 illustrates a front view of the example device or implant of FIG. 156 attached to leaflets of a native valve where a cover of the device is being broken;

FIGS. 160-161 illustrate a front view of the example device or implant of FIG. 156 attached to leaflets of a native valve where the device or implant is being expanded;

FIGS. 162-164 illustrate a front view of the example device or implant of FIG. 156 attached to leaflets of a native valve where a valve is being installed in the device or implant;

FIGS. 165-166 illustrate a front view of the example device or implant of FIG. 156 attached to leaflets of a native valve where the device or implant is being expanded by a valve that is being installed in the device or implant;

FIG. 167 shows a schematic view of a coating being laminated onto a cover material;

FIG. 168 illustrates a perspective view of an example expandable member in a substantially non-expanded configuration;

FIG. 169 illustrates a perspective view of an example frame member of an expandable coaptation element;

FIG. 170 illustrates a perspective view of an example expandable member and an example actuation mechanism for an expandable coaptation element;

FIG. 171 illustrates a cross-sectional view of the expandable member and actuation mechanism of FIG. 170;

FIG. 172 illustrates a perspective view of an assembly of the expandable member and actuation mechanism of FIG. 170 and a rail of a valve repair device;

FIG. 173 illustrates a side view of the assembly of FIG. 172;

FIG. 174 illustrates an end view of the assembly of FIG. 172;

FIG. 175 illustrates a perspective view of a valve repair device with an expandable spacer in an unexpanded configuration;

FIG. 176 illustrates a perspective view of the valve repair device of FIG. 175 with the spacer in an expanded configuration;

FIG. 177 illustrates a plan view from an atrial side of a tricuspid valve of the valve repair device of FIG. 176 installed on leaflets of the tricuspid valve;

FIG. 178 illustrates a perspective view of a valve repair device having the assembly of FIG. 172;

FIG. 179 illustrates a side sectional view of an expandable/expansion member having a polymer-based cover thereon;

FIG. 180 illustrates a perspective view of an example frame member of an expandable coaptation element having a polymer-based cover thereon;

FIG. 181 illustrates an example expandable coaptation element having a polymer-based cover thereon in a collapsed configuration;

FIG. 182 illustrates the expandable coaptation element and polymer-based cover of FIG. 181 in an expanded configuration;

FIG. 183 illustrates a perspective view of a valve repair device with an expandable spacer having a polymer-based cover thereon in an unexpanded configuration;

FIG. 184 illustrates a perspective view of the valve repair device of FIG. 183 with the spacer and polymer-based cover in an expanded configuration;

FIG. 185A is a top view of an example of a valve repair device having an expandable coaptation element or spacer in a reduced width condition;

FIG. 185B is a side view of the valve repair device illustrated by FIG. 185A;

FIG. 185C is a top view of the example valve repair device of FIG. 185A with the expandable coaptation element or spacer in an expanded width condition;

FIG. 185D is a side view of the valve repair device illustrated by FIG. 185C;

FIGS. 186A and 186B are schematic illustrations of an example valve repair device having an expandable coaptation element or spacer;

FIG. 187A illustrates an example of a valve repair device having a self-narrowing spacer in a narrowed configuration;

FIG. 187B illustrates the valve repair device of FIG. 187A in an expanded condition;

FIG. 187C is a perspective view of the valve repair device of FIG. 187A in the expanded condition;

FIG. 188A illustrates an example of a valve repair device having a self-narrowing spacer in a narrowed configuration;

FIG. 188B illustrates the valve repair device of FIG. 188A in an expanded condition;

FIG. 188C illustrates an example of a valve repair device having a self-narrowing spacer in a narrowed configuration;

FIG. 188D illustrates the valve repair device of FIG. 188A in an expanded condition;

FIG. 189A illustrates a flat pattern of an example of a valve repair device frame having an integrated frame structure made from a single or unitary piece of material;

FIG. 189B is a perspective view a valve repair device frame made from the flat pattern illustrated by FIG. 189A;

FIG. 189C is a side view of the valve repair device frame illustrated by FIG. 189B;

FIG. 189D is a front view of the valve repair device frame illustrated by FIG. 189B;

FIG. 189E is a front view of the valve repair device frame illustrated by FIG. 189B;

FIG. 190A is a front view of a valve repair device frame made from a single piece of flat or sheet material;

FIG. 190B is a side view of the valve repair device frame illustrated by FIG. 190A;

FIG. 190C is a perspective view of the valve repair device frame illustrated by FIG. 190A;

FIG. 191 illustrates an example of a valve repair device with a size adjustable spacer in a wide condition;

FIG. 192 illustrates the valve repair device of FIG. 191 in a narrow condition;

FIG. 193 illustrates an example of a valve repair device having an example spacer, where the spacer is shown without an optional cover;

FIG. 194 illustrates the valve repair device of FIG. 193, where the spacer is shown with an optional cover;

FIG. 195 is a top view of the valve repair device of FIG. 193, where the spacer is shown with an optional cover;

FIG. 196 illustrates an example of a valve repair device having an example spacer, where the spacer is shown in an elongated configuration;

FIG. 197 illustrates the valve repair device of FIG. 196, where the spacer is shown in an expanded configuration;

FIG. 198 illustrates an example of a valve repair device having an example spacer, where the spacer is shown in an elongated configuration;

FIG. 199 illustrates the valve repair device of FIG. 198, where the spacer is shown in an expanded configuration;

FIG. 200 illustrates an example of a valve repair device having an example spacer, where the spacer is shown in an elongated configuration;

FIG. 201 illustrates the valve repair device of FIG. 200, where the spacer is shown in an expanded configuration;

FIG. 201A illustrates a schematic top view of the valve repair device of FIG. 201, where the spacer is shown in the expanded configuration shown in FIG. 201;

FIG. 202 illustrates an example of a valve repair device having an example spacer, where the spacer is shown in an elongated configuration;

FIG. 203 illustrates the valve repair device of FIG. 202, where the spacer is shown in an expanded configuration;

FIG. 204 illustrates an example of a valve repair device having an example spacer, where the spacer is shown in an expanded configuration;

FIG. 205 illustrates the valve repair device of FIG. 204, where the spacer is shown in an elongated configuration;

FIG. 206 illustrates an example of a valve repair device having an example spacer, where the spacer is shown in an expanded configuration;

FIG. 207 illustrates the valve repair device of FIG. 206, where the spacer is shown in an elongated configuration;

FIG. 208 illustrates an example of a valve repair device having an example spacer, where the spacer is shown in an expanded configuration;

FIG. 209 illustrates the valve repair device of FIG. 208, where the spacer is shown in an elongated configuration;

FIG. 210 illustrates an example of a valve repair device;

FIG. 211 illustrates an example of a valve repair device;

FIG. 212 illustrates an example of a valve repair device;

FIG. 213 shows an example device being delivered within a native valve;

FIG. 214 shows an example device being delivered within a native valve, where the device includes one or more components configured to improve the visibility of the device when using an imaging device;

FIG. 215 shows a front view of an example device in a closed condition with the paddles closed to a first closed position;

FIG. 216 shows the example device of FIG. 215 viewed from the atrial side when installed in a heart valve that is in diastole and with the paddles on the ventricular side shown in broken lines;

FIG. 217 shows a front view the example device of FIG. 215 in a closed condition with the paddles closed to a second closed position;

FIG. 218 shows the example device of FIG. 217 viewed from the atrial side when installed in a heart valve that is in diastole and with the paddles on the ventricular side shown in broken lines;

FIG. 219 shows a front view of an example device with the paddles in a partially open, capture-ready position;

FIG. 220 shows a top view of the example device of FIG. 219;

FIG. 221 shows a fabric cover for the end of a gripping member of an example device with the fabric cover in an uninstalled condition;

FIG. 222 shows the fabric cover of FIG. 221 with the fabric cover folded over the end of the gripping member and stitched to install the fabric cover;

FIG. 223 shows an example of a tube-shaped fabric cover for a gripping member with a sealed end;

FIG. 224 shows an example of a tube-shaped fabric cover for a gripping member with a folded and sealed end;

FIG. 225 shows a top view of a tube-shaped fabric cover arranged over the end of the gripping member;

FIG. 226 shows a top view of the tube-shaped fabric cover of FIG. 225 with the open end stitched closed;

FIG. 227 shows a top view of a tube-shaped fabric cover arranged over the end of the gripping member, the tube-shaped fabric cover including an attachment loop;

FIG. 228 shows a top view of a tube-shaped fabric cover arranged over the end of the gripping member, the tube-shaped fabric cover including a plurality of attachment holes;

FIG. 229 shows a perspective view of a control handle of a delivery system for an implantable prosthetic device;

FIG. 230 shows a perspective view of the control handle of FIG. 229 with a portion of an outer case removed;

FIG. 231 shows a perspective view of an extendable actuation mechanism of the control handle of FIG. 229;

FIG. 232 shows an enlarged perspective view of the actuation mechanism of FIG. 231;

FIG. 233 shows an exploded perspective view of the extendable actuation mechanism of FIG. 231.

FIG. 234 shows a front view of a valve repair device implanted in the native valve, wherein two native leaflets of the native valve are coapted together by anchors of the valve repair device;

FIG. 235 shows a ventricular view of the valve repair device of FIG. 234;

FIG. 236 shows a front view of a valve repair device implanted in the native valve, wherein two native leaflets of the native valve are coapted against a coaptation element by anchors of the valve repair device; and

FIG. 237 shows a ventricular view of the valve repair device of FIG. 236.

### DETAILED DESCRIPTION

The following description refers to the accompanying drawings, which illustrate examples of the present disclosure. Other implementations having different structures and operation do not depart from the scope of the present disclosure.

Some implementations of the present disclosure are directed to systems, devices, methods, etc. for repairing a defective heart valve. For example, some implementations of devices, treatment device, repair devices, valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

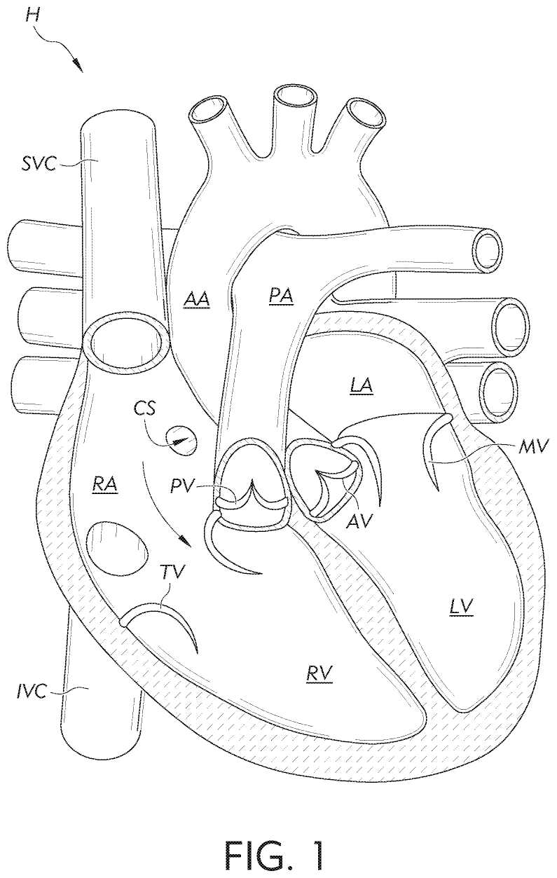

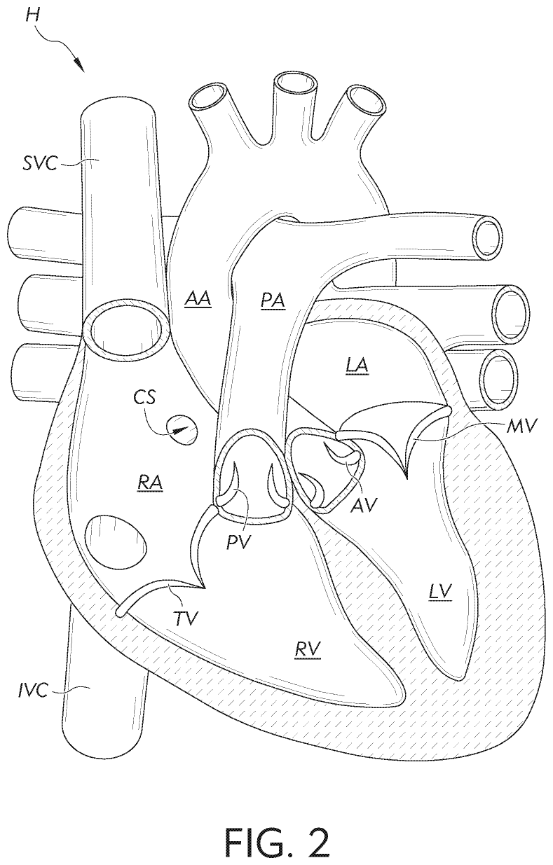

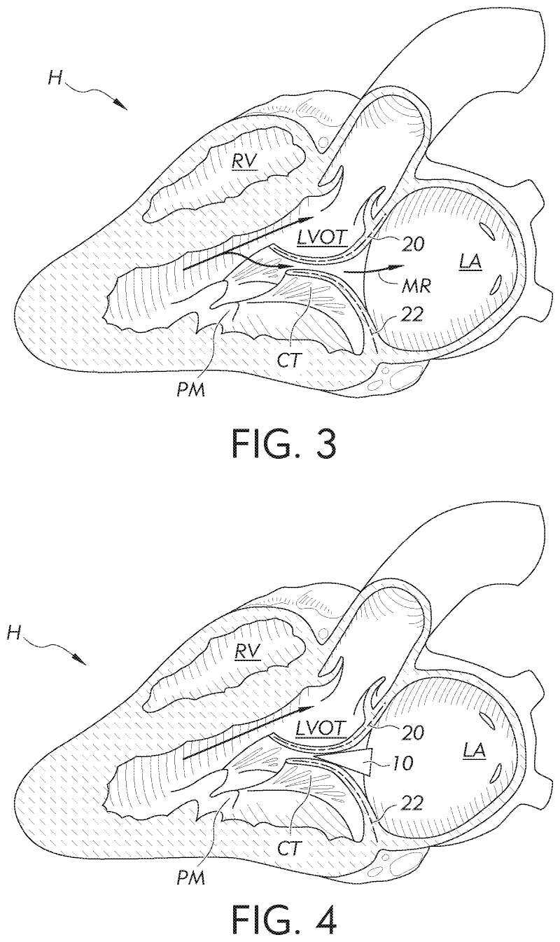

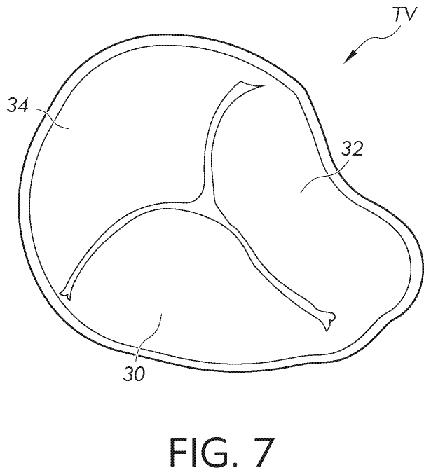

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coat” in the flow stream to form the one-way, fluid-occluding surfaces. The native valve repair and/or treatment systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

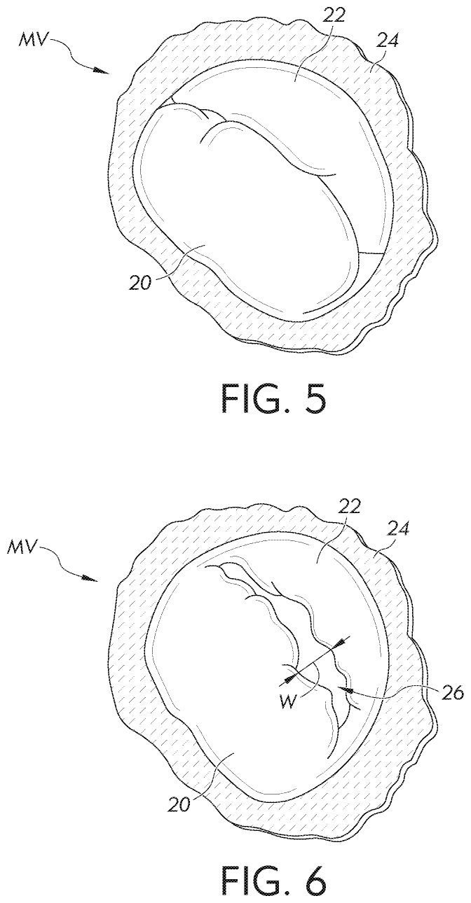

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaptation element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaptation element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaptation element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the treatment device, repair devices, implants, etc. provided herein can be centrally located between the three leaflets 30, 32, 34.

An example device (e.g., valve repair device, valve treatment device, implantable device, implant, etc.) can optionally have a coaptation element (e.g., spacer, coaptation element, gap filler, membrane, sheet, plug, wedge, balloon, etc.) and at least one anchor (e.g., one, two, three, or more). In some implementations, a device (e.g., a valve repair device, a valve treatment device, an implantable device, an implant, etc.) can have any combination or sub-combination of the features disclosed herein without a coaptation element. When included, the coaptation element (e.g., spacer, coaptation element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets.

In some implementations, the anchor can be configured for use in the tricuspid valve, the anchor is configured to secure the device to one, two, or three of the tricuspid leaflets such that the coaptation element is positioned between the three native leaflets.

In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device can be configured to be deployed and/or implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods that can be used with the concepts herein can be found in U.S. Pat. No. 8,449,599 and U.S. Patent Application Publication Nos. 2014/0222136, 2014/0067052, 2016/0331523, PCT patent application publication Nos. WO2020/076898, WO2023/278663, WO2023/004098, WO2023/091520, WO2023/107296, WO2023/086340, WO2023/003755, and WO2022/231889 each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

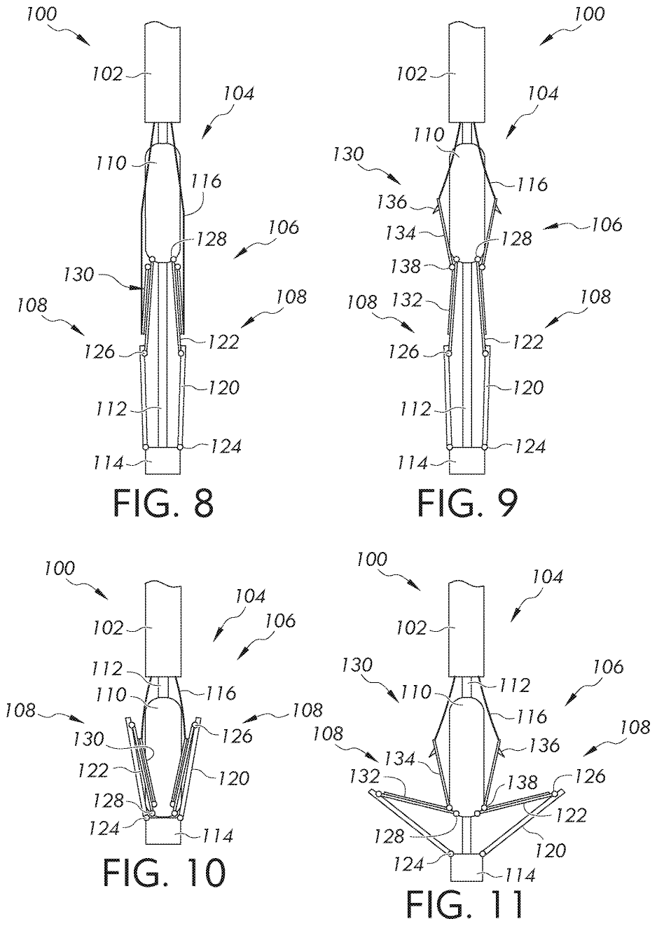

Referring now to FIGS. 8-15, a schematically illustrated device 100 (e.g., a prosthetic device, a valve repair device, valve treatment device, implantable device, implant, etc.) is shown in various stages of deployment. The device 100 and other similar devices and/or implants are described in more detail in PCT patent application publication Nos. WO2018/195215, WO2020/076898, WO2019/139904, WO2023278663, WO2023/004098, WO2023/091520, WO2023/107296, WO2023/086340, WO2023/003755, and WO2022/231889, which are incorporated herein by reference in their entirety for all purposes. The devices herein can include any other features for another device or implant discussed in the present application or the applications cited above, and the devices herein can be positioned to engage valve tissue (e.g., leaflets 20, 22, 30, 32, 34) as part of any suitable treatment and/or repair system (e.g., any treatment and/or repair system disclosed in the present application, or the applications cited herein).

The device 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device 100 includes a coaptation portion 104 and an anchor portion 106.

In some implementations, the coaptation portion 104 of the device 100 includes a coaptation element 110 that is adapted to be deployed and/or implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.).

The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets.

In some implementations, the actuation lines 116 can be actuated separately (or both separately and simultaneously) so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

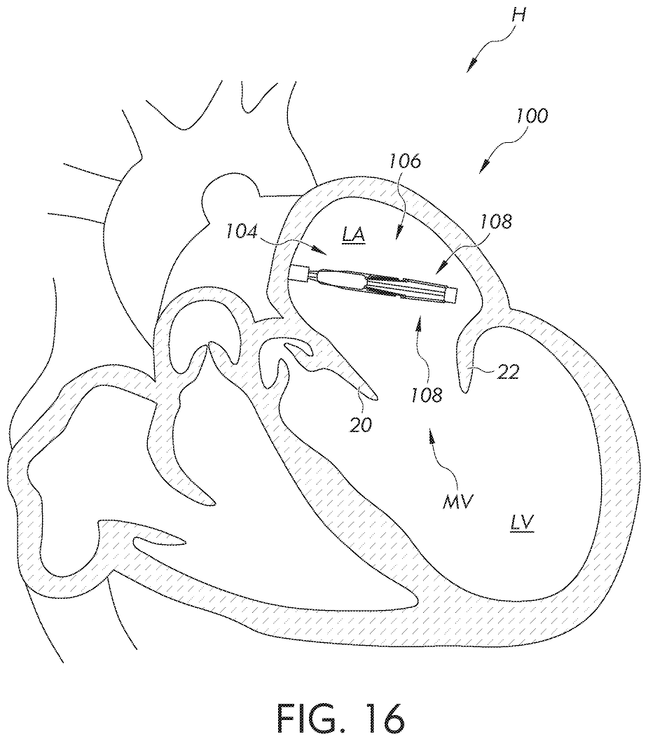

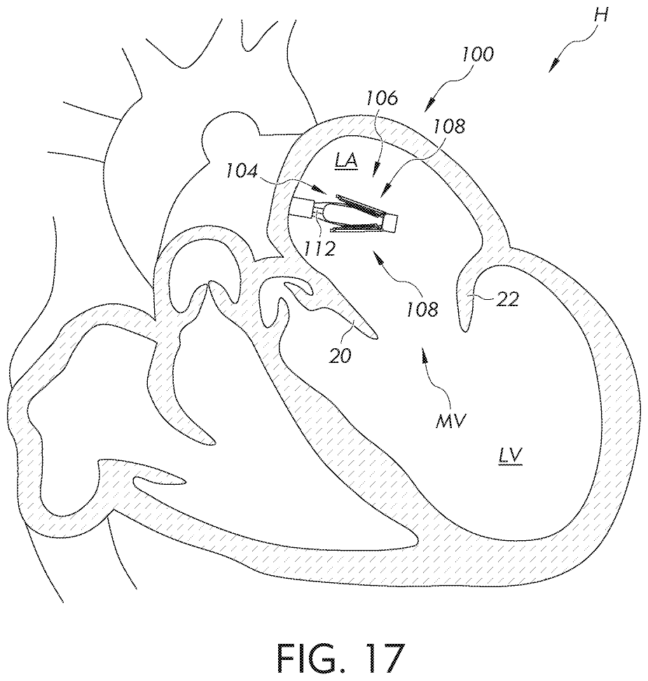

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

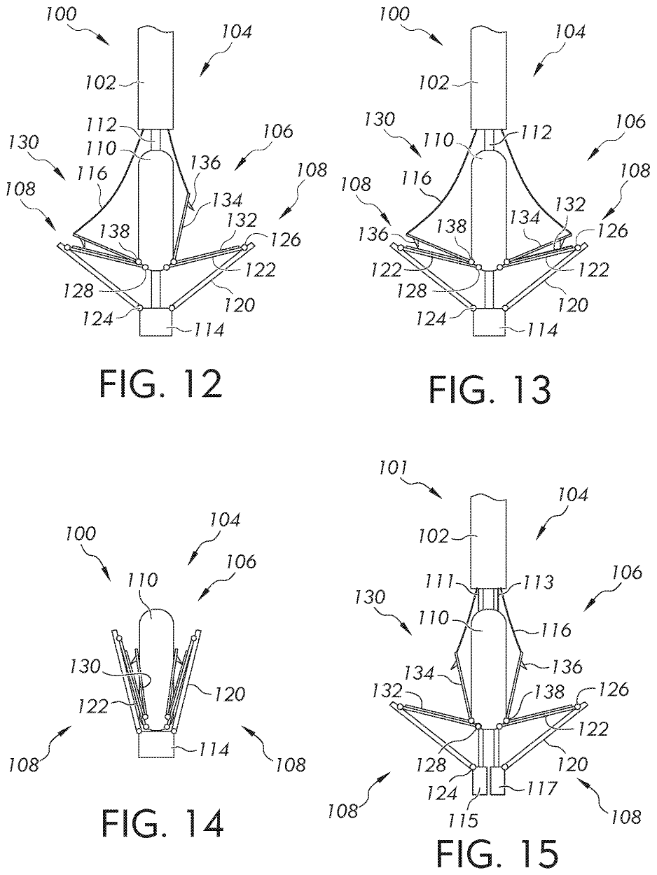

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder- and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

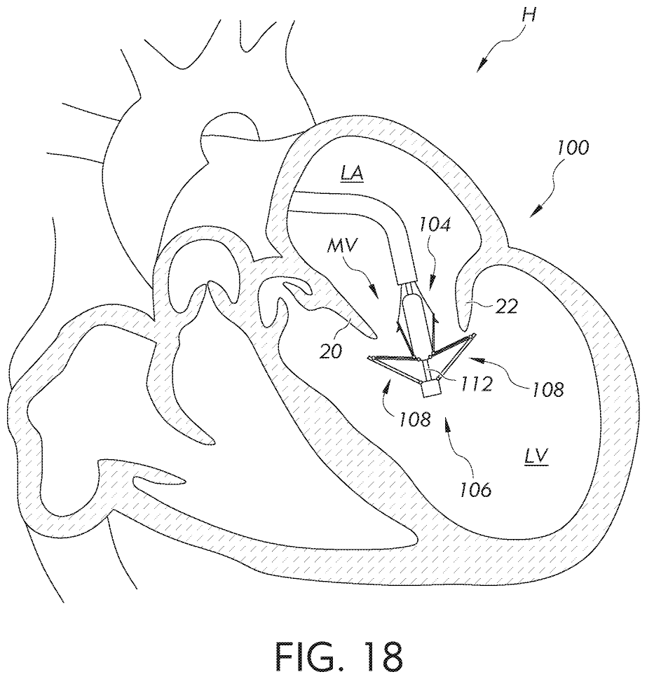

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the implant/device can be advanced from inside the steerable catheter to position the implant as illustrated by FIG. 18.

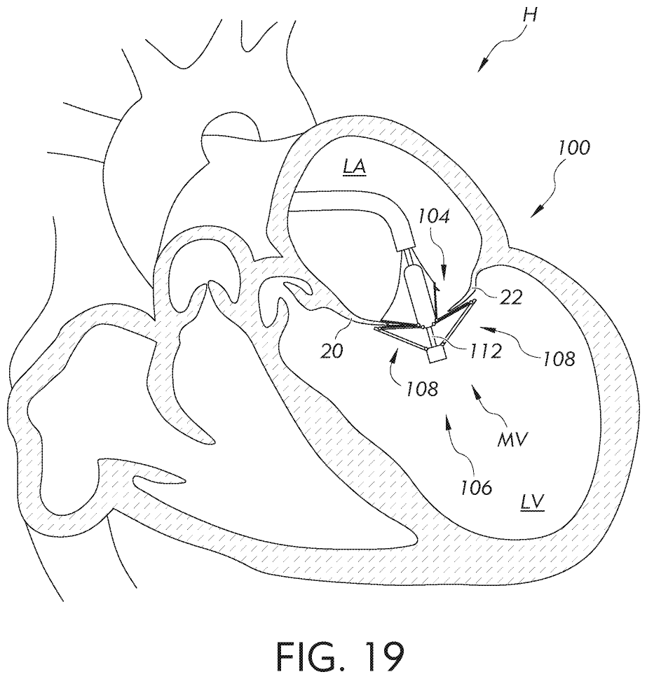

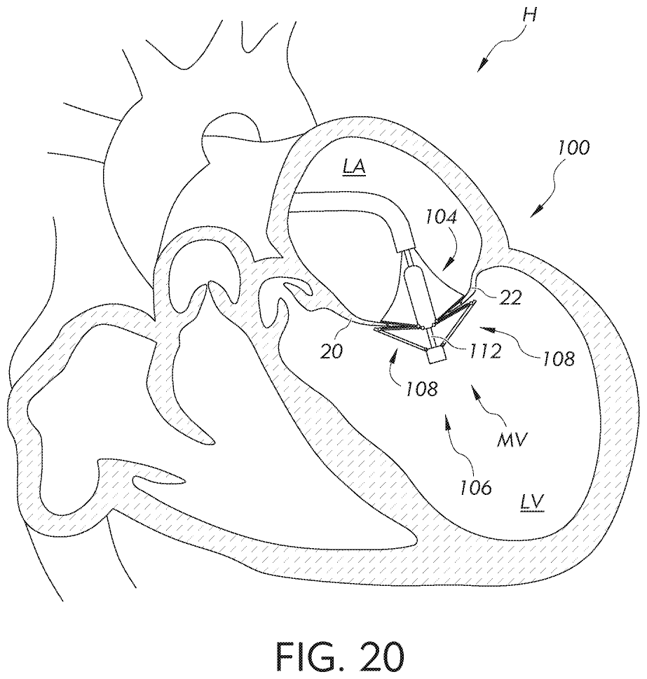

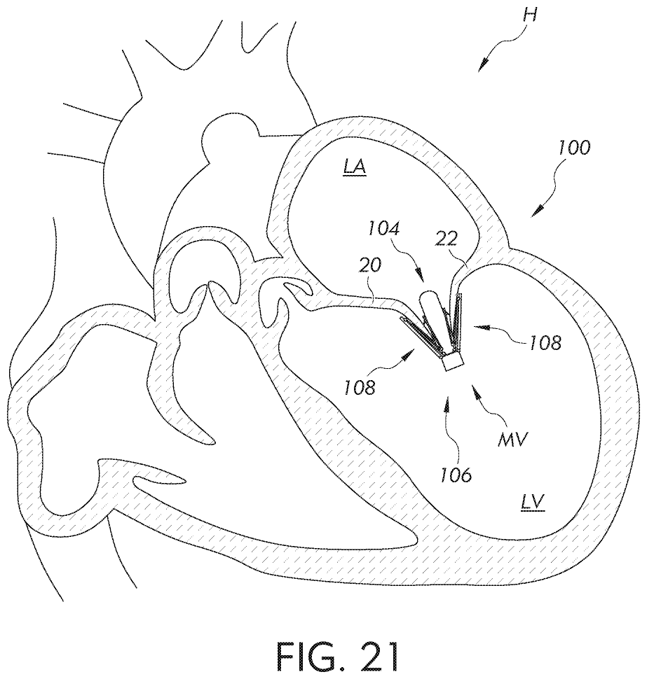

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device 100 is fully closed and deployed in the native mitral valve MV.

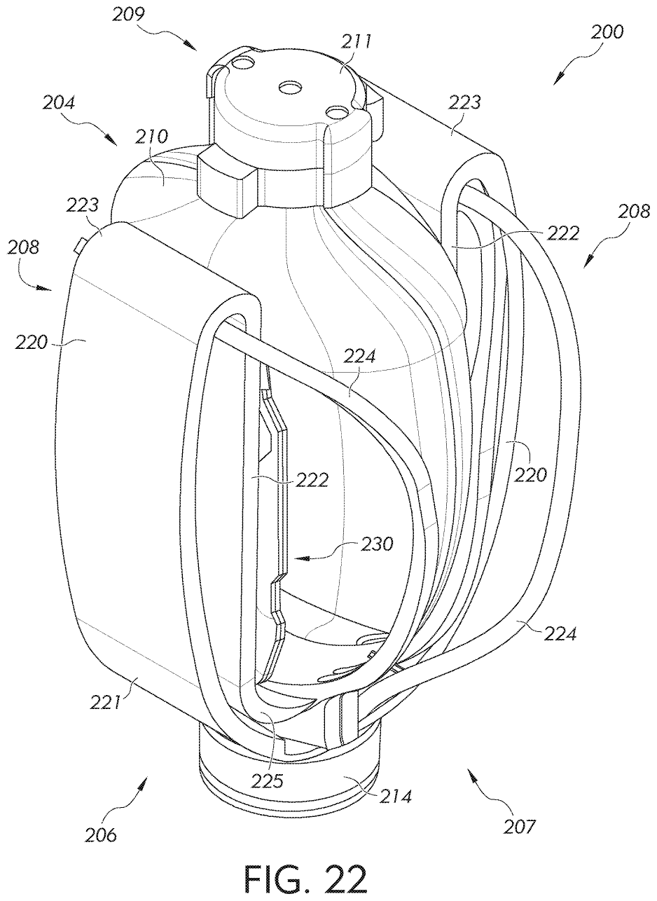

Any of the features disclosed by the present application can be used in a wide variety of different treatment devices and/or repair devices. FIGS. 22-24 illustrate examples of valve treatment and/or repair devices that can be modified to include any of the features disclosed by the present application. Any combination or sub-combination of the features disclosed by the present application can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 can be configured as an implantable device or implant or other valve treatment device (e.g., one that does not necessarily remain implanted). The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable treatment and/or repair system (e.g., any treatment and/or repair system disclosed in the present application, or the applications cited herein). The device/implant 200 can be a prosthetic spacer device, valve repair device, treatment device, or another type of implant that attaches to leaflets of a native valve.

In some implementations, the device 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaptation element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and the other applications incorporated herein. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or the other applications incorporated herein. Patent Cooperation Treaty International Application No. PCT/US2018/028189 (International Publication No. WO 2018/195215) is incorporated herein by reference in its entirety.

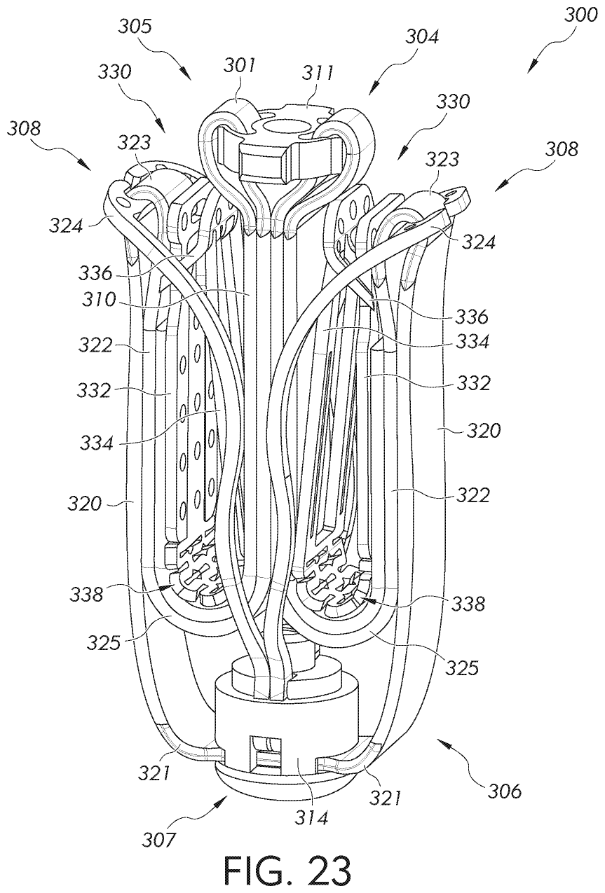

Referring now to FIG. 23, an example of a device 300 (e.g., a valve repair device, a valve treatment device, an implantable device, an implant, etc.) is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable treatment and/or repair system (e.g., any treatment and/or repair system disclosed in the present application, or the applications cited herein).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device 300 is similar in configuration and operation to the device 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and/or any other applications incorporated herein. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or any other applications incorporated herein. Patent Cooperation Treaty International Application No. PCT/US2019/055320 (International Publication No. WO 2020/076898) is incorporated herein by reference in its entirety.

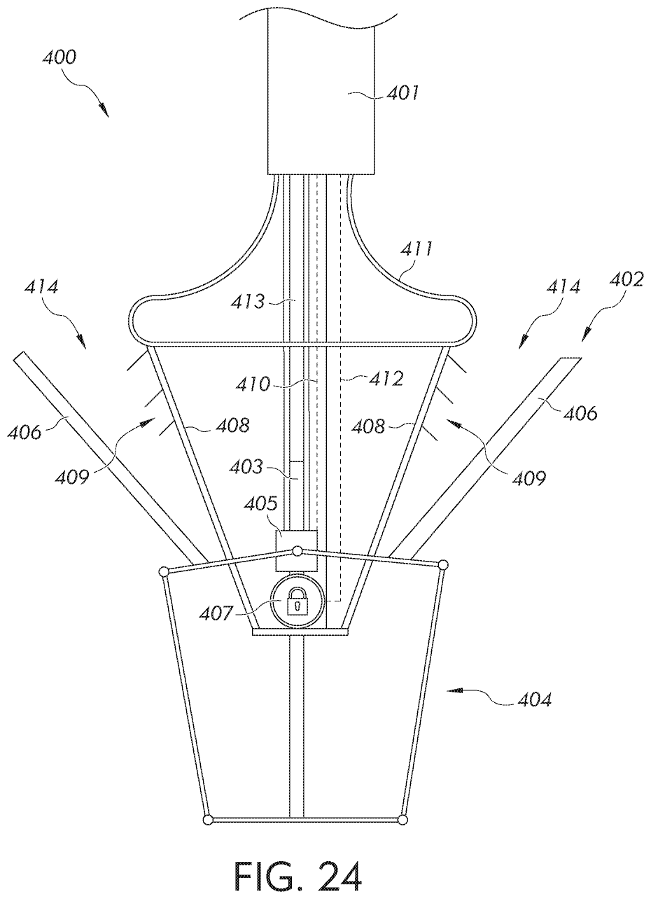

FIG. 24 illustrates an example of one of the many treatment and/or repair systems 400 for treating and/or repairing a native valve of a patient that the concepts of the present application can be applied to. The treatment and/or repair system 400 includes a delivery device 401 and a treatment and/or repair device 402.

In some implementations, the treatment device or repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the device 402. In some implementations, after the device 402 is secured to valve tissue, the placement shaft 413 can be removed from the shaft 403 to remove the device 402 from the remainder of the treatment and/or repair system 400, such that the device 402 can remain attached to the valve tissue, and the delivery device 401 can be removed from a patient's body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

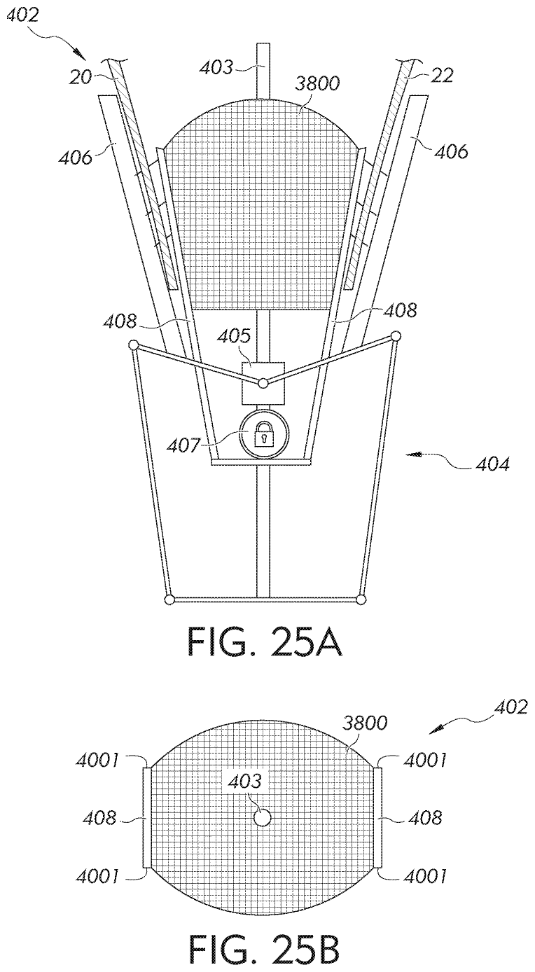

Referring to FIGS. 25A-25B, an example of a treatment and/or repair device 402 has a coaptation element 3800. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a device, treatment device, repair device, implant, etc. discussed in the present application, and the device 402 can be positioned to engage valve tissue as part of any suitable treatment and/or repair system (e.g., any treatment and/or repair system disclosed in the present application, or the applications cited herein). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

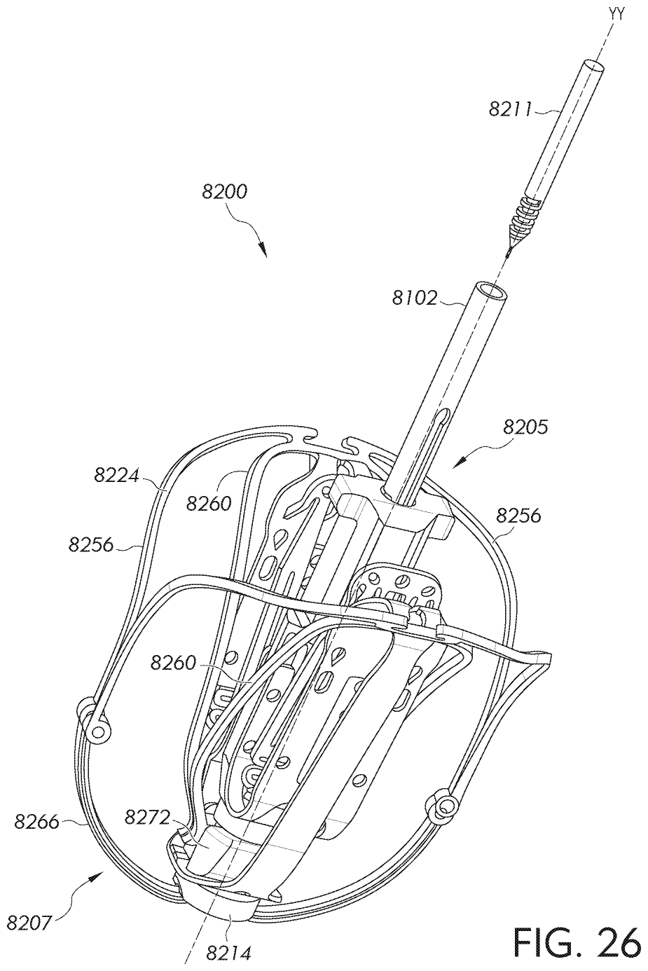

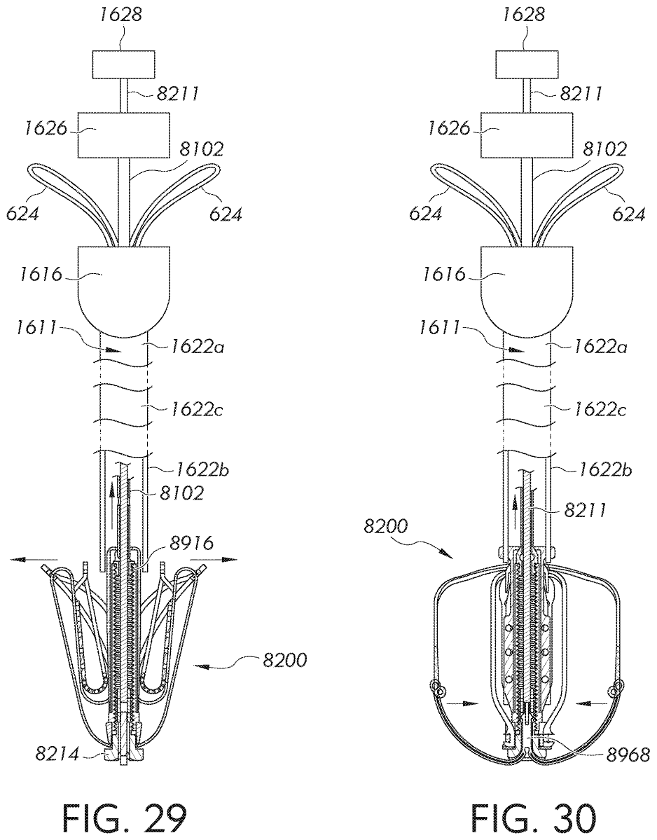

FIGS. 26-30 illustrate an example of one of the many systems for treating and/or repairing a native valve of a patient that the concepts of the present application can be applied to. Referring to FIGS. 29 and 30, the system includes a catheter assembly 1611 (e.g., a device catheter assembly, an implant catheter assembly, treatment catheter assembly, etc.) and a treatment and/or repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded configuration to the narrowed configuration by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

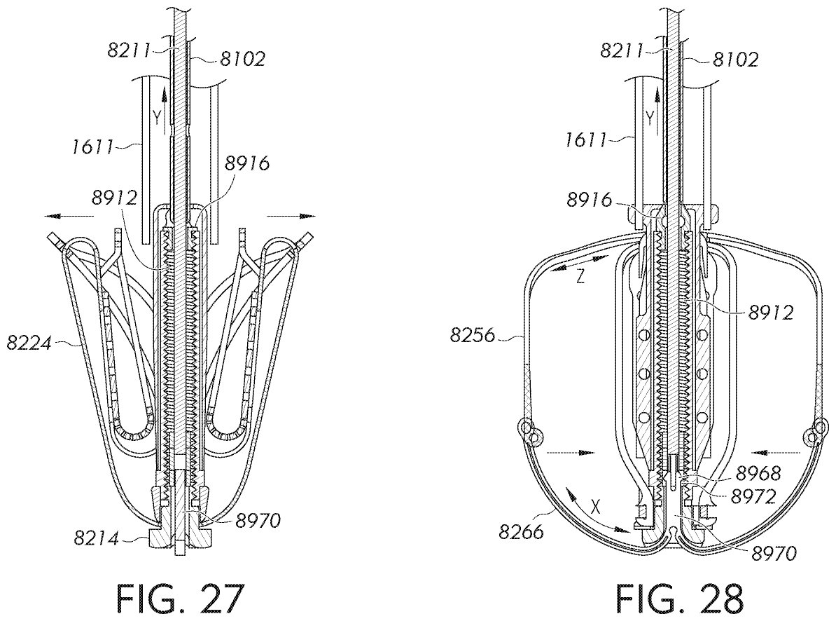

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed configuration and an expanded configuration. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed configuration and the expanded configuration can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some of the examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed configuration can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded configuration provides the anchor portion of the device 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

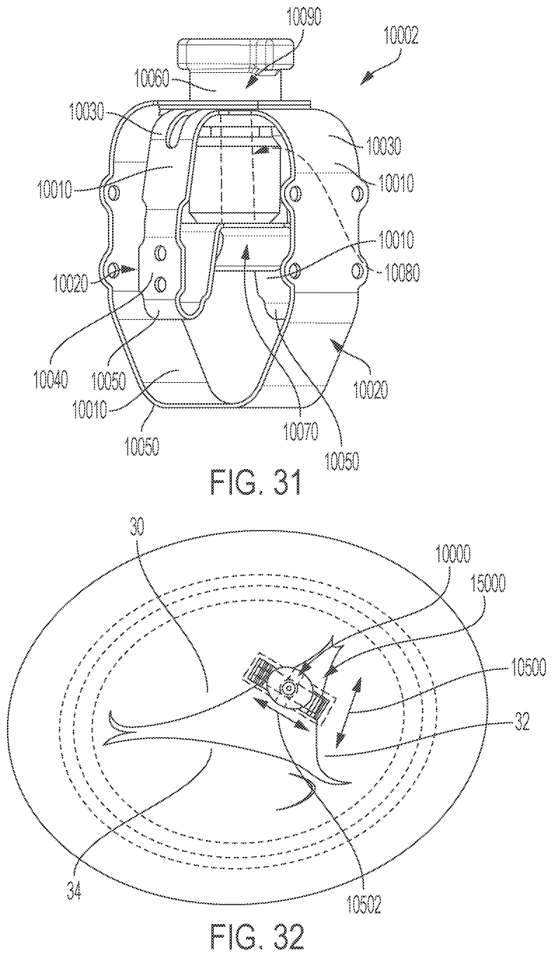

FIGS. 31-39 illustrate an example of a device 15000 (e.g., a treatment device, repair device, valve treatment device, valve repair device, implantable device, implant, etc.) having an expandable coaptation element 10000. The expandable coaptation element 10000 can be used in a variety of different treatment and/or repair devices, including but not limited to, any of the treatment and/or repair devices disclosed in the present application or in the references cited herein. For example, any expandable coaptation element disclosed herein can be used in the device that is schematically illustrated in FIGS. 8-14.

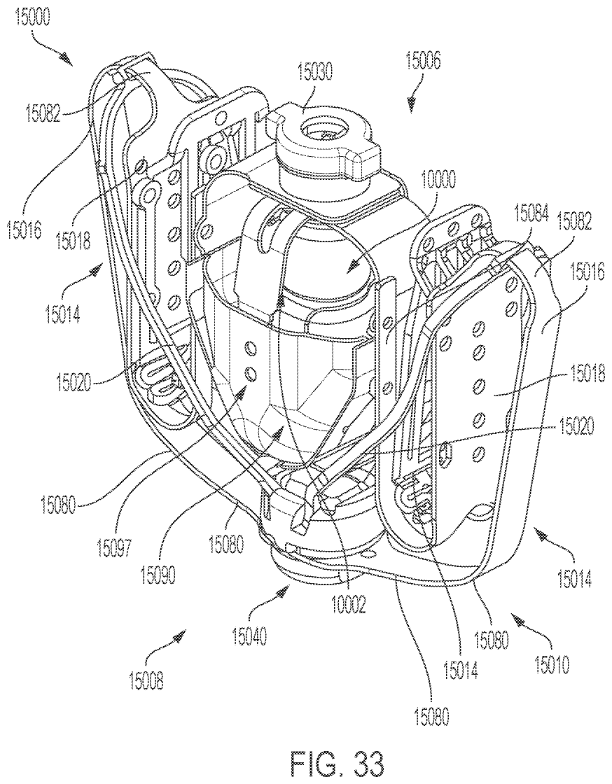

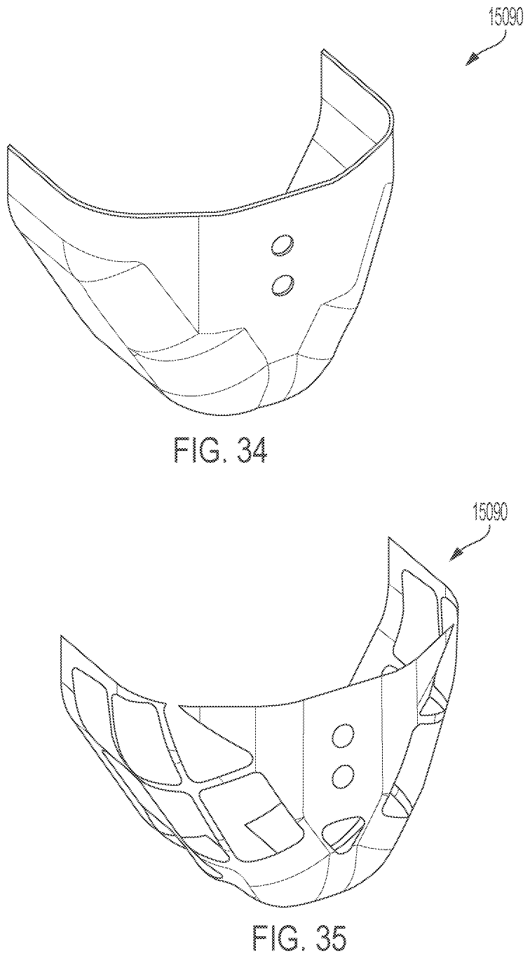

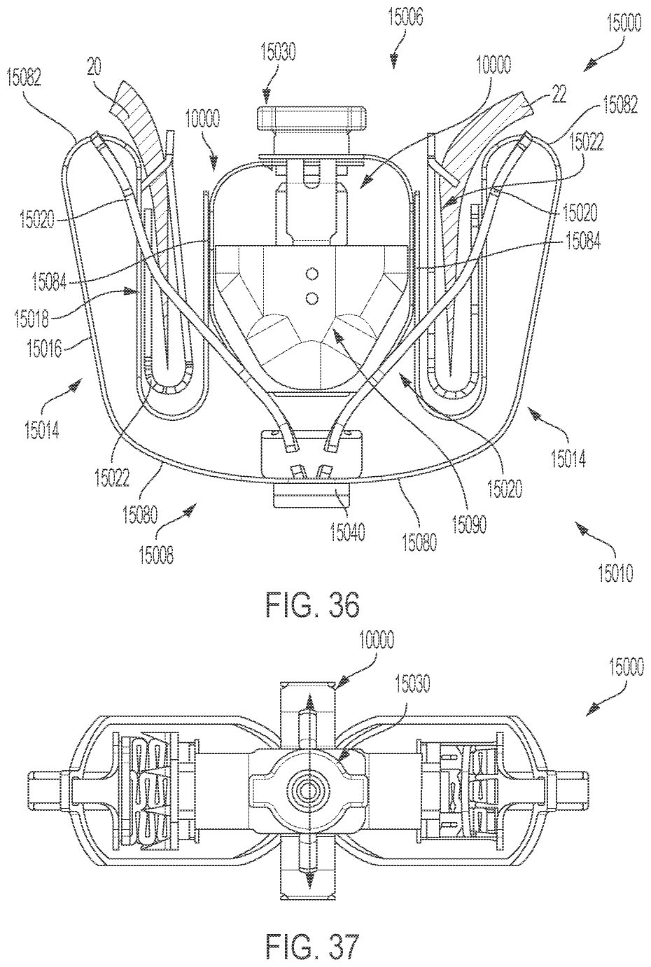

The implantable device 15000 (See FIG. 33) is one of the many different configurations that the device that is schematically illustrated in FIGS. 8-14 with an expandable coaptation element 10000 can take. The device 15000 can include any other features for an implantable device or implant discussed in the present application, and the expandable coaptation element 10000 can be positioned to engage leaflets 30, 32, 34 (see FIGS. 7 and 34) or leaflets 20, 22 (see FIGS. 6 and 36) as part of any suitable treatment and/or repair device 15000 (e.g., any treatment and/or repair device disclosed in the present application or in the references cited herein). The device 15000 can be deployed from a delivery sheath, an implant catheter, and/or an implant catheter. The device 15000 can include an expandable mechanism 10002 and an anchor portion having two or more anchors, such as the anchor portion and anchors described herein. The device 15000 can be a prosthetic spacer device, valve repair device, or another type of implant that attaches to leaflets of a native valve.

In some implementations, as shown in FIG. 31, an example expandable mechanism 10002 can be formed from one or more struts 10010. In some implementations, the struts 10010 have a plurality of rigid portions 10020 that are connected together at flexible proximal connection regions 10030 and flexible distal connection regions 10050. In some implementations, the flexible proximal connection regions 10030 and flexible distal connection regions 10050 enable the rigid portions 10020 to move relative to one another.

In some implementations, the connection regions 10030, 10050 can be solid pieces of the same material that makes up the rigid portions 10020. In some implementations, the flexible connection regions 10030, 10050 can be connected via interconnecting couplers or other means for connection.

The expandable mechanism 10002 can be used in a variety of devices, implants, valve repair devices, e.g., device 15000 illustrated in FIGS. 33-39 and/or other devices disclosed herein.

The rigid portions 10020 can include an attachment region 10040 for attaching another component of the expandable coaptation element 10000. For example, shell components 15090 and/or a sleeve 17000, which are described below can be attached to the attachment region 10040. The shell components 15090 and/or a sleeve 17000 can provide the expandable coaptation element 10000 with a substantially continuous and/or smooth outer surface.

Some or all of the struts 10010 are movable between a collapsed condition and an expanded condition. The struts can be moved between the collapsed condition and the expanded condition in a variety of different ways. In the example illustrated by FIG. 31, one pair of the struts 10010 are moved between the collapsed condition and an expanded condition via a screw 10080 and a nut 10090. Adjusting the nut 10090 extends or draws the screw 10080, which will expand and/or contract the struts 10010.

In some implementations, the screw 10080 extends through a shaft 10060 and extends to an actuation member 10070. In the example illustrated by FIG. 31, advancing the screw through the nut moves the actuation member 10070 distally. In this example, the distal movement of the actuation member 10070 forces the attachment region 10040 outward to increase the width of the expandable mechanism 10002. Conversely, retracting the screw through the nut moves the actuation member 10070 proximally. The proximal movement of the actuation member 10070 moves the attachment regions 10040 inward to decrease the width of the expandable mechanism 10002.

In the example illustrated by FIG. 31, The expandable mechanism 10002 expands in one direction, since only one pair of struts 10010 are coupled to the actuation member 10070. However, in some implementations, both pairs or different arrangements of struts can be coupled to one or more actuation members such that the expandable mechanism can expand in more than one direction.

In some implementations, adjusting the nut 10090 and screw 10080 relative to one another can expand and contract two pairs of struts 10010 of the expandable mechanism 10002 in the same direction (i.e., both expand or both contract). In some implementations, adjusting the nut 10090 relative to the screw 10080 can expand and contract the two pairs of struts 10010 of the expandable mechanism 10002 in opposite directions (i.e., one pair expands while the other contracts). The widths of both pairs of struts 10010 can be adjusted by a single adjustment mechanism (e.g., actuation member 10070, screw 10080, and nut 10090) or two independent adjustment mechanisms can be used to independently adjust each of the pairs of struts. Control can be achieved individually, simultaneously, sequentially, or otherwise.

The rigid portions 10020 and the flexible portions 10050 of the struts 10010 can include an optional cover over the struts 10010. The struts 10010 can include shaped portions and/or can include flexible or pliable portions to allow the shape of the expandable mechanism 10002 to conform to the shape of the native leaflets 20, 22 when the leaflets 20, 22 close against the expandable coaptation element 10000. This can improve engagement between the expandable coaptation element 10000 and the native leaflets 20, 22. While four struts 10010 are shown, any number of struts 10010 can be combined to provide expanding and contracting coaptation surfaces. Different struts can include rigid portions 10020 having different shapes and lengths to provide differing rates of expansion and contraction and different maximum expansion positions to form a wide variety of differently shaped expandable coaptation elements 10000.

FIG. 32 shows an expandable coaptation element 10000 of a device 15000 (e.g., a treatment device, a repair device, an implantable device, an implant, etc.) implemented between leaflets of a native valve, such as the illustrated tricuspid valve or a native mitral valve. Once the device is deployed as described above, the size of the expandable coaptation element 10000 can be adjusted in the direction 10500 and/or the direction 10502. The size of the expandable coaptation element 10000 can be adjusted based on regurgitation through the native valve during ventricular systole and/or flow through the valve during ventricular diastole. For example, the size of the expandable coaptation element 10000 can be adjusted to provide the largest reduction of regurgitation that allows the native valve to provide an acceptable flow from the atrium to the ventricle. In some implementations, the direction 10500 is a gap width of a septal-lateral annulus and the direction 10502 is along a gap length of an anterior-posterior annulus.

FIGS. 33-39 illustrate an example of a device 15000 (e.g., valve repair device, valve treatment device, implantable device, implant, etc.) having an expandable coaptation element 10000. The expandable coaptation element 10000 can be used in a variety of different devices, including but not limited to, any of the treatment and/or repair devices disclosed in the present application. For example, any expandable coaptation element disclosed herein can be used in the device that is schematically illustrated in FIGS. 8-14.

The device 15000 (See FIG. 33) is one of the many different configurations that the device that is schematically illustrated in FIGS. 8-14 with an expandable coaptation element 10000 can take. The device 15000 can include any other features for a device discussed in the present application, and the expandable coaptation element 10000 can be positioned to engage leaflets 30, 32, 34 (see FIGS. 7 and 34) or leaflets 20, 22 (see FIGS. 6 and 36) as part of any suitable device (e.g., any treatment and/or repair device disclosed in the present application).

The device 15000 can be deployed from a delivery sheath, a device catheter, and/or an implant catheter. The device 15000 can include an expandable mechanism 10002 (the expandable mechanism can be the same as or similar to any of the expandable mechanisms described anywhere in this disclosure) and an anchor portion having one or more anchors (which can be the same as or similar to any anchor portion and/or anchors described anywhere in this disclosure). The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

The device 15000 can be a prosthetic spacer device, valve repair device, valve treatment device, implant, or another type of device that attaches to leaflets of a native valve.

Referring now to FIG. 33, a device 15000 (e.g., a valve repair device, an implantable device, implant, valve treatment device, etc.) with an expandable coaptation element 10000 is shown. The implantable device 15000 is one of many different configurations that the device 100 that is schematically illustrated in FIGS. 8-14 with an expandable coaptation element added can take.

In some implementations, the device 15000 includes an expandable coaptation element 10000, a proximal or attachment portion 15006, an anchor portion 15008, and a distal portion 15010. In some implementations, the expandable coaptation element 10000 is configured for adjustable implantation between leaflets of a native valve. In some implementations, the anchor portion 15008 includes a plurality of anchors 15014. The anchors can be configured in a variety of ways. In some implementations, each of the anchors 15014 includes outer paddles 15016, inner paddles 15018, paddle extension members or paddle frames 15020, and clasps 15022. In some implementations, the attachment portion 15006 includes a first or proximal collar 15030 (or other attachment element) for engaging with a capture mechanism of a delivery system. A delivery system for the device or implant 15000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the expandable coaptation element 10000 and/or the outer and inner paddles 15016, 15018 are formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the device 15000 (see actuation element 112 in FIGS. 8-14). In some implementations, the actuation element extends through the proximal collar 15030 and expandable coaptation element 10000 to engage a cap 15040 of the distal portion 15010. The actuation element can be configured to removably engage the cap 15040 with a threaded connection, or the like, so that the actuation element can be disengaged and removed from the implant 15000 after implantation.

The expandable coaptation element 10000 extends from the proximal collar 15030 (or other attachment) to the cap 15040. In some implementations, the expandable coaptation element 10000 has a generally elongated and round shape, though other shapes and configurations are possible. In some implementations, the expandable coaptation element 10000 has an elliptical shape or cross-section when viewed from above and has a tapered shape or cross-section when seen from a front view and a rounded shape or cross-section when seen from a side view. A blend of these three geometries can result in the three-dimensional shape of the illustrated expandable coaptation element 10000.

The size and/or shape of the expandable coaptation element 10000 can be selected and/or be adjusted to minimize the number of implants that a single patient will require (preferably one), while at the same time maintaining low transvalvular gradients. In some implementations, the anterior-posterior distance at the top of the spacer or coaptation element is about 5 mm, and the medial-lateral distance of the spacer or coaptation element at its widest is about 10 mm. In some implementations, the overall geometry of the device 15000 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 15016 are jointably attached to the cap 15040 of the distal portion 15010 by connection portions 15080 and to the inner paddles 15018 by transition portions 15082. The inner paddles 15018 are jointably attached to the spacer or coaptation element by extensions 15084. In this manner, the anchors 15014 are configured similar to legs in that the inner paddles 15018 are like upper portions of the legs, the outer paddles 15016 are like lower portions of the legs, and the transition portions 15082 are like knee portions of the legs. As described above, the expandable coaptation element 10000 includes a nut 10090 and screw 10080 (see FIG. 31). Adjusting the nut 10090 adjusts the screw 10080, causing the expandable coaptation element 10000 to expand or contract.

In some implementations, the paddle frames 15020 are attached to the cap 15040 at the distal portion 15010 and extend to the transition portions 15082 between the inner and outer paddles 15018, 15016. In some implementations, the paddle frames 15020 are formed of a material that is more rigid and stiff than the material forming the paddles 15018, 15016 so that the paddle frames 15020 provide support for the paddles 15018, 15016.

The paddle frames 15020 can provide additional pinching force between the inner paddles 15018 and the expandable coaptation element 10000 and assist in wrapping the leaflets around the sides of the expandable coaptation element 10000. That is, the paddle frames 15020 can be configured with a round three-dimensional shape extending from the cap 15040 to the transition portions 15082 of the anchors 15014. The connections between the paddle frames 15020, the outer and inner paddles 15016, 15018, the cap 15040, and the expandable coaptation element 10000 can constrain each of these parts to the movements and positions of the treatment and/or repair devices described herein.

The wider configuration of the paddle frames 15020 can provide increased surface area compared to the inner paddles 15018 alone. The increased surface area can distribute the clamping force of the outer paddles 15016 and paddle frames 15020 against the native leaflets over a relatively larger surface of the native leaflets in order to further protect the native leaflet tissue.

In some implementations, the expandable coaptation element 10000 can include an expandable shell 15090. The shell 15090 can be configured as a solid surface, a lattice (e.g., a honeycomb or other pattern that leaves solid portions and cut out portions), a braided or woven material, or otherwise configured. The shell 15090 can be comprised of a single piece, two pieces, or a plurality of pieces. The shell 15090 can be composed of a rigid, semi-rigid, soft, or any other material suitable for implantation in the human body. One or more fasteners 15097 can be used to attach the shell 15090 to the expandable mechanism 10002.

In some implementations, a portion of the expandable mechanism 10002 that is covered by the shell can be selected such that the shell 15090 contacts the native valve leaflets and the expandable mechanism 10002 does not contact the native valve leaflets. For example, the expandable mechanism 10002 can be encapsulated by the shell 15090 or can be partially encapsulated by the shell 15090. In the illustrated example, the shell 15090 comprises two pieces that are opposite one another and move relative to one another. For example, one of the shell pieces can telescope into the other shell piece.

Referring now to FIGS. 34-35, examples of halves of the shell 15090 are shown. The shell 15090 can comprise two or more pieces and/or a flexible material, allowing the shell to conform to the overall size of the expandable mechanism 10002. Such a shell 15090 can provide a smooth contour for native valve leaflets to engage, either directly or through a cover.

A cover can be provided in a variety of different ways. For example, the components of the shell 15090 can be laminated with a polymer or covered with a fabric in order to improve the interaction with the leaflets and/or to seal the expandable mechanism 10002 from liquid. FIG. 34 illustrates a shell 15090 with a substantially solid surface. FIG. 35 illustrates a shell 15090 with cutouts that form a semisolid surface. In some implementations, the shell 15090 can be replaced by another structure, such as a balloon, balloon material, or other flexible material.

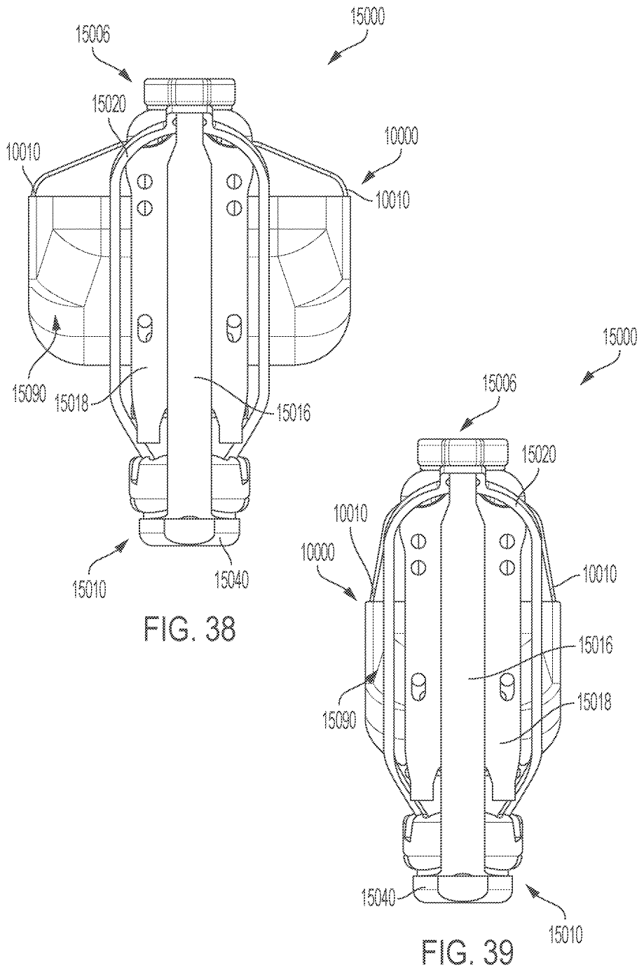

Referring now to FIG. 36, a front perspective view of the device 15000 described in FIG. 33 attached to native valve leaflets 20, 22 is shown. The expandable coaptation element 10000 is positioned between the leaflets 20, 22. A top plan view of the of the device 15000 is shown in FIG. 37 and side views of the device 15000 with the expandable coaptation element 10000 in an expanded configuration and a contracted configuration are shown in FIGS. 38 and 39 respectively. Adjusting the expandable mechanism 10002, for example by rotating the screw 10080, expands or contracts the shell 15090 to adjust flow through the native valve (reduce regurgitation through the native valve by increasing the size of the shell or increasing the flow through the native valve by reducing the size of the shell).

Referring to FIG. 38, the struts 10010 of the expandable mechanism 10002 are spread apart to move the halves of the shell 15090. As such, the shell 15090 has a wide configuration and takes up a larger space. Referring to FIG. 39, the struts 10010 of the expandable mechanism 10002 are close together to move or telescope the halves of the shell 15090 together. As such, the shell 15090 has a narrow configuration and takes up a smaller space. FIGS. 38 and 39 show a shell 15090 having two halves that are moved toward and away from one another by two struts 10010. However, in some implementations, the expandable coaptation element 10000 can have one, two, three, four or any number of struts that can be moved toward and away from a central axis of the expandable coaptation element 10000 and one, two, three, four, or any number of shell components that can be moved toward and away from the central axis of the expandable coaptation element 10000. The number of shell components can be the same or different than the number of moving struts.

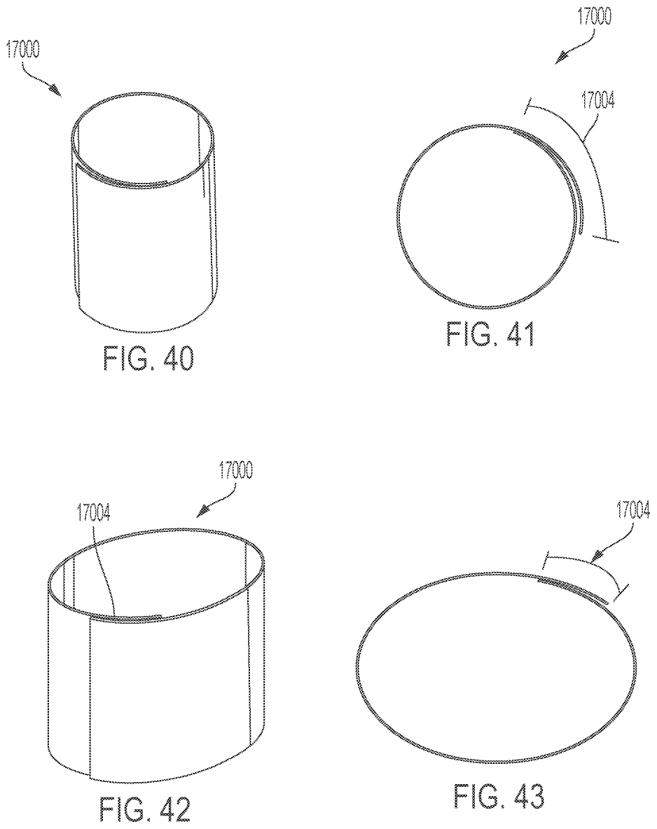

The expandable coaptation element 10000 can take a wide variety of different forms. For example, FIGS. 40-41 show an expandable sleeve 17000 that can be used instead of the shell 15090. The sleeve 17000 can be used with a wide variety of expandable mechanisms, such as the expandable mechanism 10002 described above or any other expandable mechanism described in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

FIGS. 40-41 show the expandable sleeve 17000 in various forms. The expandable sleeve 17000 can have a variety of different shapes. For example, the expandable sleeve can be cylindrical, conical, frustoconical, egg shaped, pyramid, a portion of a pyramid, diamond, combinations of these shapes, etc. Referring to FIGS. 40 and 41, in some implementations, the expandable sleeve 17000 has a circular shape with an overlap 17004. In one example, FIGS. 40 and 41 illustrate the sleeve 17000 in a contracted configuration.

FIGS. 42 and 43 illustrate the sleeve 17000 in an expanded configuration. For example, the expandable mechanism 10002 can be expanded to move the sleeve 17000 from the configuration shown in FIGS. 40 and 41 to the configuration shown in FIGS. 42 and 43. In FIGS. 42 and 43, the sleeve 17000 has the shape of an elliptical cylinder. FIGS. 42 and 43 illustrate only one of the many configurations that the expandable sleeve 17000 can take when expanded. The sleeve 17000 can be configured to expand to a wide variety of different shapes. For example, the sleeve can be configured to expand to a larger circular cylinder shape, a non-cylindrical shape, such as a shape that tapers from one end to the other end. As is illustrated by FIG. 42 the overlap 17004 decreases as the sleeve expands.

The sleeve 17000 can be made from a wide variety of different materials. For example, the expandable sleeve 17000 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

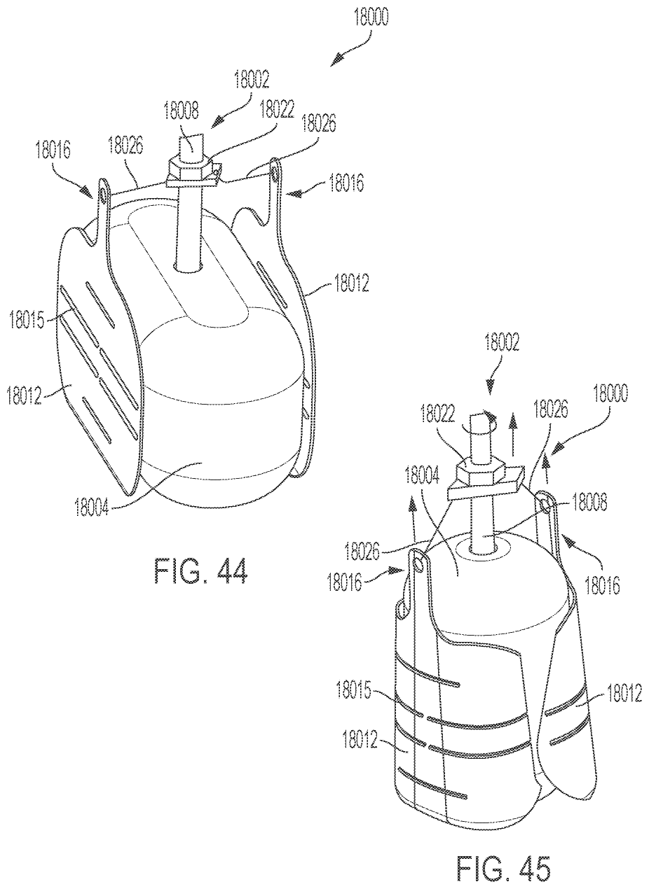

FIGS. 44-45 illustrate an example of an expandable coaptation element 18000. In the example illustrated by FIGS. 44 and 45, the expandable coaptation element 18000 can comprise one or more shape changing components 18012, an adjustment mechanism, and an optional fill material or core 18004. The shape changing component 18012 is configured to change size and/or shape when a force, such as a tensile force, is applied to the shape changing component. For example, the shape changing component 18012 can change from the flat or generally flat configuration shown in FIG. 44 to the curved configuration shown in FIG. 45 by applying a tensile force to the shape changing component 18012 with the adjustment mechanism 18002. In the example illustrated by FIGS. 44 and 45, the optional fill material or core 18004 is positioned between to shape changing components 18012. The optional fill material or core 18004 is compressed by the shape changing components 18012 as the shape changing components move from the configuration illustrated by FIG. 44 to the configuration illustrated by FIG. 45. In some implementations, the optional fill or core material 18004 keeps the shape changing components 18012 spaced apart to facilitate movement from the configuration illustrated FIG. 44 to the configuration illustrated by FIG. 45.

The shape changing components 18012 can take a wide variety of different forms having a variety of different expanded and contracted shapes. In the expanded condition illustrated by FIG. 44, the shape changing components 18012 can be generally parallel plate-like structures, as illustrated. In some implementations, the shape changing components 18012 can be cylindrical, conical, frustoconical, egg shaped, pyramid, a portion of a pyramid, diamond, combinations of these shapes, etc. in the expanded configuration. In the contracted condition illustrated by FIG. 45, the shape changing components 18012 can be generally rounded to compress the optional fill material or core 18004 to a generally cylindrical configuration, as illustrated. In some implementations, the shape changing components 18012 can be conical, frustoconical, egg shaped, pyramid, a portion of a pyramid, diamond, combinations of these shapes, etc. in the contracted configuration.

In some implementations, the shape changing components 18012 are formed from flat material and have cutouts 18015. The flat material and the cutouts 18015 are configured such that applying a force, such as a tensile force to the shape changing components 18012 causes the shape changing components two flex from a substantially flat configuration to a three-dimensional configuration. For example, kirigami techniques can be applied to the flat material to make the shape changing component 18012. The shape changing components 18012 can be made from a wide variety of different materials. For example, the shape changing components 18012 can be formed from any metal or polymeric material suitable for implantation in the human.

The shape changing components 18012 can be transformed from the expanded configuration to the compressed configuration in a wide variety of different ways. In the illustrated example, the shape changing components include a tab 18016. Applying tension to the tabs 18016 causes the shape changing components 18012 to transition from the configuration illustrated by FIG. 44 to the configuration illustrated by FIG. 45. However, the tabs 18016 are optional and other arrangements for applying force to the shape changing components 18012 can be used. As mentioned above, the shape changing components 18012 can be formed using kirigami, such that applying tension to the tabs 18016 causes the shape changing components to transform from a flat configuration to a curved, three-dimensional configuration.

A variety of different mechanisms can be used to move the shape changing components 18012 from the expanded configuration to the contracted configuration. In some implementations, a screw or bolt 18008 and a nut 18022 or other fastening arrangement can be used to move the shape changing components 18012 from the expanded configuration to the contracted configuration. Rotating the nut 18022 relative to the screw or bolt 18008 can apply tension to the shape changing components 18012 to move the shape changing components from the expanded configuration (FIG. 44) to the contracted configuration (FIG. 45). For example, a line 18026 can be connected to the tab 18016 and be coupled to the nut 18022. Rotating the nut 18022 pulls the line 18026 to move the shape changing components 18012 from the expanded configuration to the contracted configuration. In some implementations, tension on the tabs 18016 causes the shape changing components 18012 to transition from the configuration illustrated by FIG. 44 to the configuration illustrated by FIG. 45.

The optional fill material or core 18004 can take a variety of different forms. The optional fill material or core 18004 can be composed of a soft, semi-soft, semi-hard. The optional fill material or core 18004 can be hollow or solid. The optional fill material or core 18004 can be sponge-like and porous, can be composed of a material such as mesh, woven, braided, or formed in any other suitable way or a laser cut or otherwise cut flexible material. The material can be cloth, shape-memory alloy, foam, sponge—such as Poron or any other flexible material suitable for implantation in the human body. Applying force to the shape changing components 18012 causes the shape changing components to change shape and, therefore, reshape the optional fill material or core 18004.

In the illustrated example, when the shape changing components 18012 are in the non-tensioned configuration of FIG. 44, the optional fill material or core 18004 can have an oblong shape. When the shape changing components 18012 are in the tensioned configuration of FIG. 45, the optional fill material or core 18004 can have a cylindrical or generally cylindrical configuration. However, the optional fill material or core 18004 can have a variety of different shapes in the expanded and compressed configurations. For example, the optional fill material can be cylindrical, conical, frustoconical, egg shaped, pyramid, a portion of a pyramid, diamond, wedge shaped, combinations of these shapes, etc. in the expanded can compressed configurations.

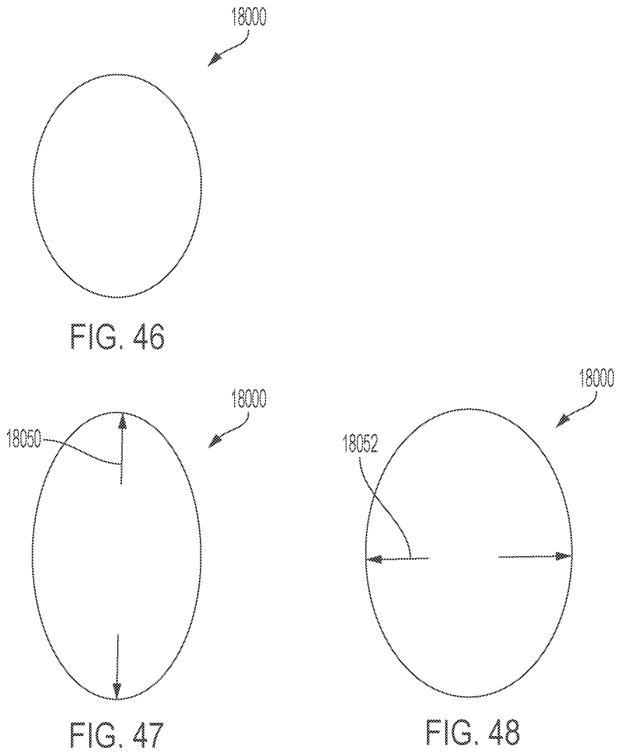

The expandable coaptation elements 18000, as well as the other expandable coaptation elements disclosed herein (10000, 17000, etc.), can be configured to expand and contract in a variety of different ways. FIGS. 46-48 are schematic top plan views of the expandable coaptation element. FIG. 46 illustrates the expandable coaptation element 18000 in a normal or starting position. The expandable coaptation element 18000 can expand and/or contract from the configuration illustrated by FIG. 46 in a variety of different ways. For example, in some implementations, the expandable coaptation element 18000 can:
Expand or contract only in the direction 18050 illustrated by FIG. 47;
Expand or contract only in the direction 18052 illustrated by FIG. 48;
Simultaneously expand and contract in the direction 18050 illustrated by FIG. 47 and the direction 18052 illustrated by FIG. 48;
Sequentially expand or contract such that the expandable coaptation element 18000 expands or contracts first in the direction 18050 illustrated by FIG. 47 and then expands or contracts in the direction 18052 illustrated by FIG. 48;
Sequentially expand or contract such that the expandable coaptation element 18000 expands or contracts first in the direction 18052 illustrated by FIG. 48 and then expands or contracts in the direction 18050 illustrated by FIG. 47;
Independently expand or contract in the direction 18050 illustrated by FIG. 47 and in the second direction 18052 illustrated by FIG. 48;
Partially expand or contract in the direction 18050 illustrated by FIG. 47 and then expand or contract in both the direction 18050 illustrated by FIG. 47 and the direction 18052 illustrated by FIG. 48; and/or
Partially expand or contract in the direction 18052 illustrated by FIG. 48 and then expand or contract in both the direction 18050 illustrated by FIG. 47 and the direction 18052 illustrated by FIG. 48.

As such, shape changing components 18012 and/or the expandable material 18004 can expand sequentially, simultaneously, or otherwise when the shape changing component 18012 is manipulated between the tensioned and non-tensioned configuration.

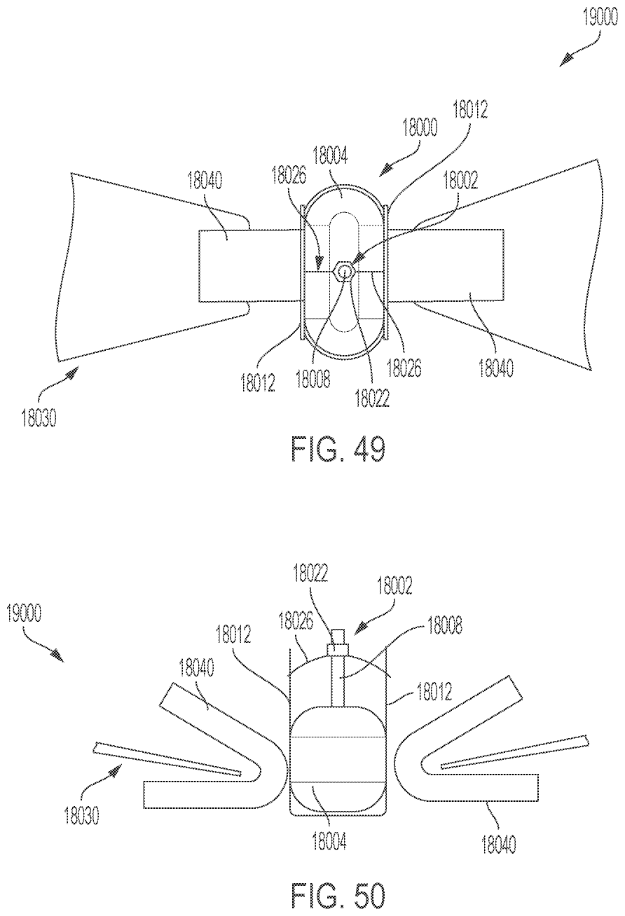

FIGS. 49-52 Illustrate an example of a device 19000 e.g., a treatment device, repair device, valve treatment device, valve repair device, implantable device, implant, etc.) that includes the expandable coaptation element 18000 illustrated by FIGS. 44 and 45. The expandable coaptation element 18000 can be used in a variety of different devices, including but not limited to, any of the treatment and/or repair devices disclosed in the present application. For example, the expandable coaptation element 18000 can be used in the device that is schematically illustrated in FIGS. 8-14. The implantable device 19000 is one of the many different configurations that the device that is schematically illustrated in FIGS. 8-14 with an expandable coaptation element 18000 can take.

The device 19000 can include any other features for a device (e.g., a treatment and/or repair device, etc.) discussed in the present application, and the expandable coaptation element 18000 can be positioned to engage leaflets 30, 32, 34 (see FIGS. 7 and 34) or leaflets 20, 22 (see FIGS. 6 and 36) as part of any suitable device 19000 (e.g., any treatment and/or repair device disclosed in the present application). The device 19000 can be deployed from a delivery sheath, a device catheter, and/or an implant catheter. The device 19000 can include an adjustment mechanism 18002 and an anchor portion having two or more anchors, such as the anchor portion and anchors described herein. In the example illustrated by FIGS. 49-52, the anchors of the device 19000 comprises clasps 18040.

In the example illustrated by FIGS. 49-52, the clasps 18040 attach the adjustable coaptation element 18000 to the leaflets of a native valve 18030 (e.g., native mitral valve or native tricuspid valve). The clasps 18040 position the adjustable coaptation element between the leaflets of the native valve 18030. In some implementations, the leaflet clasp or plurality of leaflet clasps 18040 attach to the top or atrial side and bottom or ventricular side of a leaflet of a native valve 18030.

The adjustable coaptation element is 18000 adjusted to change the shape and/or size of the area of the native valve that is blocked by the adjustable coaptation element 18000. FIGS. 49 and 50 illustrate the device 19000 attached to the native valve 18030 with the adjustable coaptation element 18000 in an expanded configuration. Relative rotation of the screw or bolt 18008 and the nut 18022 can apply tension to the shape changing components 18012. For example, a line 18026 can be connected to the tab 18016 and be coupled to the nut 18022. Rotating bolt 18008 relative to the nut 18022 pulls the line 18026 to move the shape changing components 18012 from the expanded configuration of FIG. 44 to the contracted configuration of FIG. 45.

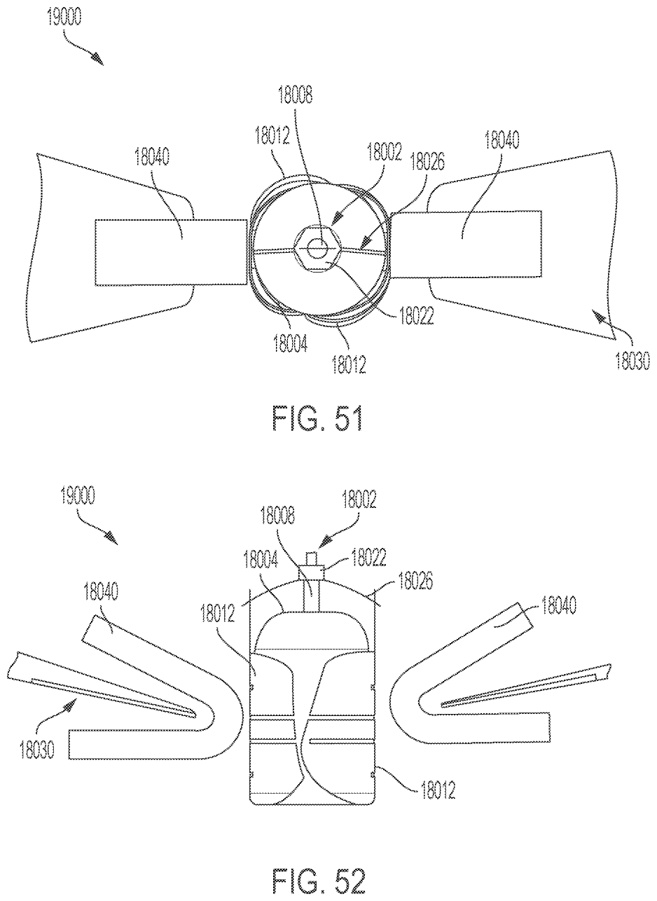

FIG. 49 shows a top plan view of the adjustable coaptation element 18000 in an expanded configuration placed between leaflets of the native valve 18030. The screw or bolt 18008 can extend into or through the spacing material 18004. FIG. 50 shows side perspective view of the expanded adjustable coaptation element 18000 between leaflets of the native valve 18030. FIG. 51 shows a top plan view of the adjustable coaptation element 18000 in a compressed configuration placed between leaflets of the native valve 18030. FIG. 52 shows side perspective view of the compressed adjustable coaptation element 18000 between leaflets of the native valve 18030.

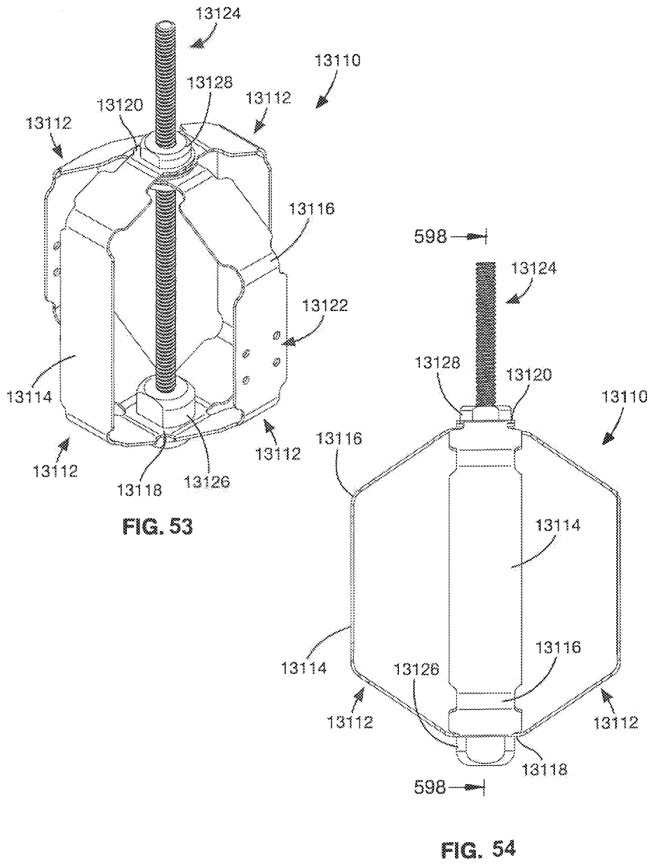

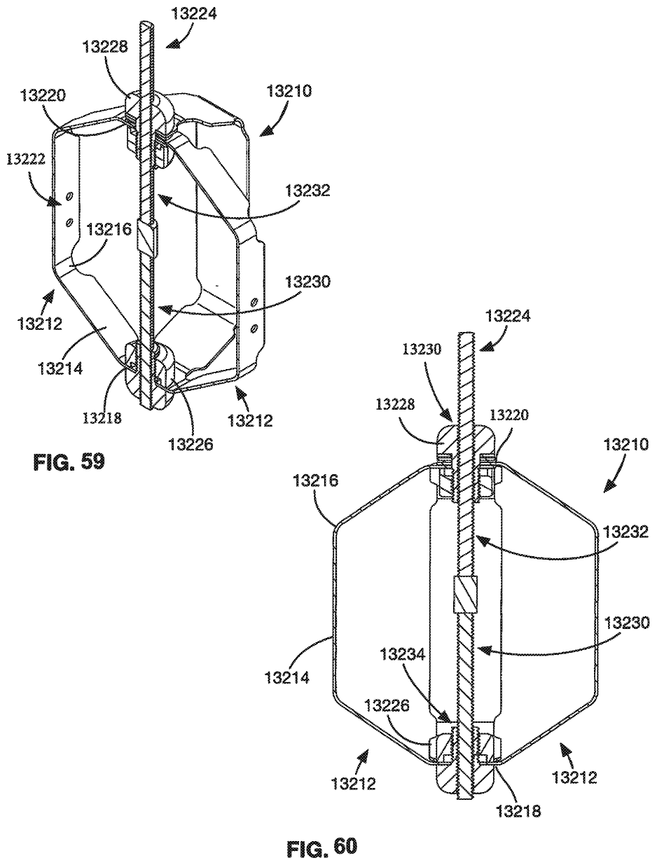

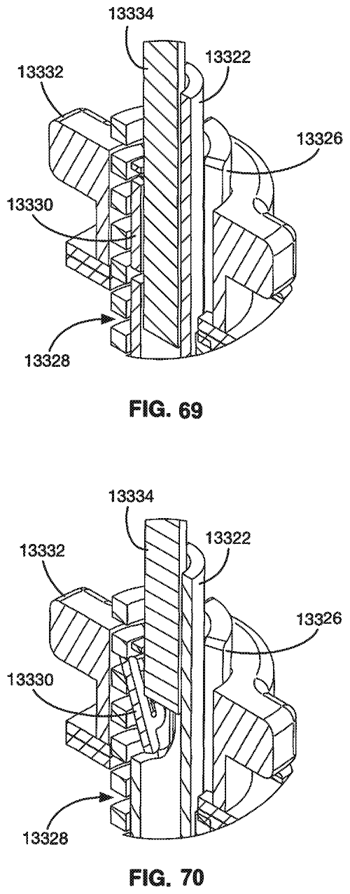

A variety of different mechanisms can be used to move the expandable or adjustable coaptation elements between expanded and contracted configurations. FIGS. 53-70 illustrate examples of expandable mechanisms that can be used with any of the devices herein. In the example illustrated by FIGS. 53-56, the expandable mechanism expands and contracts in two directions. A device (e.g., a treatment device, a repair device, an implantable device, etc.) that includes the expandable mechanism can include any other features for a device discussed in the present application, and the device can be positioned to engage valve tissue as part of any suitable treatment and/or repair system (e.g., any treatment and/or repair system disclosed in the present application, or the applications cited herein). The device can be deployed from a delivery sheath or means for delivery by a pusher, such as a rod or tube as described above.

The device can include a coaptation portion and an anchor portion having two or more anchors, such as the anchor portions and anchors described herein. The coaptation portion includes an expandable mechanism 13110 that can be actuated between a contracted condition and an expanded condition. An optional outer surface, such as the shell 15090, sleeve 17000, shape changing component 18012, etc. can be included to cover a portion or all of the expandable mechanism 13110.

The expandable mechanism 13110 can comprise and/or be formed from one or more struts 13112. The struts 13112 have a plurality of rigid portions 13114 that are connected together by hinge portions 13116 that enable the rigid portions 13114 to bend relative to each other. A fixed end portion 13118 is connected to each of the struts 13112 by the hinge portions 13116. The struts 13112 extend from the fixed end portion 13118 to a moveable end portion 13120 connected to each of the struts 13112 by one of the hinge portions 13116. One or more of the rigid portions 13114 can include an attachment region 13122 for attaching another component of the device.

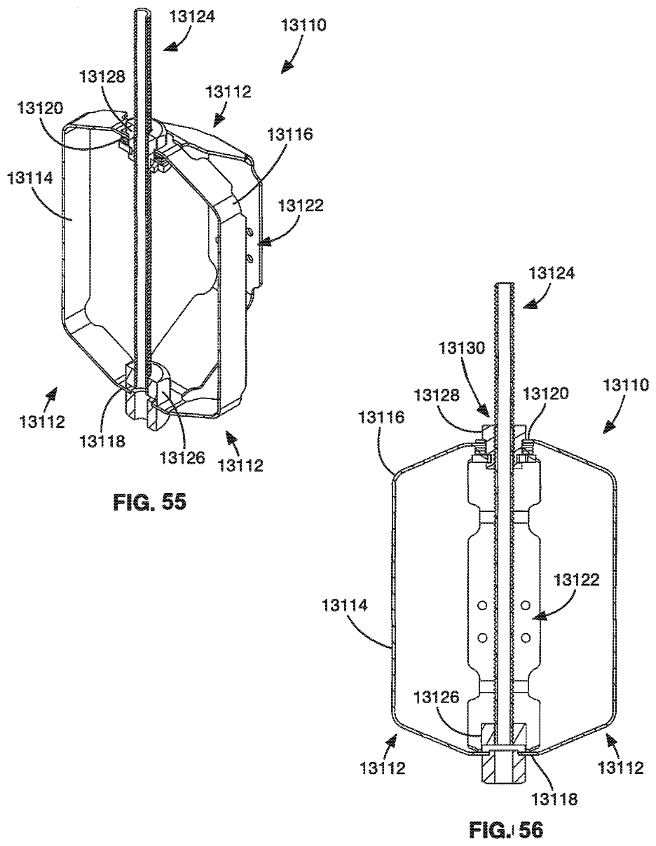

The struts 13112 are moved between a collapsed condition and an expanded condition by the actuation of a threaded actuation shaft 13124 that extends through the expandable mechanism 13110 to a fixed actuation member 13126. The fixed end portion 13118 is attached to the fixed actuation member 13126 and the moveable end portion 13120 are attached to the moveable actuation member 13128. The fixed actuation member 13126 extends proximally from the fixed actuation member 13126 and through a moveable actuation member 13128. The threaded actuation shaft 13124 can be hollow and the fixed actuation member 13126 and the moveable actuation member 13128, as shown in FIGS. 55-56, so that other actuation members and other components of the device can extend through the expandable mechanism 13110 and can be moved or actuated independently of the actuation of the expandable mechanism 13110.

The moveable actuation member 13128 includes a threaded opening 13130 that engages the threads of the threaded actuation shaft 13124 so that rotation of the threaded actuation shaft 13124 relative to the moveable actuation member 13128—or vice versa—moves the moveable actuation member 13128 toward and away from the fixed actuation member 13126. That is, rotating the moveable actuation member 13128 via an actuation shaft or other mechanism (not shown) causes the moveable actuation member 13128 to move proximally away from the fixed actuation member 13126 and distally toward the fixed actuation member 13126. As the moveable actuation member 13128 is moved along the threaded actuation shaft 13124, the struts 13112 expand and contract to change the overall width of the expandable mechanism 13110. The expandable mechanism 13110 has a minimum width when the struts 13112 are stretched to an almost nearly or straight condition and/or when the fixed end portion 13118 and the moveable end portion 13120 are moved close to each other so that the rigid portions 13114 are folded against each other. In this way, the expandable mechanism 13110 can be caused to expand and contract laterally to accommodate different size and shape gaps 26 (see FIG. 6) left between the leaflets 20, 22 during diastole when the native heart valve closes around the device.

The rigid portions 13114 of the struts 13112 and also an optional cover covering the struts 13112 can include shaped portions or can include flexible or pliable portions to allow the surface of the device to conform to the shape of the native leaflets 20, 22 when the leaflets 20, 22 close against the device to improve engagement between the struts 13112 and the native leaflets 20, 22. While four struts 13112 are shown, any number of struts 13112 can be combined to provide expanding and contracting coaptation surfaces. Different struts can include rigid portions 13114 having different shapes and lengths to provide differing rates of expansion and contraction and different maximum expansion positions to form a wide variety of differently shaped expandable mechanisms 13110.

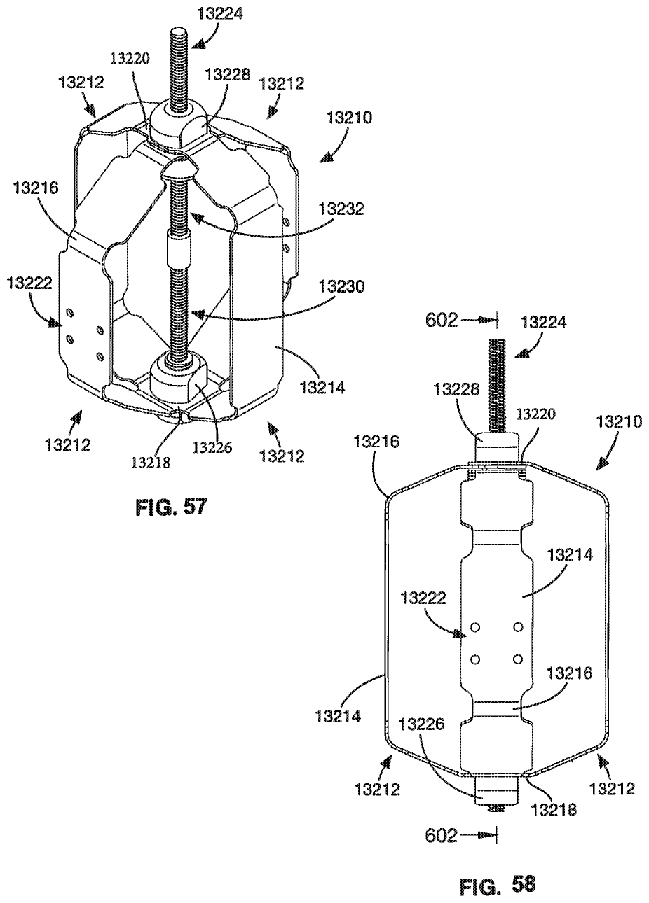

Referring now to FIGS. 57-60, an example of an expandable mechanism 13210 useable and/or for use in a device (e.g., a treatment device, a repair device, an implantable device, an implant, etc.) is shown. The device can include any other features for a device (e.g., treatment device, repair device, etc.) discussed in the present application, and the device can be positioned to engage valve tissue as part of any suitable treatment and/or repair system (e.g., any treatment and/or repair system disclosed in the present application, or the applications cited herein).

The device can be deployed from a delivery sheath or means for delivery by a pusher, such as a rod or tube as described above. The device can include a coaptation portion and an anchor portion having two or more anchors, such as the anchor portion and anchors described herein. In some implementations, the coaptation portion includes an expandable mechanism 13210 that can be actuated between a contracted condition and an expanded condition. In some implementations, an optional outer surface, such as the shell 15090, sleeve 17000, shape changing component 18012, etc. can be included to cover a portion or all of the expandable mechanism 13210.

In some implementations, the expandable mechanism 13210 can comprise and/or be formed from one or more struts 13212. In some implementations, the struts 13212 have a plurality of rigid portions 13214 that are connected together by hinge portions 13216 that enable the rigid portions 13214 to pivot relative to each other. In some implementations, a first end portion 13218 is connected to each of the struts 13212 by the hinge portions 13216. In some implementations, the struts 13212 extend from the first end portion 13218 to a second end portion 13220 connected to each strut 13212 by one of the hinge portions 13216. One or more of the rigid portions 13214 can include an attachment region 13222 for attaching another component of the device.

In some implementations, the struts 13212 are moved between a collapsed condition and an expanded condition by the actuation of a threaded actuation shaft 13224 that extends through a first actuation member 13226 and a second actuation member 13228 of the expandable mechanism 13210. In some implementations, the first end portion 13218 of the struts 13212 is attached to the first actuation member and the second end portions 13220 are attached to the second actuation member 13228.

In some implementations, the threaded actuation shaft 13224, the first actuation member 13226, and the second actuation member 13228 can be hollow so that other actuation members and other components of the device can extend through the expandable mechanism 13210 and can be moved or actuated independently of the actuation of the expandable mechanism 13210.

In some implementations, the threaded actuation shaft 13224 includes a first threaded portion 13230 extending from a distal end to a second threaded portion 13232 and the second threaded portion 13232 extends from the first threaded portion 13230 to a proximal end. In some implementations, the threads of the first threaded portion 13230 and the second threaded portion 13232 are oriented in opposite directions, i.e., if the threads of the first threaded portion 13230 are right-handed threads, then the threads of the second threaded portion 13232 are left-handed threads, and vice versa.

In some implementations, the first actuation member 13226 engages the first threaded portion 13230 and includes a threaded opening 13234 that has threads that match the orientation of the threads of the first threaded portion 13230. In some implementations, the second actuation member 13228 engages the second threaded portion 13232 and includes a threaded opening 13234 that has threads that match the orientation of the threads of the second threaded portion 13232. Consequently, rotating the threaded actuation shaft 13224 in one direction moves the first actuation member 13226 and the second actuation member 13228 in opposite directions. Because the first actuation member 13226 and the second actuation member 13228 move in opposite directions at the same rate when the threaded actuation shaft 13224 is rotated, a midplane formed between the first actuation member 13226 and the second actuation member 13228 does not move as the expandable mechanism 13210 is expanded and contracted.

In some implementations, during operation of the expandable mechanism 13210, for example, rotating the threaded actuation shaft 13224 can cause the first actuation member 13226 to move in a proximal direction and the second actuation member 13228 to move in a distal direction, thereby decreasing the distance between the first actuation member 13226 and the second actuation member 13228. In some implementations, rotating the threaded actuation shaft 13224 in the opposite direction would cause the first actuation member 13226 to move in a distal direction and the second actuation member 13228 to move in a proximal direction, thereby increasing the distance between the first actuation member 13226 and the second actuation member 13228. In this way, rotation of the threaded actuation shaft 13224 is used to expand and contract the struts 13212 of the expandable mechanism 13210. In this way, rotation of the threaded actuation shaft 13224 causes the outer perimeter or size of the expandable mechanism 13210 to increase and decrease to accommodate different size and shape gaps 26 (see FIG. 6) left between the leaflets 20, 22 during diastole when the native heart valve closes around the device.

In some implementations, the rigid portions 13214 of the struts 13212 and/or a shell, sleeve, etc. covering the struts 13212 can include shaped portions or can include flexible or pliable portions to allow the surface of the device to conform to the shape of the native leaflets 20, 22 when the leaflets 20, 22 close against the device to improve engagement between the struts 13212 and the native leaflets 20, 22. While four struts 13212 are shown, any number of struts 13212 can be combined to provided expanding and contracting coaptation surfaces. Different struts can include rigid portions 13214 having different shapes and lengths to provide differing rates of expansion and contraction and different maximum expansion positions to form a wide variety of differently shaped expandable mechanisms 13210.

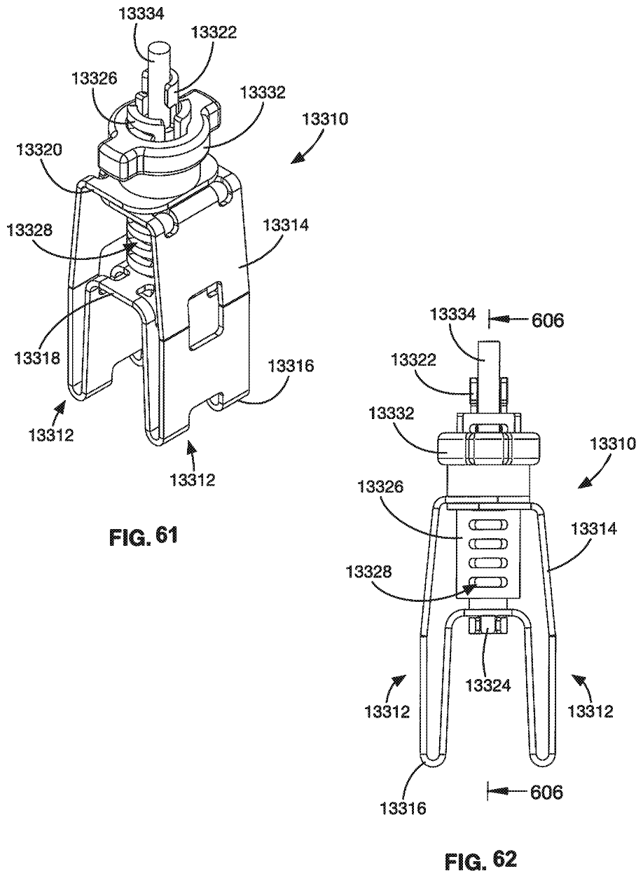

Referring now to FIGS. 61-70, an expandable mechanism 13310 useable and/or for use in a device (e.g., a treatment device, a repair device, an implantable device, an implant, etc.) is shown. The device can include any other features for a device (e.g., treatment device, repair device, etc.) discussed in the present application, and the device can be positioned to engage valve tissue as part of any suitable treatment and/or repair system (e.g., any treatment and/or repair system disclosed in the present application, or the applications cited herein). The device can be deployed from a delivery sheath or means for delivery by a pusher, such as a rod or tube as described above.

In some implementations, the device can include a coaptation portion and an anchor portion having two or more anchors, such as the anchor portion and anchors described herein. In some implementations, the coaptation portion 13304 includes an expandable mechanism 13310 that can be actuated between a contracted condition and an expanded condition. An optional outer surface, such as the shell 15090, sleeve 17000, shape changing component 18012, etc. can be included to cover a portion or all of the expandable mechanism 13310.

The expandable mechanism 13310 can comprise and/or be formed from one or more struts 13312. The struts 13312 have a plurality of rigid portions 13314 that are connected together by hinge portions 13316 that enable the rigid portions 13314 to pivot relative to each other. A first end portion 13318 is connected to each of the struts 13312 by the hinge portions 13316. The struts 13312 extend from the first end portion 13318 to a second end portion 13320 connected to each strut 13312 by one of the hinge portions 13316. One or more of the rigid portions 13314 can include an attachment region for attaching another component of the expandable mechanism 13310 or of the repair device.

The expandable mechanisms disclosed herein (and useable with any of the devices herein) can be moved to various expanded and contracted positions and held in place by a variety of different mechanisms. As is disclosed above, threaded members can be used to move the expandable mechanisms to various expanded and contracted positions and hold the expandable mechanisms in place. Additionally or alternatively, the expandable mechanisms herein can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

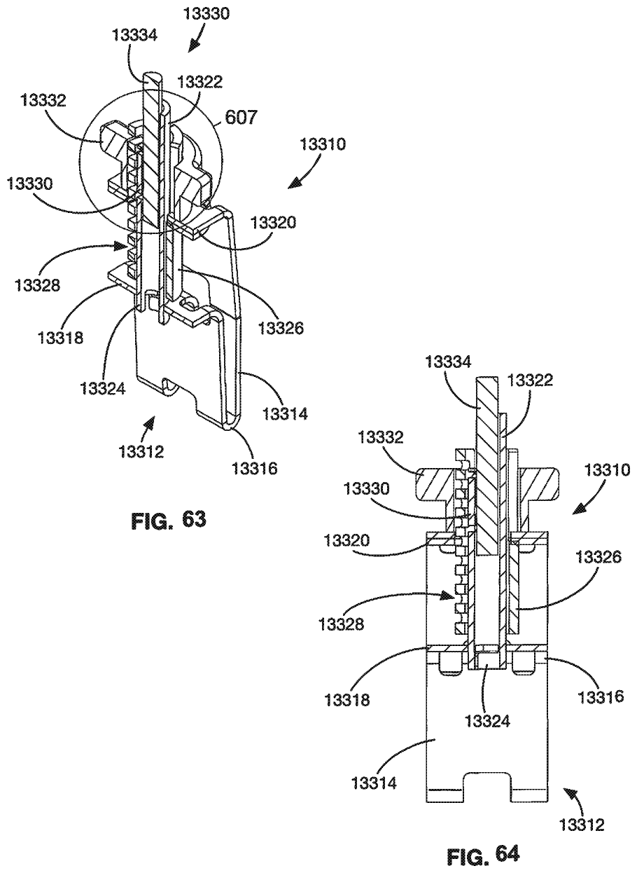

A wide variety of other mechanisms can additionally or alternatively be used. For example, FIGS. 63-64 and 67-70 illustrate an example of a mechanism for moving the expandable mechanisms to various expanded and contracted positions and holding the expandable mechanisms in place. In this example, the struts 13312 are moved between a collapsed condition and an expanded condition by the extension and retraction of an actuation tube 13322. The actuation tube 13322 extends to a distal end 13324 that is attached to the first end portion 13318 and moves within a latch tube 13326 that includes a plurality of openings 13328 for engaging with a latch member 13330 of the actuation tube 13322. A retention or securing member 13334 secures the connection between the actuation tube 13322 and latch tube 13326 by engaging the latch member 13330; the relationship between the actuation tube 13322, the latch tube 13326, the latch member 13330, and the securing member 13334 is shown in FIGS. 63-64 and 67-70 and is described below.

In some implementations, the latch tube 13326 is attached to a proximal collar or head 13332 of the device that can be coupled to a delivery mechanism for delivering the device within the native valve. In some implementations, the proximal collar or head 13332 is also attached to the second end portions 13320 of the struts 13312. Consequently, extension and retraction of the actuation tube 13322 changes the distance between the first end portion 13318 and the second end portion 13320 of the struts 13312 to cause the struts 13312 to move between the contracted and expanded conditions. The proximal collar or head 13332 can be engaged and actuated in a wide variety of ways described herein to facilitate the deployment and/or implantation of the devices described herein, such as, for example, by opening and closing paddles of the device.

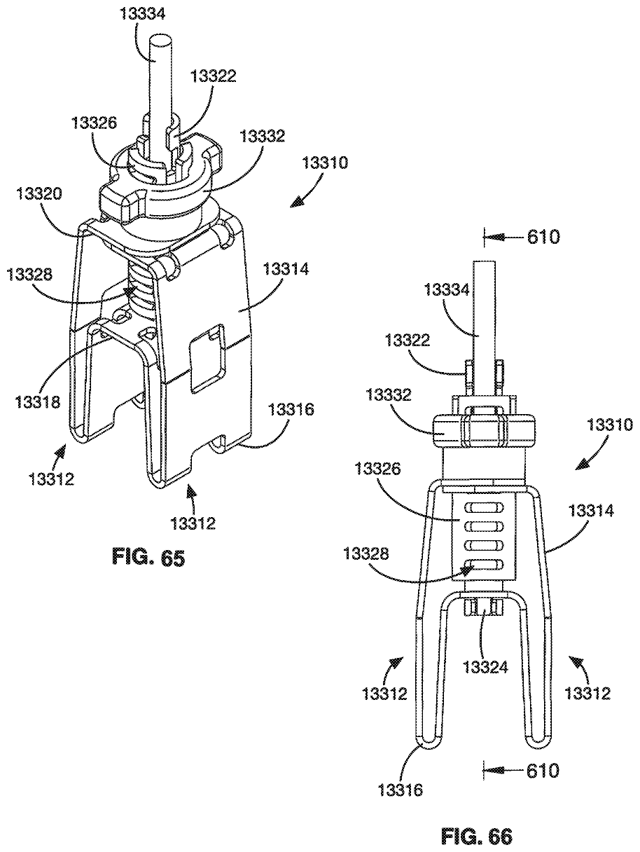

In some implementations, the latch member 13330 of the actuation tube 13322 can be moved between a latched and an unlatched condition. The actuation tube 13322 can include any number of latch members 13330 suitable for engaging corresponding openings 13328 in the latch tube 13326. Referring now to FIGS. 61-64 and 69, the latch member 13330 of the actuation tube 13322 is shown in an unlatched condition. When the latch member 13330 is in the unlatched condition, the actuation tube 13322 can be moved freely along the length of the latch tube 13326 to extend and retract the first end portion 13318, thereby expanding and contracting the expandable mechanism 13310. In the latched condition, shown in FIGS. 65-68 and 70, the latch member 13330 in the latched condition engages the openings 13328 of the latch tube 13326 to secure the position of the actuation tube 13322 relative to the latch tube 13326.

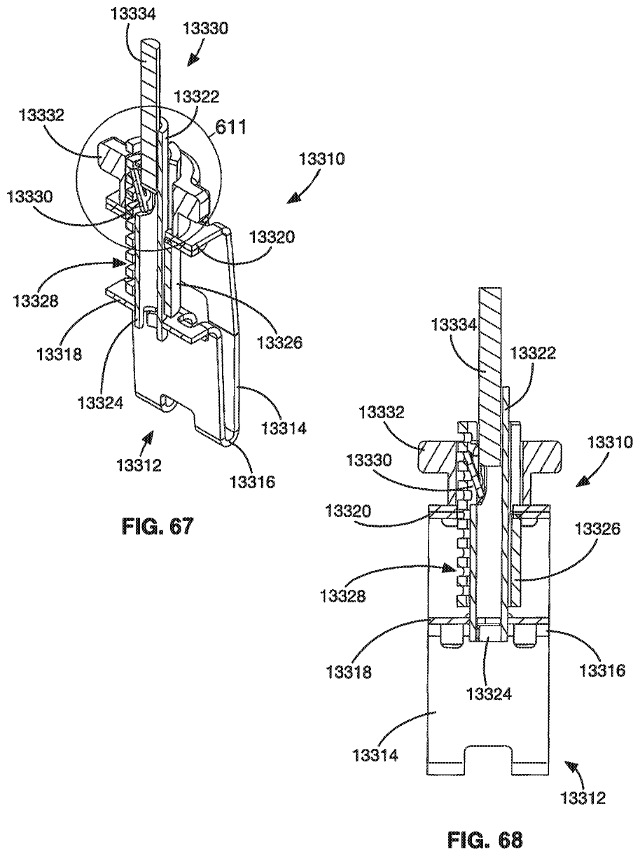

In some implementations, the latch member 13330 can optionally be integrally formed in the side of the actuation tube 13322 by laser-cutting a portion of the actuation tube 13322. The latch member 13330 is bent and shape-set in the latched condition so that in a free state the latch member 13330 is angled relative to a central axis of the actuation tube 13322. For example, as shown in FIGS. 67-68, the proximal or upper end of the latch member 13330 is biased radially outward and the opposite lower or distal end of the latch member 13330 is biased radially inward.

In some implementations, the proximal end of the latch member 13330 can be biased radially inward and the opposite distal end of the latch member 13330 can be biased radially outward. In some implementations, the expandable mechanism 13310 is biased to expand or contract and the latch member 13330 is angled outward to oppose the bias of the expandable mechanism 13310. In an example where the expandable mechanism 13310 is biased to cause the actuation tube 13322 to move in the proximal direction, the proximal end of the latch member 13330 can be biased radially outward so that the latch member 13330 engages the openings 13328 of the latch tube 13326 such that the proximal movement of the actuation tube 13322 is resisted by the latch member 13330.

In some implementations, the latch member 13330 is retained in the unlatched condition by the retaining or securing member 13334 that prohibits the latch member 13330 from pivoting into the latched condition. During deployment and/or implantation of the device the width of the expandable mechanism 13310 is adjusted by extending and retracting the actuation tube 13322. When the desired width of the expandable mechanism has been reached, the securing member 13334 is retracted from the actuation tube 13322 to allow the latch member 13330 to pivot into the latched condition to engage the openings 13328 of the latch tube 13326. In some implementations, the extension and retraction of the actuation tube 13322 causes size of the expandable mechanism 13310 to increase and decrease to accommodate different size and shape gaps 26 (see FIG. 6) left between the leaflets 20, 22 during diastole when the native heart valve closes around the coaptation element. Locking the actuation tube 13322 in place relative to the latch tube 13326 via the latch member 13330 maintains the width of the expandable mechanism 13310 against the force applied by the leaflets 20, 22.

In some implementations, the retention or securing member 13334 can optionally have a tapered distal end to facilitate re-engagement of the latch member 13330 to cause the latch member 13330 to pivot from the latched condition to the unlatched condition, thereby enabling further adjustment of the width of the expandable mechanism 13310.

In some implementations, the rigid portions 13314 of the struts 13312 and/or a shell, sleeve, etc. covering the struts 13312 can include shaped portions or can include flexible or pliable portions to allow the surface of the expandable coaptation element to conform to the shape of the native leaflets 20, 22 when the leaflets 20, 22 close against the implanted device to improve engagement between the struts 13312 and the native leaflets 20, 22. While four struts 13312 are shown, any number of struts 13312 can be combined to provided expanding and contracting coaptation surfaces. Different struts can include rigid portions 13314 having different shapes and lengths to provide differing rates of expansion and contraction and different maximum expansion positions to form a wide variety of differently shaped expandable coaptation mechanisms 13310.

Expandable coaptation elements can take a variety of different forms. In some implementations, the expandable coaptation element is made from a lattice of struts and/or can have a stent-like configuration. The lattice of struts can be formed in a variety of different ways. For example, the expandable coaptation element can be cut (e.g., laser cut) from sheet material, molded, made by additive manufacturing techniques (e.g., 3D printing), etc.

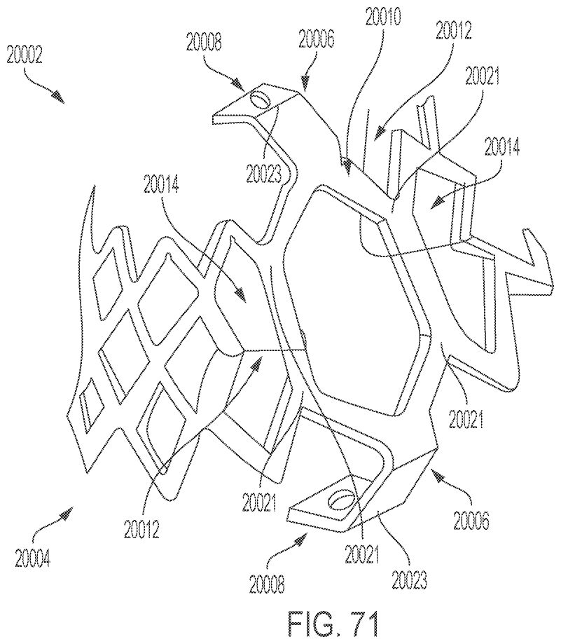

Referring now to FIGS. 71-74, example portions of an expandable coaptation element 20002 useable and/or for use in a device (e.g., a treatment device, a repair device, an implantable device, an implant, etc.) is shown. The expandable coaptation element 20002 can be used in a variety of different devices, including but not limited to, any of the treatment and/or repair devices disclosed in the present application. For example, the expandable coaptation element 20002 can be used in the device that is schematically illustrated in FIGS. 8-14. A device having the expandable coaptation element 20002 can include any other features for a device (e.g., treatment device, repair device, etc.) discussed in the present application and can be positioned to engage valve tissues as part of any suitable treatment and/or repair system (e.g., any treatment and/or repair system disclosed in the present application, or the applications cited herein). The expandable coaptation element 20002 can be deployed from a delivery sheath or means for delivery by a pusher, such as a rod or tube.

Referring to FIGS. 71-73, the coaptation element 20002 can comprise a cellular frame 20004 that can allow for control of both width and length of the coaptation element to adapt to differently sized leaflet gaps. Width and length are both relative terms, defined by the plane of the native valve. In one example, width is the dimension on an axis between the leaflets, while length is transverse to the width (e.g., between commissures). The expandable coaptation element 20002 can be composed of a shape-memory alloy—such as Nitinol—to provide shape-setting capability, or any other flexible material suitable for implantation in the human body.

The expandable coaptation element 20002 can be cellular, tubular, elliptical/bowl shaped, or any other shape conducive to implantation between leaflets of a native valve. In some implementations, the expandable coaptation element 20002 comprises a stent-like cellular frame with triangular, diamond, and/or hexagonal honeycomb cells. In some implementations, the structure can be tubular wherein the length is greater than the width with the length-ends having an arc shape. The coaptation element 20002 can have a plurality of ends (on the length of the coaptation element) and/or a plurality of sides (on the width of the coaptation element).

In some implementations, the coaptation element 20002 can have one or a plurality of tabs or extensions 20006 on the side or plurality of sides (e.g., at each point on the frame 20004 where the minor axis of the cross-section intersects the frame). The coaptation element 20002 can be expanded and/or contracted vertically and/or horizontally. Expansion and/or contraction can be achieved through actuation portions or actuation members 20008 that extend from the tabs or extensions 20006. The actuation portions or actuation members 20008 can be coupled to the frame 20004, such as being integrally formed with the frame as illustrated. In some implementations, the actuation portions or actuation members 20008 can be diametrically disposed around a central axis of the expandable coaptation element 20002. The side or plurality of sides of the expandable coaptation element 20002 referenced in FIGS. 71-74 can expand sequentially, simultaneously, or otherwise when being manipulated and/or actuated.

The expandable coaptation element 20002 can expand and/or contract in a variety of different ways. For example, the expandable coaptation element 20002 can:
Expand or contract only in length;
Expand or contract only in width;
Simultaneously expand and contract in length and width;
Sequentially expand or contract such that the expandable coaptation element 20002 expands or contracts first in length and then expands or contracts in width;
Sequentially expand or contract such that the expandable coaptation element 20002 expands or contracts first in width and then expands or contracts in length;
Independently expand or contract in length and width;
Partially expand or contract in length and then expand or contract in both length and width; and/or
Partially expand or contract in width and then expand or contract in both length and width.

As such, shape changing elements 20002 can expand sequentially, simultaneously, or otherwise.

In some implementations, one or more secondary controls 20012 can be used to change the size of one or more cells independently of the actuation portions or members 20008. The secondary control 20012 can take a wide variety of different forms. For example, the secondary control can comprise a line, such as a suture, a wire, etc., an adjustable width fastener, a spring, or any other component capable of being controlled to change the size of one or more of the cells 20014. The one or more secondary controls 20012 can be attached to the individual cells 20014 of the frame 20004. The individual cells 20014 can be specialized, such as by having a different shape (e.g., hexagonal, heptagonal, rectangular, triangular, etc.), different strut thickness, different strut width, etc. than other cells of the frame 20004. In some implementations, the secondary control 20012 can be used to adjust the width of the frame 20004 by pulling on the specialized cell to narrow the specialized cell 20014 and, as a result, the frame 20004 as a whole.

The narrowing and expanding of the individual cells 20014 with the secondary control 20012 changes the shape of the expandable coaptation element 20002 differently than the movement of the actuation portions or members 20008. By adjusting the actuation portions or members 20008 and the secondary control 20012, the expandable coaptation element can be adjusted to a variety of different shapes and sizes.

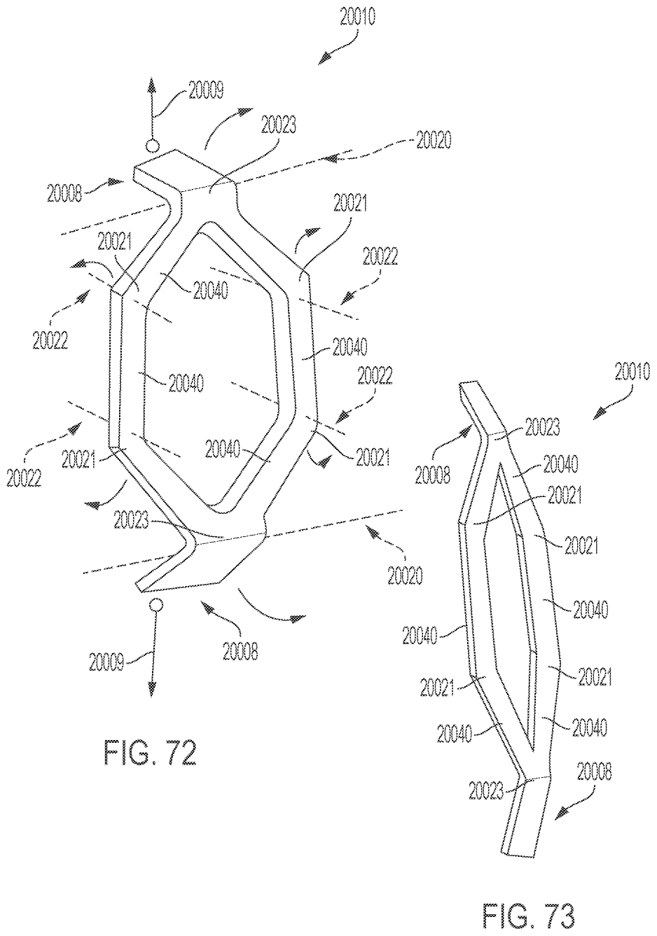

FIGS. 72-73 depict a control cell 20010 that forms part of the frame 20004 of the expandable coaptation element 20002. The control cell 20010 can expand by foreshortening, narrowing, and/or bending or flexing at certain points. The actuation portions or members 20008 are connected to the control cell 20010 via a plurality of tabs, extensions, connectors or connection portions 20006 that are coupled to the upstream and downstream ends of the cell at bending points 20023. In some implementations, the elastic properties of the material, variations in the width of the material, and/or variations the thickness of the material can be used to create the control cell 20010 with bending points 20021, 20023 that bend or flex before one another and/or before other portions of the control cell 20010 and/or bend or flex out of the plane of the control cell. In some implementations, the force applied to the control cell will initially overwhelm the bending points 20021 and/or the bending points 20023, causing the points to give and the shape of the expandable coaptation element 20002 to change.

For example, the bending points 20021, 20023 can be configured such that:
the points 20021 flex before the points 20023;
the points 20023 flex before the points 20021;
the points 20021 and the points 20023 simultaneously flex;
the points 20021 partially flex and then the points 20021 and the points 20023 flex; and/or
the points 20023 partially flex and then the points 20021 and the points 20023 flex.

FIG. 72 shows the control cell 20010 with bending points 20021 that bend about a plurality of bending axes 20022 and bending points 20023 that bend about axes 20020. A comparison of FIGS. 72 and 73 illustrates how the control cell 20010 can bend/be manipulated by adjusting the actuation portions or members 20008 in an upward or downward direction. In some implementations, by adjusting the actuation portions or members 20008 in an upward or downward direction, the bending point 20023 on the actuator will give first and elongate the control cell 20010. Then, the bending points 20021 will bend along the bending axes 20022, thus further elongating or shortening the control cell 20010. FIG. 73 depicts a control cell 20010 within a frame 20004 after the actuation portions or members 20008 have been manipulated in an upward and downward direction, thus elongating and narrowing the control cell 20010.

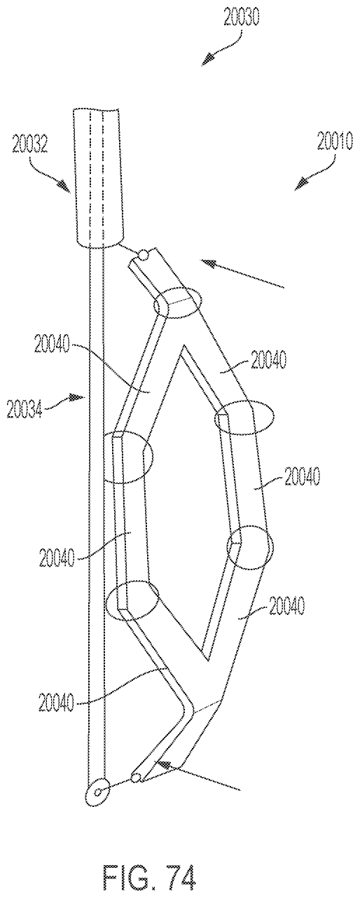

Control cells 20010 can be expanded and contracted in a variety of different ways. Any mechanism capable of moving the actuation members 20008 as illustrated by the arrows 20009 in FIG. 72 can be used. FIG. 74 shows a control cell 20010, as described in FIGS. 71-73 with an example actuation member 20030. The actuation member can comprise a catheter 20032 and rod 20034 to manipulate, in an upward and/or downward direction, and expand and/or contract the control cell 20010. When expanded and/or contracted, the catheter 20032 and rod 20034 causes a strut or a plurality of struts 20040 to elongate and/or contract, thus adjusting the size of the control cell 20010.

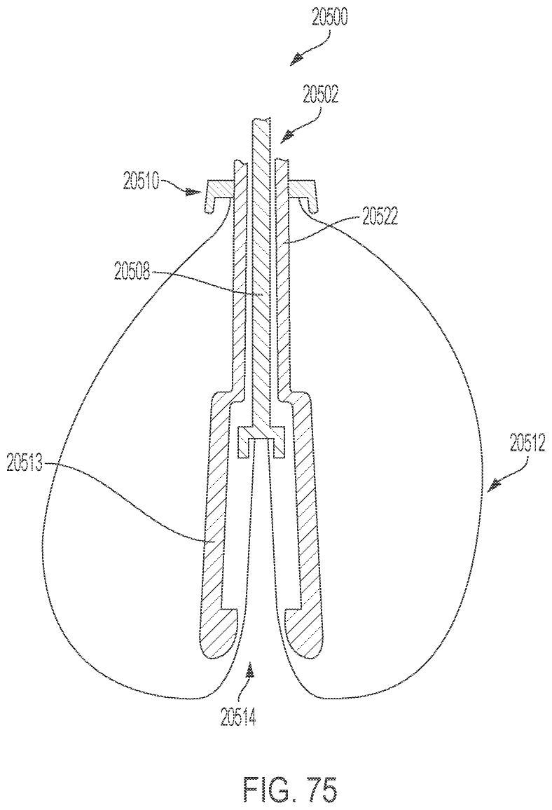

FIGS. 75-77 illustrate an example of an expandable coaptation element 20500. In the example illustrated by FIGS. 75-77, the expandable coaptation element 20500 can comprise one or more shape changing components 20512 and an adjustment mechanism 20502. The shape changing component 20512 is configured to change size and/or shape when a portion of the shape changing component 20512 is pulled into or pushed out of a receiver 20513 of the adjustment mechanism 20502.

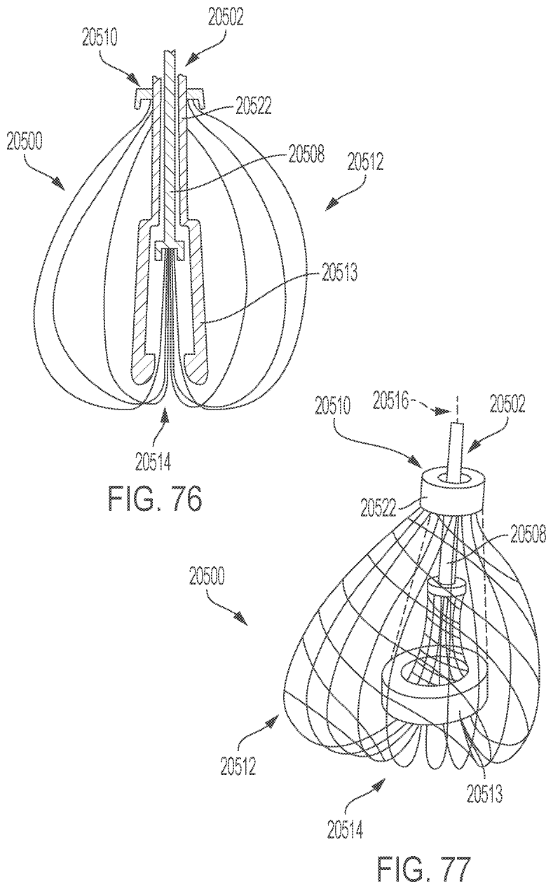

The shape changing components 20512 can take a wide variety of different forms having a variety of different expanded and contracted shapes. In the example illustrated by FIG. 76, the shape changing component 20512 can be made from individual wires having a whisk or whisk-like configuration. In the example illustrated by FIG. 77, the shape changing component 20512 can be made of a braided or mesh material. For example, the shape changing component 20512 can comprise a plurality of crossing wires. The shape changing components 20512 can have a variety of different shapes. For example, the shape changing components 20512 can be tear-drop shaped, spherical, cylindrical, conical, frustoconical, egg shaped, pyramid, a portion of a pyramid, diamond, combinations of these shapes, etc. in the expanded and/or retracted configurations. The shape changing components 20512 can be formed from a braided or woven tube of material—such as Nitinol wire—or any other flexible material suitable for implantation in the human body.

The shape changing components 20512 can be transformed from the expanded configuration to the compressed configuration in a wide variety of different ways. In the illustrated examples, the shape changing components are pulled into or pushed out of the receiver 20513 of the adjustment mechanism 20502. In the illustrated example, pushing the shape changing components 20512 out of the receiver 20513 causes the shape changing components 20512 to expand and drawing the shape changing components 20512 into the receiver causes the shape changing components to decrease in size. In some implementations, the shape changing components 20512 expand and contract laterally without increasing or substantially increasing the height of the expandable coaptation element 20500.

A variety of different mechanisms can be used to move the shape changing components 20512 from the expanded configuration to the contracted configuration. In some implementations, an inner shaft 20508 moves along an axis 20516 inside an outer shaft 20522 to move the shape changing components 20512 from the expanded configuration to the contracted configuration. In some implementations, a nut and a bolt can be used to move the shape changing components 20512 between expanded configuration and contracted configurations.

In the illustrated example, the shape changing component 20512 has a first end 20510 (e.g., proximal/upstream end) fixedly attached to the outer shaft 20522. The shape changing component 20512 has a distal end 20514 that is attached to the inner shaft 20508. The inner shaft 20508 and attached distal end 20514 are axially movably attached inside the receiver 20513. Axially adjusting the second or distal end 20514 within the receiver 20513 exposes more or less of the shape changing component 20512. The shape changing component 20512 can be biased to expand toward a generally teardrop or oval shape, such that exposure of more of the shape changing component 20512 makes the expandable coaptation element 20500 wider.

The expandable coaptation element 20500 can be used in a variety of different devices, including but not limited to, any of the treatment and/or repair devices disclosed in the present application. For example, any expandable coaptation element disclosed herein can be used in the device that is schematically illustrated in FIGS. 8-14. The expandable coaptation element 20500 can be positioned to engage leaflets 30, 32, 34 (see FIGS. 7 and 34) or leaflets 20, 22 (see FIGS. 6 and 36) as part of any suitable device (e.g., any treatment and/or repair device disclosed in the present application). The device can be deployed from a delivery sheath, a steerable catheter and/or an implant catheter. The device can include an expandable coaptation element 20500 and an anchor portion having two or more anchors, such as the anchor portion and anchors described herein. The device can be a prosthetic spacer device, valve repair device, valve treatment device, or another type of device that attaches to leaflets of a native valve.

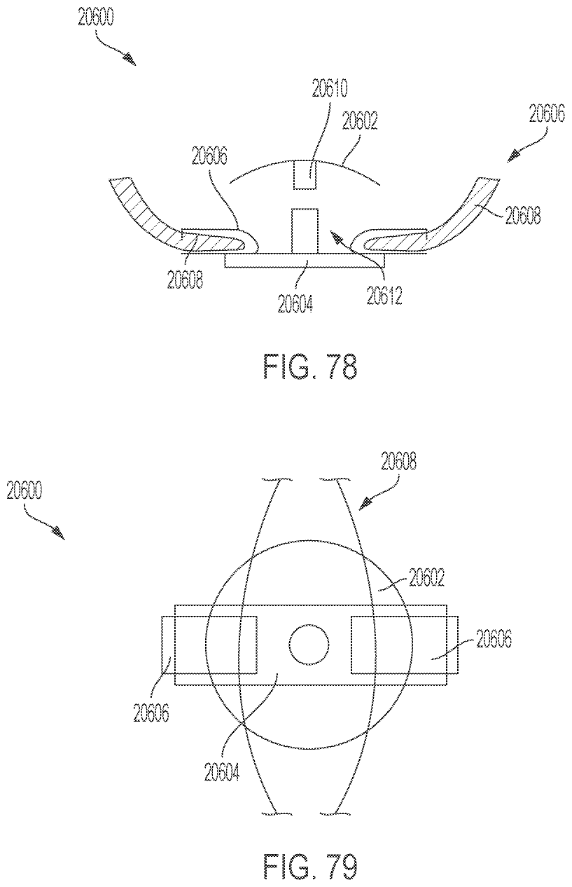

Referring to FIGS. 78-81, in some implementations, an extension or blocking member, etc. (e.g., a cap, umbrella, canopy, awning, cap-like extension, umbrella-like extension, canopy-like extension, etc.) can be attached to a device (e.g., a treatment device, a repair device, etc.) to block, divert or inhibit regurgitant blood flow through the native valve, such as a native mitral valve. The extension or blocking member can take a variety of different forms. The extension or blocking member can be sized and shaped to reduce regurgitation while maximizing flow from the atrium to the ventricle. For example, the extension or blocking member can be shaped to match or substantially match a profile of the regurgitant flow and/or have a shape that corresponds to an annulus of the native valve but is smaller than the annulus of the native valve.

Referring now to FIGS. 78-81, an example of a device 20600 e.g., a treatment device, repair device, valve treatment device, valve repair device, implantable device, implant, etc.) is shown. The device 20600 (e.g., a treatment device, a repair device, etc.) can include extension or blocking member 20602 (e.g., cap, umbrella, canopy, awning, cap-like extension, umbrella-like extension, canopy-like extension, etc.), a frame or base 20604, and a clasp or a plurality of clasps 20606. The extension 20602 can be used to cover a gap between closed leaflets to block or partially block regurgitation through the native valve. The extension 20602 can be connected to the frame or base 20604 in a variety of different ways. In the illustrated example, the extension has a stem 20610 that is secured to a complimentary socket 20612 on the frame 20604. However, the extension 20602 can be attached to the frame or base 20604 in any manner or the extension 20602 can be connected directly to the one or more clasps 20606.

In some implementations, the extension 20602 is expandable. For example, the extension can take the form of or include features of an amplatzer, occluder or plug. In some implementations, the extension has a fixed size when deployed, but can be compressed to fit inside a delivery catheter. The extension 20602 can be composed of cloth, semi-rigid material, rigid material, or shape-memory alloy wire—such as Nitinol—to provide shape-setting capability, or any other flexible material suitable for implantation in the human body.

A clasp or a plurality of clasps 20606 can be attached to the frame 20604 to attach leaflets of the native valve 20608 to one another. In some implementations, the frame 20604 can be omitted. For example, the clasps 20606 can be connected to one another, be integrally formed, and/or connect directly to the extension 20602.

FIG. 79 shows a top plan view of the example device 20600 including the extension 20602. As can be seen in FIG. 79, the extension 20602 covers a gap between closed leaflets to block or partially block regurgitation through the native valve.

The extension 20602 can be used in a variety of different devices, including but not limited to, any of the treatment and/or repair devices disclosed in the present application. As one example, any extension, blocking member, etc. disclosed herein can be used in the device that is schematically illustrated in FIGS. 8-14. The device 20600 is one of the many different configurations that the device that is schematically illustrated in FIGS. 8-14 with an extension 20602 can take. The device 20600 can include any other features for a device (e.g., a treatment device, a repair device, etc.) discussed in the present application, and the extension 20602 can be positioned to block or impede regurgitant flow through leaflets 30, 32, 34 (see FIGS. 7 and 34) or leaflets 20, 22 (see FIGS. 6 and 36) as part of any suitable device (e.g., any treatment and/or repair device disclosed in the present application). The device 20600 can be deployed from a delivery sheath, a steerable catheter, and/or an implant catheter. The device 20600 can include an anchor portion having two or more anchors, such as the anchor portions and anchors described herein.

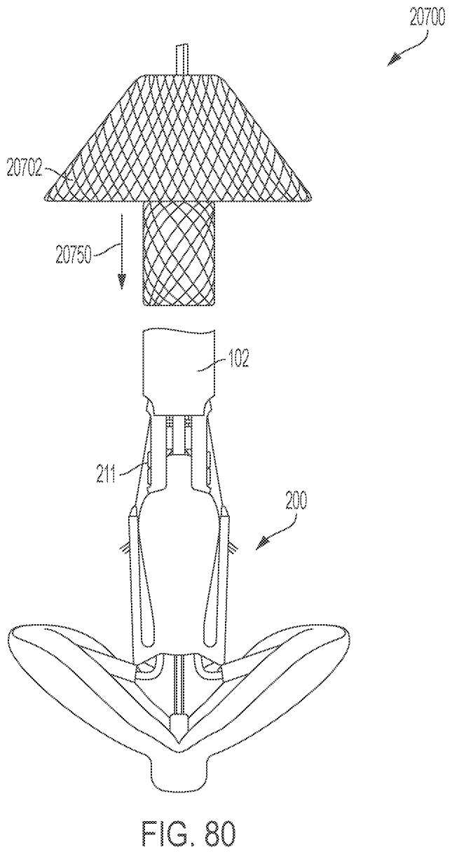

FIG. 80 shows an example valve system 20700 with an extension or blocking member 20702 (e.g., cap, umbrella, canopy, awning, cap-like extension, umbrella-like extension, canopy-like extension, etc.) being installed on a device 200 (e.g., a treatment device, a repair device, etc.). In the illustrated example, the device 200 can be the same as or similar to the device illustrated by FIG. 22. However, the extension 20702 can be installed on any device, such as any device (e.g., treatment and/or repair device, etc.) shown and described in the present application.

The extension 20702 can be installed on the device 200 in a variety of different ways. In the illustrated example, the extension 20702 is advanced as indicated by arrow 20750 over an implant catheter of the delivery system 102 that is used to deploy the device 200. Once in place, the extension 20702 is attached to the device 200. For example, the extension 20702 can be attached to the collar 211. However, the extension 20702 can be attached to the device 200 in any manner. The extension 20702 can be used to cover a gap that remains after the coaptation portion 204 of the device 200 is positioned. Covering the remaining gap can block or impede regurgitation through the native valve.

In some implementations, the extension 20702 is expandable. For example, the extension can take the form of or include features of and amplatzer occluder or plug. In some implementations, the extension has a fixed size when deployed, but can be compressed to fit inside a delivery catheter. The extension 20702 can be composed of cloth, semi-rigid material, rigid material, or shape-memory alloy wire—such as Nitinol—to provide shape-setting capability, or any other flexible material.

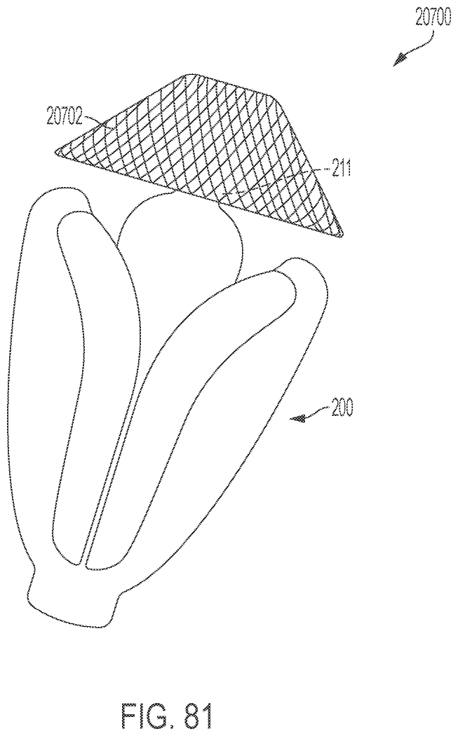

The extension 20702 can be inserted simultaneously with the device 200 or after the device 200 has been implanted. For example, the device can be positioned. Then, regurgitation can be assessed. Based on the remaining regurgitation, an extension 20702 can be selected and added to the device 200. FIG. 81 shows a device 200 with the extension 20702 secured.

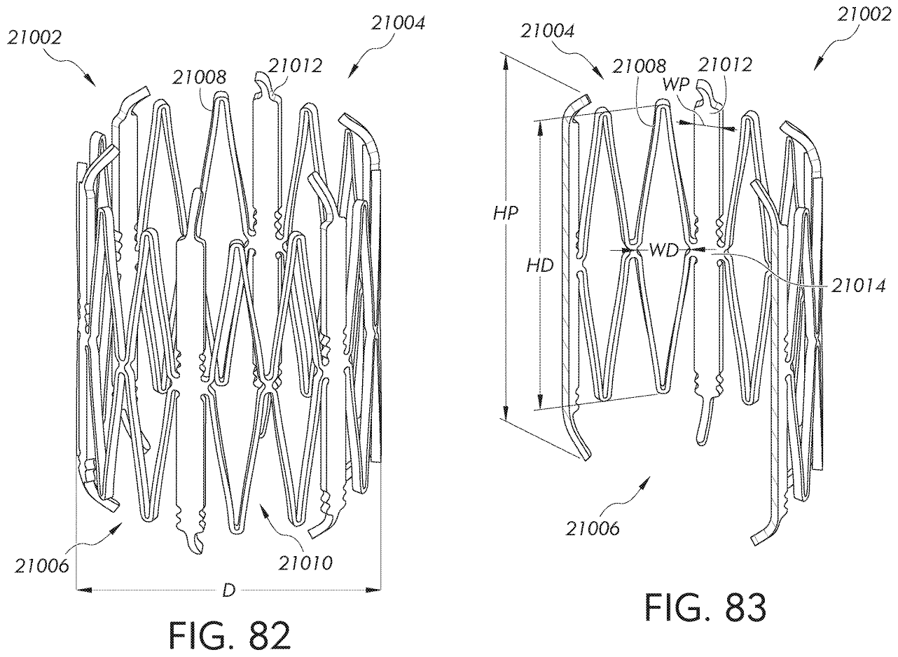

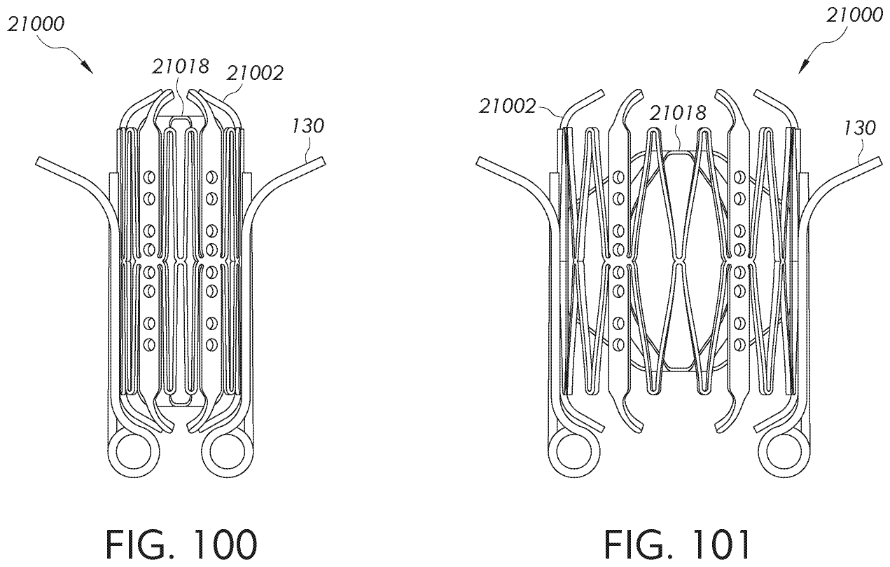

FIGS. 82-83 illustrate an example frame member 21002 (e.g., frame, body, cage, fixture, chassis, form, etc.) for an expandable coaptation element 21000 (e.g., FIG. 100-101). The expandable coaptation element 21000 (e.g., spacer, coaptation element, gap filler, membrane, sheet, plug, wedge, balloon, etc.) can be part of a device (e.g., a treatment device, a repair device, etc.) for repairing a native heart valve and can be used as part of any device described herein.

In some implementations, the frame member 21002 is configured to move between a first radially contracted configuration and a second radially expanded configuration. The frame member 21002 can be configured in a variety of ways. In some implementations, the frame member 21002 has a generally cylindrical shape (i.e., a circular cross section) having a proximal end 21004, a distal end 21006 opposite the proximal end 21004, and a diameter D that can be collapsed (i.e., in the first configuration) and expanded (i.e., in the second configuration). In some implementations, however, the frame member 21002 can have a shape other than cylindrical (e.g., a cross section that is oval, rectangular, elliptical, or other suitable shape).

In some implementations, the frame member 21002 can include a plurality of interconnected struts 21008 that are configured to flex or bend to allow the frame member 21002 to move between the first and second configuration. The interconnected struts 21008 can be configured in a variety of ways, such as for example, the number and size of the struts, the shape of each strut, the arrangement of struts relative to other struts, the interconnection of the struts, etc. Any configuration that can facilitate the expansion and contraction of the frame member 21002 can be used. In some implementations, the struts 21008 are arranged in diamond patterns 21010 having a height HD and a width WD (FIG. 83).

In some implementations, the example frame member 21002 includes a plurality of posts 21012 interconnected by the plurality of struts 21008. The posts 21012 can be configured in a variety of ways, such as for example, the number and size of the posts, the shape of each post, the arrangement and connection of posts relative to the struts, etc.

In the illustrated example, each post 21012 extends linearly from the proximal end 21004 to the distal end 21006 and has a height HP and a width WP (FIG. 83). The width WP, in the illustrated example, is larger than a width WS of each strut. In some examples, the width WP is greater than twice, or greater than three times, the width WS of each strut 21008. In some implementations, the width WP of the post is less than or equal to the width WS of each strut.

In the illustrated example, each post 21012 is connected on either side, along a midpoint 21014, to one of the diamond patterns 21010 of struts 21008. In the illustrated example, the frame member 21002 includes six (6) posts 21012 evenly positioned around the periphery of the frame member 21002. Each of the six (6) posts 21012 is separated by two interconnected diamond patterns 21010 of the struts 21008. The posts 21012 and the struts 21008 can be interconnected in any suitable manner.

In some implementations, the frame member 21002 is formed as a unitary piece. For example, the frame member 21002 can be laser-cut from a tube. In some implementations, however, some of the struts 21008 and the posts 21012 can be formed separately and connected in any suitable manner (e.g., welded together).

In some implementations, the frame member 21002 can comprise a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, when the diameter D of the frame member 21002 expands, the height HD of each of the diamond patterns 21010 decreases while the width WD increases. Conversely, the height HP of each post 21012 does not change. Thus, the frame member 21002 has a fixed height (i.e., the height HP of the posts) that does not change between the first and second configuration.

In some implementations, the frame member 21002 can be configured to change height between the first and the second configuration. For example, the posts can be replaced with diamond patterns, such that the entire frame member 21002 decreases in height as the frame member increases in width.

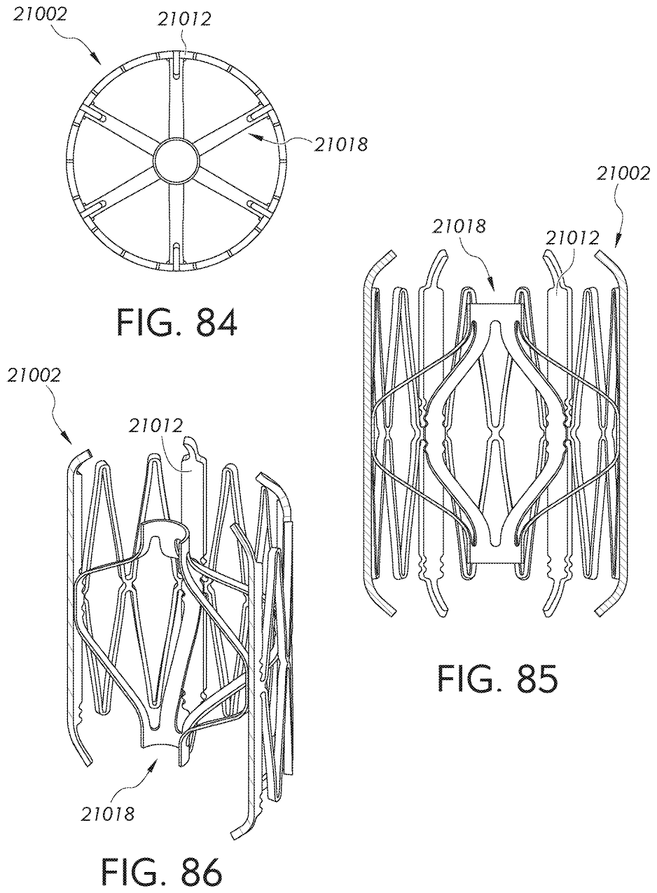

The frame member 21002 can be moved between the first and the second configurations by any suitable means. For example, the frame member 21002 can be mounted around an expandable mechanism 21016 (see FIGS. 84-86) configured to engage the frame member 21002 to move the frame member 21002 between the first and the second configurations. A variety of different mechanisms can be used to move the frame member 21002 between the first and second configurations. For example, any expandable mechanism disclosed herein can be used. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

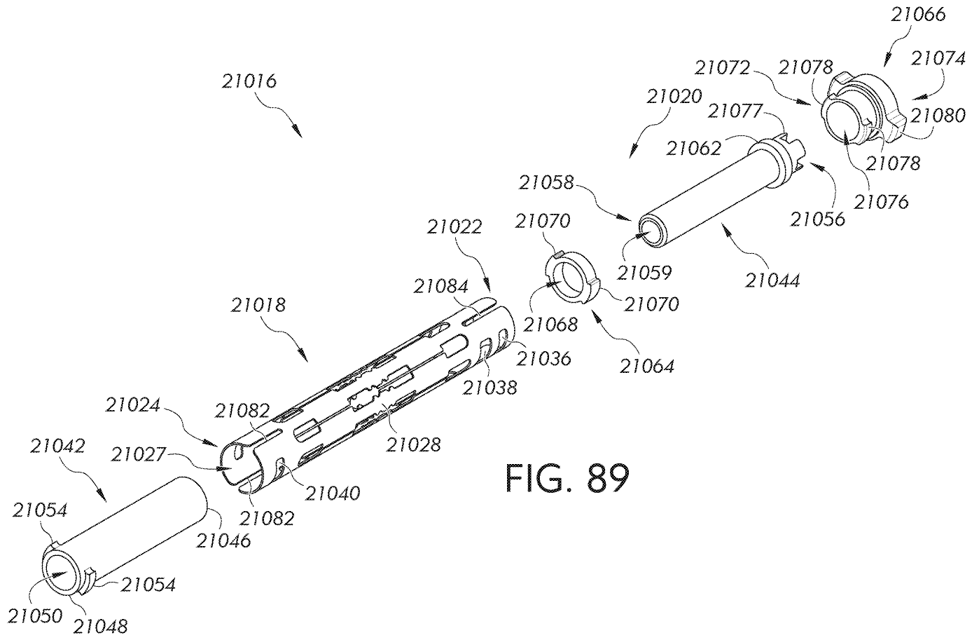

In some implementations, as shown in FIG. 89, the expandable mechanism 21016 includes an expandable member or expansion member 21018 (e.g., frame, body, strut assembly, tube, shaft, etc.) and an actuation mechanism. Referring to FIGS. 84-86, in some implementations, the frame member 21002 is mounted around the expandable/expansion member 21018. The expandable/expansion member 21018 can be configured in a variety of ways. Any configuration capable of moving the frame member 21002 between the first and the second configuration can be used.

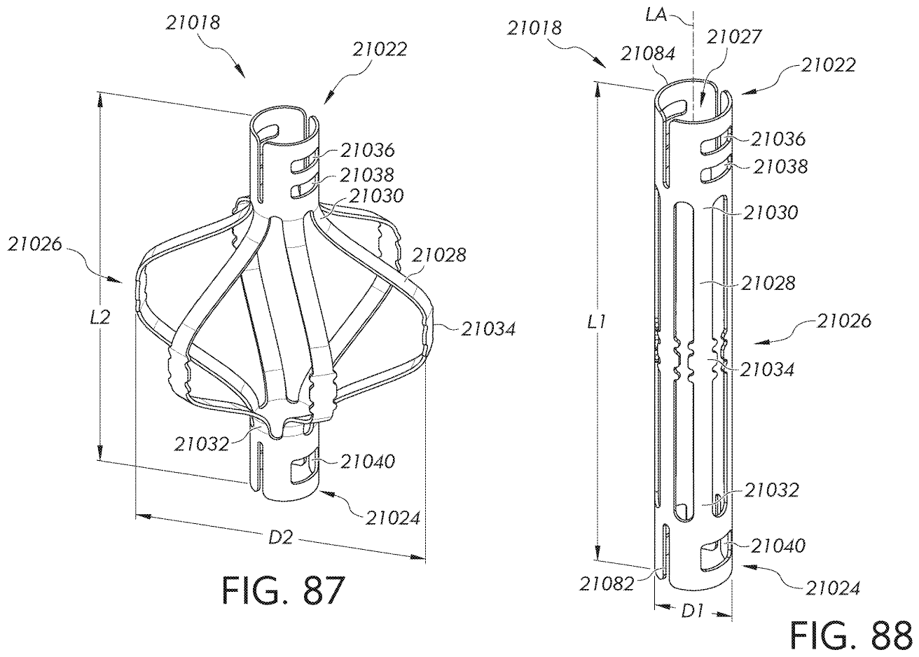

Referring to FIGS. 87-88, in some implementations, the expandable/expansion member 21018 is configured as an elongated cylindrical shape having a proximal end portion 21022, a distal end portion 21024 opposite the proximal end portion 21022, an intermediate portion 21026 connecting the proximal end portion 21022 and the distal end portion 21024, and a passage 21027 extending through the expandable/expansion member 21018 from the proximal end portion 21022 to the distal end portion 21024.

In some implementations, the intermediate portion 21026 can comprise and/or be formed from a plurality of strips 21028 that are configured to bend or flex. In some implementations, each strip 21028 has a proximal end 21030 attached to the proximal end portion 21022 of the expandable/expansion member 21018, a distal end 21032 attached to the distal end portion 21024, and an intermediate portion 21034 between the proximal end 21030 and the distal end 21032.

In some implementations, the expandable/expansion member 21018 includes six (6) strips 21028 equally spaced apart around the periphery of the expandable/expansion member 21018. In some implementations, however, the strips 21028 need not be equally spaced and/or the intermediate portion 21026 can include more or less than six (6) strips.

In some implementations, the expandable/expansion member 21018 is movable between a first configuration (i.e., collapsed or narrow configuration) (FIG. 88) and a second configuration (i.e., an expanded or wide configuration) (FIG. 87). As shown in FIG. 88, in the first configuration, each of the plurality of strips 21028 extend parallel to a longitudinal axis LA of the expandable/expansion member 21018 and the expandable/expansion member 21018 has a first length L1. In the first configuration, the proximal end portion 21022, the distal end portion 21024, and the intermediate portion 21026 have the same first diameter D1.

In some implementations, the expandable/expansion member 21018 can be formed as a unitary piece. For example, the expandable/expansion member 21018 can be laser-cut from a tube. In some implementations, the expandable/expansion member 21018 can be formed from multiple pieces, such as for example, a pair of Nitinol sheets.

In some implementations, the expandable/expansion member 21018 can comprise a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, as shown in FIG. 87, in the second configuration, the proximal end portion 21022 is moved toward the distal end portion 21024, or vice versa, causing the strips 21028 to bend or flex outward such that the intermediate portion 21026 expands to a second diameter D2 that is greater than the first diameter D1. Further, in second configuration, the expandable/expansion member 21018 has a second length L2, less than the first length L1 and the diameter of the proximal end portion 21022 and the distal end portion 21024 remain at the first diameter D1 while the intermediate portion 21026 expands to the second diameter D2.

As shown in FIGS. 84-86, in some implementations, the expandable/expansion member 21018 positioned within the periphery of the frame member 21002 to engage the frame member 21002. In some implementations, the intermediate portion 21034 of each strip 21028 is attached to the frame member 21002. In some implementations, the intermediate portion 21034 of each strip 21028 is attached to a corresponding post 21012 of the frame member 21002. The intermediate portion 21034 of each strip 21028 can attach to the frame member 21002 in any suitable manner, such as for example, welding.

In some implementations, as a result of the expandable/expansion member 21018 being positioned within the periphery of the frame member 21002 and attached to the frame member 21002, movement of the expandable/expansion member 21018 between the first configuration (i.e., collapsed or narrow configuration) (FIG. 88) and a second configuration (i.e., expanded or wide configuration) (FIG. 87) moves the frame member 21002 between the frame member's first configuration (i.e., collapsed) and second configuration (i.e., expanded).

In some implementations, the frame member 21002 and/or the expandable/expansion member 21018 can be configured to be normally in the first configuration (i.e., collapsed or narrow), in the second configuration (i.e., expanded or wide), or some intermediate position between the first and the second configuration. Thus, in some implementations, movement of the expandable/expansion member 21018 pulls the frame member 21002 inward toward the first position against a bias of the frame member 21002. In some implementations, movement of the expandable/expansion member 21018 pushes the frame member outward to the second position against a bias of the frame member 21002.

In some implementations, the actuation mechanism 21020 is configured to move the expandable/expansion member 21018 between the first and the second configurations. The actuation mechanism 21020 can be configured in a variety of ways. Any actuation mechanism capable of moving the expandable/expansion member 21018 between the first and the second configurations can be used. In some implementations, the actuation mechanism 21020 is mounted within the expandable/expansion member 21018 between the proximal end portion 21022 and the distal end portion 21024 of the expandable/expansion member 21018.

Referring to FIGS. 87-88, in some implementations, the expandable/expansion member 21018 can include structure configured to mount the actuation mechanism 21020 within the expandable/expansion member 21018. The structure can be configured in a variety of ways. In some implementations, the proximal end portion 21022 of the expandable/expansion member 21018 includes a first pair of radially opposed openings 21036, and a second pair of radially opposed openings 21038 distal to the first pair of radially opposed openings 21036 and the distal end portion 21024 of the expandable/expansion member 21018 includes a third pair of radially opposed openings 21040.

In some implementations, the actuation mechanism 21020 is configured as a threaded connection. Referring to FIG. 89, the example actuation mechanism 21020 includes a distal member 21042 (e.g., tube, body, shaft, etc.) configured to threadably couple with a proximal member 21044 (e.g., tube, body, shaft, etc.). The distal member 21042 and the proximal member 21044 can be configured in a variety of ways. In the illustrated example, the distal member 21042 is formed as a cylindrical tube having a circular cross section. In some implementations, however, the distal member 21042 can have a shape other than cylindrical (e.g., a cross section that is oval, rectangular, elliptical, or other suitable shape).

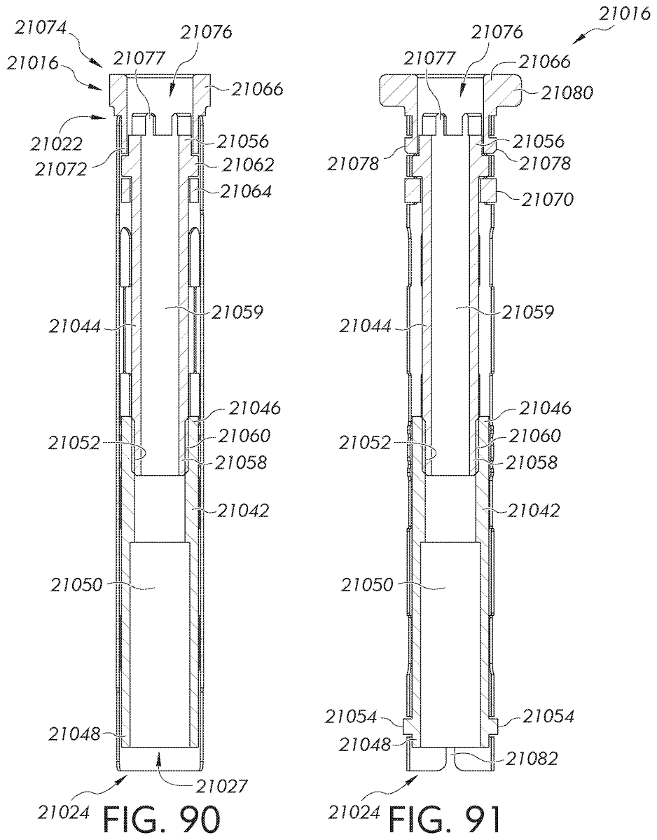

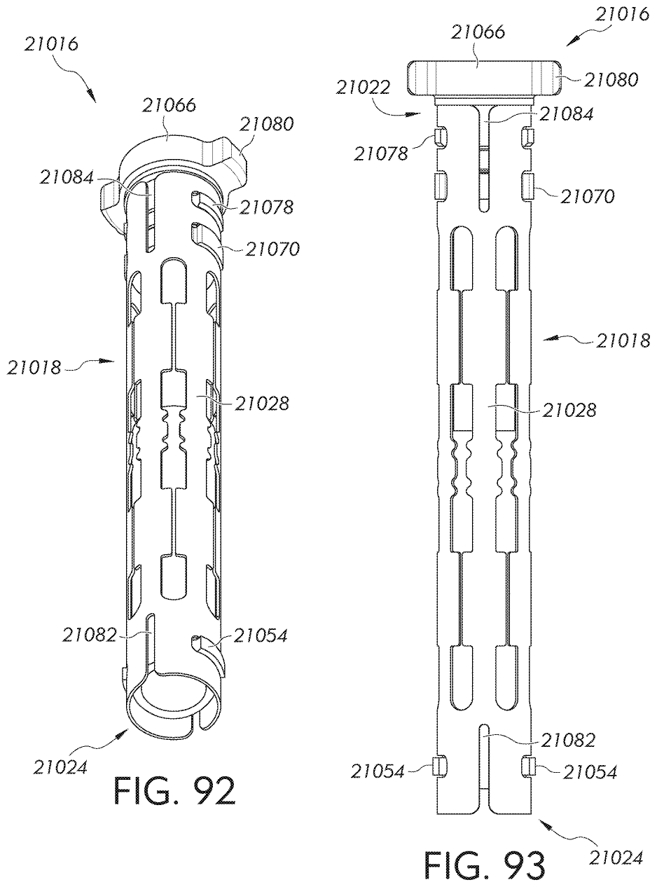

In some implementations, the distal member 21042 includes a proximal end 21046, a distal end 21048 opposite the proximal end 21046, and an inner passage 21050 extending through the distal member 21042 member from the proximal end 21046 to the distal end 21048. The inner passage 21050 includes female threads 21052 (FIGS. 90-91) for threadably coupling with the proximal member 21044.

In some implementations, the distal member 21042 is sized to be received within the passage 21027 of the expandable/expansion member 21018. In some implementations, the distal member 21042 includes structure of fixing the distal member 21042 in position within the passage 21027 relative to the expandable/expansion member 21018. In the illustrated example, the distal end 21048 includes a pair of opposed projections 21054 configured to be received within the third pair of radially opposed openings 21040, as shown in FIGS. 90-93.

In some implementations, the proximal member 21044 is formed as a cylindrical tube having a circular cross section. In some implementations, the proximal member 21044 can have a shape other than cylindrical (e.g., a cross section that is oval, rectangular, elliptical, or other suitable shape).

In some implementations, the proximal member 21044 includes a proximal end 21056, a distal end 21058 opposite the proximal end 21056, and an inner passage 21059 extending through the proximal member 21044 from the proximal end 21056 to the distal end 21058.

In some implementations, the proximal member 21044 can include male threads 21060 (FIGS. 90-91) extending along at least a portion of the exterior of the proximal member 21044. The male threads 21060 are configured to mate with the female threads 21052 of the distal member 21042.

In some implementations, the proximal member 21044 is sized to be received within the passage 21027 of the expandable/expansion member 21018. In some implementations, the actuation mechanism 21020 can include structure for restricting axial movement of the proximal member 21044 within the passage 21027 relative to the expandable/expansion member 21018.

In some implementations, the structure restricts axial movement of the proximal member 21044 within the passage 21027 relative to the expandable/expansion member 21018 while allowing rotational movement of the proximal member 21044 relative to the expandable/expansion member 21018. The structure for restricting axial movement of the proximal member 21044 can be configured in a variety of ways.

In some implementations, the actuation mechanism 21020 includes a circumferential, radially extending ridge 21062 at the proximal end 21056. In some implementations, the ridge 21062 is configured to engage with a stop 21064 and an end cap 21066 to restrict axial movement of the proximal member 21044 within the passage 21027. The stop 21064 can be configured in a variety of ways. In some implementations, the stop 21064 is configured to inhibit movement of the proximal member 21044 distally within the passage 21027.

In some implementations, the stop 21064 is formed as a ring having a central passage 21068 sized to receive a portion of the proximal member 21044 therethrough. In some implementations, the stop 21064 is further sized to be received within the passage 21027.

In some implementations, the stop 21064 can include structure configured to fix the position of the stop 21064 relative to the expandable/expansion member 21018. In some implementations, the stop 21064 includes a pair of opposed projections 21070 configured to be received within the second pair of radially opposed openings 21038 of the expandable/expansion member 21018, as shown in FIGS. 90-93.

The end cap 21066 can be configured in a variety of ways. In some implementations, the end cap 21066 is configured to inhibit movement of the proximal member 21044 proximally within the passage 21027. In some implementations, the end cap 21066 is formed as a ring having a distal end 21072, a proximal end 21074 opposite the distal end 21072, and a central passage 21076 extending through the end cap 21066 from the distal end 21072 to the proximal end 21074.

In some implementations, the central passage 21076 is sized to receive at least a portion of the proximal end 21056 of the proximal member 21044. In some implementations, the proximal end 21056 of the proximal member 21044 can further include one or more engagement surfaces 21077 accessible through the central passage 21076 of the end cap 21066. In some implementations, the one or more engagement surfaces 21077 are configured to be engaged to rotate the proximal member 21044.

In some implementations, the end cap 21066 is further sized to be received within the passage 21027. In some implementations, the end cap 21066 can include structure configured to fix the position of the end cap 21066 relative to the expandable/expansion member 21018.

In some implementations, the end cap 21066 includes a pair of opposed projections 21078 at the distal end 21072 that are configured to be received within the first pair of radially opposed openings 21036 of the expandable/expansion member 21018, as shown in FIGS. 90-93.

In some implementations, the end cap 21066 can include a radial flange 21080 at the proximal end 21074 configured to inhibit over insertion of the end cap 21066 into the passage 21076.

Referring to FIGS. 90-93, in some implementations, when assembled, the actuation mechanism 21020 can move the expandable/expansion member 21018 between the first and the second configurations. In particular, the distal member 21042 is received within the passage 21027 of the expandable/expansion member 21018.

In some implementations, the pair of opposed projections 21054 on the distal member 21042 are received within the third pair of radially opposed openings 21040 thus both axially and rotationally fixing the distal member 21042 relative to the expandable/expansion member 21018.

In some implementations, the distal end portion 21024 of the expandable/expansion member 21018 and/or the distal member 21042 can be configured to move or flex to allow the pair of opposed projections 21054 to be positioned adjacent the third pair of radially opposed openings 21040.

In some implementations, the distal end portion 21024 of the expandable/expansion member 21018 includes one or more open-ended longitudinal slots 21082 that allow the distal end portion 21024 to flex outward as the pair of opposed projections 21054 are moved into position to be received within the third pair of radially opposed openings 21040. In some implementations, once the pair of opposed projections 21054 are received within the third pair of radially opposed openings 21040, the distal end portion 21024 returns back to its normal state.

In some implementations, the stop 21064 is received within the passage 21027 such that the pair of opposed projections 21070 are received within the second pair of radially opposed openings 21038 of the expandable/expansion member 21018. In some implementations, the proximal end portion 21022 of the expandable/expansion member 21018 and/or the stop 21064 can be configured to move or flex to allow the pair of opposed projections 21070 to be positioned adjacent the second pair of radially opposed openings 21038.

In some implementations, the proximal end portion 21022 of the expandable/expansion member 21018 includes one or more open-ended longitudinal slots 21084 that allow the proximal end portion 21022 to flex outward as the pair of opposed projections 21070 are moved into position to be received within the second pair of radially opposed openings 21038. In some implementations, once the pair of opposed projections 21070 are received within the second pair of radially opposed openings 21038, the proximal end portion 21022 returns back to its normal state.

In some implementations, the proximal member 21044 is received within the passage 21027 proximate to the distal member 21042. In some implementations, the distal end 21058 of the proximal member 21044 is received in the passage 21050 in the proximal end 21046 of the distal member 21042 such that the male threads 21060 of the proximal member 21044 threadably engage the female threads 21052 of the distal member 21042. In some implementations, the proximal member 21044 is threaded into the distal member 21042 until the ridge 21062 engages the stop 21064.

In some implementations, the distal end 21072 of the end cap 21066 is received within the passage 21027 such that the pair of opposed projections 21078 are received within the first pair of radially opposed openings 21036 of the expandable/expansion member 21018. The proximal end portion 21022 of the expandable/expansion member 21018 and/or the end cap 21066 can be configured to move or flex to allow the pair of opposed projections 21078 to be positioned adjacent the first pair of radially opposed openings 21036.

In some implementations, the proximal end portion 21022 of the expandable/expansion member 21018 includes the open-ended longitudinal slots 21084 that allow the proximal end portion 21022 to flex outward as the pair of opposed projections 21078 are moved into position to be received within the first pair of radially opposed openings 21036. In some implementations, once the pair of opposed projections 21078 are received within the first pair of radially opposed openings 21036, the proximal end portion 21022 returns back to its normal state.

As shown in FIGS. 90-91, in some implementations, when assembled, the ridge 21062 is sandwiched between the end cap 21066 and the stop 21064 such that axial movement of the proximal member 21044 relative to the proximal end portion 21022 of the expandable/expansion member 21018 is inhibited. Rotational movement of the proximal member 21044, however, is allowed.

In some implementations, a rotational device or tool (not shown) can engage the one or more engagement surfaces 21077 via the passage 21076 in the end cap 21066 to rotate the proximal member 21044 in a first rotational direction (i.e., to thread the proximal member 21044 into the distal member 21042). With the proximal member 21044 axially fixed to the proximal end portion 21022 of the expandable/expansion member 21018 and the distal member 21042 axially fixed to the distal end portion 21024 of the expandable/expansion member 21018, threading the proximal member 21044 into the distal member 21042 draws the proximal end portion 21022 of the expandable/expansion member 21018 toward the distal end portion 21024. As a result, the strips 21028 of the intermediate portion 21026 bend or flex outward. Since the intermediate portion 21034 of the strips 21028 are attached to the posts 21012 of the frame member 21002, the outward expansion of the strips 21028 causes the outward expansion of the frame member 21002.

Conversely, in some implementations, rotating the proximal member 21044 in a second rotational direction (i.e., to thread the proximal member 21044 out of the distal member 21042) pushes the proximal end portion 21022 of the expandable/expansion member 21018 away from the distal end portion 21024. As a result, the strips 21028 of the intermediate portion 21026 are pulled inward causing the inward contraction of the frame member 21002.

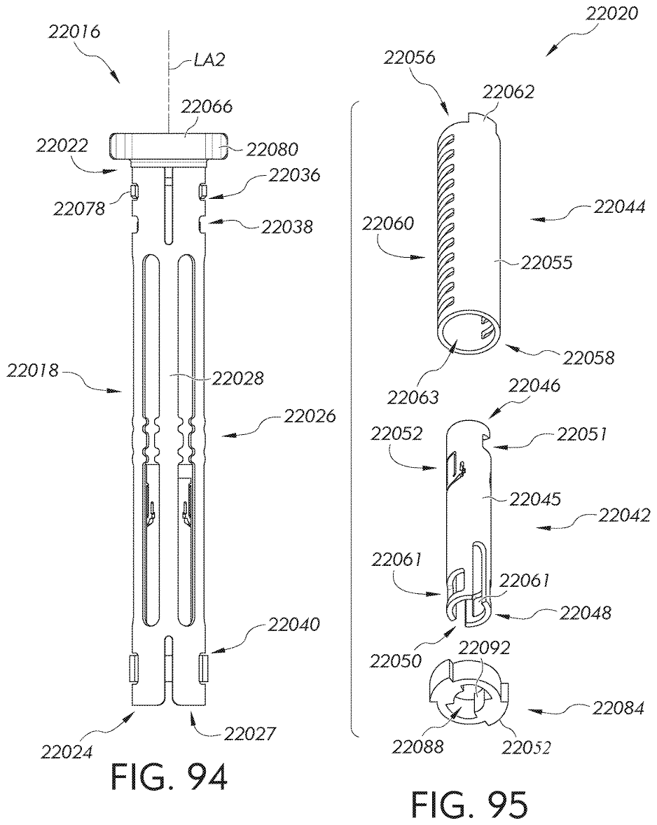

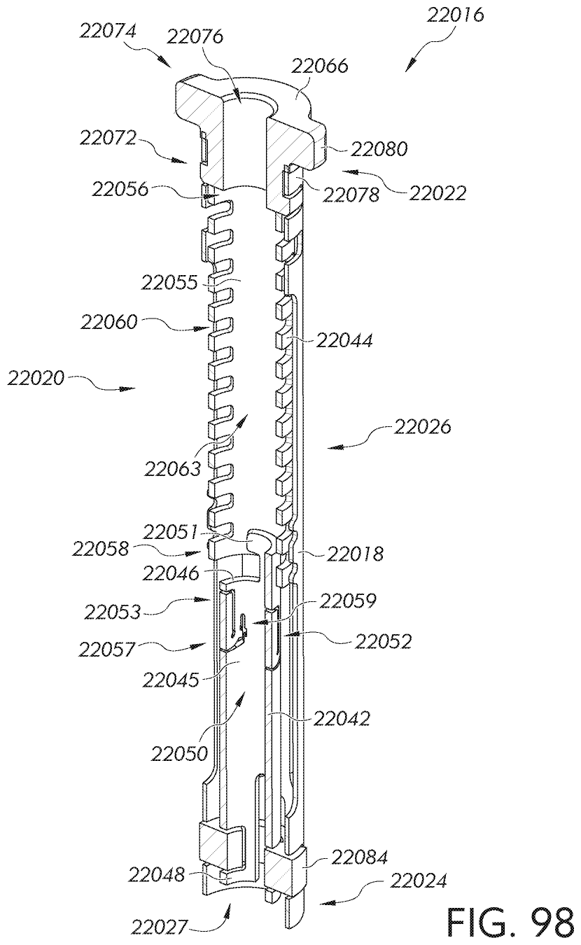

FIGS. 94-98 illustrate an example expandable mechanism 22016 usable in an expandable coaptation element. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism 22016 can be positioned and/or mounted inside an expandable frame member, such as the frame member 21002 (FIG. 82-83). The example expandable mechanism 22016 includes an expandable/expansion member 22018 and an actuation mechanism 22020 (FIGS. 94-95)

The expandable/expansion member 22018 can be configured in a variety of ways. In the illustrated example, the expandable/expansion member 22018 is substantially similar to the expandable/expansion member 21018 of FIGS. 87-88, thus the description of the expandable/expansion member 21018 applies equally to the expandable/expansion member 22018.

In some implementations, the expandable/expansion member 22018 is configured as an elongated cylindrical shape having a proximal end portion 22022, a distal end portion 22024, an intermediate portion 22026, and a passage 22027 extending through the expandable/expansion member 22018 from the proximal end portion 22022 to the distal end portion 22024.

In some implementations, the intermediate portion 22026 can comprise and/or be formed from a plurality of strips 22028 (FIG. 94) that are configured to bend or flex.

In some implementations, the expandable/expansion member 22018 can include structure configured to mount the actuation mechanism 22020 within the expandable/expansion member 22018. In some implementations, the proximal end portion 22022 of the expandable/expansion member 22018 includes a first pair of radially opposed openings 22036, and a second pair of radially opposed openings 22038 distal to the first pair of radially opposed openings 22036 and the distal end portion 22024 of the expandable/expansion member 22018 includes a third pair of radially opposed openings 22040.

In some implementations, the expandable/expansion member 22018 is movable between a first configuration (i.e., collapsed or narrow configuration) and a second configuration (i.e., expanded or wide configuration). As shown in FIG. 94, in some implementations, in the first configuration, each of the plurality of strips 22028 extend parallel to a longitudinal axis LA2 of the expandable/expansion member 22018 and, in the second configuration (see FIG. 87), the strips 22028 bend or flex outward such that the intermediate portion 22026 expands radially outward.

In some implementations, the actuation mechanism 22020 is configured to move the expandable/expansion member 22018 between the first and the second configurations. The actuation mechanism 22020 can be configured in a variety of ways. Referring to FIGS. 95-98, in some implementations, the actuation mechanism 22020 includes a distal member 22042 and a proximal member 22044.

The distal member 22042 and the proximal member 22044 can be configured in a variety of ways. In the illustrated example, the distal member 22042 is formed as a cylindrical tube having a side wall 22045 with a circular cross section. In some implementations, however, the distal member 22042 can have a shape other than cylindrical (e.g., a cross section that is oval, rectangular, elliptical, or other suitable shape).

In some implementations, the distal member 22042 includes a proximal end 22046, a distal end 22048 opposite the proximal end 22046, and an inner passage 22050 extending through the distal member 22042 member from the proximal end 22046 to the distal end 22048. In some implementations, the proximal end 22046 includes a coupling portion 22051 configured to couple to an actuation element (e.g., actuation shaft, actuation rod, actuation tube, actuation wire, etc.) (not shown).

In some implementations, the distal member 22042 includes one or more locking members 22052 configured to engage the proximal member 22044 to lock the position of the distal member 22042 relative to the proximal member 22044. The one or more locking members 22052 can be configured in a variety of ways.

In the illustrated example, each locking member 22052 can comprise and/or be formed from the side wall 22045. For example, each locking member 22052 can be formed from laser cuts through the side wall 22045. Each locking member 22052 can include a first end 22053, a second end 22057 opposite the first end 22053, and an intermediate portion between the first end 22053 and the second end 22057.

In some implementations, each locking member 22052 is configured to pivot about the intermediate portion 22059 between a first position in which the locking member 22052 is parallel with the side wall 22045, as shown in FIGS. 95-98, and a second position (not shown) in which the locking member 22052 pivots such that the first end 22053 extends external to the side wall 22045 and the second end 22057 extends into the passage 22050.

In some implementations, each locking member 22052 is biased to the second position. For example, at least a portion of the distal member 22042 can include a shape memory alloy and the one or more locking members 22052 can be shape set to the second position.

In some implementations, the distal member 22042 is sized to be received within the passage 22027 of the expandable/expansion member 21018. In some implementations, the distal member 22042 can include structure for fixing the distal member 22042 in position within the passage 22027 relative to the expandable/expansion member 22018. In the illustrated example, the distal end 22048 includes a pair of apertures 22061.

In some implementations, the proximal member 22044 is formed as a cylindrical tube having a sidewall 22055 with a circular cross section. In some implementations, however, the proximal member 22044 can have a shape other than cylindrical (e.g., a cross section that is oval, rectangular, elliptical, or other suitable shape).

In some implementations, the proximal member 22044 includes a proximal end 22056, a distal end 22058 opposite the proximal end 22056, and an inner passage 22063 extending through the proximal member 22044 from the proximal end 22056 to the distal end 22058. In some implementations, the proximal member 22044 includes structure configured to interact with the one or more locking members 22052 of the distal member 22042. In some implementations, the structure is formed as two series of slots 22060 formed in the sidewall 22055 on opposite sides of each other. Each slot 22060 can be arranged transverse (e.g., perpendicular) to the longitudinal axis LA2 and each series of slots 22060 can be aligned with a locking member 22052.

In some implementations, the proximal member 22044 is sized to be received within the passage 22027 of the expandable/expansion member 22018. In some implementations, the proximal member 22044 includes structure for restricting rotational movement of the proximal member 22044 within the passage 22027 relative to the expandable/expansion member 22018. The structure for restricting axial movement of the proximal member 21044 can be configured in a variety of ways. In some implementations, the proximal member 22044 includes one or more tabs 22062 extending axially from the proximal end 22056.

In some implementations, the actuation mechanism 22020 includes an end cap 22066. The end cap 22066 can be configured in a variety of ways. In some implementations, the end cap 22066 is configured to inhibit movement of the proximal member 22044 proximally and rotationally within the passage 22027.

In some implementations, the end cap 22066 is formed as a ring having a distal end 22072, a proximal end 22074 opposite the distal end 22072, and a central passage 22076 extending through the end cap 22066 from the distal end 22072 to the proximal end 22074. In some implementations, the end cap 22066 is configured to receive each of the one or more tabs 22062 in a groove or recess 22077 to inhibit rotation of the proximal member 22044 within the passage 22027.

In some implementations, the distal end 22072 of the end cap 22066 is sized to be received within the passage 22027. In some implementations, the end cap 22066 can include structure configured to fix the position of the end cap 22066 relative to the expandable/expansion member 22018.

In some implementations, the end cap 22066 includes a pair of opposed projections 22078 at the distal end 22072 that are configured to be received within the first pair of radially opposed openings 22036 of the expandable/expansion member 22018, as shown in FIGS. 90-93. In some implementations, the end cap 22066 can include a radial flange 22080 at the proximal end 22074 configured to inhibit over insertion of the end cap 22066 into the passage 22076.

In some implementations, the actuation mechanism 22020 includes a stop 22084 (FIG. 95) to restrict movement of the distal member 22042 relative to the expandable/expansion member 22018. The stop 22084 can be configured in a variety of ways. In some implementations, the stop 22084 is formed as a ring having a central passage 22088 sized to receive the distal end 22048 of the distal member 22042 therein.

In some implementations, the stop 22084 is further sized to be received within the passage 22027 of the expandable/expansion member 22018. In some implementations, the stop 22084 can include structure configured to fix the position of the distal member 22042 relative to the expandable/expansion member 22018. In some implementations, the stop 22084 includes a pair of opposed first projections 22090 extending radially outward and configured to be received within the third pair of radially opposed openings 22040 of the expandable/expansion member 22018, as shown in FIGS. 94 and 97. In some implementations, the stop 22084 includes a pair of opposed second projections 22092 extending radially inward and configured to be received within the pair of apertures 22061 at the distal end 22048 of the distal member 22042.

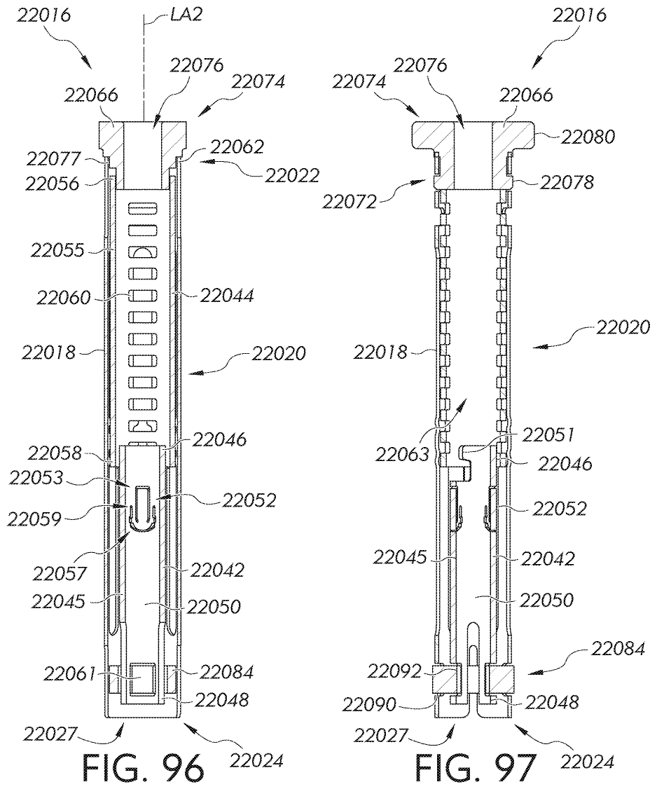

In some implementations, when assembled, the actuation mechanism 22020 can move the expandable/expansion member 22018 between the first and the second configurations. Referring to FIGS. 96-98, the distal member 22042 is received within the passage 22027 of the expandable/expansion member 21018. In some implementations, the stop 22084 is positioned within the 22027 such that the pair of opposed first projections 22090 are received within the third pair of radially opposed openings 22040 of the expandable/expansion member 22018 and the pair of opposed second projections are received within the pair of apertures 22061 at the distal end 22048 of the distal member 22042. As a result, the distal member 22042 is held in position relative to the expandable/expansion member 22018.

Further, in some implementations, the proximal member 22044 is received within the passage 22027 of the expandable/expansion member 21018 and the proximal end 22046 of the distal member 22042 is received within the passage 22063 of the proximal member 22044. In some implementations, the distal end 22072 of the end cap 22066 is secured to the proximal member.

In some implementations, the distal end 22072 of the end cap 22066 can be received within the passage 22027 such that the projections 22078 of the end cap 22066 are received in the first pair of radially opposed openings 22036 of the expandable/expansion member 22018. As a result, the end cap 22066 fixes the proximal member 22044 at the proximal end portion 22022 of the expandable/expansion member 22018.

In some implementations, the proximal member 22044 is separate from the end cap 22066 and the proximal member 22044 is separately attached to the expandable/expansion member 22014. For example, the proximal member 22044 can be secured to the expandable/expansion member 22014 in the same or similar manner that the proximal member 21044 is attached to the expandable/expansion member 22014 in the implementation illustrated by FIGS. 89-93.

In some implementations, the device (e.g., device 100 or another device herein) can be delivered by a delivery system (e.g., delivery system 102). In some implementations, the delivery system can include a first actuation element (e.g., actuation shaft, actuation rod, actuation tube, actuation wire, etc.) (not shown) that extends through the passage 22076 in the end cap 22066, through the passage 22063 in the proximal member 22044, and couples to the coupling portion 22051 at the proximal end 22046 of the distal member 22042.

In some implementations, the delivery system can include an optional second actuation element (e.g., actuation shaft, actuation rod, actuation tube, actuation wire, etc.) (not shown) that extends coaxially through the first actuation element (not shown) and into the passage 22050 of the distal member 22042.

In some implementations, with the first and/or second actuation member (not shown) in position within the passage 22050 adjacent the one or more locking members 22052, the locking members 22052 are held in the first configuration by the first actuation member and/or the second actuation member. Conversely, when the first actuation member and/or the second actuation member (not shown) is moved within the passage 22050 such that the first and/or second actuation member (not shown) is not adjacent the one or more locking members 22052, the locking members 22052 move to their second configuration (e.g., the outwardly extending, locking configuration).

In some implementations, to move the expandable/expansion member 22018 between the first configuration and the second configuration, the distal member 22042 can be moved axially within the passage 22063 of the proximal member 22044.

In some implementations, the distal member 22042 can be moved proximally within the passage 22063 by pulling on the first actuation element (not shown) which is coupled to the proximal end 22046 of the distal member 22042. Since the distal end 22048 of the distal member 22042 is fixed to the distal end portion 22024 of the expandable/expansion member 22018 by the stop 22084 and the end cap 22066 fixes the proximal member 22044 in place relative to the expandable/expansion member 22018, pulling on the first actuation element (not shown) with sufficient force moves the distal end portion 22024 of the expandable/expansion member 22018 closer to the proximal end portion 22022 of the expandable/expansion member 22018. As a result, the strips 22028 of the intermediate portion 22026 bend or flex outward. Since the strips 22028 are attached to the frame member (e.g., frame member 21002), the outward expansion of the strips 22028 causes the outward expansion of the frame member.

In some implementations, once the expandable/expansion member 22018, and thus the expandable coaptation element, has been expanded to the desired width or diameter, the distal member 22042 can be locked in position relative to the proximal member 22044. In particular, the first actuation member and/or second actuation member (not shown) can be moved axially within the passage 22050 such that the first actuation member and/or the optional second actuation member (not shown) is not adjacent the one or more locking members 22052. As a result, one or more locking members 22052 pivot to their second configuration resulting in the first end 22053 of each of the locking members 22052 engaging with one of the slots 22060 in the proximal member 22044. In some implementations, the engagement of the first end 22053 of each of the locking members 22052 with a corresponding slot 22060 locks the distal member 22042 from moving axially within the passage 22063 in the proximal direction.

In some implementations, the frame member (e.g., frame member 21002) and/or the expandable/expansion member 22018 can be configured to be normally in the first configuration (i.e., collapsed or narrow), in the second configuration (i.e., expanded or wide), or some intermediate position between the first and the second configuration. In some implementations, movement of the distal member 22042 relative to the proximal member 22044 pulls the frame member and/or the expandable/expansion member 22018 inward toward the first position against a bias of the frame member and/or the expandable/expansion member 22018 or, in some implementations, pushes the frame member and/or the expandable/expansion member 22018 outward to the second position against a bias of the frame member and/or the expandable/expansion member 22018.

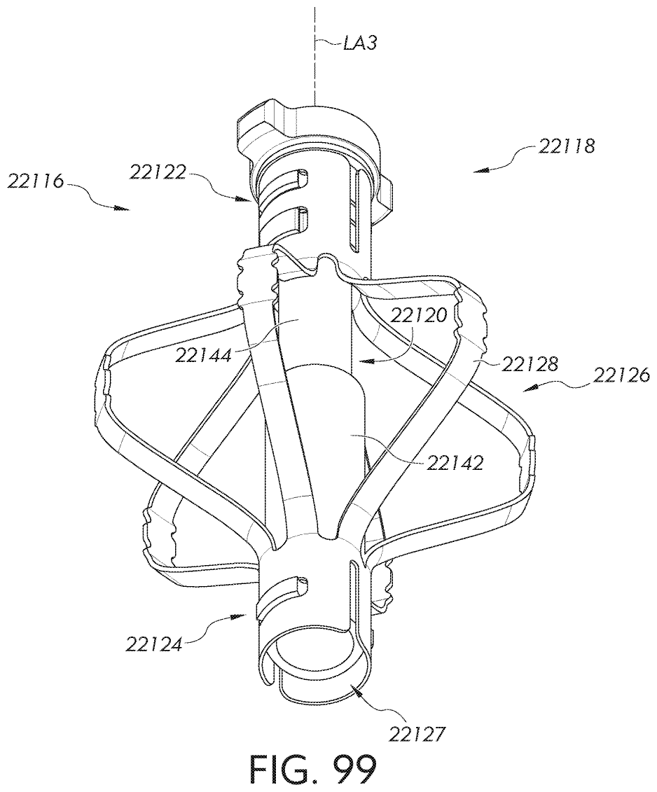

FIG. 99 illustrates an example expandable mechanism 22116 useable and/or for use in an expandable coaptation element. The expandable mechanism 22116 can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism 22116 can be positioned and/or mounted inside an expandable frame member, such as the frame member 21002 (FIG. 82-83). The example expandable mechanism 22116 includes an expandable/expansion member 22118 and an actuation mechanism 22120. The expandable/expansion member 22118 can be configured in a variety of ways, e.g., the same as or similar to any other expandable/expansion members herein. The actuation mechanism 22120 can be configured in a variety of ways, e.g., the same as or similar to any other actuation mechanisms herein.

In the illustrated example, the expandable/expansion member 22118 is substantially similar to the expandable/expansion member 21018 of FIGS. 87-88, thus the description of the expandable/expansion member 21018 applies equally to the expandable/expansion member 22118. In particular, the expandable/expansion member 22118 is configured as an elongated cylindrical shape having a proximal end portion 22122, a distal end portion 22124, an intermediate portion 22126, and a passage 22127 extending through the expandable/expansion member 22118 from the proximal end portion 22122 to the distal end portion 22124. In some implementations, the intermediate portion 22126 can comprise and/or be formed from a plurality of strips 22128 that are configured to bend or flex.

In some implementations, the expandable/expansion member 22118 is movable between a first configuration (i.e., collapsed or narrow configuration) (e.g., FIG. 88 for expandable/expansion member 21018) and a second configuration (i.e., expanded or wide configuration), as shown in FIG. 99. In some implementations, in the first configuration, each of the plurality of strips 22128 extend parallel to a longitudinal axis LA3 of the expandable/expansion member 22118 and, in the second configuration, the strips 22128 bend or flex outward such that the intermediate portion 22126 expands radially outward.

The actuation mechanism 22120 can be configured in a variety of ways, e.g., in any of the ways other actuation mechanisms are configured herein. In the illustrated example, the actuation mechanism 22120 includes a distal member 22142 and a proximal member 22144. In some implementations, the distal member 22142 and proximal member 22144 are arranged coaxially in a telescoping fashion such that a portion of the proximal member 22144 is received within the distal member 22142 and can move axially relative to the distal member 22142 or vice versa. In some implementations, the movement of the distal member 22142 and the proximal member 22144 axially relative to each other moves the expandable/expansion member 22118 between a first configuration and a second configuration. The distal member 22142 and the proximal member 22144 are movable relative to each other by any suitable means, such as for example, any of the actuation mechanisms disclosed herein.

FIGS. 100-101 illustrate an example expandable coaptation element 21000 as part of a device e.g., a treatment device, repair device, valve treatment device, valve repair device, implantable device, implant, etc.) that is configured to be positioned within a native heart valve to allow the native heart valve to form a more effective seal. The expandable coaptation element 21000 is shown attached to gripping members (e.g., gripping arms, clasp arms, etc.), such as for example, gripping members 130.

In some implementations, the gripping members 130 can be used to attach the expandable coaptation element 21000 to native leaflets of a native heart valve. For example, the gripping members 130 can be opened to accept leaflets of a native mitral valve or a native tricuspid valve and then closed to capture the leaflets and thereby attach the expandable coaptation element 21000 to the native heart valve.

In some implementations, the expandable coaptation element 21000 includes the frame member 21002 and the expandable/expansion member 21018. FIG. 100 illustrates the expandable coaptation element 21000 in a first configuration (i.e., collapsed or narrow) and FIG. 101 illustrates the expandable coaptation element 21000 in a second configuration (i.e., expanded or wide). The expandable coaptation element 21000 can be moved between the first configuration and the second configuration by any suitable means, such as for example, any of the actuation mechanisms disclosed herein.

In some implementations, the expandable coaptation element 21000 can include an optional cover over the frame member 21002. The cover can cover a portion or all of the frame member 21002.

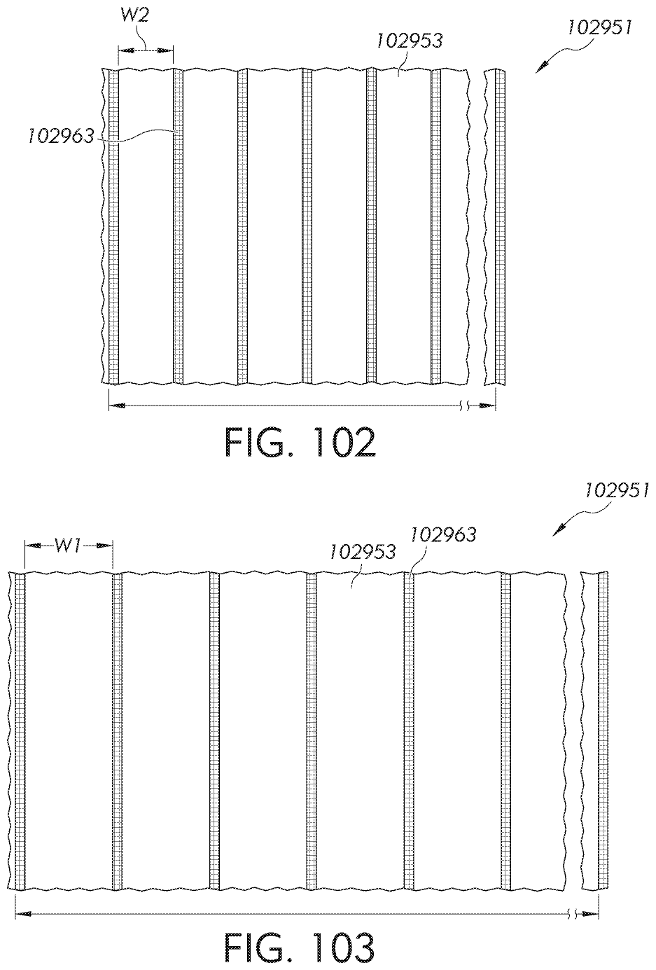

Referring to FIGS. 102-110, an example of a cover 102951 for the expandable coaptation element 21000 is shown. The cover 102951 can be used with any suitable expandable coaptation element 21000, such as, for example, any expandable coaptation element 21000 described in the present application. In some implementations, the cover 102951 is configured to be attached to frame member 21002. However, it should be understood that the cover 102951 can be configured for connecting to any component of the device.

The cover 102951 can be configured in a variety of ways. In some implementations, the cover 102951 can include a sheet, material, fabric, layer or membrane that is attached to the frame member 21002 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth. The cover 102951 can be configured to cover or not cover any component or portion of the device.

In some implementations, the cover 102951 can have one or more stretchable portions 102953 that allow the cover 102951 to maintain a substantially taut state when in a normal position, while also allowing the cover 102951 to stretch to an expanded state. This is advantageous for situations in which the cover 102951 is attached to components of the device, such as the expandable coaptation element 21000, that are movable between narrowed and expanded configurations. That is, the cover 102951 maintaining a taut state when in a normal position, and when expandable coaptation element 21000 are in a narrowed configuration, reduces any excess material on the device that can contact vasculature.

In some implementations, the stretchability of the cover 102951 then allows the expandable coaptation element 21000 to move to an expanded state with the cover 102951 maintaining a substantially taut state and maintaining a covering for the device, which can help prevent or inhibit regurgitation of blood. The stretchable portions 102953 can take a wide variety of different forms. Any material that can stretch and return to its original size or substantially to its original size can be used.

Referring to FIGS. 102-103, a portion of an example cover 102951 is shown as a flat sheet of material or formed as a flat sheet. In some implementations, the cover 102951 includes different shaped segments or portions to attach to different portions of the frame member 21002. In some implementations, the cover 102951 can be shaped to smooth transitions between various portions of the device to reduce catch points and provide a smoother exterior to the device.

In the illustrated example, the cover 102951 includes a plurality of plain weave portions 102963 spaced apart and connected by stretchable portions 102953. In some implementations, the plain weave portions 102963 are configured to attach to the frame member 21002. For example, each of the plain weave portions 102963 can attach to a corresponding post 21012 of the frame member 21002.

In some implementations, when the frame member 21002 expands from the first configuration (i.e., collapsed or narrow), as shown in FIG. 102, to the second configuration (i.e., expanded or wide), as shown in FIG. 103, the stretchable portions 102953 stretch laterally from a width W2 to a width W1, which is larger than the width W2 to accommodate the expansion of the frame member 21002.

Referring to FIG. 105, the plain weave portions 102963 can include weft yarns 102965 and warp yarns 102967 that are woven in a perpendicular weaving pattern. However, the plain weave portions 102963 can take any other suitable form, or the cover 102951 can include any other type of main weaving pattern with the stretchable portions 102953 positioned therein. In some implementations, the plain weave portions 102963 is made of a woven biocompatible fabric that is configured to promote tissue ingrowth. The plain weave portions 102963 can also be configured to reduce blood regurgitation.

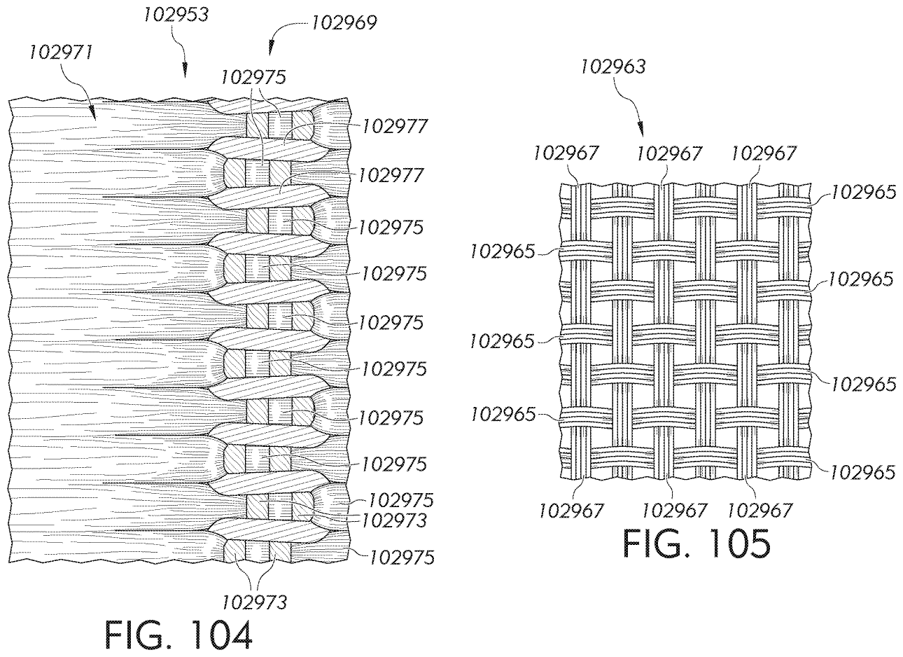

Referring to FIGS. 104 and 106-107, in some implementations, the stretchable portions 102953 can include a pair of transition portions 102969, such as Leno weaves, with a stretchable material or float 102971 connected therebetween. In some implementations, the transition portion, such as a Leno weave, provides a transition, connection or interface between the plain weave 102963 and the stretchable material or float.

Referring to FIG. 104, the float 102971 can include a portion of threads that are not interlaced with any other threads. In some implementations, the floats 102971 include textured yarn. However, other configurations are contemplated.

In some implementations, the Leno weaves 102969 can include warp yarns 102973 and weft yarns 102975 that are woven in a perpendicular weaving pattern with at least one Leno yarn 102977 wrapping the warp yarns 102973.

In the illustrated example, a single Leno yarn 102977 wraps around four warp yarns 102973, and the weft yarns 102975 are extensions of the yarns from the float 102971. In some implementations, the weft yarns 102975 from the Leno weave 102969 extends to the weft yarns 102965 of the plain weave 102963 (FIG. 105). In some implementations, the plain weave 102963 is separate from the Leno weave 102969, and the weft yarns 102975 of the Leno weave 102969 are folded back over or otherwise connected to the warp yarns 102973 and/or Leno yarn 102977 of the Leno weave 102969. However, other configurations for the stretchable portions 102953 are contemplated. The Leno weave 102969 produces an open fabric that prevents or inhibits slippage or misplacement of threads. The Leno weave 102969 can provide further resistance to blood regurgitation.

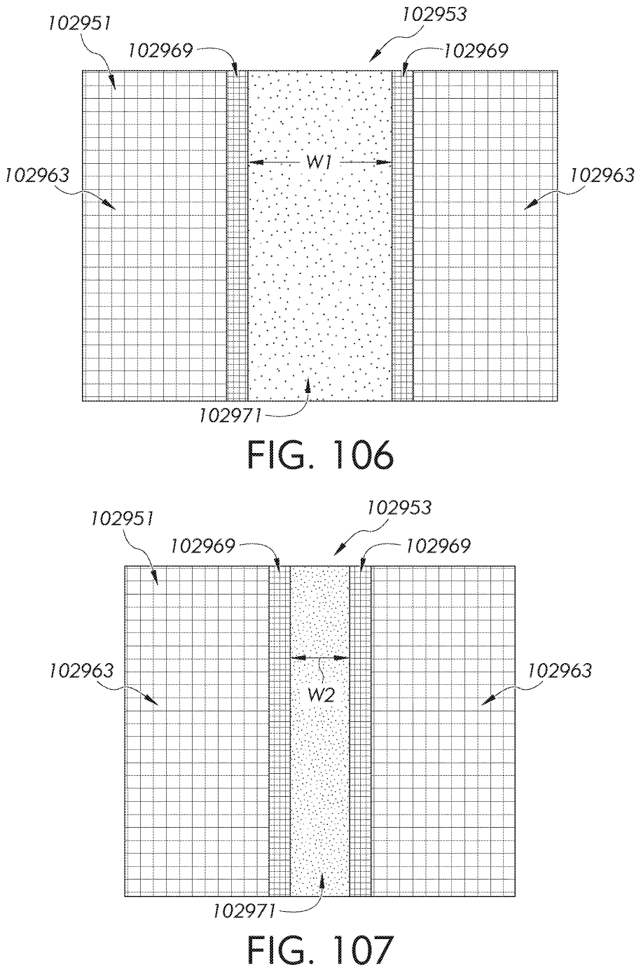

Referring to FIGS. 106-107, in some implementations, the stretchable portions 102953 can be created by taking a cover 102951 that includes the plain weave 102963, two or more Leno weaves 102969, and one or more floats 102971 described above in an initial state (as shown in FIG. 106) and heating the cover 102951 such that the floats 102971 shrink to a narrowed state (as shown in FIG. 107). For example, the yarn of the floats 102971 can crimp and/or curl during heating and cause the floats 102971 to move to the narrowed state. Heat setting or heat pressing the cover 102951 can cause the floats 102971 to shrink to the narrowed state.

In some implementations, once the floats 102971 shrink to the narrowed state, the cover 102951 normally has a width that is based on the floats being in the narrowed state, but the floats can be pulled to expand to a stretched state such that allows the width of the cover 102951 to expand when tension is applied to the cover. For example, pulling the stretchable material or float can temporarily straighten out the curled and/or kinked strands of the stretchable material or float.

In some implementations, removal of the tension from the cover 102951 causes the floats to move back to the narrowed state such that the cover 102951 moves back to the normal position. That is, the strands pull back to the curled and/or kinked heat set configuration.

Heating the cover 102951 can also provide advantages to the plain weave 102963. For example, heat setting or heat pressing the plain weave 102963 can reduce the pore size and/or increase the density of the plain weave 102963, which can be advantageous in preventing or inhibiting blood regurgitation.

In some implementations, the floats 102971 can have a width W1 (FIG. 106) when in the pre-heated state and a width W2 (FIG. 107) after being heated. In some implementations, the width W1 can be between about 3 mm and about 7 mm or any subrange, and the width W2 can be between about 1 mm and about 3 mm or any subrange. The ratio of the width W1 to the width W2 can be between about 1.1 to 1 and about 7 to 1 or any subrange. However, the width W1 and the width W2 can take any other suitable sizes based on the portion of the device that the cover 102951 is connected to or the desired amount of stretch for the cover 102951.

In some implementations, the cover 102951 is configured to maintain a substantially taut state when in a normal position, while also allowing the cover 102951 to stretch to an expanded state, but the cover 102951 does not include the discrete stretchable portions 102953.

The cover 102951 can be configured to be stretchable and/or resilient in a variety of different ways. In some implementations, the cover 102951 is made to be stretchable by rotating the material of the cover such that the horizontal and vertical yarns of the weave are no longer horizontal and vertical before the cover is cut from the material. For example, the material that forms the cover can be rotated between 30 degrees and 60 degrees, such as between 40 and 50 degrees, such as about 45 degrees or 45 degrees. The rotation of the fabric that forms the material of the cover allows the cover to stretch as the frame member is moved between the narrow and wide configurations. It should be understood, however, that the cover 102951 can be configured to stretch in a wide variety of different ways.

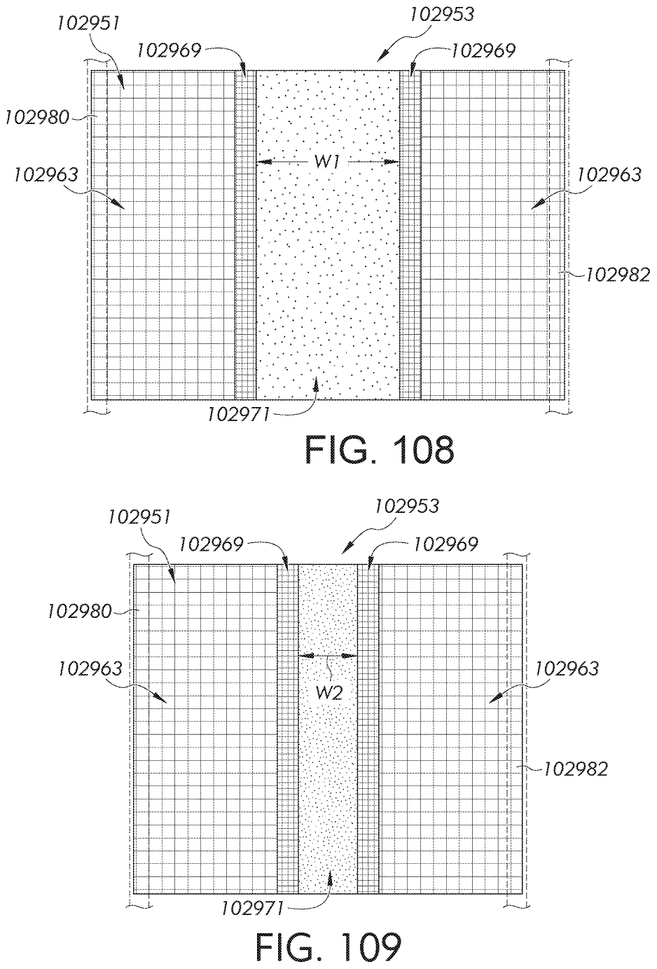

Referring to FIGS. 108-109, in some implementations, the cover 102951 can be attached to a first post 102980 and a second post 102982 of a frame member for an expandable coaptation device (e.g., frame member 21002 of FIG. 82). In the illustrated example, the cover 102951 includes a single stretchable portion 102953 that takes the form of the stretchable portion shown in FIGS. 106-107. However, the cover 102951 can have any suitable number of stretchable portions 102953 between the first and second posts 102980, 102982. Referring to FIG. 108, when the posts 102980, 102982 are in an expanded state, the stretchable portion 102953 can stretch such that the float 102971 has a width W1, which can be substantially identical to or less than the width W1 of the pre-heated float 102971 shown in FIG. 106. Referring to FIG. 109, when the posts 102980, 102982 are in a narrowed state, the stretchable portion 102953 move back to the normal position such that the float 102971 has a width W2, which can be substantially identical to or greater than the width W2 of the heat set float 102971 shown in FIG. 107.

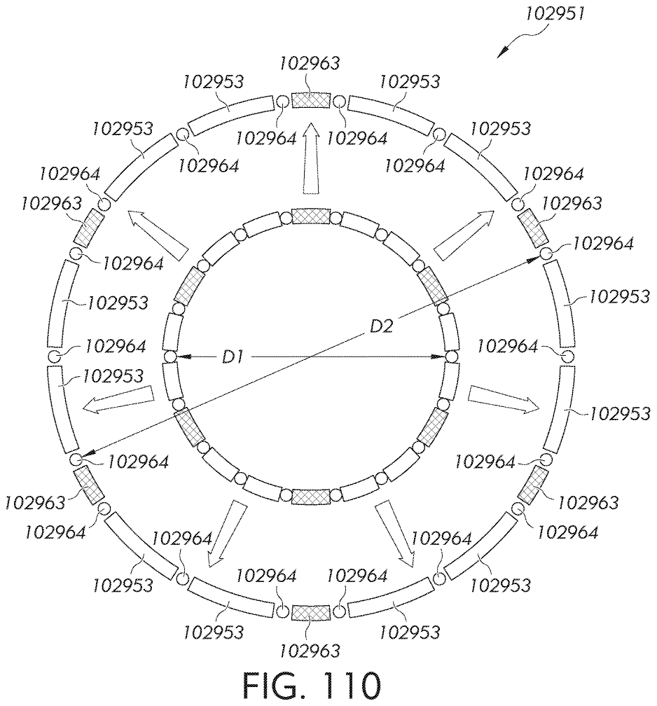

FIG. 110 shows a schematic top view of an example cover 102951 for an expandable coaptation element in both the narrowed state and the expanded state. In some implementations, the cover 102951 includes a plurality of plain weave portions 102963 spaced apart and connected to one or more stretchable portions 102953. In the illustrated example, the cover 102951 includes six plain weave portions 102963 evenly spaced apart. In some implementations, however, the cover 102951 can include more or less than six plain weave portions 102963 and/or the plain weave portions 102963 can be unevenly spaced apart. In some implementations, two or more plain weave portions 102963 can be connected side-by-side.

In the illustrated example, each plain weave portions 102963 is connected to another plain weave portion 102963 by a pair of stretchable portions 102953. In some implementations, however, the cover 102951 can include more or less than two stretchable portions 102953 connecting two plain weave portions 102963. Plain weave portions 102963 can be connected to stretchable portions 102953 or other plain weave portions 102963 and stretchable portions 102953 can be connected to other stretchable portions 102953 in any suitable manner. In the illustrated example, Leno stitches 102964 are used to connect two stretchable portions 102953 together and plain weave portions 102963 to stretchable portions 102953.

As shown in FIG. 110, the cover 102951 can stretch from a first diameter D1 in the first state to a second diameter D2 in the second state. In some implementations, the ratio of D2:D1 is in the range to 1.5:1 to 3:1. As shown in FIG. 110, the stretchable portions 102953 account for the majority of the increase in diameter of the cover 102951 when expanded. For example, in some implementations, the plain weave portion 102963 expand in length less than 5%, less than 4%, less than 3% or less than 2% when the cover expands from the first state to the second state.

Any of the covers disclosed herein, such as the cover 102951 illustrated by FIG. 110 can be formed in a variety of different ways. In some implementations the cover is woven as a tube. For example, the material of the cover 102951 can be woven as a tube around a mandrel and/or by a circular weaving machine.

In some implementations, the cover 102951 can be woven as a flat sheet. Cover material woven as a flat sheet can be formed into a tube or another shape, for example, by connecting two opposed ends of the flat sheet together (e.g., with stitches).

Any of the covers disclosed herein can be woven as a tube, woven as a flat sheet, woven in some other shape/configured, and/or some portion(s) of the cover can be woven as a tube, while other portion(s) of the cover can be woven as a sheet, etc.

In some implementations, a cover, such as any of the covers described herein can be configured and/or treated to reduce blood flow through the cover. In some implementations, covers configured and/or treated to reduce blood flow through the cover can be expandable/stretchable or non-expandable/non-stretchable.

The cover can be configured and/or treated to reduce blood flow through the cover in a variety of different ways. For example, the cover can be configured and/or treated to reduce blood flow through the cover by reducing pore size. The pore size can be reduced in a variety of different ways. For example, the pore size can be reduced by increasing the density of the fabric, by laminating the fabric, by coating the fabric, by layering the fabric, and/or by other means.

The density of the fabric can be increased in a variety of different ways. In some implementations, the ends per inch (EPI) and/or the picks per inch (PPI) of one or more portions of the fabric can be increased to increase the density of the fabric and reduce the permeability of the fabric. For example, the EPI×PPI can be between 160×152 and 400×500 or any sub range. The EPI×PPI for a polyethylene terephthalate (PET) yarn can be between 160×152 and 400×500 or any sub range. The EPI×PPI for a 50% polyethylene terephthalate (PET) and 50% polyolefin (PO) yarn can be between 160×152 and 400×500 or any sub range. The EPI×PPI can be 160×196. The EPI×PPI can be 160×256. The EPI×PPI can be 160×304. The EPI×PPI can be 160×152. The EPI×PPI can be 160×196 for a PET yarn. The EPI×PPI can be 160×256 for a PET yarn. The EPI×PPI can be 160×304 for a PET yarn. The EPI×PPI can be 160×152 for a 50/50 PET and PO yarn.

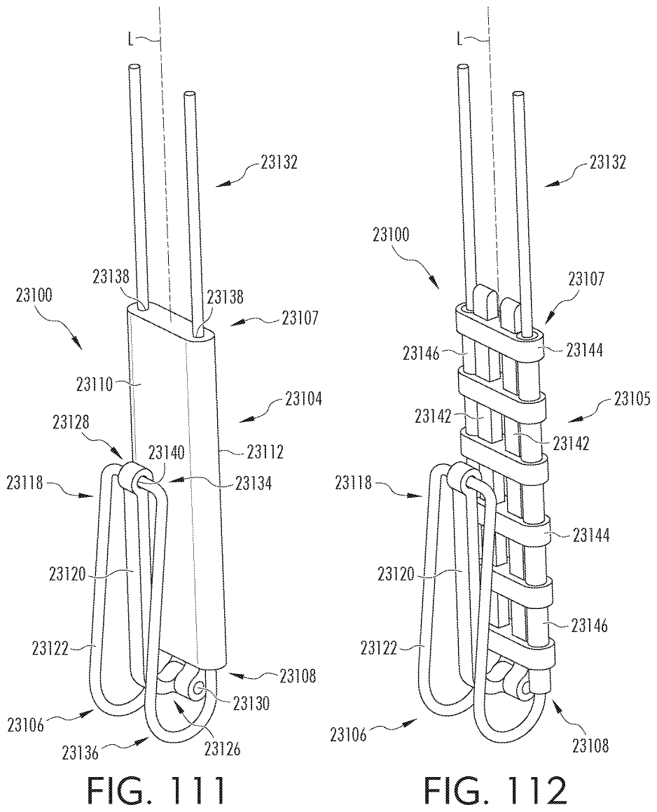

Referring now to FIG. 111, a device or implant 23100 (e.g., a treatment device, a repair device, a prosthetic device, a valve repair device, implantable device, implant, etc. or component thereof). The device 23100 can include any other features for another device or implant discussed in the present application or the applications cited above, and the device 23100 can be positioned to engage valve tissue (e.g., leaflets 20, 22, 30, 32, 34) as part of any suitable treatment and/or repair system (e.g., any treatment and/or repair system disclosed in the present application, or the applications cited herein). Device 23100 can be used as a component (e.g., anchor portion, etc.) of a larger device or implant.

In some implementations, the device or implant 23100 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 23100 includes a body portion 23104 and an anchor portion 23106.

In some implementations, the body portion 23104 of the device or implant 23100 can function as a coaptation element that is adapted to be implanted between leaflets of a native valve (e.g., a native mitral valve, native tricuspid valve, etc.). In some implementations, the body portion 23104 can be attached to a coaptation element (e.g., spacer, coaptation element, gap filler, membrane, sheet, plug, wedge, balloon, frame, etc.) such as any suitable coaptation element (e.g., any coaptation element disclosed in the present application, or the applications cited above).

In some implementations, the body portion 23104 supports the anchor portion 23106. In some implementations, the body portion 23104 can guide movement of the anchor portion 23106 relative to the body portion 23104. The body portion 23104 can be configured in a variety of ways. In the illustrated example, the body portion 23104 includes a proximal portion 23107, a distal portion 23108 opposite the proximal portion 23107, an inner face 23110 extending between the proximal portion 23107 and the distal portion 23108, and an outer face 23112 opposite the inner face 23110.

In some implementations, the inner face 23110 and the outer face 23112 are planar and parallel to each other. In some implementations, however, the inner face 23110 and the outer face 23112 need not planar and/or parallel.

In some implementations, the anchor portion 23106 can include one or more anchors 23118 that are actuatable between open and closed conditions. The anchors 23118 can take a wide variety of forms, such as, for example, links, paddles, gripping elements, or the like. In the illustrated example, the device or implant 23100 includes a single anchor 23118. In the illustrated example, anchor portion 23106 and/or anchors 23118 include a first link or paddle 23120, a second link or paddle 23122 operatively connected to the first link 23120, and a gripping member 23124 (FIG. 114).

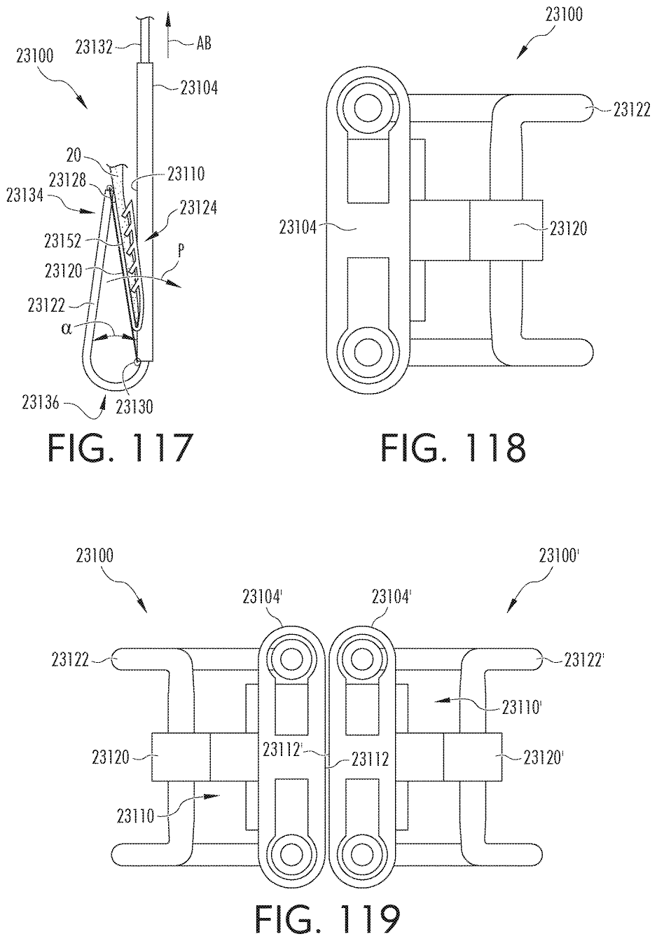

The first link 23120 is connected to both the second link 23122 and the body portion 23104 and is movable between an open configuration (FIG. 114) and a closed configuration (FIG. 117). The first link 23120 can be configured in a variety of ways. In the illustrated example, the first link 23120 has a rigid, elongated body with a rectangular cross-section. In some implementations, however, the first link 23120 can have a non-rectangular cross-section (e.g., oval, circular, etc.). The first link 23120 has a first end 23126 and a second end 23128 opposite the first end 23126.

In some implementations, as shown in the illustrated example, the first end 23126 is connected to the body portion 23104 via a pivot connection 23130 so that the first link 23120 can pivot between open and closed conditions. The pivot connection 23130 can be configured in a variety of ways, such as for example, a pivot pin. In some implementations, the pivot connection 23130 is a bendable or flexible portion of the first end 23126 such that the first end 23126 bends to allow movement of the first link 23120 between open and closed conditions.

In some implementations, as shown in the illustrated example, the second end of the first link 23120 is connected to the second link 23122. In some implementations, the second link 23122 is configured to be movable relative to the body portion 23104 in order to move the anchors 23118 between open and closed conditions. The second link 23122 can be configured in a variety of ways. In the illustrated example, the second link 23122 can comprise and/or be formed from single elongated rod or wire having a circular cross-section. In some implementations, however, the second link 23122 can be formed from two or more pieces and can be shaped other than a circular cross-section.

In some implementations, as shown in the illustrated example, the second link 23122 includes a first portion 23132 that extends through the body portion 23104, a distal portion 23134 that is connected to the second portion 23128 of the first link 23120, and a flexible intermediate portion 23136 connecting the first portion 23132 to the second portion 23128. As shown in FIG. 111, in the illustrated example, the first portion 23132 includes two parallel, linear portions of the second link 23122 extending through passages 23138 in the body portion 23104. In some implementations, the passages 23138 act as guides allowing the second link 23122 to move axially with respect to the body portion 23104.

In some implementations, the second portion 23134 of the second link 23122 is pivotably connected to the second portion 23128 of the first link 23120. The second portion 23134 of the second link 23122 can be pivotably connected to the second portion 23128 of the first link 23120 in any suitable manner. As shown in FIG. 111, in the illustrated example, the second portion 23128 of the first link 23120 includes a passage 23140 through which the second portion 23134 of the second link 23122 passes through.

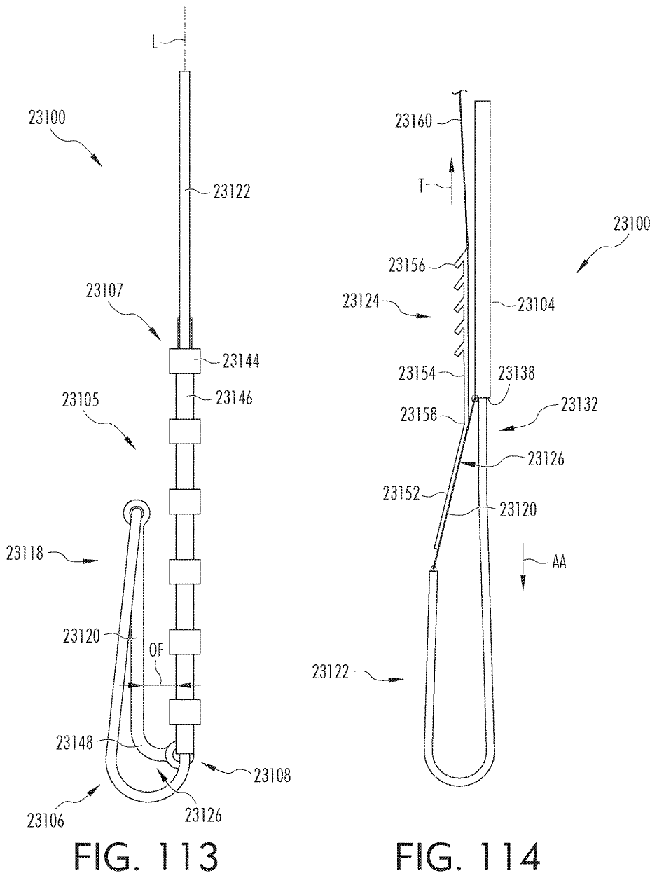

Referring to FIGS. 112-113, the device or implant 23100 is illustrated with a body portion 23105 having a different configuration than that body portion 23104 illustrated in FIG. 111. Other than the configuration of the body portion 23105, the rest of the device illustrated in FIG. 112 is identical to the device illustrated in FIG. 111, thus that reference numbers for like elements are kept the same the description of the device in FIG. 111 applies equally to the device in FIG. 112.

Referring to FIG. 112, the device 23100 includes the body portion 23105 and an anchor portion 23106. The body portion 23105 supports the anchor portion 23106. In some implementations, the body portion 23105 can guide movement of the anchor portion 23106 relative to the body portion 23105. In some implementations, the anchor portion 23106 can include one or more anchors 23118 that are actuatable between open and closed conditions. In the illustrated example, anchor portion 23106 and/or anchors 23118 include a first link 23120, a second link 23122 operatively connected to the first link 23120, and a gripping member 23124 (FIG. 114).

In some implementations, the body portion 23105 includes a proximal portion 23107, a distal portion 23108 opposite the proximal portion 23107, and a longitudinal axis L. The body portion 23105 includes one or more first supports 23142 that extend axially from the proximal portion 23107 to a distal portion 23108. In the illustrated example, the body portion 23105 includes a pair of spaced-apart first supports 23142. In some implementations, the body portion 23105 includes one or more second supports 23144 that extend laterally and connect to the one or more first supports 23142. In the illustrated example, the body portion 23105 includes six spaced-apart second supports 23144.

In some implementations, the body portion 23105 also includes passages 23138 through which the second link 23122 extends. In the illustrated example, the passages 23138 are defined by a pair of tubes 23146 that extend axially from the proximal portion 23107 to a distal portion 23108 and through the second supports 23144.

As best illustrated in FIG. 113, in some implementations, the first end 23126 of the first link 23120 includes a bend or curved section 23148 that provides an offset OF between the second end 23128 of the first link 23120 and the inner face 23110 of the body portion 23104 when the device 23100 is in the closed condition. In some implementations, the first link 23120 extends parallel to and offset from the inner face 23110 when in the closed condition.

Referring to FIG. 114, the device 23100, including the gripping member 23124, is illustrated in an elongated or open condition. The elongated or open condition is suitable for delivery of the device 23100 through a catheter, a sheath, a tube, a channel, a pathway, or the like. The gripping member 23124 can be configured in a variety of ways, such as any gripping member 23124 disclosed herein.

In the illustrated example, the gripping member 23124 is formed as a clasp that includes a base or fixed arm 23152, a moveable arm 23154, optional friction-enhancing elements, other securing structures 23156 (e.g., barbs, protrusions, ridges, grooves, textured surfaces, adhesive, etc.), and a joint portion 23158. The fixed arm 23152 is attached to the first link 23120. In some implementations, the fixed arm 23152 is attached to the first link 23120 with the joint portion 23158 disposed proximate the first end 23126 of the first link 23120.

In some implementations, the joint portion 23158 provides a spring force between the fixed and moveable arms 23152, 23154. The joint portion 23158 can be any suitable joint, such as a flexible joint, a spring joint, a pivot joint, or the like, the fixed arm 23152 is attached to the first link 23120 and remains stationary or substantially stationary relative to the first link 23120 when the moveable arm 23154 are opened to open the gripping member 23124 and expose the optional barbs or other friction-enhancing elements 23156.

In some implementations, the gripping member 23124 is opened by applying tension, as shown by arrow T in FIG. 114, to one or more actuation lines 23160 attached to the moveable arm 23154, thereby causing the moveable arm 23154 to articulate, flex, or pivot at the joint portion 23158. The one or more actuation lines 23160 extend through the delivery system (e.g., through a steerable catheter and/or an implant catheter). Other actuation mechanisms are also possible. The actuation line 23160 can take a wide variety of forms, such as, for example, a line, a suture, a wire, a rod, a catheter, or the like. The gripping member 23124 can be spring loaded so that in the closed position the gripping member 23124 continues to provide a pinching force on the grasped native leaflet.

In the elongated or open condition, the second link 23122 in an extended position relative to the body portion 23104. Movement of the second link 23122 to the extended position relative to the body portion 23104, as shown by arrow AA can be accomplished in a variety of ways. For example, in some implementations, the first portion 23132 of the second link 23122 can be engaged by an actuation element (not shown) that can move the first portion 23132 axially within the passages 23138 in the body portion 23104. The actuation element can take a wide variety of different forms (e.g., as a wire, rod, shaft, tube, screw, suture, line, strip, combination of these, etc.), be made of a variety of different materials, and have a variety of configurations, such as any of the actuation elements disclosed herein.

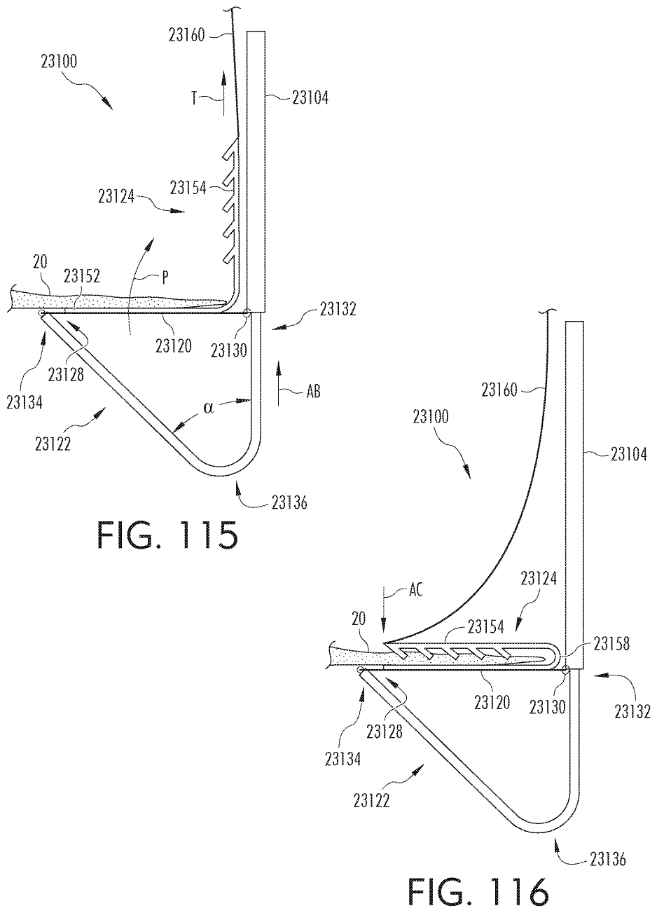

In some implementations, once the device 23100 is positioned within the native valve adjacent a leaflet (e.g., leaflets 20, 22, 30, 32, 34), the device 23100 can be moved to a capture ready condition (i.e., partially closed) as shown in FIG. 115. In some implementations, to move from the elongated condition to the capture ready condition, the second link 23122 is moved proximally relative to the body portion 23104 as shown by arrow AB in FIG. 115 (i.e., the first portion 23132 of the second link 23122 moves axially relative to the body portion 23104 in the proximal direction).

In some implementations, since the second portion 23134 of the second link 23122 is connected to the second portion 23128 of the first link 23120, moving the second link 23122 proximally relative to the body portion 23104 causes the second portion 23128 of the first link 23120 to move with the second portion 23134 of the second link 23122.

As a result, the first link 23120 pivots about the pivot connection 23130, as shown by arrow P in FIG. 115 and the flexible intermediate portion 23136 of the second link 23122 flexes open such that an angle α between the first portion 23132 and the second portion 23134 of the second link 23122 increases. In some implementations, the fixed arm 23152 of the gripping member 23124 moves with the first link 23120 to partially close the gripping member 23124 while the moveable arm 23154 is held open by the actuation line 23160. In some implementations, the gripping member 23124 is spring loaded to the closed condition and thus helps pivot the first link 23120 in the direction of arrow P.

Once the leaflet 20 is in a satisfactory position to be grasped, as shown in FIG. 116, tension in the actuation line 23160 can be released. Releasing tension in the actuation line 23160 allows the spring force associated with the joint portion 23158 to close the gripping member 23124 by moving the moveable arm 23154 toward the fixed arm 23152 and into contact with the leaflet 20, as shown by arrow AC in FIG. 116. to pinch the leaflet 20 between the moveable arm 23154 and the fixed arm 23152.

In some implementations, once the leaflet 20 is captured in the gripping member 23124, the device 23100 can be moved to the closed condition as shown in FIG. 117. To move the device 23100 to the closed position, the second link 23122 is moved proximally relative to the body portion 23104 as shown by arrow AB in FIG. 117 (i.e., the first portion 23132 of the second link 23122 moves axially relative to the body portion 23104 in the proximal direction).

In some implementations, since the second portion 23134 of the second link 23122 is connected to the second portion 23128 of the first link 23120, moving the second link 23122 proximally relative to the body portion 23104 causes the second portion 23128 of the first link 23120 to move with the second portion 23134 of the second link 23122.

As a result, the first link 23120 pivots about the pivot connection 23130, as shown by arrow P in FIG. 115 and the flexible intermediate portion 23136 of the second link 23122 relaxes closed such that the angle α between the first portion 23132 and the second portion 23134 of the second link 23122 decreases from the capture ready condition of FIGS. 115-116.

In some implementations, since the fixed arm 23152 of the gripping member 23124 is attached to the first link 23120, the closed gripping member 23124 moves with the first link 23120. In some implementations where the first link 23120 has the offset OF, as shown in FIG. 113, the closed griping member 23124 can be positioned between the body portion 23104 and the first link 23120 when the device 23100 is in the closed condition. In some implementations where the first link 23120 does not include the offset OF, the body portion 23104 can be contoured to receive a portion of the closed gripping member 23124. For example, the inner face 23110 of the body portion 23104 can have an opening or recess (not shown) the receives a portion of the gripping member 23124.

FIGS. 118-121 illustrate various arrangements for using one or more of the implantable devices or implants 23100. Referring to FIG. 118, a single device or implant 23100 can be used without use of another device or implant 23100. For example, the single device 23100 can be used to capture a single leaflet (e.g., leaflets 20, 22, 30, 32, 34) as part of an overall repair strategy. The single device 23100 can be used with a coaptation element, such as any of the coaptation elements disclosed herein. The single repair device 23100 can also be used to capture multiple leaflets in the anchor 23118, such as both leaflets 20, 22 of the mitral valve MV (FIG. 5) or two or more leaflets 30, 32, 34 of the tricuspid valve TV (FIG. 7).

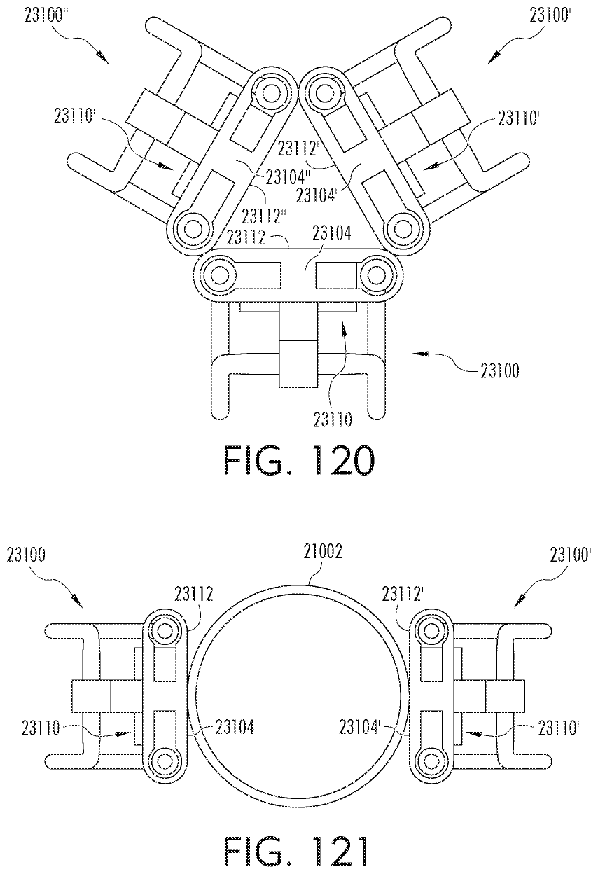

Referring to FIG. 119, in some arrangements, two devices 23100, 23100′ can be arranged back-to-back and secured together. For example, the outer faces 23112, 23112′ of two devices 23100, 23100′ can be attached together so that the two devices 23100, 23100′ are arranged with the inner faces 23110, 23110′ facing in opposite directions. The attached devices 23100, 23100′ can be implanted in a native heart valve where each device 23100, 23100′ can capture a leaflet of the heart valve. For example, the two devices 23100, 23100′ can be implanted in a mitral valve MV where a first device 23100 can capture the anterior leaflet 20 and the second device 23100′ can capture the posterior leaflet 22. Similarly, the two devices 23100, 23100′ can be implanted in a tricuspid valve TV where the two devices 23100, 23100′ capture two or three of the leaflets 30, 32, 34 of the tricuspid valve TV.

In some implementations, each of the devices 23100, 23100′ is independently actuatable. Thus, for example, one of the two devices 23100, 23100′ can be moved to a closed condition to grasp a valve leaflet while the other of the two devices 23100, 23100′ remains in an open or partially closed position.

In some implementations, the two devices 23100, 23100′ can be tethered together. The tethered devices can be separately attached to two separate leaflets, drawn together by the tethers, and then fixed together or setting a distance between the two devices (e.g., by tying the tethers off). The tethered devices can be separately attached to one leaflet and another structure (e.g., chordae tendinea, papillary muscle, etc.), drawn together by the tethers, and then fixed relative to one another (e.g., by tying the tethers off).

Referring to FIG. 120, in some arrangements, three devices 23100, 23100′, 23100″ can be arranged in a triangular pattern and secured together. For example, the outer faces 23112, 23112′, 23112″ of three devices 23100, 23100′, 23100″ can be arranged to face inward toward each other so that the inner faces 23110, 23110′, 23110″ face outward. The three devices 23100, 23100′, 23100″ can be to each other in any suitable manner. In some implementations, the attached devices 23100, 23100′, 23100″ can be implanted in a tricuspid valve TV where each device 23100, 23100′, 23100″ can capture a leaflet of the tricuspid valve TV. For example, a first device 23100 can capture the anterior leaflet 30, the second device 23100′ can capture the septal leaflet 32, and the third device 23100″ can capture the posterior leaflet 34. In some implementations, each of the devices 23100, 23100′, 23100″ is independently actuatable. Thus, for example, one of the three devices 23100, 23100′, 23100″ can be moved to a closed condition to grasp a valve leaflet while the other two of the devices 23100, 23100′, 23100″ remain in an open or partially closed position.

Referring to FIG. 121, in some arrangements, two devices 23100, 23100′ can be arranged back-to-back with a coaptation element (e.g., coaptation element of FIG. 84) between the two devices 23100, 23100′. Any suitable coaptation element can be used in the arrangement, such as any coaptation element disclosed herein. Each of the two devices 23100, 23100′ can be attached to the coaptation element. For example, in some implementations, the outer faces 23112, 23112′ of the two devices 23100, 23100′ can be attached to the coaptation element such that the inner faces 23110, 23110′ are facing outward. In some implementations, the two devices 23100, 23100′ can be attached to the coaptation element on opposite sides of the coaptation element.

In some implementations, the attached devices 23100, 23100′ can be implanted in a native heart valve where each device 23100, 23100′ can capture a leaflet of the heart valve. For example, the two devices 23100, 23100′ can be implanted in a mitral valve MV where a first device 23100 can capture the anterior leaflet 20 and the second device 23100′ can capture the posterior leaflet 22. Similarly, the two devices 23100, 23100′ can be implanted in a tricuspid valve TV where the two devices 23100, 23100′ capture two or three of the leaflets 30, 32, 34 of the tricuspid valve TV. In some implementations, each of the devices 23100, 23100′ is independently actuatable. Thus, for example, one of the two devices 23100, 23100′ can be moved to a closed condition to grasp a valve leaflet while the other of the two devices 23100, 23100′ remains in an open or partially closed position.

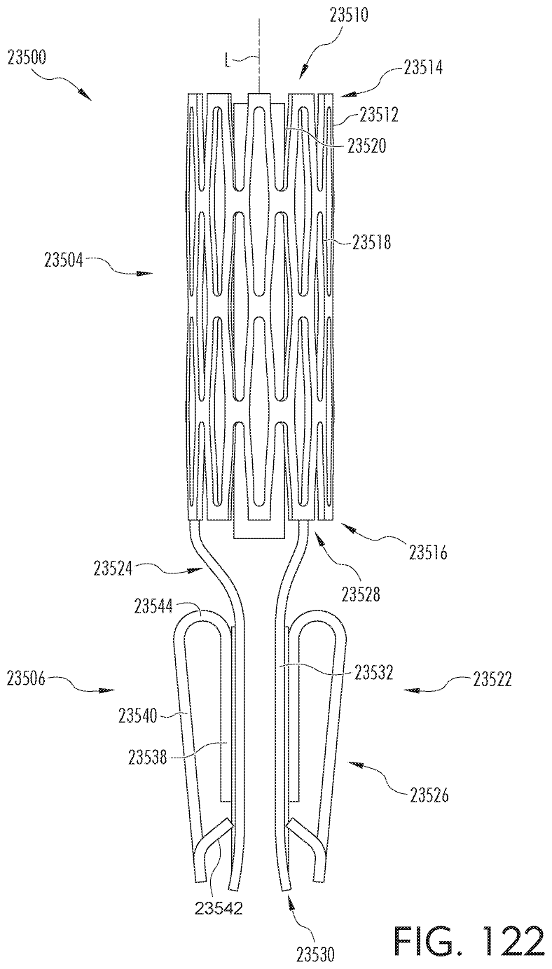

FIGS. 122-128 illustrate an example implantable device or implant 23500 (e.g., a treatment device, repair device, valve treatment device, valve repair device, prosthetic device, implantable device, implant, etc.) in various stages of deployment. The device 23500 can include any other features for another device or implant discussed in the present application or the applications cited above, and the device 23500 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device or implant 23500 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 23500 includes a coaptation portion 23504 and an anchor portion 23506.

In some implementations, the coaptation portion 23504 of the device or implant 23500 includes a coaptation element 23510 that is adapted to be implanted between leaflets of a native valve (e.g., a native mitral valve, native tricuspid valve, etc.). The coaptation element 23510 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23510 is expandable.

In some implementations, the expandable coaptation element 23510 includes an expandable frame member 23512 (e.g., frame, body, cage, fixture, chassis, form, etc.) that is configured to move between a first radially contracted condition and a second radially expanded condition. The frame member 23512 can be configured in a variety of ways, such as any of the expandable frame members discloses herein.

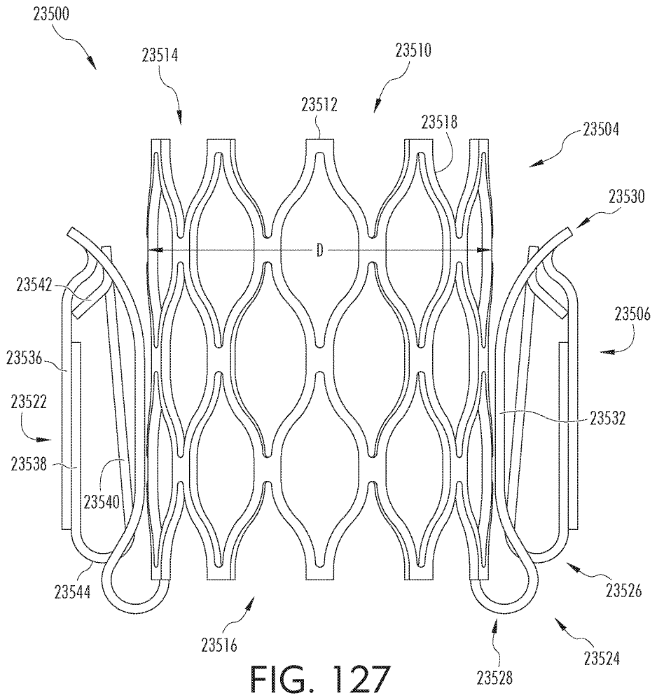

In some implementations, as shown in the illustrated example, the frame member 23512 has a generally cylindrical shape (i.e., a circular cross section) having a longitudinal axis L, a proximal end 23514, a distal end 23516 opposite the proximal end 23514, and a diameter D that can be collapsed (i.e., in the first condition as shown in FIG. 126) and expanded (i.e., in the second condition as shown in FIG. 127). In some implementations, however, the frame member 23512 can have a shape other than cylindrical (e.g., a cross section that is oval, rectangular, elliptical, or other suitable shape).

In some implementations, the frame member 23512 can include a plurality of interconnected struts 23518 that are configured to flex or bend to allow the frame member 23512 to move between the first and second condition. The interconnected struts 23518 can be configured in a variety of ways, such as for example, the number and size of the struts, the shape of each strut, the arrangement of struts relative to other struts, the interconnection of the struts, etc. Any configuration that can facilitate the expansion and contraction of the frame member 23512 can be used. In some implementations, the struts 23518 are arranged in diamond, oval, lens, or similar shape.

In some implementations, the frame member 23512 is formed as a unitary piece. For example, the frame member 23512 can be laser-cut from a tube. In some implementations, however, some of the struts 23518 can be formed separately and connected in any suitable manner (e.g., welded together). In some implementations, the frame member 23512 can comprise and/or be formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

The frame member 23512 can be moved between the first and the second conditions by any suitable means. For example, the frame member 23512 can be mounted around an expandable mechanism 23520 (illustrated schematically in FIG. 122) configured to engage the frame member 23512 to move the frame member 23512 between the first and the second conditions. A variety of different mechanisms can be used to move the frame member 23512 between the first and second conditions. For example, any expandable mechanism disclosed herein can be used, such as, but not limited to, the expandable mechanism 21016 of FIGS. 89-93, the expandable mechanism 22016 of FIGS. 92-98, and the expandable mechanism 22116 of FIG. 99.

In some implementations, the anchor portion 23506 includes one or more anchors 23522 that are actuatable between open and closed conditions and can take a wide variety of forms, such as, for example, paddles, gripping elements, links, or the like. The anchor portion 23506 can be actuated to open and close in a variety of ways. In some implementations, an actuation element (not shown) is used to move the anchors between open and closed conditions.

The actuation element (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 23506 relative to the coaptation portion 23504. Or, the actuation element can be unthreaded, such that pushing or pulling the actuation element moves the anchor portion 23506 relative to the coaptation portion 23504.

In some implementations, the anchor portion 23506 and/or the one or more anchors 23522 of the device or implant 23500 include paddles 23524 and attachment portions or gripping members 23526. In some implementations, the paddles 23524 can be opened and closed, for example, to grasp the native leaflets (e.g., native mitral valve leaflets, etc.). The paddles 23524 can be configured in a variety of ways, such as, for example, any of the paddles disclosed herein. In the illustrated example, the device 23500 includes a pair of paddles 23524 attached to, and extending from, the distal end 23516 of the frame member 23512 on opposite sides of the frame member 23512. In some implementations, the device 23500 can include more or less than a pair of paddles 23524.

In some implementations, as shown in the illustrated example, each paddle 23524 includes a proximal portion 23528 that is attached to, and extends from, the distal portion 23516 of the frame member 23512, a distal portion 23530 opposite the proximal portion 23528, and an intermediate portion 23532 connecting the proximal portion 23528 to the distal portion 23530. The paddles 23524 can have a variety of shapes. In some implementations, each paddle 23524 has U-shape such that the proximal portion 23528 attached to the frame member 23512 at two spaced apart locations and the distal portion 23530 has a closed end.

The proximal portion 23528 can attach to the distal end 23516 of the frame member 23512 in any suitable manner (e.g., welded together). In some implementations, the paddles 23524 are formed as a unitary piece with the frame member 23512 (e.g., laser-cut from a tube with the frame member 23512). Each paddle 23524 can be jointed and/or flexible to move between all the positions described below regarding FIGS. 122-128. In the illustrated example, the paddle 23524 is flexible or bendable at, or proximate to, the proximal portion 23528. In some implementations, the paddle 23524 can be flexible or bendable at any suitable location along the intermediate portion 23532.

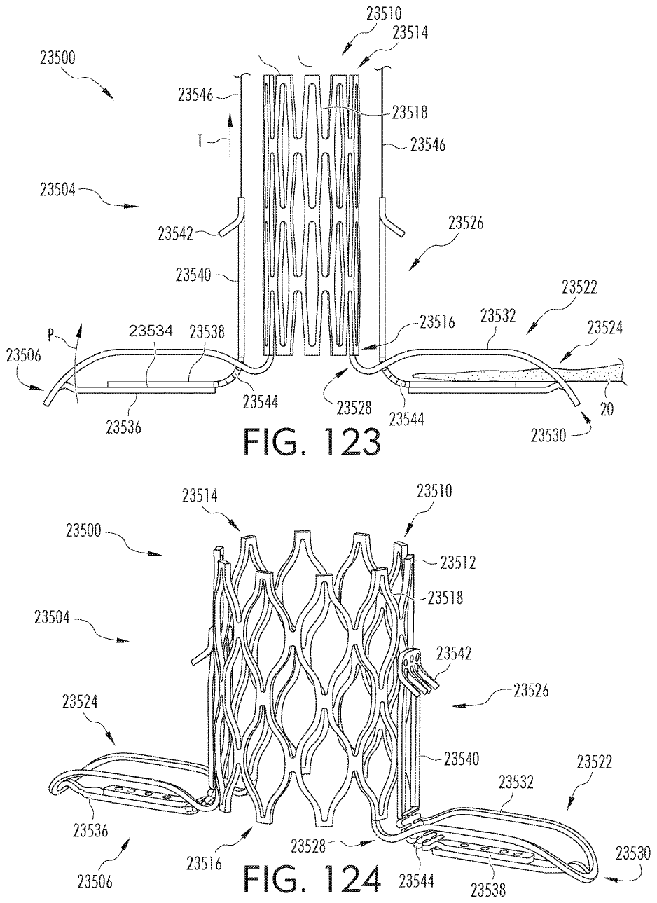

In some implementations, each paddle 23524 includes a mounting surface 23534 (FIG. 123) to which one or more gripping members 23526 can be attached. The mounting surface 23534 can be configured in a variety of way, including position on the paddle 23524, size, orientation, etc. In the illustrated example, the mounting surface 23534 is disposed on a mounting arm 23536 that extends proximally from the distal portion 23516. In some implementations, the mounting arm 23536 can extend parallel to, and offset from, the intermediate portion 23532, as shown in FIG. 123.

In some implementations, each paddle 23524 has a gripping member 23526 attached. In some implementations, the gripping member 23526 is attached to the mounting arm 23536. The gripping member 23526 can be configured in a variety of ways, such as any gripping member disclosed herein. In the illustrated example, the gripping member 23526 is formed as a clasp that includes a base or fixed arm 23538, a moveable arm 23540, optional friction-enhancing elements, other securing structures 23542 (e.g., barbs, protrusions, ridges, grooves, textured surfaces, adhesive, etc.), and a joint portion 23544. The fixed arm 23538 is attached to the mounting arm 23536. In some implementations, the fixed arm 23538 is attached to the mounting arm 23536 with the joint portion 23544 disposed proximate the proximal portion 23528 of the paddle 23524.

In some implementations, the joint portion 23544 provides a spring force between the fixed and moveable arms 23538, 23540. The joint portion 23544 can be any suitable joint, such as a flexible joint, a spring joint, a pivot joint, or the like. The fixed arm 23538 is attached to the paddle 23524 and remains stationary or substantially stationary relative to the paddle 23524 when the moveable arm 23540 are opened to open the gripping member 23526 and expose the optional barbs or other friction-enhancing elements 23542.

In some implementations, the gripping member 23526 is opened by applying tension, as shown by arrow T in FIG. 123, to one or more actuation lines 23546 attached to the moveable arm 23540, thereby causing the moveable arm 23540 to articulate, flex, or pivot at the joint portion 23544. The one or more actuation lines 23546 extend through the delivery system (e.g., through a steerable catheter and/or an implant catheter). Other actuation mechanisms are also possible. The actuation line 23546 can take a wide variety of forms, such as, for example, a line, a suture, a wire, a rod, a catheter, or the like. The gripping member 23526 can be spring loaded so that in the closed position the gripping member 23526 continues to provide a pinching force on the grasped native leaflet.

Referring to FIG. 122, in the elongated or open condition, the paddle 23524 is in an extended positioned relative to the coaptation portion 23504 and the gripping members 23526 are in a closed condition. Movement of the paddle 23524 to the extended condition relative to the coaptation portion 23504 can be accomplished in a variety of ways. For example, in some implementations, the paddle 23524 can be engaged by the actuation element (not shown) described above that can move the paddle 23524 to the extended condition. In some implementations, the paddles 23524 can be loaded into a catheter of the delivery system in the elongated condition. In some implementations, the paddles 23524 can be shape set to the closed condition, or partially closed condition, such that when the device 23500 exits the catheter, the paddles 23524 move, or are biased, to the closed or partially closed position.

Once the device 23500 is positioned within the native valve adjacent a leaflet (e.g., leaflets 20, 22, 30, 32, 34), the anchor portion 23506 can move to a capture ready condition (i.e., partially closed) as shown in FIGS. 123-124. As indicated above, the device 23500 can be configured to move the anchor portion 23506 between the open condition, the capture ready condition, and the closed condition in a variety of ways, such as an actuation element (not shown) or/and shape setting the anchor portion 23506. In the capture ready condition, the paddles 23524 partially close by flexing or bending proximate to, or at, the proximal portion 23528. In some implementations, the paddles 23524 extend perpendicular, or near perpendicular, to the longitudinal axis L.

In the capture ready position, the gripping member 23526 is in an open condition. In some implementations, the fixed arm 23538 of the gripping member 23526 moves with the paddle 23524 to partially close the gripping member 23526 while the moveable arm 23540 is held open by the actuation line 23546. In some implementations, the gripping member 23526 is spring loaded to the closed condition and thus helps pivot the paddle 23524 in the direction of arrow P.

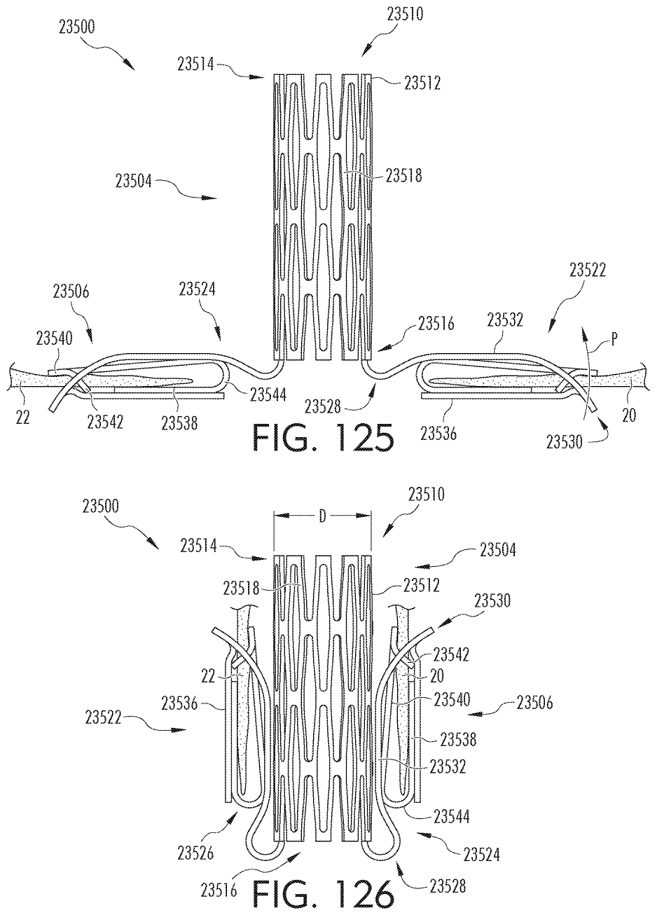

In some implementations, once the leaflet 20 is in a satisfactory position to be grasped, as shown in FIG. 123, tension in the actuation line 23546 can be released. Releasing tension in the actuation line 23546 allows the spring force associated with the joint portion 23544 to close the gripping member 23526 by moving the moveable arm 23540 toward the fixed arm 23538 and into contact with the leaflet 20, as shown in FIG. 125, to pinch the leaflet 20 between the moveable arm 23540 and the fixed arm 23538. Once one leaflet is grasped in one of the anchor portions 23506, the device 23500 can be positioned to grasp a second leaflet (e.g., leaflet 22) in the other anchor portion 23506. For example, with one leaflet is grasped in one of the anchor portions 23506, the frame member 23512 can be expanded or contracted to position the other anchor portion 23506 to capture a second leaflet.

In some implementations, once the leaflets are captured in the gripping members 23526, the device 23500 can be moved to the closed condition as shown in FIG. 126. When moving to the closed condition, the paddles 23524 further bend or flex proximate to, or at, the proximal portion 23528, as shown by arrow P in FIG. 125, such that the anchor portions 23506 extend along the coaptation portion 23504. In some implementations, the paddles 23524 and/or the moveable arm 23540 can contact the coaptation portion 23504 in the closed condition.

Referring to FIG. 127, the coaptation portion 23504 can be expanded from the first radially contracted condition (e.g., FIG. 126) to the second radially expanded condition (FIG. 127) such that the diameter D of the frame member 23512 increases. FIG. 127 shows the coaptation portion 23504 in the expanded condition with the anchor portions 23506 in the closed condition. The coaptation portion 23504, however, can moved between the expanded condition and the contracted position when the anchor portions 23506 are in any position (e.g., open condition and partially closed condition). The frame member 23512 can be moved between the first and the second conditions by the expandable mechanism 23520 (illustrated schematically in FIG. 122) or any suitable means.

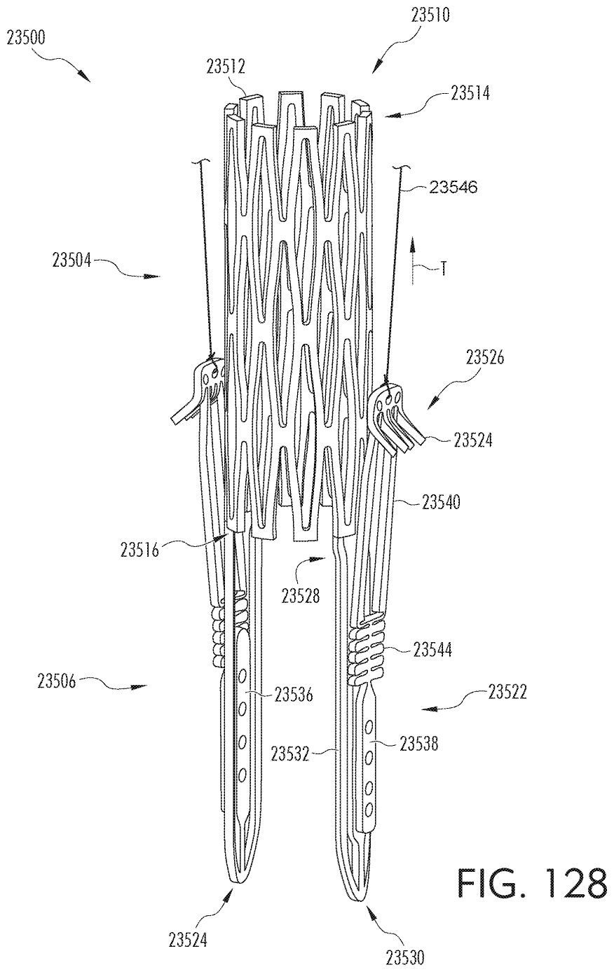

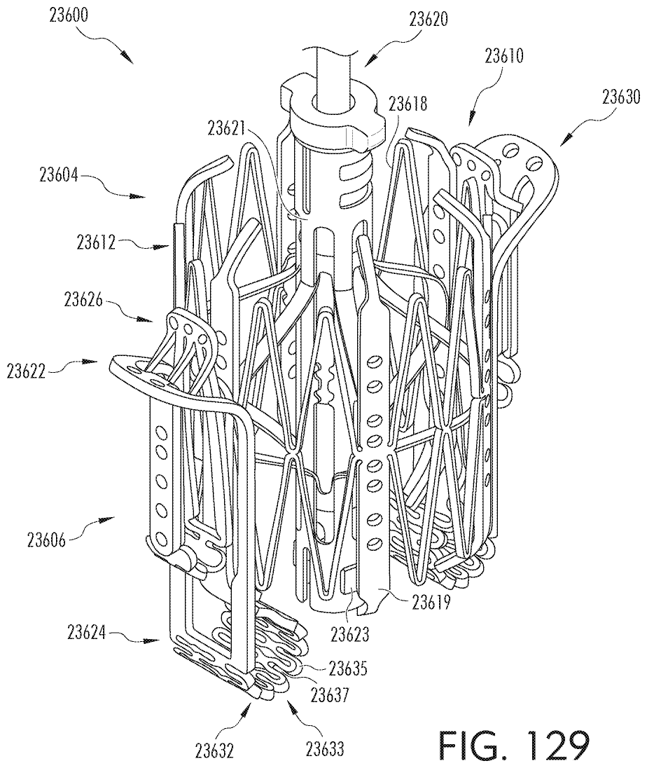

Referring to FIG. 128, the device 23500 is shown in a release condition that allows release of a leaflet previously captured by the device 23500. Thus, the release condition, for example, allows the device 23500 to release a leaflet that may not be satisfactorily captured, be repositioned, and recapture the leaflet. In the release condition, the paddles 23524 are moved to the extended condition relative to the coaptation portion 23504, similar to the open condition of FIG. 122, such that the paddles 23524 extend distally from the distal end 23516 of the frame member 23512. With the paddles 23524 extended, the gripping members 23526 are opened by applying tension, as shown by arrow T in FIG. 128, to the actuation lines 23546 attached to the moveable arms 23540, thereby causing the moveable arms 23540 to articulate, flex, or pivot at the joint portion 23544.

FIG. 129-133 illustrate an example implantable device or implant 23600 (e.g., a prosthetic device, a valve repair device, implantable device, etc.) in various stages of deployment. The device 23600 can include any other features for another device or implant discussed in the present application or the applications cited above, and the device 23600 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device or implant 23600 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 23600 includes a coaptation portion 23604 and an anchor portion 23606.

In some implementations, the coaptation portion 23604 of the device or implant 23600 includes a coaptation element 23610 that is adapted to be implanted between leaflets of a native valve (e.g., a native mitral valve, native tricuspid valve, etc.). The coaptation element 23610 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23610 is expandable. In some implementations, the expandable coaptation element 23610 includes an expandable frame member 23612 (e.g., frame, body, cage, fixture, chassis, form, etc.) that is configured to move between a first radially contracted condition and a second radially expanded condition. The frame member 23612 can be configured in a variety of ways, such as any of the expandable frame members discloses herein.

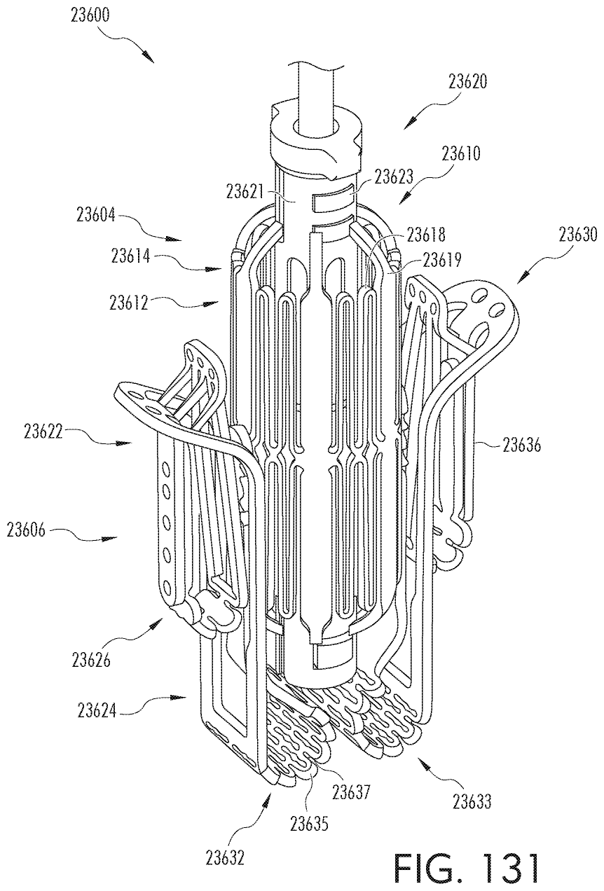

In the illustrated example, the frame member 23612 is substantially similar to the frame member 21002 of FIGS. 82-83 and the description of the frame member 21002 applies equally to frame member 23612. For example, the frame member 23612 has a generally cylindrical shape (i.e., a circular cross section) having a longitudinal axis L, a proximal end 23614, a distal end 23616 opposite the proximal end 23614, and a diameter D that can be collapsed (i.e., in the first condition as shown in FIG. 131) and expanded (i.e., in the second condition as shown in FIG. 129). In some implementations, however, the frame member 23612 can have a shape other than cylindrical (e.g., a cross section that is oval, rectangular, elliptical, or other suitable shape).

The frame member 23612 can include a plurality of interconnected struts 23618 that are configured to flex or bend to allow the frame member 23612 to move between the first and second condition. The frame member 23612 can also include a plurality of posts 23619 interconnected by the plurality of struts 23618. In the illustrated example, each post 23619 is connected on either side, along a midpoint, to a diamond pattern formed by the struts 23618.

In some implementations, the frame member 23612 is formed as a unitary piece. For example, the frame member 23612 can be laser-cut from a tube. In some implementations, however, some of the struts 23618 and the posts 23619 can be formed separately and connected in any suitable manner (e.g., welded together). In some implementations, the frame member 23612 can comprise and/or be formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

The frame member 23612 can be moved between the first and the second conditions by any suitable means. For example, the frame member 23612 can be mounted around an expandable mechanism 23620 configured to engage the frame member 23612 to move the frame member 23612 between the first and the second conditions. A variety of different mechanisms can be used to move the frame member 23612 between the first and second conditions. I

In the illustrated example, the expandable mechanism 23620 is substantially similar to the expandable mechanism 21016 (see FIGS. 84-86) and the description of the expandable mechanism 21016 applies equally to the expandable mechanism 23620. For example, the expandable mechanism 23620 can include an expandable member 23621 (e.g., frame, body, strut assembly, tube, shaft, etc.) and an actuation mechanism 23623. The expandable member 23621 and actuation mechanism 23623 can be configured in a variety of ways, such as any expandable member (e.g., expandable member 21018) and actuation mechanism (e.g., actuation mechanism 21020) disclosed herein.

In some implementations, the anchor portion 23606 includes one or more anchors 23622 that are actuatable between open and closed conditions and can take a wide variety of forms, such as, for example, paddles, gripping elements, links, or the like. The anchor portion 23606 can be actuated to open and close in a variety of ways. In some implementations, an actuation element (not shown) is used to move the anchors between open and closed conditions. The actuation element (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 23606 relative to the coaptation portion 23604. Or, the actuation element can be unthreaded, such that pushing or pulling the actuation element moves the anchor portion 23606 relative to the coaptation portion 23604.

In some implementations, the anchor portion 23606 and/or the one or more anchors 23622 of the device or implant 23600 include paddles 23624 and attachment portions or gripping members 23626. The paddles 23524 can be opened and closed, for example, to grasp the native leaflets (e.g., native mitral valve leaflets, etc.). The paddles 23624 can be configured in a variety of ways, such as, for example, any of the paddles disclosed herein. In the illustrated example, the device 23600 includes a pair of paddles 23624 attached to the frame member 23612 and extending downward past the distal end 23616 on opposite sides of the frame member 23612. In some implementations, the device 23600 can include more or less than a pair of paddles 23624.

In the illustrated example, each paddle 23624 includes a proximal portion 23628 that is attached to the frame member 23612, a distal portion 23630 opposite the proximal portion 23628, and an intermediate portion 23632 connecting the proximal portion 23628 to the distal portion 23630.

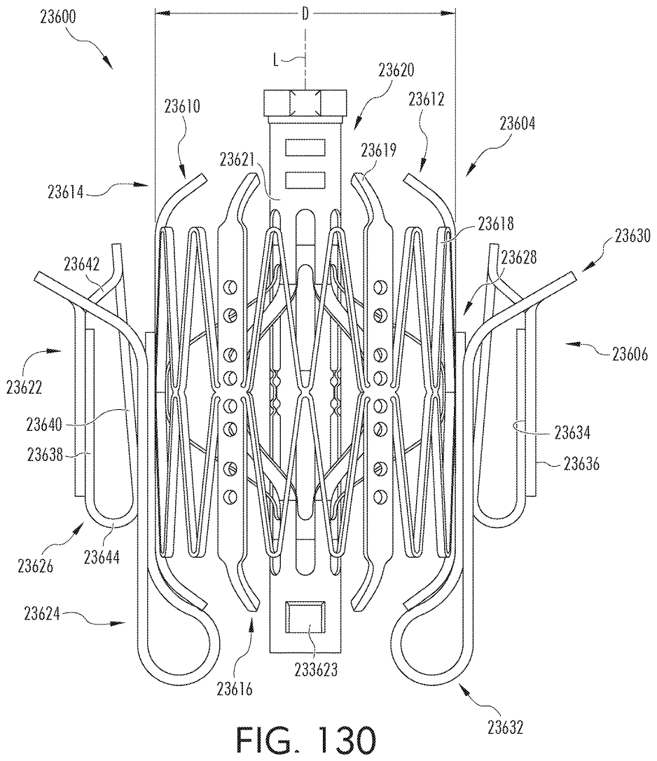

The proximal portion 23628 can attach to the distal end 23616 of the frame member 23612 in any suitable manner (e.g., welded together). In some implementations, the paddles 23624 are formed as a unitary piece with the frame member 23612 (e.g., laser-cut from a tube with the frame member 23612). In some implementations, each paddle 23624 can be jointed and/or flexible to move between all the positions described below regarding FIGS. 129-133. In the illustrated example, the intermediate portion 23632 forms a patterned hinge portion 23633 (FIGS. 129, 131) formed by a plurality of torsional spring segments 23635 arranged in a repeating pattern and separated by open and closed cutouts 23637. In some implementations, each torsional spring segment 23635 is joined with other torsional spring segments 23635 to form the repeating pattern. Joining multiple torsional spring segments 23635 together allows the hinge portion 23633 to bend a considerable amount, as shown in FIGS. 129-130, while avoiding plastic deformation of the material as the individual torsional spring segments 23635 are twisted.

In some implementations, each paddle 23624 includes a mounting surface 23634 (FIG. 130) to which one or more gripping members 23626 can be attached. The mounting surface 23634 can be configured in a variety of way, including position on the paddle 23624, size, orientation, etc. In the illustrated example, the mounting surface 23634 is disposed on a mounting arm 23636 that extends proximally from the distal portion 23630. In some implementations, the mounting arm 23636 can extend parallel to, and offset from, the intermediate portion 23632, as shown in FIG. 130.

In some implementations, each paddle 23624 has a gripping member 23626 attached. In some implementations, the gripping member 23626 is attached to the mounting arm 23636. The gripping member 23626 can be configured in a variety of ways, such as any gripping member disclosed herein. In the illustrated example, the gripping member 23626 is formed as a clasp that includes a base or fixed arm 23638, a moveable arm 23640, optional friction-enhancing elements, other securing structures 23642 (e.g., barbs, protrusions, ridges, grooves, textured surfaces, adhesive, etc.), and a joint portion 23644. The fixed arm 23638 is attached to the mounting arm 23636.

In some implementations, the joint portion 23644 provides a spring force between the fixed and moveable arms 23638, 23640. The joint portion 23644 can be any suitable joint, such as a flexible joint, a spring joint, a pivot joint, or the like. In the illustrated example, the joint portion 23644 has a similar patterned hinge configuration as the patterned hinge portion 23633 of the paddles 23624. Thus, the description of the patterned hinge portion 23633 applies equally to the joint portion 23644.

In some implementations, the fixed arm 23638 is attached to the paddle 23624 and remains stationary or substantially stationary relative to the paddle 23624 when the moveable arm 23640 is opened to open the gripping member 23626 and expose the optional barbs or other friction-enhancing elements 23642.

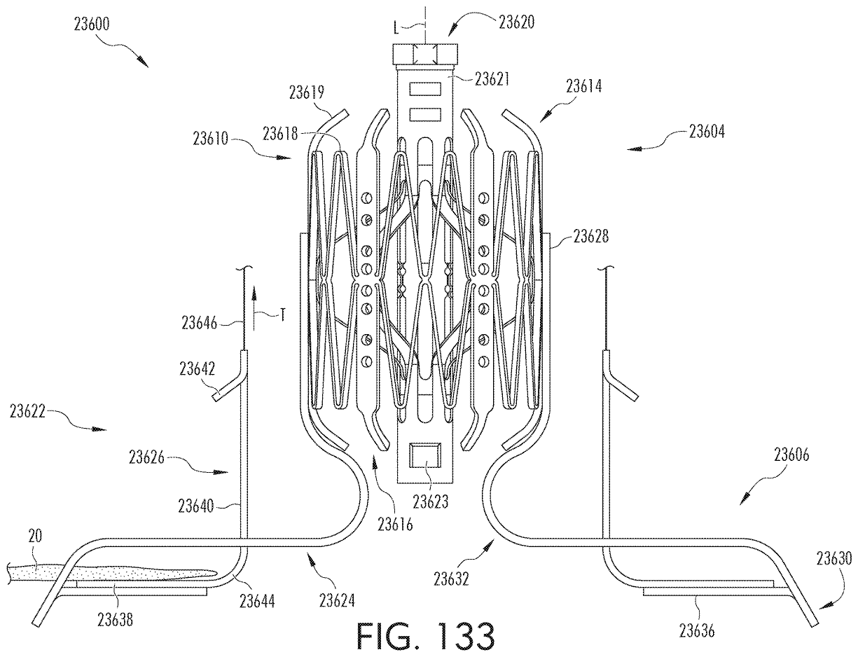

In some implementations, the gripping member 23626 is opened by applying tension, as shown by arrow T in FIG. 133, to one or more actuation lines 23646 attached to the moveable arm 23640, thereby causing the moveable arm 23640 to articulate, flex, or pivot at the joint portion 23644. The one or more actuation lines 23646 extend through the delivery system (e.g., through a steerable catheter and/or an implant catheter). Other actuation mechanisms are also possible. The actuation line 23646 can take a wide variety of forms, such as, for example, a line, a suture, a wire, a rod, a catheter, or the like. The gripping member 23626 can be spring loaded so that in the closed position the gripping member 23626 continues to provide a pinching force on the grasped native leaflet.

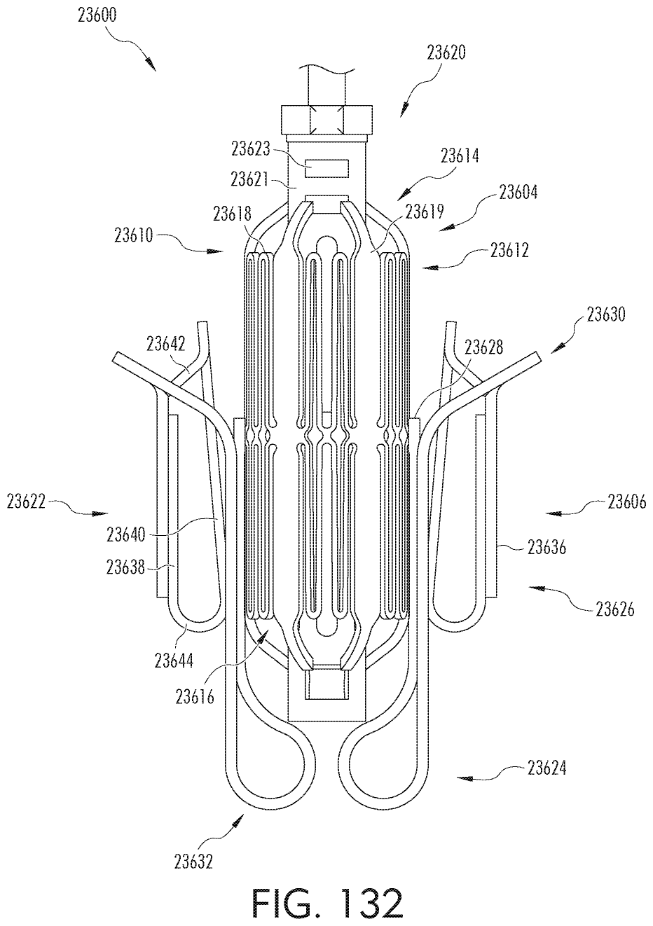

Referring to FIG. 133, the device 23600 is shown in a capture ready condition within the native valve adjacent a leaflet (e.g., leaflets 20, 22, 30, 32, 34). FIG. 133 illustrates the frame member 23612 in the expanded condition but the frame member 23612 is moveable independent of the condition of the anchor portions 23606. Thus, the frame member 23612 can be in the contracted condition (e.g., FIG. 132), the expanded condition, or any condition therebetween.

In some implementations, the device 23600 can be delivered to the native valve in an elongated, open condition (not shown) similar to the open condition illustrated in FIG. 122 for device 23500. For example, the paddles 23624 can be moved to an extended condition relative to the coaptation portion 23604 (extending distally from the coaptation portion 23604) and the gripping members 23526 are in a closed condition.

In some implementations, once the device 23600 is positioned within the native valve adjacent a leaflet (e.g., leaflets 20, 22, 30, 32, 34), the anchor portion 23606 can move to a capture ready condition (i.e., partially closed) as shown in FIG. 133. As indicated above, the device 23600 can be configured to move the anchor portion 23606 between the open condition, the capture ready condition, and the closed condition in a variety of ways, such as an actuation element (not shown) or/and shape setting the anchor portion 23606. In the capture ready condition, the paddles 23624 partially close by flexing or bending the hinge portion 23633. In some implementations, the paddles 23624 extend perpendicular, or near perpendicular, to the longitudinal axis L.

In the capture ready position, the gripping member 23626 is in an open condition. In some implementations, the fixed arm 23638 of the gripping member 23626 and the paddle 23624 are partially closed while the moveable arm 23640 is held open by the actuation line 23646. In some implementations, the gripping member 23626 is spring loaded to the closed condition and thus helps pivot the paddle 23524 in the direction of arrow P.

In some implementations, once the leaflet 20 is in a satisfactory position to be grasped, as shown in FIG. 133, the gripping member 23626 can be closed by releasing tension of in the actuation line 23646 to allow the moveable arm 23640 to close and pinch the leaflet 20 between the moveable arm 23640 and the fixed arm 23638. Once one leaflet is grasped in one of the anchor portions 23606, the device 23600 can be positioned to grasp a second leaflet (e.g., leaflet 22) in the other anchor portion 23606. For example, with one leaflet is grasped in one of the anchor portions 23606, the frame member 23612 can be expanded or contracted to position the other anchor portion 23606 to capture a second leaflet.

In some implementations, once the leaflets are captured in the gripping members 23626, the device 23600 can be moved to the closed condition as shown in FIGS. 129-130. When moving to the closed condition, the paddles 23624 further bend or flex at the hinge portion 23633 such that the anchor portions 23606 extend along the coaptation portion 23604. In some implementations, the paddles 23624 and/or the moveable arm 23640 can contact the coaptation portion 23604 in the closed condition.

Referring to FIGS. 131-132, the device 23600 is shown in the closed condition similar to FIGS. 129-130 except that the frame member 23612 is shown in the contracted condition. Since the frame member 23612 is moveable independent of the condition of the anchor portions 23606, the frame member 23612 can be moved between the expanded and contracted conditions when the device 23600 is closed, open, or partially closed.

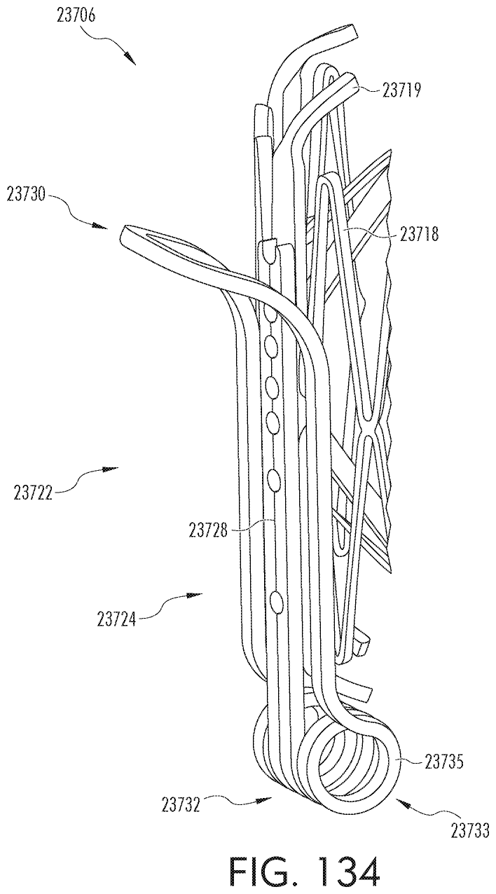

FIG. 134-138 illustrate an example implantable device or implant 23700 (e.g., a treatment device, a repair device, a prosthetic device, a valve repair device, an implantable device, an implant, etc.) in various stages of deployment. The device 23700 can include any other features for another device or implant discussed in the present application or the applications cited above, and the device 23700 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device or implant 23700 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In some implementations, the device or implant 23700 includes a coaptation portion 23704 and an anchor portion 23706.

In some implementations, the coaptation portion 23704 of the device or implant 23700 includes a coaptation element 23710 that is adapted to be implanted between leaflets of a native valve (e.g., a native mitral valve, native tricuspid valve, etc.). The coaptation element 23710 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23710 is expandable. In some implementations, the expandable coaptation element 23710 includes an expandable frame member 23712 (e.g., frame, body, cage, fixture, chassis, form, etc.) that is configured to move between a first radially contracted condition and a second radially expanded condition. The frame member 23712 can be configured in a variety of ways, such as any of the expandable frame members discloses herein.

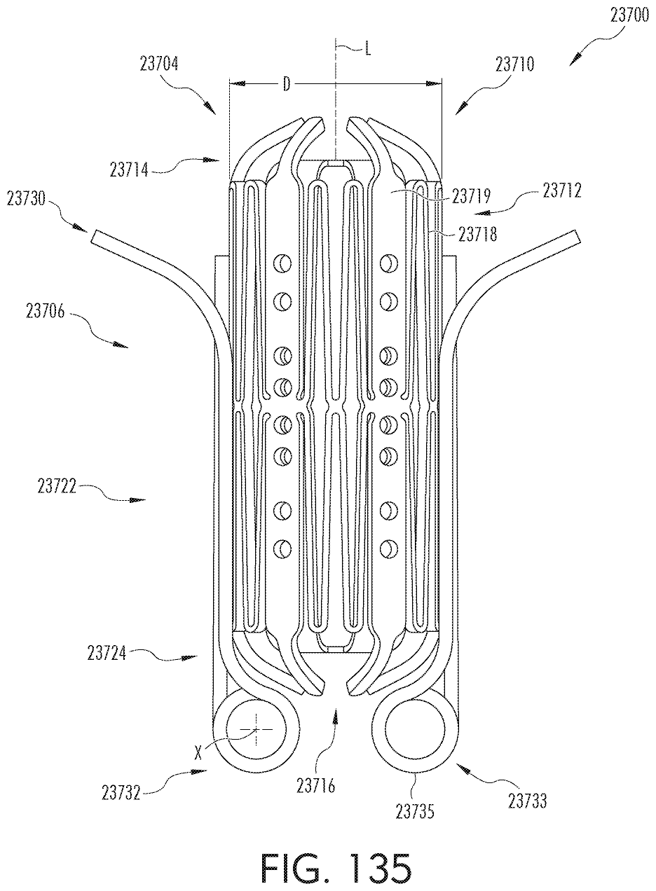

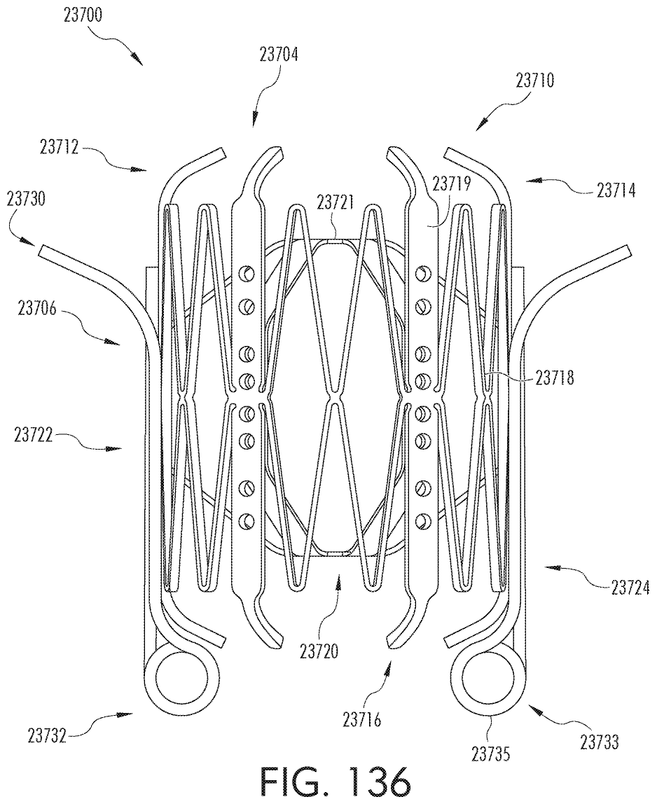

In the illustrated example, the frame member 23712 is substantially similar to the frame member 21002 of FIGS. 82-83 and the description of the frame member 21002 applies equally to frame member 23712. For example, the frame member 23712 has a generally cylindrical shape (i.e., a circular cross section) having a longitudinal axis L, a proximal end 23714, a distal end 23716 opposite the proximal end 23714, and a diameter D that can be collapsed (i.e., in the first condition as shown in FIG. 135) and expanded (i.e., in the second condition as shown in FIG. 136). In some implementations, however, the frame member 23712 can have a shape other than cylindrical (e.g., a cross section that is oval, rectangular, elliptical, or other suitable shape).

In some implementations, the frame member 23712 can include a plurality of interconnected struts 23718 that are configured to flex or bend to allow the frame member 23712 to move between the first and second condition. In some implementations, the frame member 23712 can also include a plurality of posts 23719 interconnected by the plurality of struts 23718. In the illustrated example, each post 23718 is connected on either side, along a midpoint, to a diamond pattern formed by the struts 23718.

In some implementations, the frame member 23712 is formed as a unitary piece. For example, the frame member 23712 can be laser-cut from a tube. In some implementations, however, some of the struts 23718 and the posts 23719 can be formed separately and connected in any suitable manner (e.g., welded together). In some implementations, the frame member 23712 can comprise and/or be formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

The frame member 23712 can be moved between the first and the second conditions by any suitable means. For example, the frame member 23712 can be mounted around an expandable mechanism 23720 (FIG. 136) configured to engage the frame member 23712 to move the frame member 23712 between the first and the second conditions. A variety of different mechanisms can be used to move the frame member 23712 between the first and second conditions.

In the illustrated example, the expandable mechanism 23720 is substantially similar to the expandable mechanism 21016 (see FIGS. 84-86) and the description of the expandable mechanism 21016 applies equally to the expandable mechanism 23720. For example, the expandable mechanism 23720 can include an expandable member 23721 (FIG. 136) (e.g., frame, body, strut assembly, tube, shaft, etc.) and an actuation mechanism (not shown). The expandable member 23721 and actuation mechanism can be configured in a variety of ways, such as any expandable member (e.g., expandable member 21018) and actuation mechanism (e.g., actuation mechanism 21020) disclosed herein.

In some implementations, the anchor portion 23706 includes one or more anchors 23722 that are actuatable between open and closed conditions and can take a wide variety of forms, such as, for example, paddles, gripping elements, links, or the like. The anchor portion 23706 can be actuated to open and close in a variety of ways. In some implementations, an actuation element (not shown) is used to move the anchors between open and closed conditions. The actuation element (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 23706 relative to the coaptation portion 23704. Or, the actuation element can be unthreaded, such that pushing or pulling the actuation element moves the anchor portion 23706 relative to the coaptation portion 23704.

In some implementations, the anchor portion 23706 and/or the one or more anchors 23722 of the device or implant 23700 include paddles 23724 and attachment portions or gripping members (not shown for ease of illustration). The attachment portions or gripping members can be configured in a variety of ways, such as any attachment portions or gripping members disclosed herein.

In some implementations, the paddles 23724 can be opened and closed, for example, to grasp the native leaflets (e.g., native mitral valve leaflets, etc.). The paddles 23724 can be configured in a variety of ways, such as, for example, any of the paddles disclosed herein. In the illustrated example, the device 23700 includes a pair of paddles 23724 attached to the frame member 23712 and extending downward past the distal end 23716 on opposite sides of the frame member 23712. In some implementations, the device 23700 can include more or less than a pair of paddles 23724.

In some implementations, as shown in the illustrated example, each paddle 23724 includes a proximal portion 23728 that is attached to the frame member 23712, a distal portion 23730 opposite the proximal portion 23728, and am intermediate portion 23732 connecting the proximal portion 23728 to the distal portion 23730. The proximal portion 23728 can attach to the frame member 23712 in any suitable manner (e.g., welded together). In some implementations, the proximal portion 23728 is attached to a post 23719 of the frame member 23712. In some implementations, the paddles 23724 are formed as a unitary piece with the frame member 23712 (e.g., laser-cut from a tube with the frame member 23712).

In some implementations, each paddle 23724 is jointed and/or flexible to move between all the positions described below regarding FIGS. 134-138. In the illustrated example, the intermediate portion 23732 includes a torsional spring hinge portion 23733 about which the distal portion 23730 can pivot relative to the proximal portion 23728. In some implementations, the torsional spring hinge portion 23733 provides a spring force that biases the distal portion 23730 and the proximal portion 23728 together (i.e., to the closed condition).

The torsional spring hinge portion 23733 can be configured in a variety of ways. In the illustrated example, each torsional spring hinge portion 23733 include two coils 23735. In some implementations, however, each torsional spring hinge portion 23733 can include more or less than two coil. In the closed condition, as shown in FIG. 135, the coils 23735 are wound about a coil axis X that is traverse to the longitudinal axis L. In the illustrated example, in the closed condition, the coils 23735 are wound about the coil axis X (FIG. 135) which is perpendicular to the longitudinal axis L.

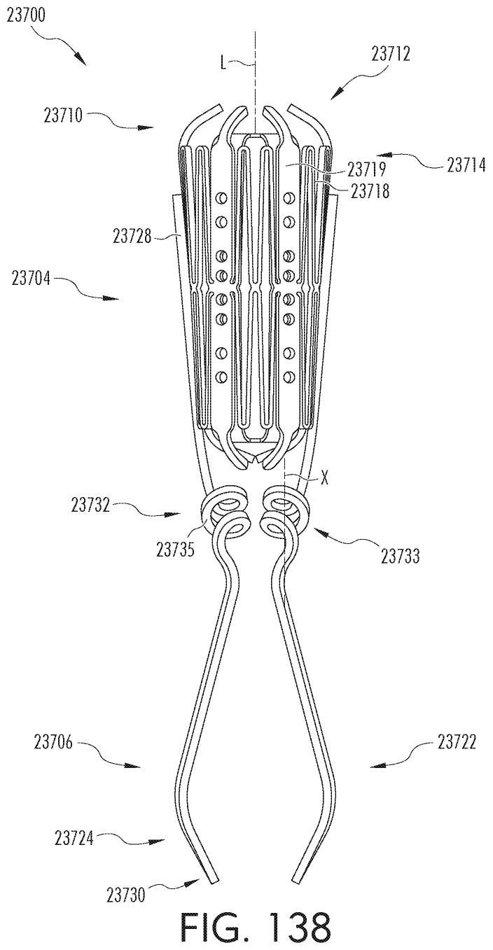

Referring to FIG. 138, in the elongated or open condition, the paddle 23724 is in an extended positioned relative to the coaptation portion 23704. Movement of the paddle 23724 to the extended condition relative to the coaptation portion 23704 can be accomplished in a variety of ways. For example, in some implementations, the paddle 23724 can be engaged by the actuation element (not shown) described above that can move the paddle 23724 to the extended condition. In some implementations, the paddles 23724 can be loaded into a catheter of the delivery system in the elongated condition. In the illustrated example, in the coils 23735 displace both longitudinally and rotationally. For example, in some implementations, the coil axis X rotates in the range of 45 degrees to 90 degrees in the direction of the longitudinal axis L. In some implementations, in the elongated or open condition, the coil axis X is parallel to the longitudinal axis L. In some implementations, however, the coils 23735 need not displace longitudinally and/or rotationally such that the orientation of the coil axis X remains the same, or similar, to the orientation in the closed condition (FIG. 135).

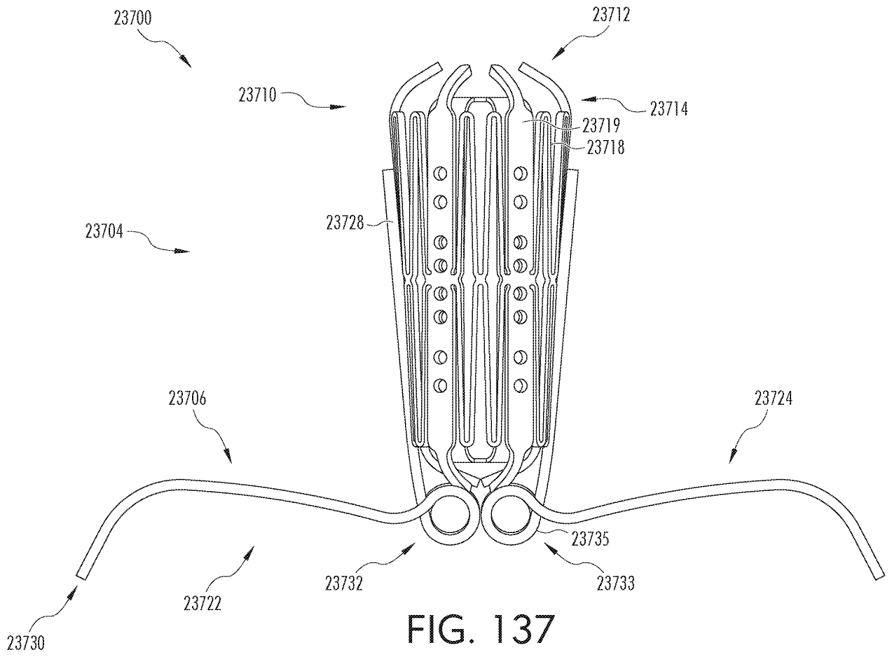

Referring to FIG. 137, once the device 23700 is positioned within the native valve adjacent a leaflet, the anchor portion 23706 can move to a capture ready condition (i.e., partially closed) (FIG. 137). FIG. 137 illustrates the frame member 23712 in the closed condition. The frame member 23712, however, is moveable independent of the condition of the anchor portions 23706; thus, the frame member 23712 can be in the expanded condition (e.g., FIG. 136), the expanded condition, or any condition therebetween.

In some implementations, the device 23700 can be configured to move the anchor portion 23706 between the open condition, the capture ready condition, and the closed condition in a variety of ways, such as an actuation element (not shown) (e.g., any actuation element disclosed herein) or/and shape setting the anchor portion 23706. In the capture ready condition, the paddles 23724 are partially closed by bending at the hinge portion 23733. In some implementations, the paddles 23724 extend perpendicular, or near perpendicular, to the longitudinal axis L.

In the capture ready condition, the device can capture the leaflet as described above (e.g., with gripping members). In some implementations, once one leaflet is grasped in one of the anchor portions 23706, the device 23700 can be positioned to grasp a second leaflet (e.g., leaflet 22) in the other anchor portion 23706. For example, with one leaflet is grasped in one of the anchor portions 23706, the frame member 23712 can be expanded or contracted to position the other anchor portion 23706 to capture a second leaflet.

In some implementations, once the leaflets are captured by the device 23730, the device can be moved to the closed condition as shown in FIG. 136. When moving to the closed condition, the paddles 23724 further bend at the hinge portion 23733 such that the anchor portions 23706 extend along the coaptation portion 23704. In some implementations, the paddles 23724 can contact the coaptation portion 23704 in the closed condition.

Referring to FIGS. 135, the device 23700 is shown in the closed condition similar to FIG. 136 except that the frame member 23712 is shown in the contracted condition. Since the frame member 23712 is moveable independent of the condition of the anchor portions 23706, the frame member 23712 can be moved between the expanded and contracted conditions when the device 23700 is closed, open, or partially closed.

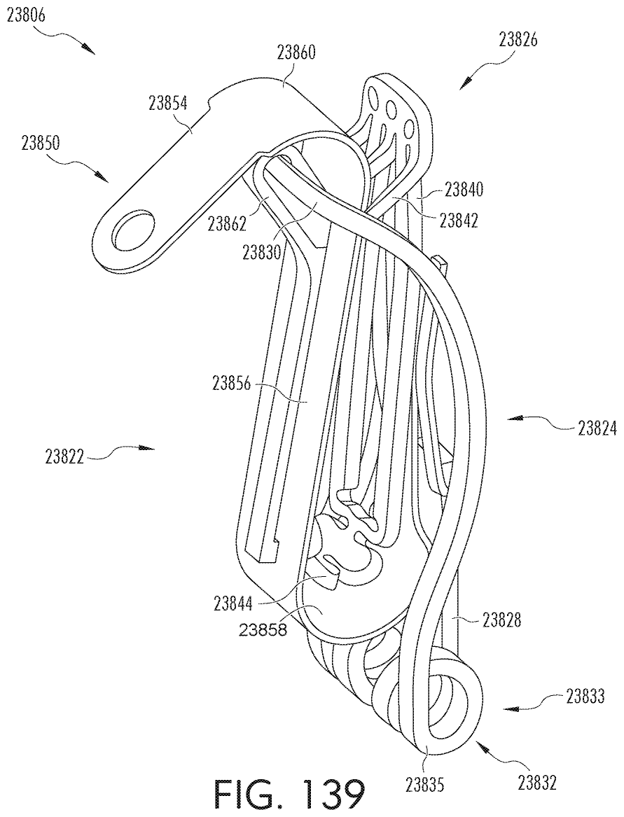

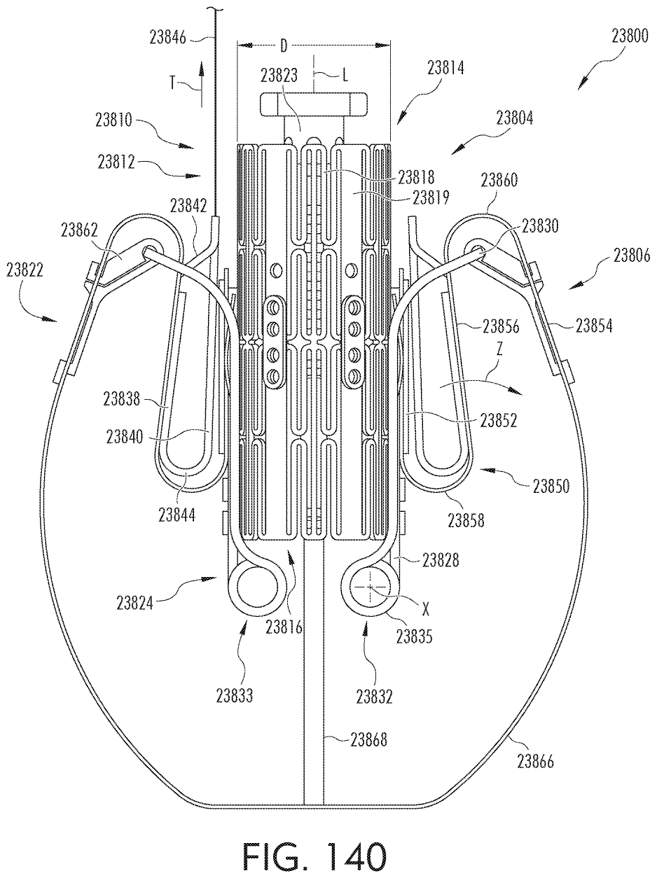

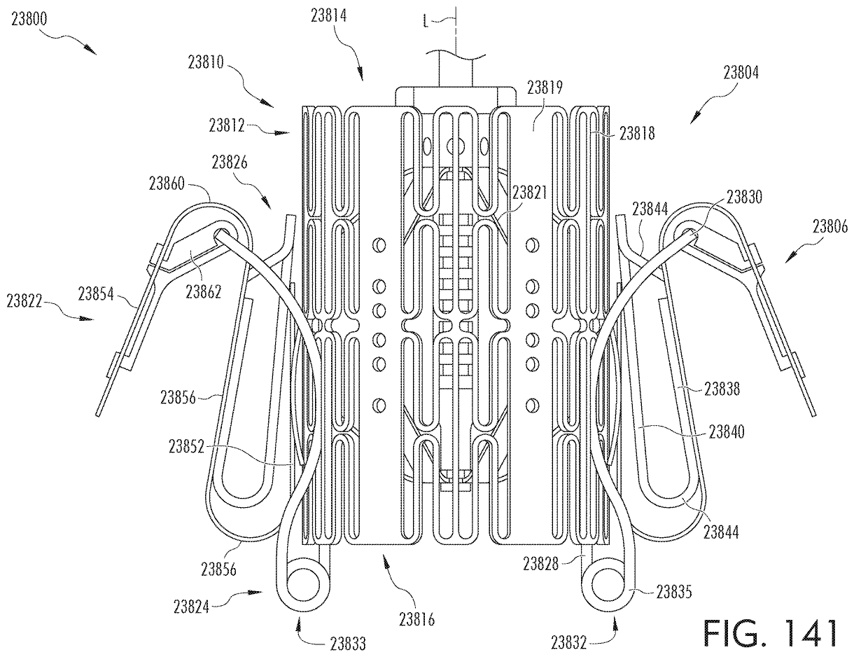

FIGS. 139-143 illustrate an example implantable device or implant 23800 (e.g., a treatment device, a repair device, a prosthetic device, a valve repair device, an implantable device, an implant, etc.) in various stages of deployment. The device 23800 can include any other features for another device or implant discussed in the present application or the applications cited above, and the device 23800 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device or implant 23800 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 23800 includes a coaptation portion 23804 and an anchor portion 23806.

In some implementations, the coaptation portion 23804 of the device or implant 23800 includes a coaptation element 23810 that is adapted to be implanted between leaflets of a native valve (e.g., a native mitral valve, native tricuspid valve, etc.). The coaptation element 23810 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23810 is expandable. In some implementations, the expandable coaptation element 23810 includes an expandable frame member 23812 (e.g., frame, body, cage, fixture, chassis, form, etc.) that is configured to move between a first radially contracted condition and a second radially expanded condition. The frame member 23812 can be configured in a variety of ways, such as any of the expandable frame members discloses herein.

In the illustrated example, the frame member 23812 has a generally cylindrical shape (i.e., a circular cross section) having a longitudinal axis L, a proximal end 23814, a distal end 23816 opposite the proximal end 23814, and a diameter D that can be collapsed (i.e., in the first condition as shown in FIG. 140) and expanded (i.e., in the second condition as shown in FIG. 141). In some implementations, however, the frame member 23812 can have a shape other than cylindrical (e.g., a cross section that is oval, rectangular, elliptical, or other suitable shape).

In some implementations, the frame member 23812 can include a plurality of interconnected struts 23818 that are configured to flex or bend to allow the frame member 23812 to move between the first and second condition. In some implementations, the frame member 23812 can also include a plurality of posts 23819 interconnected by the plurality of struts 23818. In the illustrated example, each post 23819 is connected on either side, along a midpoint, to a diamond pattern formed by the struts 23818.

In some implementations, the frame member 23812 is formed as a unitary piece. For example, the frame member 23812 can be laser-cut from a tube. In some implementations, however, some of the struts 23818 and the posts 23819 can be formed separately and connected in any suitable manner (e.g., welded together). In some implementations, the frame member 23812 can comprise and/or be formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

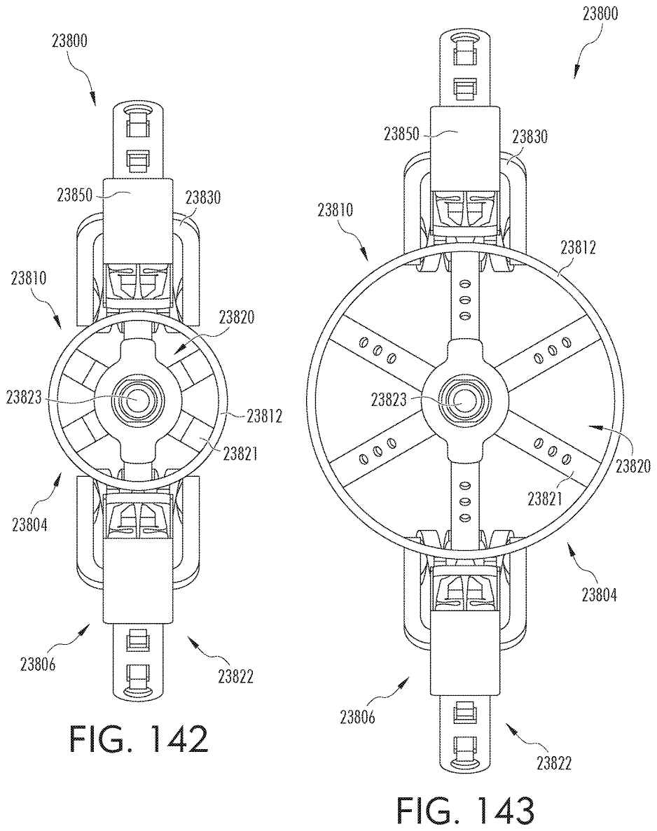

The frame member 23812 can be moved between the first and the second conditions by any suitable means. For example, the frame member 23812 can be mounted around an expandable mechanism 23820 (FIGS. 142-143) configured to engage the frame member 23812 to move the frame member 23812 between the first and the second conditions. A variety of different mechanisms can be used to move the frame member 23812 between the first and second conditions.

In some implementations, as shown in the illustrated example, the expandable mechanism 23820 is the same as or substantially similar to the expandable mechanism 21016 (see FIGS. 84-86) and the description of the expandable mechanism 21016 applies equally to the expandable mechanism 23820. For example, the expandable mechanism 23820 can include an expandable member 23821 (FIG. 136) (e.g., frame, body, strut assembly, tube, shaft, etc.) and an actuation mechanism 23823. The expandable member 23821 and actuation mechanism can be configured in a variety of ways, such as any expandable member (e.g., expandable member 21018) and actuation mechanism (e.g., actuation mechanism 21020) disclosed herein.

In some implementations, the anchor portion 23806 includes one or more anchors 23822 that are actuatable between open and closed conditions and can take a wide variety of forms, such as, for example, paddles, gripping elements, links, or the like. In some implementations, the anchor portion 23806 and/or the one or more anchors 23822 of the device or implant 23800 include paddles 23824 and attachment portions or gripping members 23826.

In some implementations, the paddles 23824 can be opened and closed, for example, to grasp the native leaflets (e.g., native mitral valve leaflets, etc.). The paddles 23824 can be configured in a variety of ways, such as, for example, any of the paddles disclosed herein. In the illustrated example, the paddles 23824 are substantially similar to the paddles 23724 of FIGS. 134-138 and the description of the paddles 23724 applies equally to the paddles 23824.

In the illustrated example, each paddle 23824 includes a proximal portion 23828 that is attached to the frame member 23812, a distal portion 23830 opposite the proximal portion 23828, and am intermediate portion 23832 connecting the proximal portion 23828 to the distal portion 23830. The proximal portion 23828 can attach to the frame member 23812 in any suitable manner (e.g., directly, indirectly, welding, sutures, adhesive, links, latches, integrally formed, a combination of some or all of these, etc.). In some implementations, the proximal portion 23828 is attached to a post 23819 of the frame member 23812. In some implementations, the paddles 23824 are formed as a unitary piece with the frame member 23812 (e.g., laser-cut from a tube with the frame member 23812).

In some implementations, as shown in the illustrated example, the intermediate portion 23832 includes a torsional spring hinge portion 23833 about which the distal portion 23830 can pivot relative to the proximal portion 23828. In some implementations, the torsional spring hinge portion 23833 provides a spring force that biases the distal portion 23830 and the proximal portion 23828 together (i.e., to the closed condition). In the illustrated example, each torsional spring hinge portion 23833 include two coils 23835. In some implementations, however, each torsional spring hinge portion 23833 can include more or less than two coil. In the closed condition, as shown in FIG. 140, the coils 23835 are wound about a coil axis X that is traverse to the longitudinal axis L. In the illustrated example, in the closed condition, the coils 23735 are wound about coil axis X (FIG. 135) which is perpendicular to the longitudinal axis L.

In some implementations, the gripping members 23826 are actuatable between open and closed conditions. The gripping member 23826 can be configured in a variety of ways, such as any gripping member disclosed herein. In the illustrated example, the gripping member 23826 is formed as a clasp that includes a base or fixed arm 23838 (FIG. 140), a moveable arm 23840, optional friction-enhancing elements, other securing structures 23842 (e.g., barbs, protrusions, ridges, grooves, textured surfaces, adhesive, etc.), and a joint portion 23844.

In some implementations, the joint portion 23844 provides a spring force between the fixed and moveable arms 23838, 23840. The joint portion 23844 can be any suitable joint, such as a flexible joint, a spring joint, a pivot joint, or the like. In the illustrated example, the joint portion 23844 has a similar patterned hinge configuration as the patterned hinge portion 23633 of the paddles 23624. Thus, the description of the patterned hinge portion 23633 applies equally to the joint portion 23844.

In some implementations, each anchor 23822 can include a link 23850 (e.g., strip, band, bar, ribbon, rod, etc.) that connects the paddle 23824 to the gripping member 23826. The link 23850 can be configured in a variety of ways. In the illustrated example, the link 23850 is a generally S-shaped band having a proximal portion 23852, a distal portion 23854 opposite the proximal portion 23852, and an intermediate portion 23856 between the distal portion 23854 and the proximal portion 23852. In the illustrated example, the proximal portion 23852 is connected to the intermediate portion 23856 by a first curved section 23858 and the distal portion 23854 is connected to the intermediate portion 23856 by a second curved section 23860. In some implementations, the first curved section 23858 and the second curved section 23860 are configured to bend or flex when the device 23800 moves between the open and closed conditions.

In some implementations, for each anchor 23822, the fixed arm 23838 of the gripping member 23826 is attached to the intermediate portion 23856 of the link 23850. The fixed arm 23838 can be attached to the link 23850 by any suitable means (e.g., directly, indirectly, welding, sutures, adhesive, links, latches, integrally formed, a combination of some or all of these, etc.). The proximal portion 23852 of the link 23850 is attached to the frame member 23812. The proximal portion 23852 can be attached to the frame member by any suitable means (e.g., directly, indirectly, welding, sutures, adhesive, links, latches, integrally formed, a combination of some or all of these, etc.). In some implementations, the proximal portion 23852 is attached to the proximal portion 23828 of the paddle 23824 which is attached to the frame member 23812.

In some implementations, the distal portion 23830 of the paddle 23824 is attached to the distal portion 23854 of link 23850 by a link connector 23862. The link connector 23862 can be configured in a variety of ways. In the illustrated example, the link connector 23862 extends from the distal portion 23854 inward toward the intermediate portion 23856 such that the distal portion 23830 of the paddle 23824 connects to the link connector 23862 between the distal portion 23854 and the intermediate portion 23856 proximate the second curved section 23860.

The anchor portion 23806 can be actuated to open and close in a variety of ways. In some implementations, the device 23800 includes a connector 23866 and an actuation element 23868. The actuation element 23868 can be configured in a variety of ways (e.g., actuation shaft, actuation post, actuation rod, actuation tube, actuation line, etc.), such as any actuation element disclosed herein. In some implementations, the connector 23866 is attached the actuation element 23868 on one end and attached to the distal portion 23854 of the link 23850 on the opposite end. The connector 23866 can be configured in a variety of ways, such as, for example, the connector 8266 of FIG. 28. In some implementations, the connector 23866 is a single piece. In some implementations, however, the connector 23866 can be two or more pieces.

FIG. 140 illustrates the device 23800 in the closed configuration. In some implementations, to move the anchor portion 23806 to the open condition, the actuation element 23868 is moved proximally along the longitudinal axis L. As a result, the connector 23866 is pulled with the actuation element 23868 which pulls the distal portion 23854 of the link 23850 distally. In some implementations, as the distal portion 23854 of the link 23850 moves distally, the first curved section 23858 flexes or bends open moving the intermediate portion 23856 in the direction of arrow Z in FIG. 140. Since the distal portion 23830 of the paddle 23824 is attached to the distal portion 23854 of link 23850, the distal movement of the distal portion 23854 of the link 23850 pivots the paddle 23824 about the torsional spring hinge portion 23833 toward the open condition, against the bias of the coils 23835.

In addition, since the fixed arm 23838 of the gripping member 23826 is attached to the intermediate portion 23856, the fixed arm 23838 moves with the intermediate portion 23856 in the direction of arrow Z. The moveable arm 23840 of the gripping member 23826 can be opened by applying tension, as shown by arrow T in FIG. 140, to one or more actuation lines 23846 attached to the moveable arm 23840, thereby causing the moveable arm 23840 to articulate, flex, or pivot at the joint portion 23844. The one or more actuation lines 23846 extend through the delivery system (e.g., through a steerable catheter and/or an implant catheter). Other actuation mechanisms are also possible. The actuation line 23846 can take a wide variety of forms, such as, for example, a line, a suture, a wire, a rod, a catheter, or the like.

In some implementations, the gripping member 23826 can be spring loaded so that in the closed position the gripping member 23826 continues to provide a pinching force on the grasped native leaflet.

In some implementations, to move from the open condition to the closed or partially closed condition, the tension in the actuation line 23846 can be released allowing the spring force associated with the joint portion 23844 to close the gripping member 23826 and move the moveable arm 23840 to close. Further, the actuation element 23868 can be moved distally along the longitudinal axis L allowing the link 23850 to return to the position shown in FIG. 140. The spring force of the torsional spring hinge portion 23833 and the link 23850, which are biased to the closed condition, aid movement of the device 23800 to the closed condition.

Referring to FIGS. 141 and 143, the expandable frame member 23812 is shown in the expanded condition. The frame member 23812 can move between expanded and contracted conditions as described above regarding any of the expandable frame members disclosed herein. Since the frame member 23812 is moveable independent of the condition of the anchor portions 23806, the frame member 23812 can be moved between the expanded and contracted conditions when the device 23800 is closed, open, or partially closed.

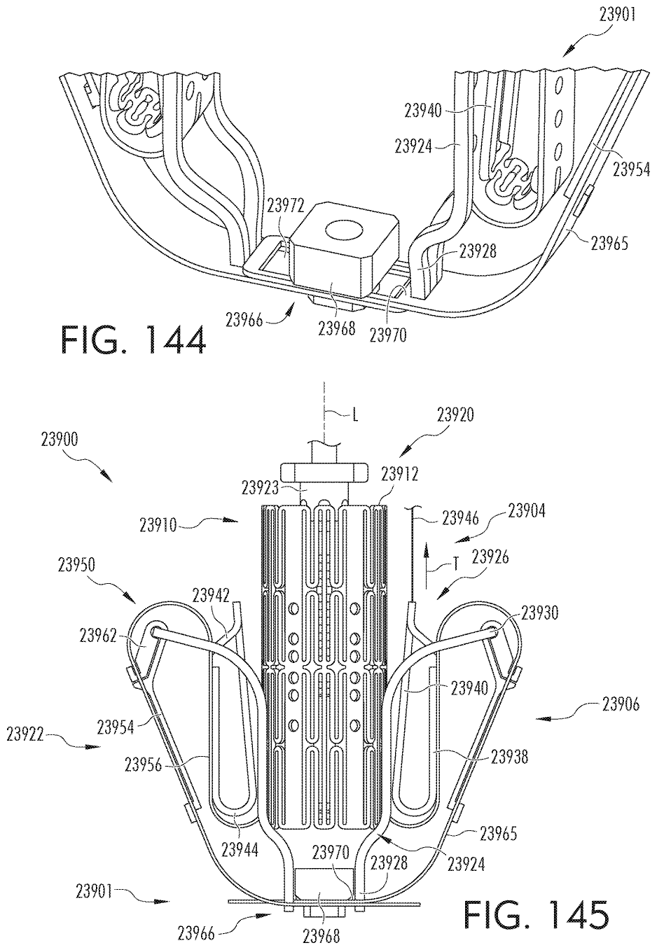

FIGS. 144-146 illustrate an example implantable device or implant 23900 (e.g., a treatment device, a repair device, a prosthetic device, a valve repair device, an implantable device, an implant, etc.) in various stages of deployment. The device 23900 can include any other features for another device or implant discussed in the present application or the applications cited above, and the device 23900 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device or implant 23900 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. In the illustrated example, the device or implant 23900 is substantially similar to the device 23800 except for the configuration of a distal portion 23901 of the device 23900. Thus, apart from the different configuration of the distal portion 23901, the description of the device 23800 applies equally to the device 23900.

In some implementations, the device or implant 23900 includes a coaptation portion 23904 and an anchor portion 23906. In some implementations, the coaptation portion 23904 of the device or implant 23900 includes a coaptation element 23910 that is adapted to be implanted between leaflets of a native valve (e.g., a native mitral valve, native tricuspid valve, etc.). The coaptation element 23910 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23910 is expandable. In some implementations, the expandable coaptation element 23910 includes an expandable frame member 23912 (e.g., frame, body, cage, fixture, chassis, form, etc.) that is configured to move between a first radially contracted condition and a second radially expanded condition.

In some implementations, the frame member 23912 can be moved between the first and the second conditions by any suitable means. For example, the frame member 23912 can be mounted around an expandable mechanism 23920 (FIG. 146) configured to engage the frame member 23912 to move the frame member 23912 between the first and the second conditions. A variety of different mechanisms can be used to move the frame member 23912 between the first and second conditions. In the illustrated example, the expandable mechanism 23920 includes an expandable member 23921 (FIG. 146) (e.g., frame, body, strut assembly, tube, shaft, etc.) and an actuation mechanism 23923.

In some implementations, the anchor portion 23906 includes one or more anchors 23922 that are actuatable between open and closed conditions and can take a wide variety of forms, such as, for example, paddles, gripping elements, links, or the like. In the illustrated example, the anchors 23922 include paddles 23924 and attachment portions or gripping members 23926. The paddles 23924 can be opened and closed, for example, to grasp the native leaflets (e.g., native mitral valve leaflets, etc.). The paddles 23924 can be configured in a variety of ways, such as, for example, any of the paddles disclosed herein. In the illustrated example, each paddle 23924 includes a proximal portion 23928 and a distal portion opposite the proximal portion 23928.

In some implementations, the gripping members 23926 are actuatable between open and closed conditions. The gripping member 23926 can be configured in a variety of ways, such as any gripping member disclosed herein. In the illustrated example, the gripping member 23926 is formed as a clasp that includes a base or fixed arm 23938, a moveable arm 23940, optional friction-enhancing elements, other securing structures 23942 (e.g., barbs, protrusions, ridges, grooves, textured surfaces, adhesive, etc.), and a joint portion 23944.

In some implementations, each anchor 23922 can include a link 23950 (e.g., strip, band, bar, ribbon, rod, etc.) that connects the paddle 23924 to the gripping member 23926. The link 23950 can be configured in a variety of ways. In the illustrated example, the link 23950 is a generally S-shaped band having a proximal portion (not shown), a distal portion 23954 opposite the proximal portion, and an intermediate portion 23956 between the distal portion 23954 and the proximal portion. For each anchor 23922, the fixed arm 23938 of the gripping member 23926 is attached to the intermediate portion 23956 of the link 23950.

In some implementations, the proximal portion of the link 23950 is attached to the frame member 23912. The proximal portion can be attached to the frame member 23912 by any suitable means (e.g., directly, indirectly, welding, sutures, adhesive, links, latches, integrally formed, a combination of some or all of these, etc.). The distal portion 23930 of the paddle 23924 is attached to the distal portion 23954 of link 23950 by a link connector 23962.

In some implementations, as in the illustrated example, the device 23900 includes the connector 23965 and an anchor actuation element 23966. The anchor actuation element 23966 can be configured in a variety of ways. In the illustrated example, the anchor actuation element 23966 includes an actuation cap 23968. In some implementations, the actuation cap 23968 is configured similar to the actuation cap 8214 of FIG. 27.

In some implementations, each anchor 23922 includes a separate connector 23965 which is attached to the actuation cap 23968 at a proximal portion 23970 of each connector 23965 and attached to the distal portion 23954 of the link 23950 on the opposite end. In the illustrated example, the proximal portion 23928 of each paddle 23924 is attached a proximal portion 23970 of a corresponding connector 23965. The proximal portion 23928 can be attached to the proximal portion 23970 by any suitable means (e.g., directly, indirectly, welding, sutures, adhesive, links, latches, integrally formed, a combination of some or all of these, etc.).

In some implementations, the proximal portion 23970 of each connector 23965 includes structure configured to allow radial movement of the connector 23965 relative to the actuation cap 23968 when the frame member 23912 expands and contracts. Thus, each connector 23965 can move with the frame member 23912 as the frame member 23912 expands and contracts. The structure for allowing radial movement of the connectors 23965 can be configured in a variety of ways. In the illustrated example, the proximal portion 23970 of each connector 23965 includes a radially extending slot 23972 (FIG. 144). The radially extending slots 23942 on each proximal portion 23970 overlap such that a portion of the actuation cap 23968 is received through both slots 23942.

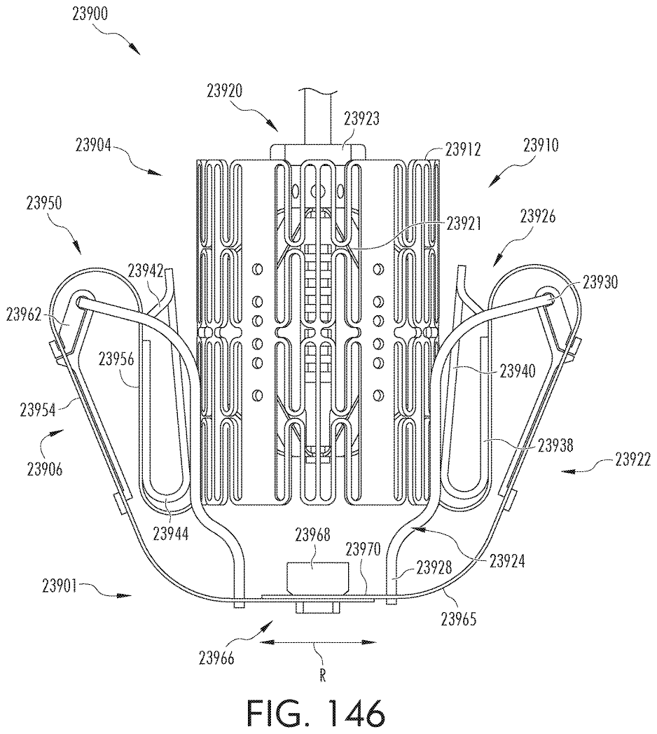

FIG. 145 illustrates the device 23900 in the closed configuration and the coaptation element 23910 in the collapsed condition. In some implementations, the anchor portion 23906 and the gripping members 23926 can move between the open condition and the closed condition in the same manner as described above regarding the device 23800. In some implementations, the anchor portions 23906 can be opened by moving the actuation element 23968 proximally along the longitudinal axis L. In addition to opening the paddles 23924, proximal movement of the actuation element 23968 also moves the fixed arms 23940 of the gripping members 23926. In some implementations, the moveable arm 23940 of the gripping member 23826 can be opened by applying tension, as shown by arrow T in FIG. 145, to one or more actuation lines 23946 attached to the moveable arm 23940,

Referring to FIGS. 146, the expandable frame member 23912 is shown in the expanded condition. The frame member 23912 can move between expanded and contracted conditions as described above regarding any of the expandable frame members disclosed herein. In some implementations, when the frame member 23912 expands, the proximal portion 23970 of each connector 23965 slides radially outward, as shown by arrow R in FIG. 146, so that the anchor portions 23906 move with the expanding coaptation element 23910. Since the frame member 23912 is moveable independent of the condition of the anchor portions 23906, the frame member 23912 can be moved between the expanded and contracted conditions when the device 23900 is closed, open, or partially closed.

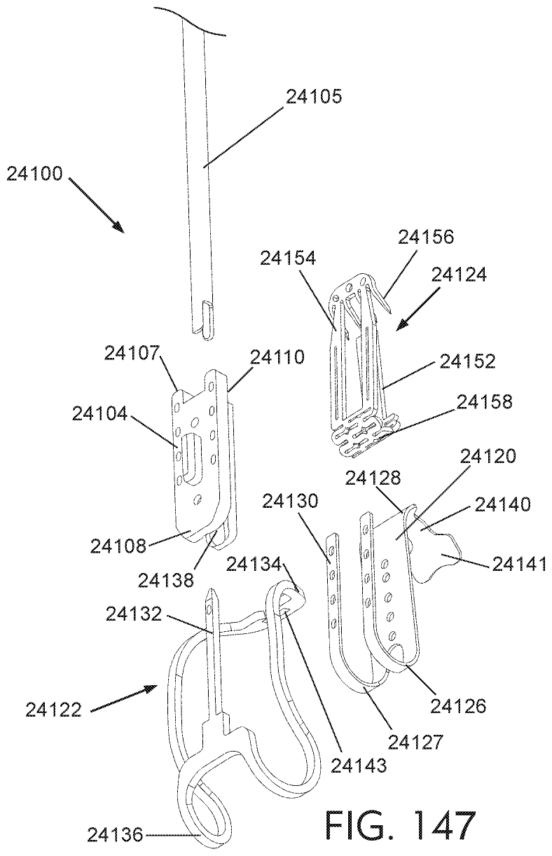

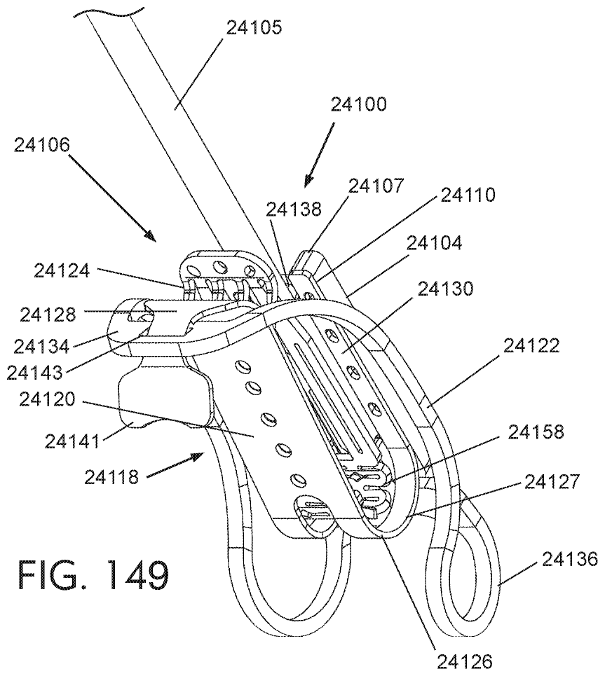

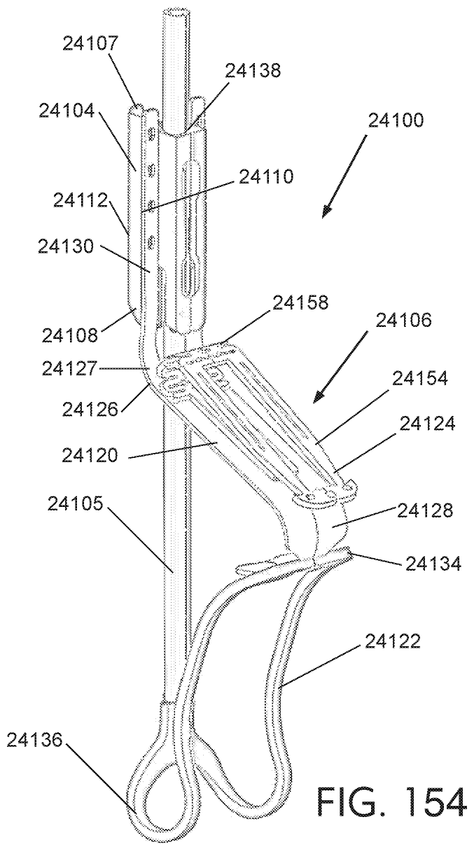

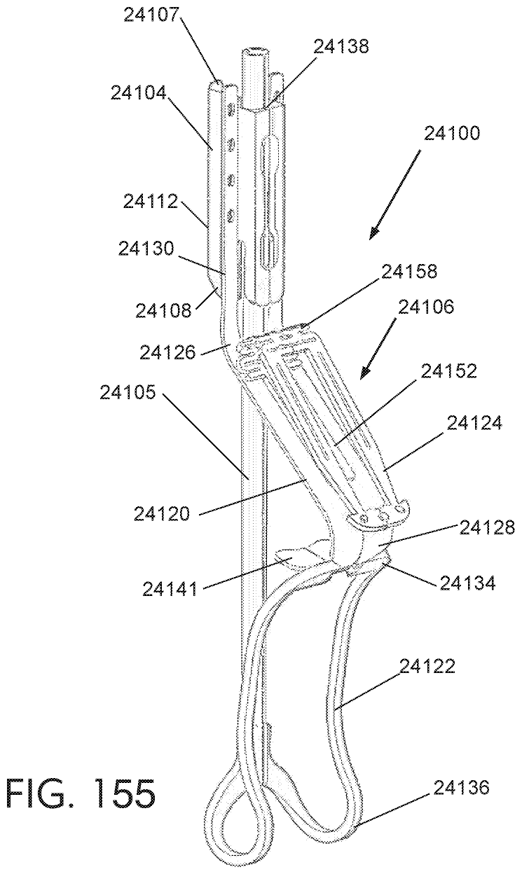

Referring now to FIGS. 147-155, a device or implant 24100 (e.g., a treatment device, a repair device, a prosthetic device, a valve repair device, an implantable device, an implant, etc.) that can be the same as or similar to the device or implant 23100 is shown. The device 24100 can operate in the same or similar manner to the device 23100, but one or more of the components can be made from a sheet material, such as sheet metal, such as sheet steel, sheet nitinol, etc. The device 24100 can include any other features for another device or implant discussed in the present application or the applications cited above, and the device 24100 can be positioned to engage valve tissue (e.g., leaflets 20, 22, 30, 32, 34) as part of any suitable treatment and/or repair system (e.g., any treatment and/or repair system disclosed in the present application, or the applications cited herein). Device 24100 can be configured as a component (e.g., anchor portion, etc.) of a larger device or implant.

In some implementations, the device or implant 24100 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 24100 includes a body portion 24104 and an anchor portion 24106.

In some implementations, the body portion 24104 of the device or implant 24100 can function as a coaptation element that is adapted to be implanted between leaflets of a native valve (e.g., a native mitral valve, native tricuspid valve, etc.). In some implementations, the body portion 24104 can be attached to a coaptation element (e.g., spacer, coaptation element, gap filler, membrane, sheet, plug, wedge, balloon, frame, etc.) such as any suitable coaptation element (e.g., any coaptation element disclosed in the present application, or the applications cited above).

In some implementations, the body portion 24104 supports the anchor portion 24106. In some implementations, the body portion 24104 can guide movement of the anchor portion 24106 and/or an actuation element 24105 (e.g., tube, rod, shaft, etc.) relative to the body portion 24104. The body portion 24104 can be configured in a variety of ways. In the illustrated example, the body portion 24104 includes a proximal portion 24107, a distal portion 24108 opposite the proximal portion 24107, an inner face 24110 extending between the proximal portion 24107 and the distal portion 24108, and an outer face 24112 opposite the inner face 24110. In some implementations, the inner face 24110 and the outer face 24112 are planar and parallel to each other. In some implementations, however, the inner face 24110 and the outer face 24112 need not planar and/or parallel.

In some implementations, the anchor portion 24106 can include one or more anchors 24118 that are actuatable between open and closed conditions. The anchors 24118 can take a wide variety of forms, such as, for example, links, paddles, gripping elements, or the like. In the illustrated example, the device or implant 24100 includes a single anchor 24118. In the illustrated example, anchor portion 24106 and/or anchors 24118 include a first link or paddle 24120, a second link or paddle 24122 operatively connected to the first link 24120, and a gripping member 24124.

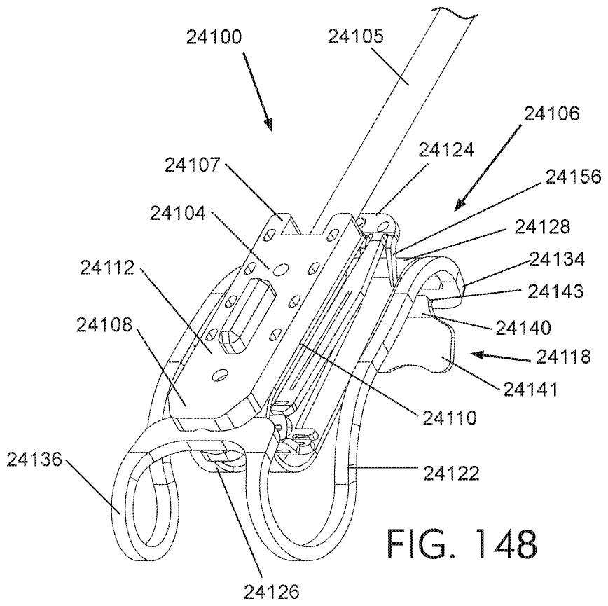

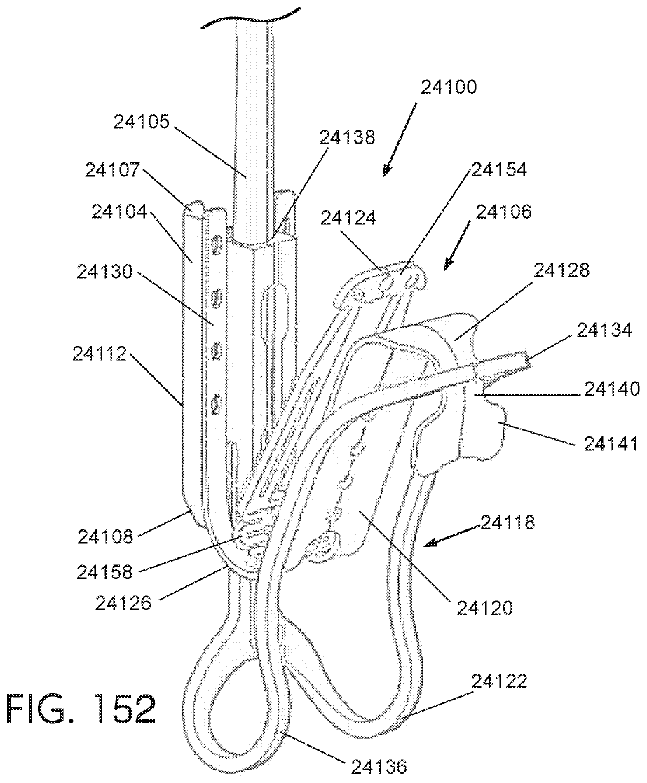

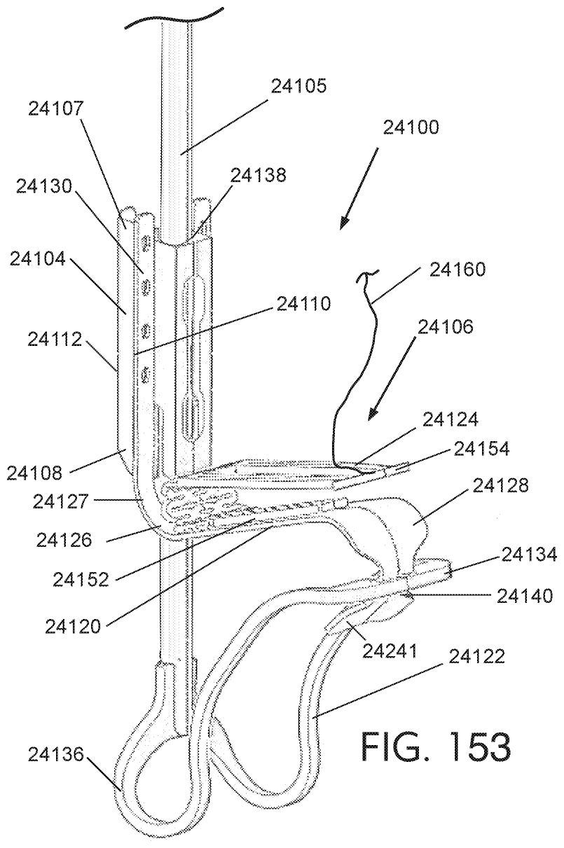

In some implementations, the first link 24120 is connected to both the second link 24122 and the body portion 24104 and is movable between an open configuration (FIGS. 152-155) and a closed configuration (FIGS. 148-150). The first link 24120 can be configured in a variety of ways. In the illustrated example, the first link 24120 has a rigid, elongated sheet portion. The first link 24120 has a first end portion 24126 and a second end portion 24128 opposite the first end portion 24126. In the illustrated example, the first end portion 24126 is connected to the body portion 24104 via a pair of struts or legs 24130.

In some implementations, the first end portion 24126 includes a flexible hinge portion 24127 so that the first link 24120 can move pivot between open and closed conditions. The flexible hinge portion 24127 can be configured in a variety of ways, such as for example, a bendable or flexible portion of the first end portion 24126 such that the first end portion 24126 bends to allow movement of the first link 24120 between open and closed conditions.

In some implementations, as in the illustrated example, the second end portion 24128 of the first link 24120 is connected to the second link 24122. In some implementations, the second link 24122 is configured to be movable relative to the body portion 24104 in order to move the anchors 24118 between open and closed conditions. The second link 24122 can be configured in a variety of ways. In the illustrated example, the second link 24122 can comprise and/or be formed from a single piece of sheet material that is cut and shape set. In some implementations, cross-sections of the second link 24122 are rectangular and have different sizes depending on where the section is taken. The different section sizes can be selected to control the bending of the second link 24122. In some implementations, however, the second link 24122 can be formed from two or more pieces and/or can be shaped other than a rectangular cross-section.

In some implementations, as in the illustrated example, the second link 24122 includes a first portion 24132 that extends through the body portion 24104, a second portion 24134 that is connected to the second portion 24128 of the first link 24120, and a flexible intermediate portion 24136 connecting the first portion 24132 to the second portion 24128. As shown in FIG. 147, in some implementations, the first portion 24132 is linear or substantially linear and extends through passages 24138 in the body portion 24104. In some implementations, the passage 24138 acts as a guide allowing the second link 24122 to move axially with respect to the body portion 24104.

In some implementations, the second portion 24134 of the second link 24122 is flexibly and/or pivotably connected to the second portion 24128 of the first link 24120. In some implementations, the second portion 24134 of the second link 24122 can be pivotably connected to the second portion 24128 of the first link 24120 in any suitable manner. As shown in FIGS. 148-155, in some implementations, the second portion 24128 of the first link 24120 includes a neck 24140 and a tab 24141 and the second portion 24134 of the second link 24122 includes a slot or cutout 24143. In some implementations, the neck 24140 fits in the slot or cutout 24143 to couple the first link 24120 to the second link 24122.

Referring to FIGS. 147-150, in some implementations, the flexible hinge portion 24127 of the first link 24120 is bent or curved and provides an offset between the second end 24128 of the first link 24120 and the inner face 24110 of the body portion 24104 when the device 24100 is in the closed condition. In some implementations, the first link 24120 extends parallel to or substantially parallel to and offset from the inner face 24110 when in the closed condition.

Referring to FIG. 155, the device 24100, is illustrated in an elongated or open condition with the gripping member 24124 in a closed position. The elongated or open condition is suitable for delivery of the device 24100 through a catheter, a sheath, a tube, a channel, a pathway, or the like. The gripping member 24124 can be configured in a variety of ways, such as any gripping member 24124 disclosed herein.

In some implementations, the gripping member 24124 is formed as a clasp that includes a base or fixed arm 24152, a moveable arm 24154, optional friction-enhancing elements or other securing structures 24156 (e.g., barbs, protrusions, ridges, grooves, textured surfaces, adhesive, etc.), and a joint portion 24158. The fixed arm 24152 is attached to the first link 24120.

In some implementations, the joint portion 24158 provides a spring force between the fixed and moveable arms 24152, 24154. In some implementations, the joint portion 24158 can be any suitable joint, such as a flexible joint, a spring joint, a pivot joint, or the like, the fixed arm 24152 is attached to the first link 24120 and remains stationary or substantially stationary relative to the first link 24120 when the moveable arm 24154 are opened to open the gripping member 24124 and expose the optional barbs or other friction-enhancing elements 24156.

In some implementations, the gripping member 24124 is opened by applying tension to one or more actuation lines attached to the moveable arm 24154, thereby causing the moveable arm 24154 to articulate, flex, or pivot at the joint portion 24158. In some implementations, the one or more actuation lines extend through the delivery system (e.g., through a steerable catheter and/or an implant catheter). Other actuation mechanisms are also possible. The actuation line can take a wide variety of forms, such as, for example, a line, a suture, a wire, a rod, a catheter, or the like. The gripping member 24124 can be spring loaded so that in the closed position the gripping member 24124 continues to provide a pinching force on the grasped native leaflet.

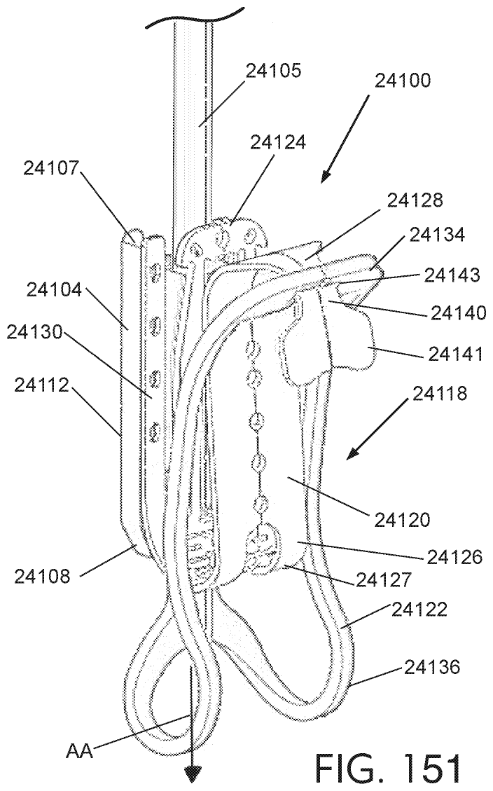

Referring to FIG. 155, in the elongated condition, the second link 24122 is in an extended position relative to the body portion 24104. Movement of the second link 24122 to the extended position (See FIGS. 151-155) relative to the body portion 24104, as shown by arrow AA in FIG. 151 can be accomplished in a variety of ways. For example, in some implementations, the body portion 24104 can be coupled to and/or held in place by a catheter (not shown) and the first portion 24132 of the second link 24122 can be engaged by the actuation element 24105 to move the first portion 24132 axially within the passage 24138 in the body portion 24104. The actuation element 24105 can take a wide variety of different forms (e.g., as a wire, rod, shaft, tube, screw, suture, line, strip, combination of these, etc.), be made of a variety of different materials, and have a variety of configurations, such as any of the actuation elements disclosed herein.

In some implementations, once the device 24100 is positioned within the native valve adjacent a leaflet (e.g., leaflets 20, 22, 30, 32, 34), the device 24100 can be moved to a capture ready condition (i.e., partially closed) as shown in FIG. 153 (except the clasps would be opened, instead of closed). To move from the elongated condition to the capture ready condition, the second link 23122 is moved proximally relative to the body portion 23104 (i.e., see the progression from FIG. 155 to FIG. 153).

In some implementations, since the second portion 24134 of the second link 24122 is connected to the second portion 24128 of the first link 24120, moving the second link 24122 proximally relative to the body portion 24104 causes the second portion 24128 of the first link 23120 to move with the second portion 24134 of the second link 24122.

As a result, the first link 24120 flexes and/or pivots the flexible intermediate portion 24136 of the second link 24122 flexes open such that an angle between the proximal portion 24132 and the second portion 24134 of the second link 24122 increases. The fixed arm 24152 of the gripping member 24124 moves with the first link 24120. In some implementations, the gripping member 24124 is spring loaded to the closed condition.

In some implementations, once the leaflet 20 is in a satisfactory position to be grasped, tension in the actuation line 24160 can be released (see FIG. 153). Releasing tension in the actuation line 24160 allows the spring force associated with the joint portion 24158 to close the gripping member 24124 by moving the moveable arm 24154 toward the fixed arm 24152 and into contact with the leaflet 20, to pinch the leaflet 20 between the moveable arm 24154 and the fixed arm 24152.

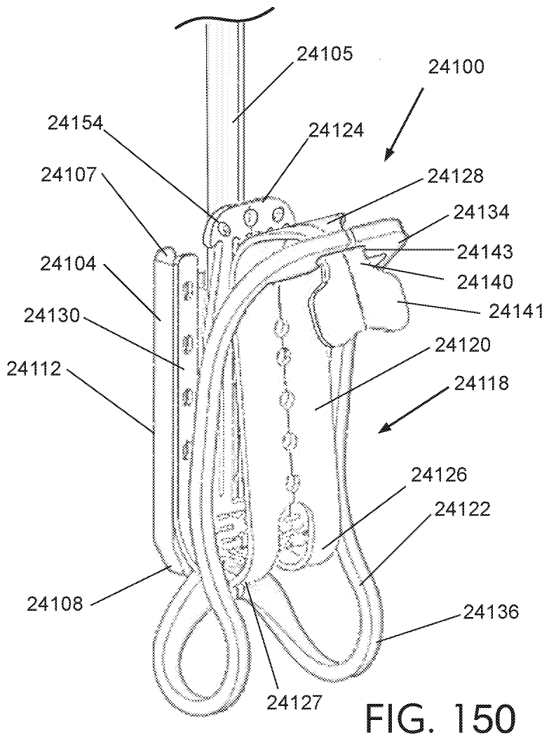

In some implementations, once the leaflet 20 is captured in the gripping member 24124, the device 24100 can be moved to the closed condition as shown in FIG. 150. To move the device 24100 to the closed position, the second link 24122 is moved proximally relative to the body portion 24104 (i.e., the proximal portion 24132 of the second link 24122 moves axially relative to the body portion 24104 in the proximal direction).

In some implementations, since the distal portion 24134 of the second link 24122 is connected to the distal portion 24128 of the first link 24120, moving the second link 24122 proximally relative to the body portion 24104 causes the distal portion 24128 of the first link 24120 to move with the distal portion 24134 of the second link 24122.

As a result, the first link 24120 moves and/or pivots about the connection 23130 and the flexible intermediate portion 24136 of the second link 24122 relaxes closed such that the angle between the proximal portion 24132 and the distal portion 24134 of the second link 24122 decreases from the capture ready condition of FIG. 153.

In some implementations, since the fixed arm 24152 of the gripping member 24124 is attached to the first link 24120, the closed gripping member 24124 moves with the first link 24120. In some implementations where the first link 24120 has the offset, as shown in FIG. 150, the closed griping member 24124 can be positioned between the body portion 24104 and the first link 24120 when the device 24100 is in the closed condition. In some implementations where the first link 24120 does not include the offset, the body portion 24104 can be contoured to receive a portion of the closed gripping member 24124. For example, the inner face 24110 of the body portion 24104 can have an opening or recess (not shown) the receives a portion of the gripping member 24124.

In some implementations, a single device or implant 24100 can be used without use of another device or implant 24100. For example, the single device 24100 can be used to capture a single leaflet (e.g., leaflets 20, 22, 30, 32, 34) as part of an overall repair strategy. In some implementations, the single device 24100 can be used with a coaptation element, such as any of the coaptation elements disclosed herein. In some implementations, the single repair device 24100 can also be used to capture multiple leaflets in the anchor 24118, such as both leaflets 20, 22 of the mitral valve MV (FIG. 5) or two or more leaflets 30, 32, 34 of the tricuspid valve TV (FIG. 7).

In some arrangements, two devices 24100 can be arranged back-to-back and secured together. For example, the outer faces 24112 of two devices 24100 can be attached together so that the two devices 24100 are arranged with the inner faces 24110 facing in opposite directions. In some implementations, the attached devices 24100 can be deployed or implanted in a native heart valve where each device 24100 can capture a leaflet of the heart valve. For example, the two devices 24100 can be deployed or implanted in a mitral valve MV where a first device 24100 can capture the anterior leaflet 20 and the second device can capture the posterior leaflet 22. Similarly, the two devices 24100 can be deployed or implanted in a tricuspid valve TV where the two devices 24100 capture two or three of the leaflets 30, 32, 34 of the tricuspid valve TV.

In some implementations, each of the devices 24100 is independently actuatable. Thus, for example, one of the two devices 24100 can be moved to a closed condition to grasp a valve leaflet while the other of the two devices remains in an open or partially closed position.

In some implementations, the two devices 24100 can be tethered together. In some implementations, the tethered devices can be separately attached to two separate leaflets, drawn together by the tethers, and then fixed together or setting a distance between the two devices (e.g., by tying the tethers off). In some implementations, the tethered devices can be separately attached to one leaflet and another structure (e.g., chordae tendinea, papillary muscle, etc.), drawn together by the tethers, and then fixed relative to one another (e.g., by tying the tethers off).

In some arrangements, three devices 24100 can be arranged in a triangular pattern and secured together. For example, the outer faces 24112 of three devices 24100 can be arranged to face inward toward each other so that the inner faces 24110 face outward. The three devices 24100 can be attached to each other in any suitable manner. In some implementations, the attached devices 24100 can be implanted in a tricuspid valve TV where each device 24100 can capture a leaflet of the tricuspid valve TV. For example, a first device 24100 can capture the anterior leaflet 30, the second device can capture the septal leaflet 32, and the third device can capture the posterior leaflet 34. In some implementations, each of the devices 24100 is independently actuatable. Thus, for example, one of the three devices 24100 can be moved to a closed condition to grasp a valve leaflet while the other two of the devices remain in an open or partially closed position.

In some implementations, two devices 24100 can be arranged back-to-back with a coaptation element (e.g., coaptation element of FIG. 84) between the two devices 24100. Any suitable coaptation element can be used in the arrangement, such as any coaptation element disclosed herein.

In some implementations, each of the two devices 24100 can be attached to the coaptation element. For example, in some implementations, the outer faces 24112 of the two devices 24100 can be attached to the coaptation element such that the inner faces 24110 are facing outward. In some implementations, the two devices 24100 can be attached to the coaptation element on opposite sides of the coaptation element.

In some implementations, the attached devices 24100 can be implanted in a native heart valve where each device 24100 can capture a leaflet of the heart valve. For example, the two devices 24100 can be implanted in a mitral valve MV where a first device 24100 can capture the anterior leaflet 20 and the second device 23100′ can capture the posterior leaflet 22. Similarly, the two devices 24100 can be implanted in a tricuspid valve TV where the two devices 24100 capture two or three of the leaflets 30, 32, 34 of the tricuspid valve TV.

In some implementations, each of the devices 24100 is independently actuatable. Thus, for example, one of the two devices 24100 can be moved to a closed condition to grasp a valve leaflet while the other of the two devices remains in an open or partially closed position.

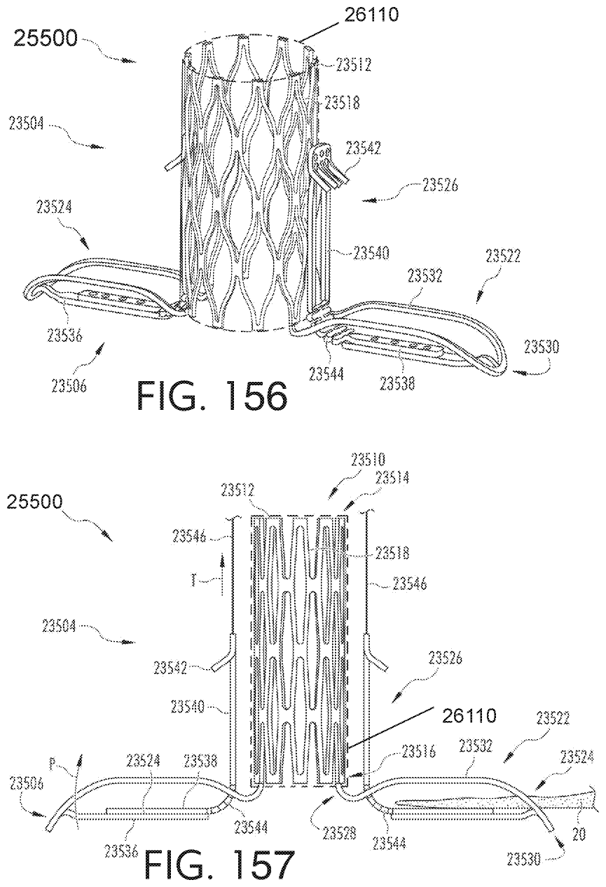

Referring to FIGS. 156-166, in some implementations, a device 25500 (e.g., a treatment device, a repair device, a prosthetic device, a valve repair device, an implantable device, an implant, etc.) is configured to be expanded and act as a dock for a replacement valve 26100. For example, a device 25500 may be effective for a period but may become less effective due to heart disease (e.g., a disease that causes enlargement of the heart or portions of the heart) or a due to an event (e.g., a heart attack). In some implementations, the device 25500 serves two functions. First, the device 25500 repairs a native valve by connecting two leaflets of the native valve together and placing the coaptation element between the leaflets to block regurgitant flow. Second, the device 25500 converts to a dock that accepts a replacement valve 26100 and secures the replacement valve inside the native valve.

FIGS. 156-166 illustrate an example of a device or implant 25500 (e.g., a treatment device, a repair device, a prosthetic device, a valve repair device, an implantable device, an implant, etc.). The device 25500 can include any other features for another device or implant discussed in the present application or the applications cited above, and the device 25500 can be positioned to engage valve tissue (e.g., leaflets 20, 22, 30, 32, 34) as part of any suitable treatment and/or repair system (e.g., any treatment and/or repair system disclosed in the present application, or the applications cited herein).

In some implementations, the device 25500 is the same or substantially similar to the device 23500 described above, except the device 25500 includes a frangible or breakable cover 26110. However, the device 25500 can take a wide variety of different forms. For example, any of the devices or implants disclosed herein can be modified to be expanded and act as a dock for a replacement valve 26100. Another optional difference between the device 25500 and the device 23500 is that the device 25500 can be configured such that the coaptation portion 23504 of the device 25500 is optionally only expandable to accept the replacement valve 26100 (e.g., not to change the size of the coaptation portion 23504 in the native valve to optimize the performance of the coaptation portion). In some implementations, the device 25500 can both be expanded to optimize the performance of the coaptation portion and can be expanded to accept the replacement valve.

The device or implant 25500 is deployed from a delivery system (e.g., delivery system 102). The delivery system can comprise one or more of a catheter, a sheath, a guide catheter/sheath, a delivery catheter/sheath, a steerable catheter, an implant catheter, a tube, a channel, a pathway, combinations of these, etc. The device or implant 25500 includes a coaptation portion 23504 and an anchor portion 23506.

In some implementations, the coaptation portion 23504 of the device or implant 25500 includes a coaptation element 23510 that is adapted to be implanted between leaflets of a native valve (e.g., a native mitral valve, native tricuspid valve, etc.). The coaptation element 23510 (e.g., spacer, coaptation element, gap filler, membrane, sheet, plug, wedge, balloon, frame, etc.) can be any suitable coaptation element (e.g., any coaptation element disclosed in the present application, or the applications cited above). In some implementations, the coaptation element 23510 is expandable. In some implementations, the expandable coaptation element 23510 includes an expandable frame member 23512 (e.g., frame, body, cage, fixture, chassis, form, etc.) that is configured to move between a first radially contracted condition and a second radially expanded condition. The frame member 23512 can be configured in a variety of ways, such as any of the expandable frame members discloses herein.

In the illustrated example, the frame member 23512 has a generally cylindrical shape (i.e., a circular cross section) having a longitudinal axis L, a proximal end 23514, a distal end 23516 opposite the proximal end 23514, and a diameter D that can be collapsed and expanded. In some implementations, however, the frame member 23512 can have a shape other than cylindrical (e.g., a cross section that is oval, rectangular, elliptical, or other suitable shape).

The frame member 23512 can include a plurality of interconnected struts 23518 that are configured to flex or bend to allow the frame member 23512 to move between the first and second condition. The interconnected struts 23518 can be configured in a variety of ways, such as for example, the number and size of the struts, the shape of each strut, the arrangement of struts relative to other struts, the interconnection of the struts, etc. Any configuration that can facilitate the expansion and contraction of the frame member 23512 can be used. In some implementations, the struts 23518 are arranged in diamond, oval, lens, or similar shape.

In some implementations, the frame member 23512 is formed as a unitary piece. For example, the frame member 23512 can be laser-cut from a tube. In some implementations, however, some of the struts 23518 can be formed separately and connected in any suitable manner (e.g., welded together). In some implementations, the frame member 23512 can comprise and/or be formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

The frame member 23512 can be moved between the first and the second conditions by any suitable means. For example, the frame member 23512 can be mounted around an expandable mechanism 23520 (illustrated schematically in FIG. 122) configured to engage the frame member 23512 to move the frame member 23512 between the first and the second conditions. A variety of different mechanisms can be used to move the frame member 23512 between the first and second conditions. For example, any expandable mechanism disclosed herein can be used, such as, but not limited to, the expandable mechanism 21016 of FIGS. 89-93, the expandable mechanism 22016 of FIGS. 92-98, the expandable mechanism 22116 of FIG. 99, an inflatable balloon, an expandable stent, such as the stent of a replacement valve, etc.

The cover 26110 can take a wide variety of different forms. The cover 26110 can be made from any material that blocks or inhibits the flow of blood through the cover and that can be cut, ruptured, broken, ablated, etc. to allow blood to flow through the frame member 23512. For example, the cover 26110 can be a cloth material, a film, such as a plastic film, tissue, such as bovine or porcine tissue, etc. The cover 26110 can be provided on or in the coaptation portion 23504 in any manner that prevents or inhibits the flow of blood through the coaptation portion 23504. For example, the cover 26110 can be provided on one or more of the proximal end of the coaptation portion 23504, the distal end of the coaptation portion, and the sides or outer periphery of the coaptation portion.

The anchor portion 23506 includes one or more anchors 23522 that are actuatable between open and closed conditions and can take a wide variety of forms, such as, for example, paddles, gripping elements, links, or the like. The anchor portion 23506 can be actuated to open and close in a variety of ways. In some implementations, an actuation element (not shown) is used to move the anchors between open and closed conditions. The actuation element (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 23506 relative to the coaptation portion 23504. Or, the actuation element can be unthreaded, such that pushing or pulling the actuation element moves the anchor portion 23506 relative to the coaptation portion 23504.

The anchor portion 23506 and/or the one or more anchors 23522 of the device or implant 23500 include paddles 23524 and attachment portions or gripping members 23526. The paddles 23524 can be opened and closed, for example, to grasp the native leaflets (e.g., native mitral valve leaflets, etc.). The paddles 23524 can be configured in a variety of ways, such as, for example, any of the paddles disclosed herein. In the illustrated example, the device 23500 includes a pair of paddles 23524 attached to, and extending from, the distal end 23516 of the frame member 23512 on opposite sides of the frame member 23512. In some implementations, the device 23500 can include more or less than a pair of paddles 23524.

In the illustrated example, each paddle 23524 includes a proximal portion 23528 that is attached to, and extends from, the distal portion 23516 of the frame member 23512, a distal portion 23530 opposite the proximal portion 23528, and an intermediate portion 23532 connecting the proximal portion 23528 to the distal portion 23530. The paddles 23524 can have a variety of shapes. In some implementations, each paddle 23524 is U-shape such that the proximal portion 23528 attached to the frame member 23512 at two spaced apart locations and the distal portion 23530 has a closed end.

The proximal portion 23528 can attach to the distal end 23516 of the frame member 23512 in any suitable manner (e.g., welded together). In some implementations, the paddles 23524 are formed as a unitary piece with the frame member 23512 (e.g., laser-cut from a tube with the frame member 23512). Each paddle 23524 can be jointed and/or flexible to move between all the positions described below regarding FIGS. 122-128. In the illustrated example, the paddle 23524 is flexible or bendable at, or proximate to, the proximal portion 23528. In some implementations, the paddle 23524 can be flexible or bendable at any suitable location along the intermediate portion 23532.

Each paddle 23524 includes a mounting surface 23534 to which one or more gripping members 23526 can be attached. The mounting surface 23534 can be configured in a variety of way, including position on the paddle 23524, size, orientation, etc. In the illustrated example, the mounting surface 23534 is disposed on a mounting arm 23536 that extends proximally from the distal portion 23516. In some implementations, the mounting arm 23536 can extend parallel to, and offset from, the intermediate portion 23532.

In some implementations, each paddle 23524 has a gripping member 23526 attached. In some implementations, the gripping member 23526 is attached to the mounting arm 23536. The gripping member 23526 can be configured in a variety of ways, such as any gripping member disclosed herein. In the illustrated example, the gripping member 23526 is formed as a clasp that includes a base or fixed arm 23538, a moveable arm 23540, optional friction-enhancing elements, other securing structures 23542 (e.g., barbs, protrusions, ridges, grooves, textured surfaces, adhesive, etc.), and a joint portion 23544. The fixed arm 23538 is attached to the mounting arm 23536. In some implementations, the fixed arm 23538 is attached to the mounting arm 23536 with the joint portion 23544 disposed proximate the proximal portion 23528 of the paddle 23524.

The joint portion 23544 provides a spring force between the fixed and moveable arms 23538, 23540. The joint portion 23544 can be any suitable joint, such as a flexible joint, a spring joint, a pivot joint, or the like. The fixed arm 23538 is attached to the paddle 23524 and remains stationary or substantially stationary relative to the paddle 23524 when the moveable arm 23540 are opened to open the gripping member 23526 and expose the optional barbs or other friction-enhancing elements 23542.

In some implementations, the gripping member 23526 is opened by applying tension, as shown by arrow T in FIG. 123, to one or more actuation lines 23546 attached to the moveable arm 23540, thereby causing the moveable arm 23540 to articulate, flex, or pivot at the joint portion 23544. The one or more actuation lines 23546 extend through the delivery system (e.g., through a steerable catheter and/or an implant catheter). Other actuation mechanisms are also possible. The actuation line 23546 can take a wide variety of forms, such as, for example, a line, a suture, a wire, a rod, a catheter, or the like. The gripping member 23526 can be spring loaded so that in the closed position the gripping member 23526 continues to provide a pinching force on the grasped native leaflet.

Once the device 23500 is positioned within the native valve adjacent a leaflet (e.g., leaflets 20, 22, 30, 32, 34), the anchor portion 23506 can move to a capture ready condition (i.e., partially closed). As indicated above, the device 23500 can be configured to move the anchor portion 23506 between the open condition, the capture ready condition, and the closed condition in a variety of ways, such as an actuation element (not shown) or/and shape setting the anchor portion 23506. In the capture ready condition, the paddles 23524 partially close by flexing or bending proximate to, or at, the proximal portion 23528. In some implementations, the paddles 23524 extend perpendicular, or near perpendicular, to the longitudinal axis L.

In the capture ready position, the gripping member 23526 is in an open condition. The fixed arm 23538 of the gripping member 23526 moves with the paddle 23524 to partially close the gripping member 23526 while the moveable arm 23540 is held open by the actuation line 23546. In some implementations, the gripping member 23526 is spring loaded to the closed condition and thus helps pivot the paddle 23524 in the direction of arrow P.

Once the leaflet 20 is in a satisfactory position to be grasped, tension in the actuation line 23546 can be released. Releasing tension in the actuation line 23546 allows the spring force associated with the joint portion 23544 to close the gripping member 23526 by moving the moveable arm 23540 toward the fixed arm 23538 and into contact with the leaflet 20, as shown in FIG. 125, to pinch the leaflet 20 between the moveable arm 23540 and the fixed arm 23538. Once one leaflet is grasped in one of the anchor portions 23506, the device 23500 can be positioned to grasp a second leaflet (e.g., leaflet 22) in the other anchor portion 23506. For example, with one leaflet grasped in one of the anchor portions 23506, the frame member 23512 can be expanded or contracted to position the other anchor portion 23506 to capture a second leaflet.

Once the leaflets are captured in the gripping members 23526, the device 25500 can be moved to the closed condition. When moving to the closed condition, the paddles 23524 further bend or flex proximate to, or at, the proximal portion 23528, such that the anchor portions 23506 extend along the coaptation portion 23504. In some implementations, the paddles 23524 and/or the moveable arm 23540 can contact the coaptation portion 23504 in the closed condition.

The coaptation portion 23504 can be expanded from the first radially contracted condition to the second radially expanded condition such that the diameter D of the frame member 23512 increases. The coaptation portion 23504 can be expanded from the first radially contracted condition to the second radially expanded condition to block more regurgitant flow through the native valve (i.e., to enhance the performance of the coaptation portion 23504) and/or to allow the coaptation portion 23504 to accept a replacement valve 26100. The coaptation portion 23504 can be in the expanded condition with the anchor portions 23506 in the closed condition. The coaptation portion 23504, however, can moved between the expanded condition and the contracted position when the anchor portions 23506 are in any position (e.g., open condition and partially closed condition). The frame member 23512 can be moved between the first and the second conditions by an optional expandable mechanism 23520 (illustrated schematically in FIG. 122), a balloon, by an expandable valve, or any suitable means.

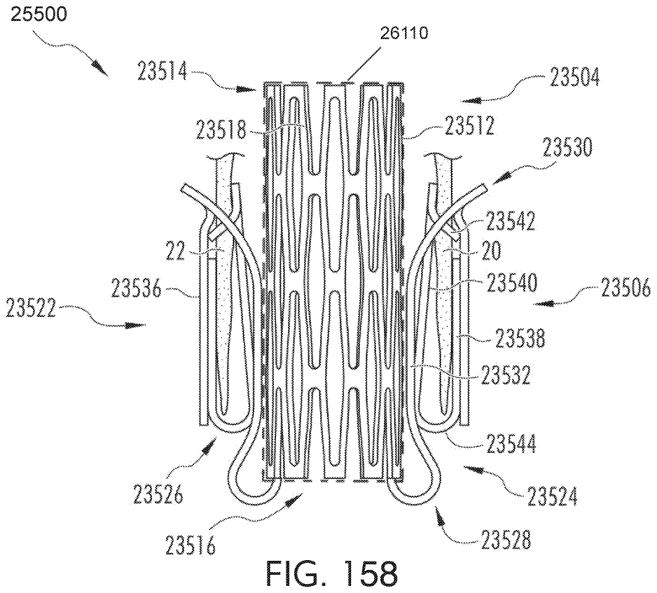

Referring to FIGS. 156-166, in some implementations, the device 25500 converts to a dock that accepts a replacement valve 26100 and secures the replacement valve 26100 inside the native valve. FIGS. 156-157 illustrate the device 25500 in an open, capture ready condition. FIG. 158 illustrates the device 25500 implanted on a pair of native valve leaflets 20, 22.

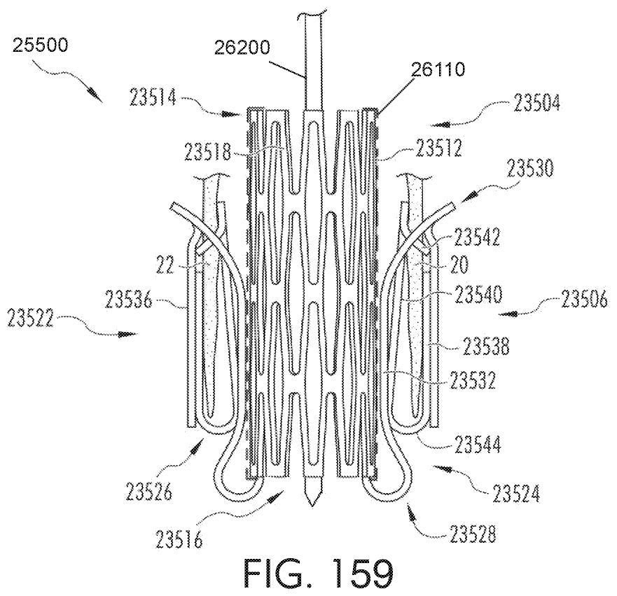

FIG. 159 illustrates a tool 26200 modifying the device 25500 to allow the device to accept the replacement valve 26100. For example, the tool 26200 can be configured to cut, rupture, break, or ablate the cover 26110 and/or remove a portion of the cover. The tool can be configured to modify the device in any manner that allows blood to flow through the coaptation portion 23504.

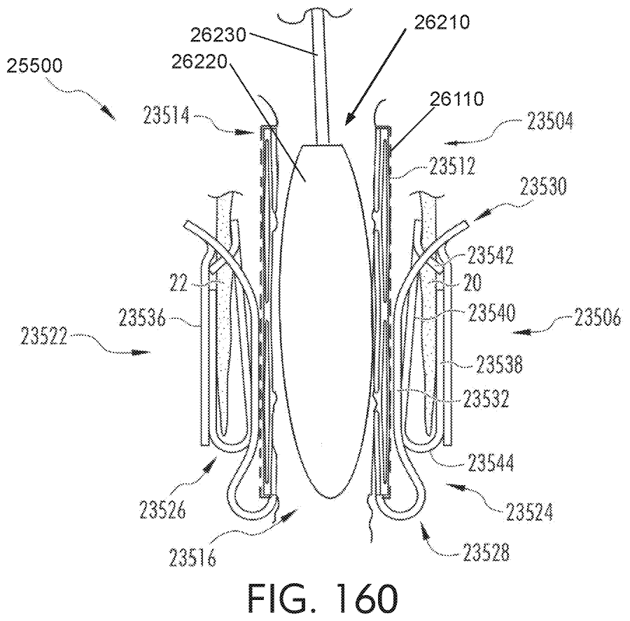

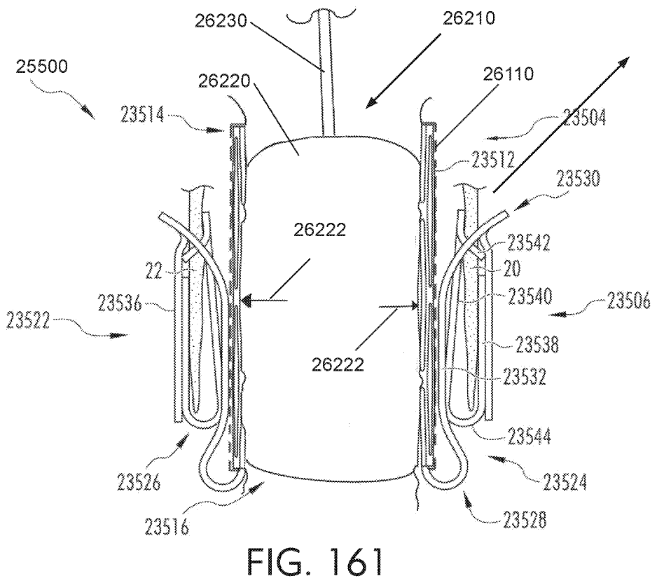

FIG. 160 illustrates a tool 26210 expanding the device 25500 to allow the device to accept the replacement valve 26100. The tool can take a variety of different forms. For example, the tool 26210 can include a positioning element 26230 (e.g., a rod, shaft, tube, catheter, etc.) and an expansion portion 26220 (e.g., balloon, expandable frame, etc.). Referring to FIGS. 160, the positioning element 26230 can be configured to position the expansion portion 26220 in the coaptation portion 23504 after the tool 26200 has cut, removed, or otherwise modified the cover 26110. Referring to FIG. 161, the expansion portion 26220 can be configured to expand (see arrows 26222) the coaptation portion 23504 to a size that can accept a replacement valve.

In some implementations, the tool 26210 can be part of the tool (i.e., the tool 26200 performs the steps shown in both of FIGS. 159 and 160. In some implementations, the tool 26200 can be configured to both cut, rupture, break, or ablate the cover 26110 and/or remove a portion of the cover and expand the coaptation portion 23504.

The expansion portion 26220 can take a variety of different forms. The expansion portion 26220 can be a balloon, any of the expansion mechanisms disclosed herein, any mechanism with controllably expanded struts and/or links, etc. The expansion portion 26220 can take any form that can expand the coaptation portion 23504 to a size that can accept a replacement valve 26100.

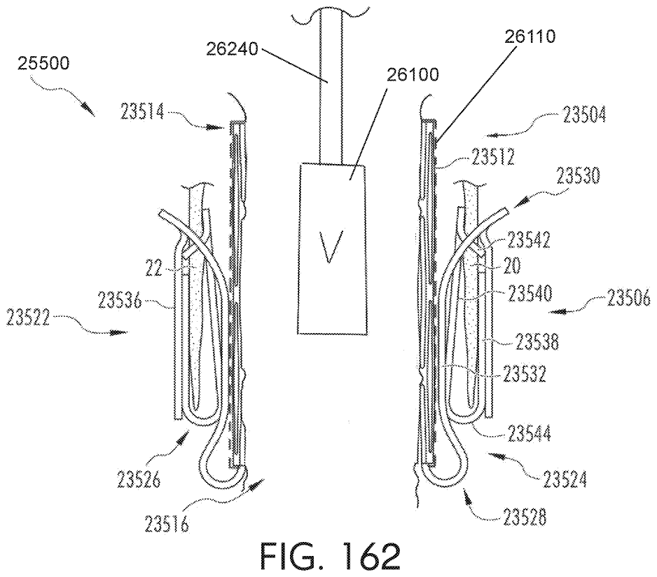

FIG. 162 illustrates a tool 26240 positioning a replacement valve 26100 in the expanded device 25500. The tool 26240 can take a variety of different forms. For example, the tool 26240 can be a rod, a shaft, a tube, a catheter, etc. Any tool 26240 that can position the replacement valve 26100 in the coaptation portion 23504 can be used.

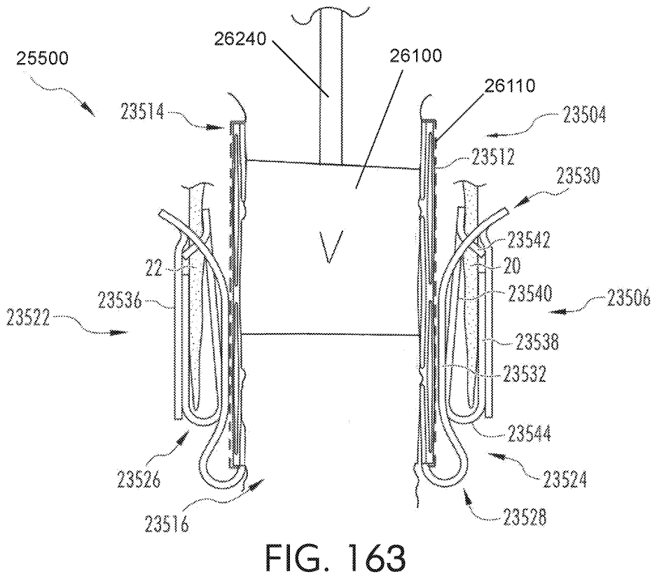

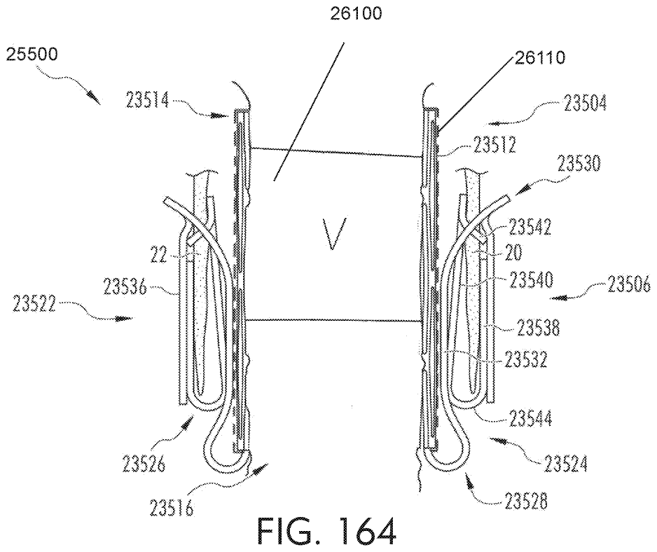

FIG. 163 illustrates the replacement valve 26100 expanding in the device 25500. In some implementations, the expansion of the replacement valve 26100 secures the replacement valve 26100 in the coaptation portion 23504 of the device 25500. The replacement valve 26100 can be expanded in a variety of different ways. For example, the replacement valve 26100 can be expanded by a balloon or the replacement valve can be self-expandable. A self-expandable replacement valve can be shape set to the expanded configuration and is held in a compressed condition (e.g., by a sleeve, catheter, etc.). When the self-expandable replacement valve is released (e.g., by pushing the replacement valve out of the sleeve, catheter, etc. or pulling the sleeve, catheter, etc. off of the replacement valve), the replacement valve 26100 self-expands into contact with the coaptation portion 23504. Referring to FIG. 164, the tool 26240 is removed and the replacement valve 26100 is left in the device 25500.

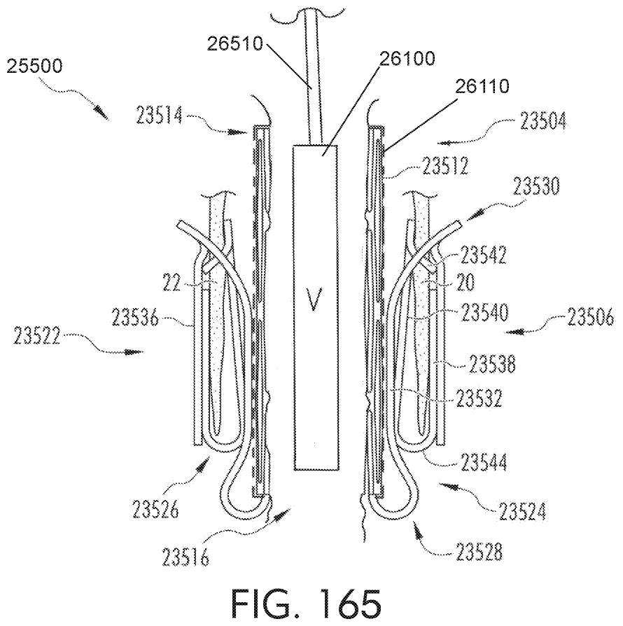

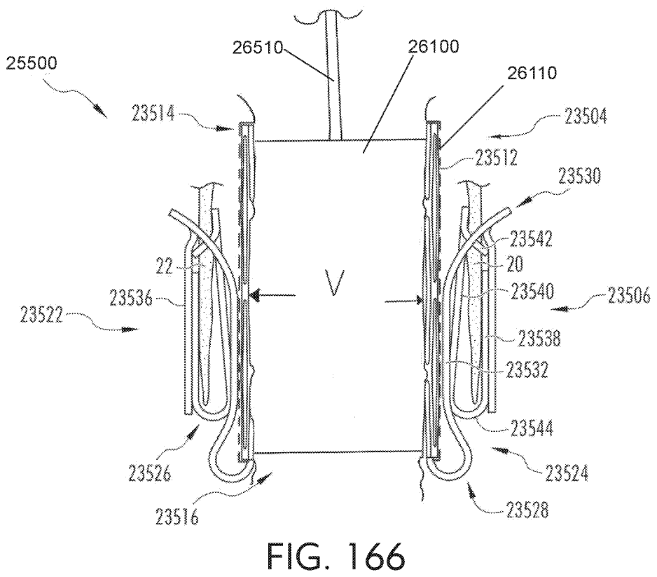

Referring to FIGS. 165 and 166 in some implementations, the device 25500 is expanded by the expansion of the replacement valve 26100, instead of by some other mechanism (e.g., balloon, etc.) before expansion of the replacement valve 26100. FIG. 165 illustrates a tool 26510 positioning a replacement valve 26100 in the device 25500 in an unexpanded condition. The tool 26510 can take a variety of different forms. For example, the tool 26510 can be a rod, a shaft, a tube, a catheter, etc. Any tool 26510 that can position the replacement valve 26100 in the coaptation portion 23504 can be used.

Referring to FIG. 166 the replacement valve 26100 is expanded, which also expands the device 25500. In some implementations, the expansion of the replacement valve 26100 secures the replacement valve 26100 in the coaptation portion 23504 of the device 25500. The replacement valve 26100 can be expanded in a variety of different ways. For example, the replacement valve 26100 can be expanded by a balloon or the replacement valve can be self-expandable. A self-expandable replacement valve can be shape set to the expanded configuration and is held in a compressed condition (e.g., by a sleeve, catheter, etc.). When the self-expandable replacement valve is released (e.g., by pushing the replacement valve out of the sleeve, catheter, etc. or pulling the sleeve, catheter, etc. off of the replacement valve), the replacement valve 26100 self-expands into contact with the coaptation portion 23504. The tool 26510 is removed and the replacement valve 26100 is left in the device 25500.

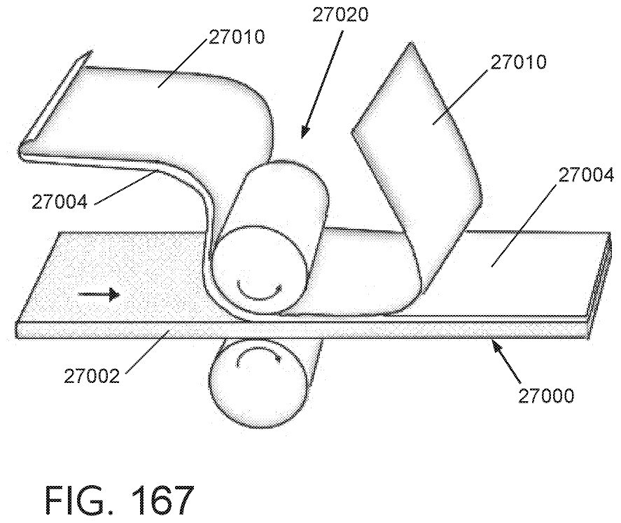

Referring to FIG. 167, in some implementations a cover material 27000 comprises a material 27002 (e.g., a fabric, polymer, etc.) that is laminated with a coating 27004 to decrease the permeability of the cover material. The coating 27004 can be a variety of different materials that are configured to reduce the permeability of the cover material 27000. For example, the coating 27004 can be a silicone, TPU, polyolefin and/or other polymer-based coating (e.g., any of the various polymers described anywhere herein). The material 27002 can take a variety of different forms. For example, the material 27002 can be any of the expandable and/or non-expandable fabrics or polymers disclosed herein.

Application of the coating 27004 to the fabric 27002 can be done in a variety of ways (e.g., spray coating, dip coating, lamination, etc.). In the example illustrated by FIG. 167 a release paper 27010 is coated with the coating 27004. The coated release paper 27010 and the fabric 27002 are passed through a pair of nip rollers 27020. The nip rollers 27020 press and transfer the coating from the release paper 27010 to the fabric 27002. The release paper 27010 is separated from the fabric 27002, leaving the coated cover material 27000 with a reduced permeability.

In some implementations, a yarn that reduces permeability can be woven into a fabric of the cover material. For example, a TPU, silicone, polyolefin and/or elastic yarn can be woven into any of the cover materials disclosed by the present application.

In some implementations a cover material comprises a material (e.g., fabric, polymer, mandrel, mold, etc.) that is dip coated to decrease the permeability of the cover material. The coating can be a variety of different materials that are configured to reduce the permeability of the cover material. For example, the dip coating can be a silicone, TPU, polyolefin and/or other polymer-based coating (e.g., any other polymers used elsewhere herein). The material can take a variety of different forms. For example, the material can be any of the expandable and/or non-expandable fabrics or polymers disclosed herein.

In some implementations, the cover material is optionally dip coated by first dipping material (e.g., fabric, polymer, mandrel, mold, etc.) in acetone. Then, the material is optionally dipped in water or distilled water. In some implementations, the material is then dipped in a coating or polymer base, such as polyurethane, TPU, polyester polyurethane, polycarbonate-based urethane, fluoropolymers, fluoro-oligomers, polyethylenes, polyacrylates, polysiloxanes, or another polymer material or a mixture/combination of any of the preceding (these materials can also be used in other coating and/or cover forming steps described elsewhere herein). Then, excess polymer or coating is optionally removed from the material. For example, the material can optionally be passed through a nip roller to press out the excess coating. In some implementations, the coated material can optionally be dried.

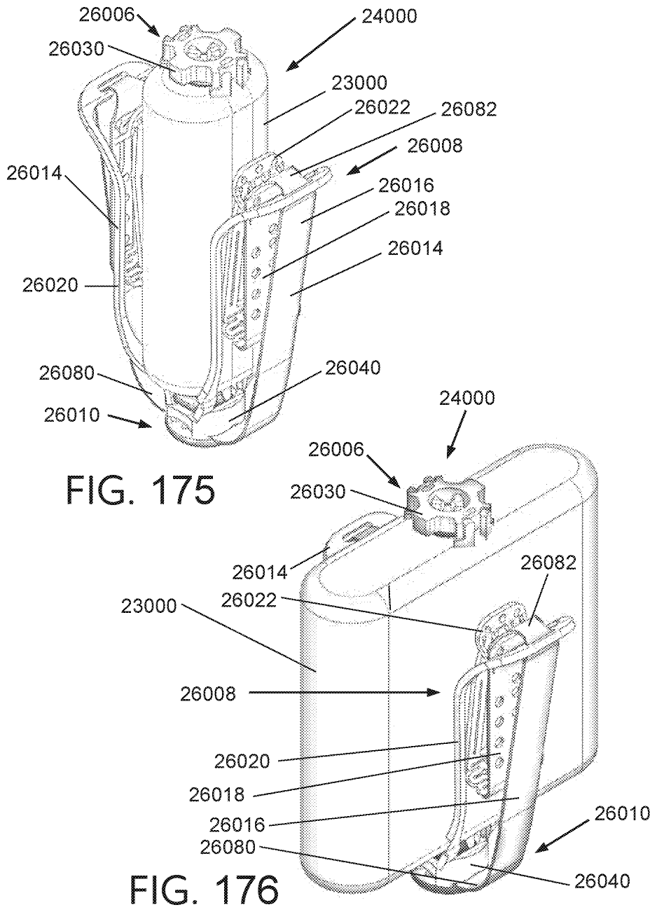

FIG. 169 illustrates an example frame member 23002 (e.g., frame, body, cage, fixture, chassis, form, etc.) for an expandable coaptation element 23000 (e.g., FIG. 175-177). The expandable coaptation element 23000 (e.g., spacer, coaptation element, gap filler, membrane, sheet, plug, wedge, balloon, etc.) can be part of a device or valve repair device 24000 for repairing a native heart valve and can be used as part of any device or valve repair device described herein.

In some implementations, the frame member 23002 is configured to move between a first contracted configuration (see FIG. 175) and a second expanded configuration (see FIG. 176). The frame member 23002 can be configured in a variety of ways. In some implementations, the frame member 23002 has a generally cylindrical shape (i.e., a circular cross section) in the contracted condition and an oval, elliptical, oblong, etc. shape (i.e., oval, elliptical, oblong, etc. cross section) in the expanded configuration. In some implementations, however, the frame member 23002 can have other shapes in the contracted and/or expanded configurations.

In some implementations, the frame member 23002 can include a plurality of interconnected struts 23008 that are configured to flex or bend to allow the frame member 23002 to move between the first and second configurations. The interconnected struts 23008 can be configured in a variety of ways, such as for example, the number and size of the struts, the shape of each strut, the arrangement of struts relative to other struts, the interconnection of the struts, etc. Any configuration that can facilitate the expansion and contraction of the frame member 23002 can be used. In some implementations, the struts 23008 are arranged in diamond patterns.

In some implementations, the example frame member 23002 includes a plurality of posts 23012 interconnected by the plurality of struts 23008. The posts 23012 can be configured in a variety of ways, such as for example, the number and size of the posts, the shape of each post, the arrangement and connection of posts relative to the struts, etc. In the illustrated example, each post 23012 extends linearly from a proximal end 23004 to a distal end 23006. In the illustrated example, each post 23012 is connected on either side, along a midpoint 23014, to one of the diamond patterns 23010 of struts 23008.

In some implementations, the frame member 23002 is formed as a unitary piece. For example, the frame member 23002 can be laser-cut from a tube. In some implementations, however, some of the struts 23008 and the posts 23012 can be formed separately and can be connected in any suitable manner (e.g., welded together).

In some implementations, the frame member 23002 can comprise a flexible material that can be a metal fabric, such as a mesh, woven, braided, or formed in any other suitable way or a laser cut or otherwise cut flexible material. The material can comprise cloth, shape-memory alloy wire—such as Nitinol—to provide shape-setting capability, or any other flexible material suitable for implantation in the human body.

In some implementations, when the frame member 23002 expands, the height of each of the diamond patterns 23010 decreases while the width increases. Conversely, the height of each posts 23012 do not change. Thus, the frame member 23002 has a fixed height (i.e., the height of the posts) that does not change between the first and second configurations.

In some implementations, the frame member 23002 can be configured to change height between the first and the second configuration. For example, the posts can be replaced with diamond patterns, such that the entire frame member 23002 decreases in height as the frame member increases in width.

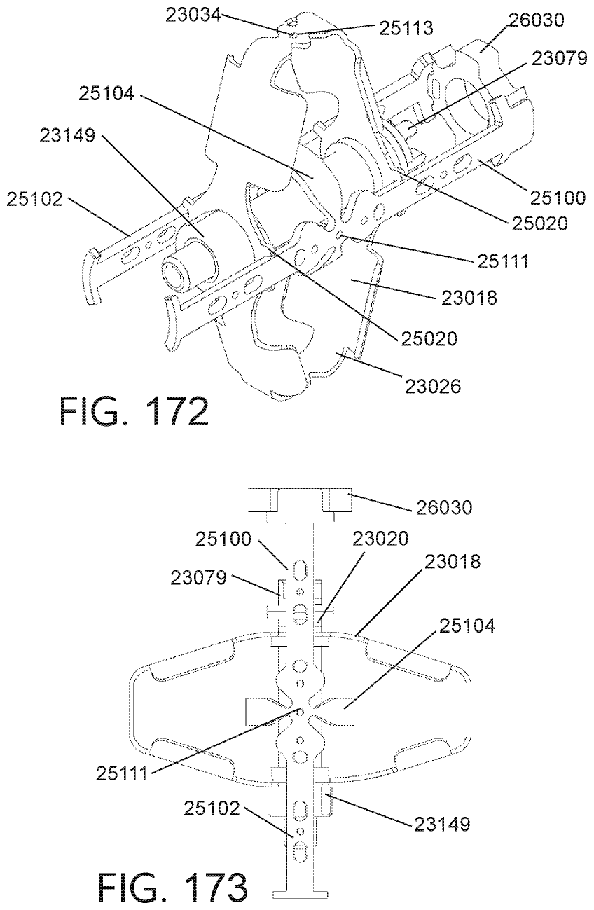

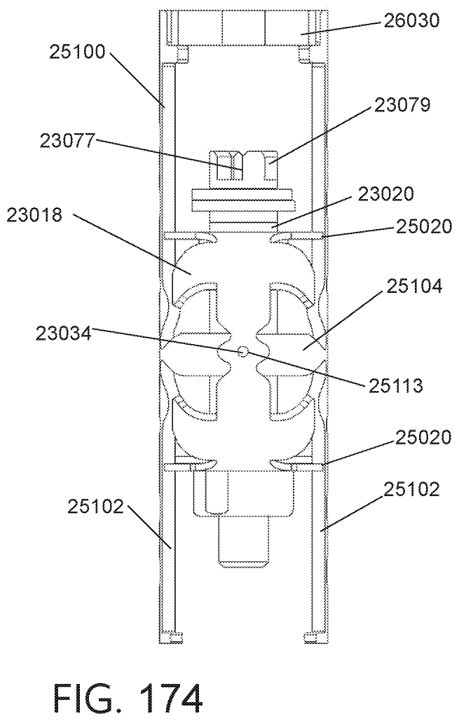

The frame member 23002 can be moved between the first and the second configurations by any suitable means. For example, the frame member 23002 can be mounted around an expandable mechanism 23016 (see FIGS. 172-174) configured to engage the frame member 23002 to move the frame member 23002 between the first and the second configurations. A variety of different mechanisms can be used to move the frame member 23002 between the first and second configurations. For example, any expandable mechanism disclosed herein can be used.

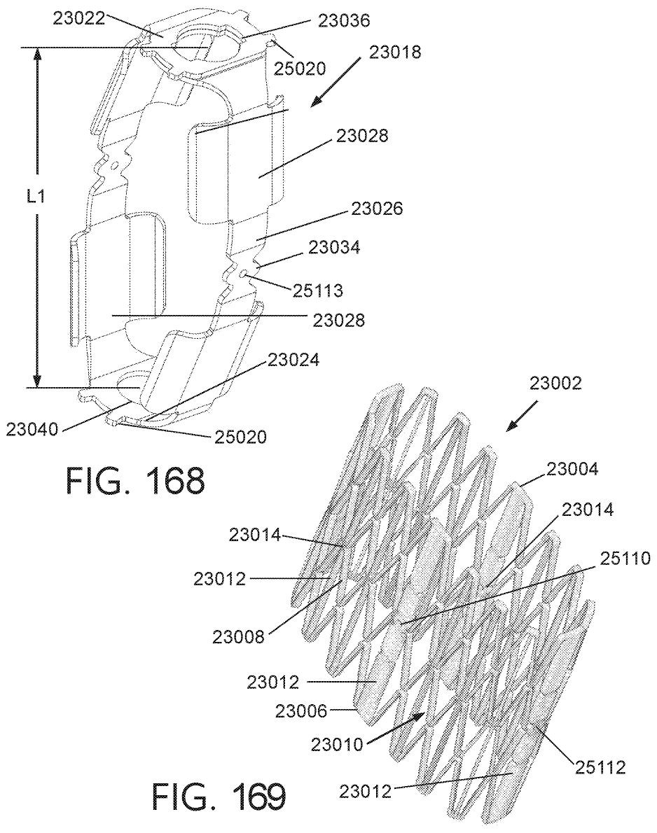

In some implementations, as shown in FIGS. 168 and 170-174, the expandable mechanism 23016 includes an expandable member 23018 (e.g., frame, body, strut assembly, tube, shaft, etc.) and an actuation mechanism 23020. In some implementations, the frame member 23002 is mounted around the expandable member 23018. The expandable member 23018 can be configured in a variety of ways. Any configuration capable of moving the frame member 23002 between the first configuration and the second configuration can be used.

Referring to FIG. 168, in some implementations, the expandable member 23018 is configured as an elongated shape having a proximal end portion 23022, a distal end portion 23024 opposite the proximal end portion 23022, and an intermediate portion 23026 connecting the proximal end portion 23022 and the distal end portion 23024. In some implementations, the intermediate portion 23026 comprises and/or is formed from a pair of strips 23028 that are configured to bend or flex. In some implementations, the expandable member 23018 includes two opposed strips 23028. In some implementations, however, the strips 23028 need not be equally spaced and/or the intermediate portion 23026 can include more or less than two (2) strips.

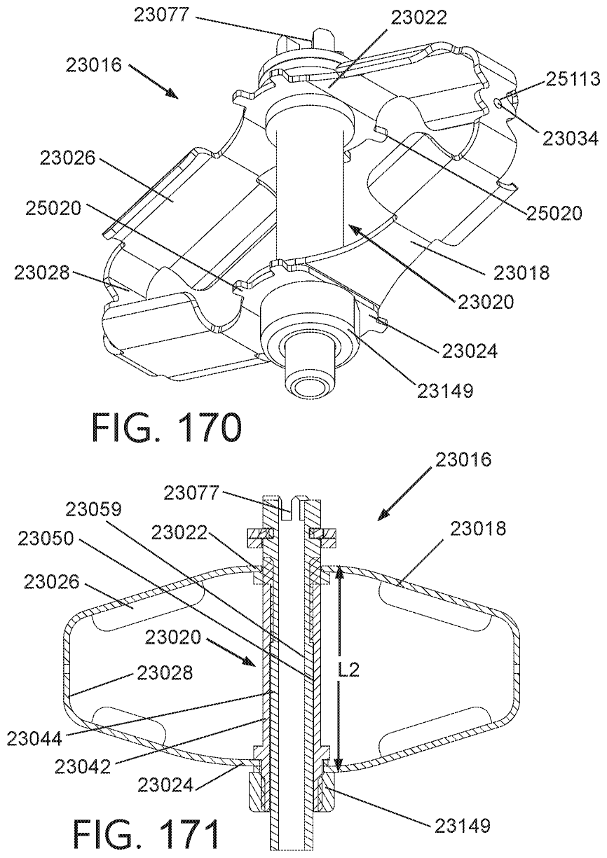

In some implementations, the expandable member 23018 is movable between a first configuration (i.e., collapsed or narrow configuration) (FIG. 168) and a second configuration (i.e., an expanded or wide configuration) (FIG. 170). As shown in FIG. 168, in the first configuration, each of the plurality of strips 23028 extend parallel or generally parallel to a longitudinal axis of the expandable member 23018 and the expandable member 23018 has a first length L1. In the first configuration, the proximal end portion 23022, the distal end portion 23024, and the intermediate portion 23026 have the same or substantially the same width.

In some implementations, the expandable member 23018 can be formed as a unitary piece. For example, the expandable member 23018 can be laser-cut from a single piece of sheet material. In some implementations, the expandable member 23018 can be formed from multiple pieces, such as for example, a pair of Nitinol sheets.

In some implementations, the expandable member 23018 can comprise a flexible material that can be a metal fabric, such as a mesh, a woven material, a braided material, or a material formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, as shown in FIG. 170, in the second configuration, the proximal end portion 23022 is moved toward the distal end portion 23024, or vice versa, or the proximal end portion and the distal end portion simultaneously move toward one another, causing the strips 23028 to bend or flex outward such that the intermediate portion 23026 expands to a second width that is greater than the first width. Further, in the second configuration, the expandable member 23018 has a second length L2, that is less than the first length L1 and the width of the proximal end portion 23022 and the distal end portion 23024 remain at the first width while the intermediate portion 23026 expands to the second width.

In some implementations, the expandable member 23018 positioned within the periphery of the frame member 23002 to engage the frame member 23002. In some implementations, a midpoint 23034 of each of the two strips 23028 is attached to the frame member 23002. In some implementations, the midpoint 23034 of each strip 23028 is attached to one of the posts 23012 of the frame member 23002. The midpoint 23034 of each strip 23028 can attach to the frame member 23002 in any suitable manner, such as for example, welding, sutures, fasteners, etc.

In some implementations, as a result of the expandable member 23018 being positioned within the periphery of the frame member 23002 and attached to the frame member 23002, movement of the expandable member 23018 between the first configuration (i.e., collapsed or narrow configuration) (FIG. 168) and a second configuration (i.e., expanded or wide configuration) (FIG. 170) moves the frame member 23002 between the frame member's first configuration (i.e., collapsed-FIG. 175) and second configuration (i.e., expanded-FIG. 176).

In some implementations, the frame member 23002 and/or the expandable member 23018 can be configured to be normally in the first configuration (i.e., collapsed or narrow), in the second configuration (i.e., expanded or wide), or some intermediate position between the first configuration and the second configuration. Thus, in some implementations, movement of the expandable member 23018 pulls the frame member 23002 inward toward the first position against a bias of the frame member 23002. In some implementations, movement of the expandable member 23018 pushes the frame member outward to the second position against a bias of the frame member 23002.

Referring to FIGS. 170-171, in some implementations, an actuation mechanism 23020 is configured to move the expandable member 23018 between the first and the second configurations. The actuation mechanism 23020 can be configured in a variety of ways. Any actuation mechanism capable of moving the expandable member 23018 between the first and the second configurations can be used. In some implementations, the actuation mechanism 23020 is mounted within the expandable member 23018.

Referring to FIGS. 170-174, in some implementations, the expandable member 23018 can include structure configured to mount the actuation mechanism 23020 within the expandable member 23018. The structure can be configured in a variety of ways. In some implementations, the proximal end portion 23022 of the expandable member 23018 includes a first opening 23036 and the distal end portion 23024 of the expandable member 23018 includes a second opening 23040.

In some implementations, the actuation mechanism 23020 is configured as a threaded connection. Referring to FIGS. 170-171, the example actuation mechanism 23020 includes a distal member or outer member 23042 (e.g., tube, body, shaft, etc.) configured to threadably couple with a proximal member or inner member 23044 (e.g., tube, body, shaft, etc.). The proximal or inner member 23044 and the distal or outer member 23042 can be configured in a variety of ways. In the illustrated example, the distal member 23042 is formed as a cylindrical tube having a circular cross section. In some implementations, however, the distal member 23042 can have a shape other than cylindrical (e.g., a cross section that is oval, rectangular, elliptical, or other suitable shape).

In some implementations, the distal member 23042 includes a proximal end, a distal end opposite the proximal end, and an inner passage 23050 extending through the distal member 23042 member from the proximal end to the distal end. The inner passage 23050 can include female threads for threadably coupling with the proximal member.

In some implementations, the distal member 23042 is sized to be received within the expandable member 23018. In some implementations, the assembly includes structure for fixing the distal member 23042 in position within the expandable member 23018. In the illustrated example, a nut or collar 23149 fixes the distal end of the distal member 23042 to the distal end portion 23024 of the expandable member 23018, as shown in FIGS. 170-171.

In some implementations, the proximal member 23044 is formed as a cylindrical tube having a circular cross section. In some implementations, the proximal member 23044 can have a shape other than cylindrical (e.g., a cross section that is oval, rectangular, elliptical, or other suitable shape). In some implementations, the proximal member 23044 includes a proximal end, a distal end opposite the proximal end, and an inner passage 23059 extending through the proximal member 23044 from the proximal end to the distal end. In some implementations, the inner passage is sized to accommodate an actuation element (e.g., actuation element 112 for opening and closing paddles of the valve repair device).

In some implementations, the proximal member 23044 can include male threads extending along at least a portion of the exterior of the proximal member 23044. The male threads are configured to mate with the female threads of the distal member 23042. In some implementations, the proximal member 23044 is sized to be received within the expandable member 23018. In some implementations, the actuation mechanism 23020 can include structure for restricting axial movement of the proximal member 23044 relative to the expandable member 23018.

In some implementations, the structure restricts axial movement of the proximal member 23044 within the expandable member 23018 while allowing rotational movement of the proximal member 23044 relative to the expandable member 23018. The structure for restricting axial movement of the proximal member 23044 can be configured in a variety of ways. For example, a fastener, such as a clip, pin, etc. can be used to restrict axial movement of the proximal member 23044 within the expandable member 23018 while allowing rotational movement of the proximal member 23044 relative to the expandable member 23018.

Referring to FIGS. 168, 170, and 171, in some implementations, when assembled, the actuation mechanism 23020 can move the expandable member 23018 between the first and the second configurations. In particular, the distal member 23042 is received within the expandable member 23018. In some implementations, the distal member 23042 can be both axially and rotationally fixed to the expandable member 23018.

In the example illustrated by FIGS. 170 and 171, the expandable mechanism 23016 is configured to move in one plane (i.e., the two strips 23028 move apart). In some implementations, the expandable mechanism can move in two planes. For example, a second pair of strips can be disposed orthogonally to the first pair of strips. The second pair of strips can move in a second plane that is orthogonal to the first plane. However, the second plane can be at any angle to the first plane. As such, the frame or spacer 23002 can be expanded in more than one direction. In some implementations, the expandable mechanism can be configured such that expansion in the first plane can be controlled independently of the expansion in the second plane (e.g., the width of the two pairs of strips is independently controllable).

In some implementations, the proximal member 23044 is received in the passage 23050 of the distal member 23042 such that the male threads of the proximal member 23044 threadably engage the female threads of the distal member 23042. In some implementations, a rotational device or tool (not shown) can engage the one or more engagement surfaces 23077 to rotate the proximal member 23044 in a first rotational direction (i.e., to thread the proximal member 23044 into the distal member 23042). With the proximal member 23044 axially fixed to the proximal end portion 23022 of the expandable member 23018 and the distal member 23042 axially fixed to the distal end portion 23024 of the expandable member 23018, threading the proximal member 23044 into the distal member 23042 draws the proximal end portion 23022 of the expandable member 23018 toward the distal end portion 23024. As a result, the strips 23028 bend or flex outward. Since the midpoint 23034 of the strips 23028 are attached to the posts 23012 of the frame member 23002, the outward expansion of the strips 23028 causes the outward expansion of the frame member 23002.

Conversely, in some implementations, rotating the proximal member 23044 in a second rotational direction (i.e., to thread the proximal member 23044 out of the distal member 23042) pushes the proximal end portion 23022 of the expandable member 23018 away from the distal end portion 23024. As a result, the strips 23028 are pulled inward causing the inward contraction of the frame member 23002. In some implementations, the actuation mechanism illustrated by FIGS. 94-98 can be used in place of the actuation mechanism 23020.

Referring to FIGS. 172-174, the expandable mechanism 23016 can be assembled with a frame 25100 of the valve repair device 24000. The frame 25100 can take a wide variety of different forms. In some implementations, the frame 25100 includes a pair of rails 25102 that are connected together by a central ring 25104. In some implementations, the proximal end 23022 and/or the distal end 23024 of the expandable member 23018 are slidably coupled to the rails 25102. The slidable coupling(s) allow proximal end 23022 and/or the distal end 23024 to slide relative to the frame. The proximal end 23022 and/or the distal end 23024 can be slidably coupled to the rails 25102 in a variety of different ways. Any slidable coupling can be used.

In some implementations, the proximal end 23022 includes a pair of tabs 25020 that fit around the rails 25102 to slidably couple the proximal end 23022 to the frame 25100. In some implementations, the distal end 23024 includes a pair of tabs 25020 that fit around the rails 25102 to slidably couple the distal end 23024 to the frame 25100.

In some implementations, the expandable frame member 23002 or spacer is attached to the central ring 25104 and the expandable member 23018. Since the central ring 25104 is fixed in size, the length (when viewed from an end) of the expandable frame member 23002 or spacer is fixed by the connection to the ring. Since the expandable member 23018 is adjustable in size, the width of the expandable frame member 23002 or spacer is adjustable by adjusting the width of the expandable member 23018.

The expandable frame member 23002 or spacer can be attached to the central ring 25104 and the expandable member 23018 in a variety of different ways. In some implementations, the expandable frame member 23002 or spacer can include two opposed attachment points 25110, such as holes, in two of the posts 23012 for connection to attachment points 25111 of the ring 25104. In some implementations, the expandable frame member 23002 or spacer can include two opposed attachment points 25112, such as holes, in two of the posts 23012 for connection of attachment points 25113 of the expandable member 23018.

In some implementations, the attachment of the expandable frame member 23002 or spacer to both the ring 25104 and the expandable member 23018 keeps the expandable mechanism 23020 centered relative to the frame 25100, regardless of the amount of expansion of the expandable member 23018. When the expandable mechanism 23020 is expanded, the proximal end 23022 slides along the rails 25102 toward the distal end 23024 and the distal end 23024 slides along the rails 25102 toward the proximal end 23022. In some implementations, the connections between the attachment points 25110 and the attachment points 25111 (i.e., the connections between the expandable frame member 23002 and the ring 25104) and the connections between the attachment points 25112 and the attachment points 25113 (i.e., the connections between expandable frame member 23002 and the expandable member 23018) keep the midpoint 23034 of the expandable member 23018 aligned with the ring 25104 as the expandable mechanism is moved between the contracted and expanded configurations.

FIGS. 175-178 illustrate an example of a device 24000 (e.g., valve repair device, valve treatment device, implantable device, implant, etc.) having an expandable coaptation element 23000. The expandable coaptation element 23000 can be used in a variety of different devices or valve repair devices, including but not limited to, any of the devices and valve repair devices disclosed in the present application. For example, any expandable coaptation element disclosed herein can be used in the device that is schematically illustrated in FIGS. 8-14. The implantable device 24000 is one of the many different configurations that the device that is schematically illustrated in FIGS. 8-14 with an expandable coaptation element 23000 can take. The device 24000 can include any other features for a device or valve repair device discussed in the present application, and the expandable coaptation element 23000 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application). The device 24000 can be deployed from a delivery sheath, and/or an implant catheter. The device 24000 can include an expandable mechanism 23016, an expandable frame or spacer 23002, and an anchor portion having two or more anchors, such as the anchor portion and anchors described herein. The device 24000 can be a prosthetic spacer device, valve repair device, valve treatment device, implant, or another type of device that attaches to leaflets of a native valve.

Referring now to FIGS. 175-178, a device 24000 (e.g., a valve repair device, an implantable device, implant, valve treatment device, etc.) with an expandable mechanism 23016 and an expandable frame or spacer 23002 is shown. The implantable device 24000 is one of many different configurations that the device 100 that is schematically illustrated in FIGS. 8-14 with an expandable mechanism 23016 and an expandable frame or spacer 23002 added can take.

In some implementations, the device or valve repair device 24000 includes an expandable mechanism 23016, an expandable frame or spacer 23002, a proximal or attachment portion 26006, an anchor portion 26008, and a distal portion 26010. In some implementations, the expandable mechanism 23016 and expandable frame or spacer 23002 are configured for adjustable implantation between leaflets of a native valve. In some implementations, the anchor portion 26008 includes a plurality of anchors 26014. The anchors can be configured in a variety of ways. In some implementations, the anchors 26014 include outer paddles 26016, inner paddles 26018, paddle extension members or paddle frames 26020, and clasps 26022. In some implementations, the attachment portion 26006 includes a first or proximal collar 26030 (or other attachment element) for engaging with a capture mechanism or coupler of a delivery system. A delivery system for the implant 24000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the expandable mechanism 23016, the expandable frame or spacer 23002 and/or the outer and inner paddles 26016, 26018 are formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the device 24000 (see actuation element 112 in FIGS. 8-14). In some implementations, the actuation element extends through the proximal collar 26030, the expandable mechanism 23016 and the expandable frame or spacer 23002 to engage a cap 26040 of the distal portion 26010. The actuation element can be configured to removably engage the cap 26040 with a threaded connection, or the like, so that the actuation element can be disengaged and removed from the implant 24000 after implantation.

The frame 25100, that the expandable mechanism 23016 is mounted to, extends from the proximal collar 26030 (or other attachment) to the cap 26040. The size and/or shape of the expandable frame or spacer 23002 can be selected and/or be adjusted to minimize the number of implants that a single patient will require (preferably one), while at the same time maintaining low transvalvular gradients.

In some implementations, the outer paddles 26016 are jointably attached to the cap 26040 of the distal portion 26010 by connection portions 26080 and to the inner paddles 26018 by transition portions 26082. In some implementations, the paddle frames 26020 are attached to the cap 26040 at the distal portion 26010 and extend to the transition portions 26082 between the inner and outer paddles 26018, 26016. In some implementations, the paddle frames 26020 are formed of a material that is more rigid and stiff than the material forming the paddles 26018, 26016 so that the paddle frames 26020 provide support for the paddles 26018, 26016.

The paddle frames 26020 can provide additional pinching force between the inner paddles 26018 and the expandable frame or spacer 23002. The connections between the paddle frames 26020, the outer and inner paddles 26016, 26018, the cap 26040, and the expandable frame or spacer 23002 can constrain each of these parts to the movements and positions of the devices or valve repair devices described herein.

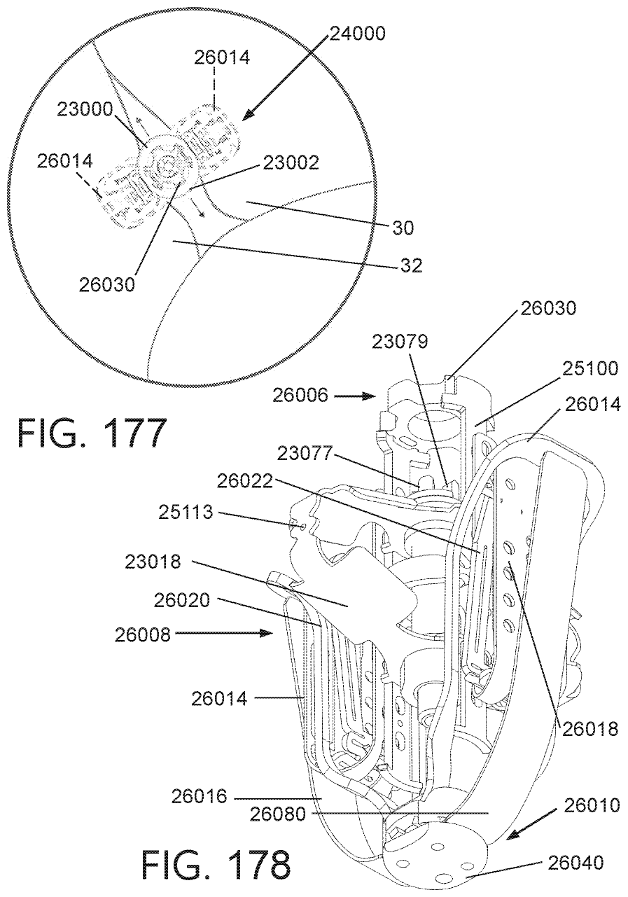

Referring now to FIG. 177, the example expandable frame or spacer 23002 is positioned between the leaflets 30, 32 as shown in the top plan view. The anchors 26014 are shown in dashed lines in FIG. 177, since, with the exception of the clasps, the installed anchors 26014 are disposed on the ventricular side of the native leaflets. FIG. 176 shows the device 24000 with the expandable frame or spacer 23002 in an expanded configuration. FIGS. 175 and 121 show the device 24000 with the expandable frame or spacer 23002 in a contracted configuration. Adjusting the expandable mechanism 23016, for example by rotating the engagement surfaces 23077 of a head 23079, expands or contracts the expandable frame or spacer 23002 to adjust flow through the native valve (reduce regurgitation through the native valve by increasing the size of the shell or increasing the flow through the native valve by reducing the size of the shell).

Referring to FIG. 178, the strips 23028 of the expandable mechanism 23016 are spread apart to move the expandable frame or spacer 23002. As such, the expandable frame or spacer 23002 has a wide configuration and takes up a larger space. Referring to FIG. 175, the strips 23028 of the mechanism 23016 are close together to move the expandable frame or spacer 23002 to a contracted configuration. As such, the expandable frame or spacer 23002 has a narrow configuration and takes up a smaller space.

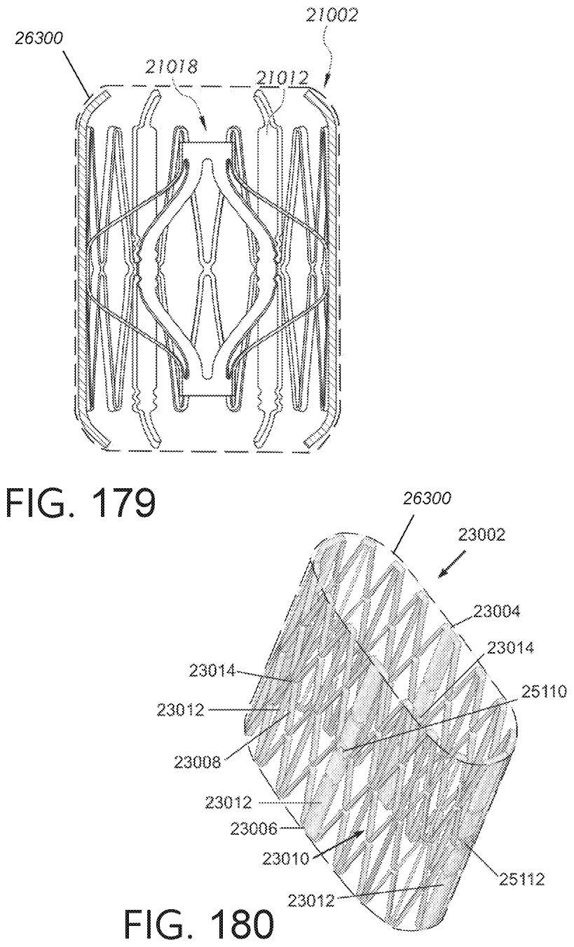

Turning now to FIGS. 179-184, in some implementations, the expandable coaptation element can include an optional polymer-based cover over the expandable coaptation element, such as over the frame member 21002. The cover can cover a portion or all of the expandable coaptation element or frame member 21002. The polymer-based cover 26300 can provide a softer material for interaction with the native leaflets which can, in turn, reduce friction. The polymer-based cover 26300 can also reduce blood turbulence and improve impermeability of the expandable coaptation element.

The polymer-based cover 26300 can be formed from any one of a variety of polymeric materials. In some implementations, the polymeric material has a Shore A durometer of from about 70 to about 95, from about 70 to about 90, from about 70 to about 85, from about 70 to about 80, or from about 70 to about 75, as measured in accordance with ASTM D-2240. The polymeric material of some implementations can be selected based on its biostability, biocompatibility, radial elongation, and ability to withstand deployment forces. Suitable polymeric materials can include, by way of example and not limitation, thermoplastic polyurethanes, polyester polyurethanes, polycarbonate-based urethanes, fluoropolymers (e.g., polytetrafluoroethylene (PTFE) and expanded PTFE (ePTFE)), fluoro-oligomers, polyethylenes (e.g., ultra-high molecular weight polyethylene (UHMWPE), low-density polyethylene (LDPE), and high-density polyethylene (HDPE)), polyacrylates, polysiloxanes, and mixtures thereof. Thermoplastic polyurethanes can include, for example, those polyurethanes including, fluorine, silicone, or both in the polymeric structure. The polymeric material can also include, for example, polymers formed from inorganic backbones and including fluoro-groups. In some implementations, the polymeric material is a thermoplastic polyurethane including siloxane in polymer backbone.

The polymer-based cover 26300 can be configured in a variety of ways. For example, as shown in FIG. 179, the polymer-based cover 26300 can at least partially extend around, or encapsulate, the frame member 21002 including that is mounted around the expandable/expansion member 21018. As described in greater detail hereinabove, the expandable/expansion member 21018 can be configured in a variety of ways.

FIG. 180 illustrates an example frame member 23002 (e.g., frame, body, cage, fixture, chassis, form, etc.) including a polymer-based cover 26300. The example frame member 23002 can be part of a device or valve repair device 24000 for repairing a native heart valve and can be used as part of any device or valve repair device described herein.

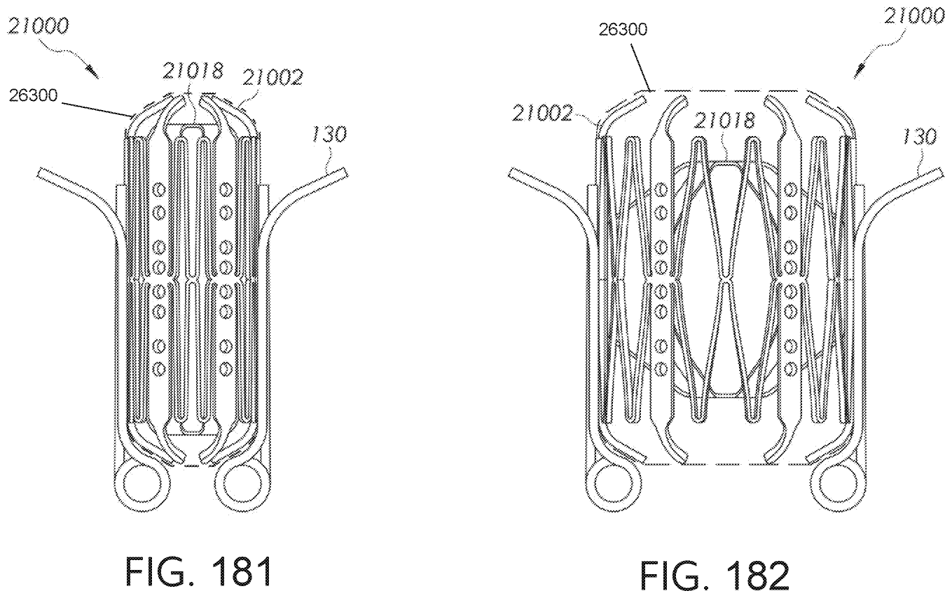

FIGS. 181-182 illustrate an example expandable coaptation element 21000 including a polymer-based cover 26300 as part of a device e.g., a treatment device, repair device, valve treatment device, valve repair device, implantable device, implant, etc.) that is configured to be positioned within a native heart valve to allow the native heart valve to form a more effective seal. The polymer-based cover 26300 is shown at least partially enclosing or encapsulating the frame member 21002 and the expandable/expansion member 20108, which is attached to gripping members (e.g., gripping arms, clasp arms, etc.), such as for example, gripping members 130.

FIG. 181 illustrates the example expandable coaptation element 21000 in a first configuration (i.e., collapsed or narrow) and FIG. 182 illustrates the expandable coaptation element 21000 in a second configuration (i.e., expanded or wide). The expandable coaptation element 21000 can be moved between the first configuration and the second configuration by any suitable means, such as for example, any of the actuation mechanisms disclosed herein. As shown in FIGS. 181-182, the polymer-based cover 26300 is configured to expand and collapse with the expandable coaptation element 21000.

In some implementations, the frame member 23002 can comprise a flexible material that can be a metal fabric, such as a mesh, woven, braided, or formed in any other suitable way or a laser cut or otherwise cut flexible material. The material can comprise cloth, shape-memory alloy wire—such as Nitinol—to provide shape-setting capability, or any other flexible material suitable for implantation in the human body. The polymer-based cover 26300 can be formed as a distinct layer on the material to form a multi-layered structure, or the polymer-based cover 26300 can incorporate the material, such as by impregnating the polymeric material into the material. Accordingly, in some implementations, the polymer-based cover 26300 comprises a fiber- or textile-reinforced polymeric material.

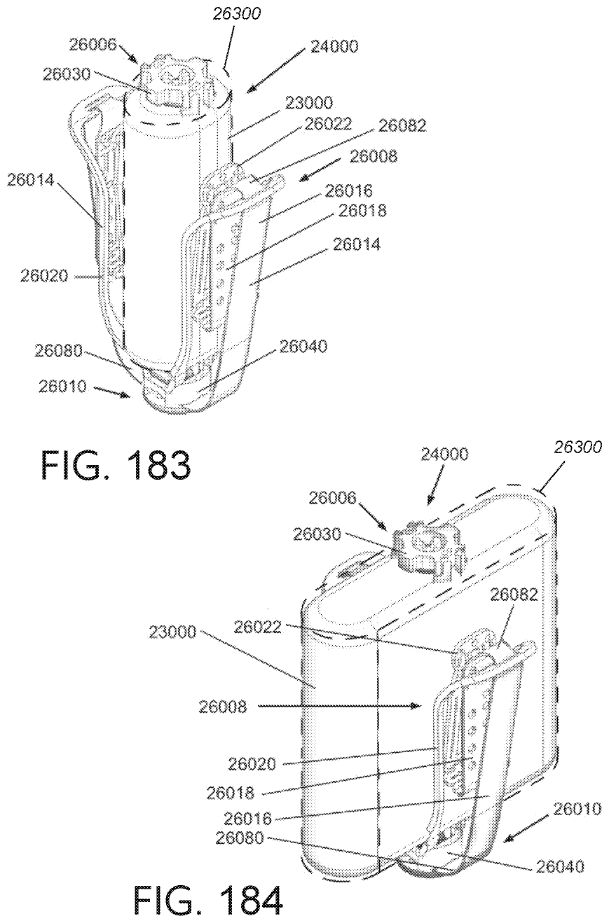

In FIGS. 183-184, an example of a device 24000 (e.g., valve repair device, valve treatment device, implantable device, implant, etc.) having an expandable coaptation element 23000 having a polymer-based cover 26300 thereon is illustrated. In some implementations, the device or valve repair device 24000 includes a proximal or attachment portion 26006, an anchor portion 26008, and a distal portion 26010.

In some implementations, the anchor portion 26008 includes a plurality of anchors 26014. The anchors can be configured in a variety of ways. In some implementations, the anchors 26014 include outer paddles 26016, inner paddles 26018, paddle extension members or paddle frames 26020, and clasps 26022. In some implementations, the attachment portion 26006 includes a first or proximal collar 26030 (or other attachment element) for engaging with a capture mechanism or coupler of a delivery system.

A delivery system for the implant 24000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the expandable mechanism 23016, the expandable frame or spacer 23002 and/or the outer and inner paddles 26016, 26018 are a covered with a polymer-based cover 26300. In implementations in which the expandable mechanism 23016, the expandable frame or spacer 23002 and/or the outer and inner paddles 26016, 26018 are formed from a flexible material that can be a metal fabric, such as mesh, woven, braided, or formed in any other suitable way or a laser cut or otherwise cut flexible material, the polymer-based cover 26300 can be formed as a separate layer on the material, or can be impregnated into the material.

The polymer-based cover can be formed through a variety of processes, including, but not limited to, dip coating and extrusion processes. In some implementations, the polymer-based cover is formed by a dip coating process in which the expandable frame or spacer is dipped into a coating composition before being dried and annealed. In some implementations, the orientation of the expandable frame or spacer (e.g., the directionality for the dip coating) is selected to obtain a desired elongation or other expansion properties of the resultant polymer-based cover. When the polymer-based cover is formed through dip coating, the polymeric material can be melted or dispersed in a solvent to form the coating composition.

In some implementations, the polymer-based cover is formed through a melt blown balloon extrusion process, which can enable to the polymer-based cover to be formed without the use of a solvent.

In some implementations, the polymeric material is extruded into a tubing and inserted into a glassform or mold. One end is welded up and the other end is coupled to a compressed-air supply. A pair of heated jaws close around the part, while the tubing is kept in the balloon form for a warm-up time at a constant inner pressure. Then, a high forming pressure is applied and held for a forming time while the tubing is axially stretched at both ends. Following forming, the glassform is blown with compressed air to cool the tubing, which can be cut to size to form the polymer-based cover. Other processes can also be used to form the polymer-based cover.

In some implementations, valve repair devices can optionally have a reduced echogenic profile and/or a reduced echogenic profile during a portion of an implantation procedure. A reduced echogenic profile is one that reduces the amount of image shadow caused by device components such as, for example, the coaptation element or spacer in the tissue or leaflet capture area. A reduced echogenic or image profile can increase the ability to see the tissue or leaflet during capture and/or device implantation. The optional reduced echogenic profile can shorten the implantation or treatment time due to the increased visibility of the tissue or leaflet.

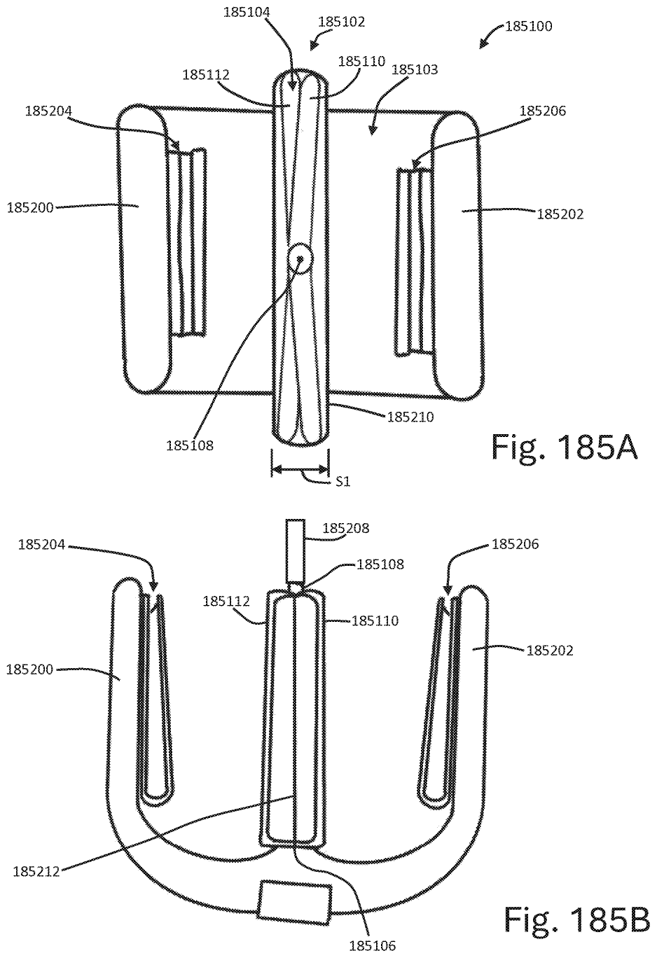

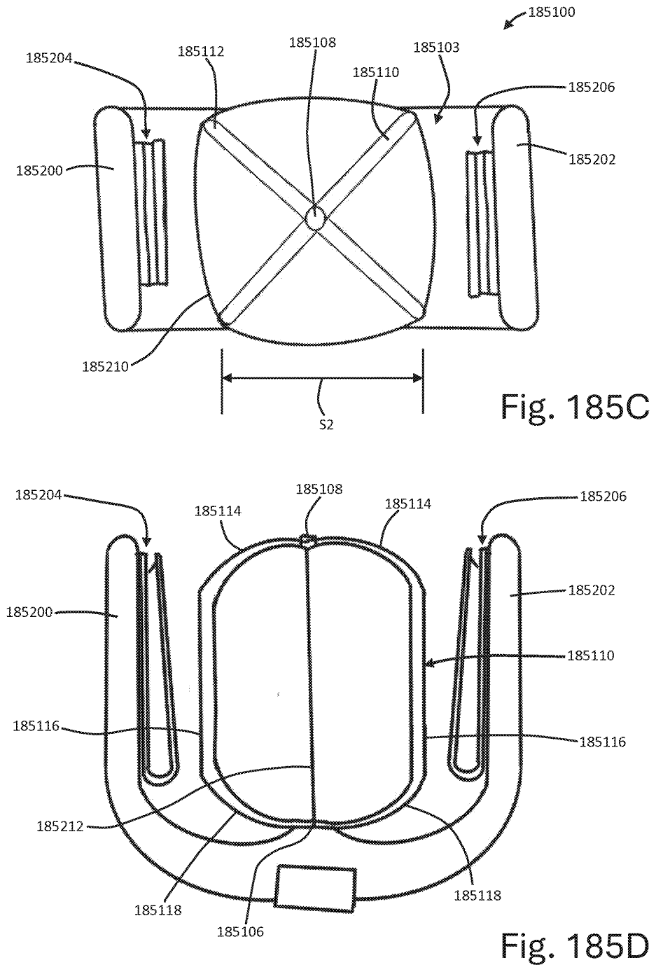

Referring now to FIGS. 185A, 185B, 185C, and 185D, an example of a valve repair device 185100 is shown. The device 185100 can have a spacer that can be narrowed or reduced in width. This narrowing or reduction in width can reduce the echogenic profile during positioning and/or implanting of the device. The coaptation element or spacer 185102 can be moved between a collapsed condition (FIGS. 185A and 185B) and an expandable condition (FIGS. 185C and 185D). In some implementations, the collapsed condition (e.g., FIGS. 185A and 185B) has a minimal or transport volume that is conducive to the device 185100 passing through a catheter or similar apparatus and can have a small echogenic footprint. The spacer assembly can also include an expanded condition (e.g., FIGS. 185C and 185D) having a larger or repair volume that is conducive to the ability of the device to block regurgitant flow. The reduced spacer volume assists in allowing device 185100 to travel through a catheter due to its reduced volume to the implantation area. And, once at the implantation area, the spacer volume can be increased to assist with the blocking of regurgitant blood flow.

The collapsed condition can provide the spacer with a smaller or narrower echogenic profile that reduces the amount of image shadow caused by the spacer during implantation. This increases the ability to see the tissue or leaflet during capture and/or device implantation. This, in turn, can shorten the implantation or treatment time due to the increased visibility of the tissue or leaflet.

In some implementation, the device includes a spacer 185102 disposed in a space 185103 between paddle portions 185200 and 185202. Also shown proximate to the paddle portions 185200 and 185202 are optional clasp portions 185204 and 185206. The paddle and clasp portions can be according to any of the previously described examples and implementations. The spacer 185102 can include a frame 185104 having first and second frame members 185110 and 185112. In some implementations, each frame member can include first and second cross portions 185114 and 185118 and a middle portion 185116 (see FIG. 185D). The frame 185104 can also include first and second pivots 185106 and 185108 to thereby provide an axis of rotation. This axis of rotation can provide rotational functionality to the one or more first and second frame members 185110 and 185112 thereby providing the spacer with the collapsed and expanded configurations and/or volumes. A cover 185210 is provided over the spacer 185102 and an actuation element 185208 is provided to rotate the spacer frame members 185110 and 185112 from the collapsed condition to the expanded condition.

FIGS. 185A and 185B show the spacer 185102 in the collapsed or transport position whereby it occupies a small portion of the volume of space 185103. In the example shown, frame members 185110 and 185112 are disposed in a collapsed cross of “X” configuration. In this collapsed position, spacer 185102 has a width of S1 (compared to the larger width of S2 in FIG. 185C showing the expanded configuration). The frame members 185110 and 185112 are pivoted around first and second pivots 185106 and 185108. The pivots 185106 and 185108 form a rotational axis for the frame members 185110 and 185112 that allows them to rotate from the collapsed position to the expanded configuration. The pivots 185106 and 185108 can be formed by individual pivot pins and corresponding holes in the cross portions 185114 and 185118 of frame members 185110 and 185112, a pivot common pivot pin/rod/stem/bushing (e.g., 185212), and/or any other suitable pivoting arrangement. The exact manner in which rotation is provided is not critical so long as rotation can be accomplished.

Referring now to FIGS. 185C and 185D, spacer 185102 is shown in its expanded or repair position. As previously described, this is accomplished by rotation of the frame members 185110 and 185112. In this state, spacer 185102 has expanded its width (e.g., see distance S2, which is greater than distance S1 in FIG. 185A) to thereby increase its volume. As shown in FIG. 185D, in one example, the frame members 185110 and 185112 include a generally rectangular shape with rounded or arched cross frame portions 185114 and 185118 and linear middle portions 185116. In some implementations, shapes that are more rectangular or square can be used with more or less arching. In some implementations, shapes that are circular, elliptical and/or sinuous can be used. And, in some implementations, other polygonal shapes, conical, frustoconical and/or circular or elliptical shapes can be used and/or combined. The exact shape of the frame members 185110 and 185112 is not critical so long as the shape(s) provide the spacer 185102 with a greater volume in the expanded state compared to the collapsed state and provide the proper amount of gap filling when expanded.

Thus, the spacer 185102 can be provided in a collapsed state (FIGS. 185A and 185B) and in an expanded or repair state (FIGS. 185C and 185D). The collapsed state assists in the device 185100 traveling through a catheter (or similar device) until it reaches the repair area. At the repair area, the collapsed state can also provide a reduced echogenic image profile to allow more visibility into the tissue or leaflet capture area where the paddles and capture arms operate. The spacer 185102 can be expanded to an increased volume, which is greater than the collapsed volume, to assist in the final repair. In the example of FIGS. 185A-185D, a rotational force or actuation is provided to rotate spacer frame members 185110 and 185112 from a collapsed position to an expanded configuration. A locking or similar device can be included to lock the spacer in its expanded configuration. The locking device can be in the form of a detent mechanism, latch or other mechanism.

The spacer 185102 can be used in a variety of different valve repair devices. For example, the spacer 185102 can be used in any of the valve repair devices described herein. For example, the spacer 185102 shown and described with respect to FIGS. 185A-185D can be used with the valve repair device shown and described with respect to FIGS. 25A and 25B. That is, the spacer 185102 replaces the spacer 3800. The spacer 185102 operates in the same manner as in FIGS. 185A-185D and the valve repair device operates in the same manner as in FIGS. 25A and 25B.

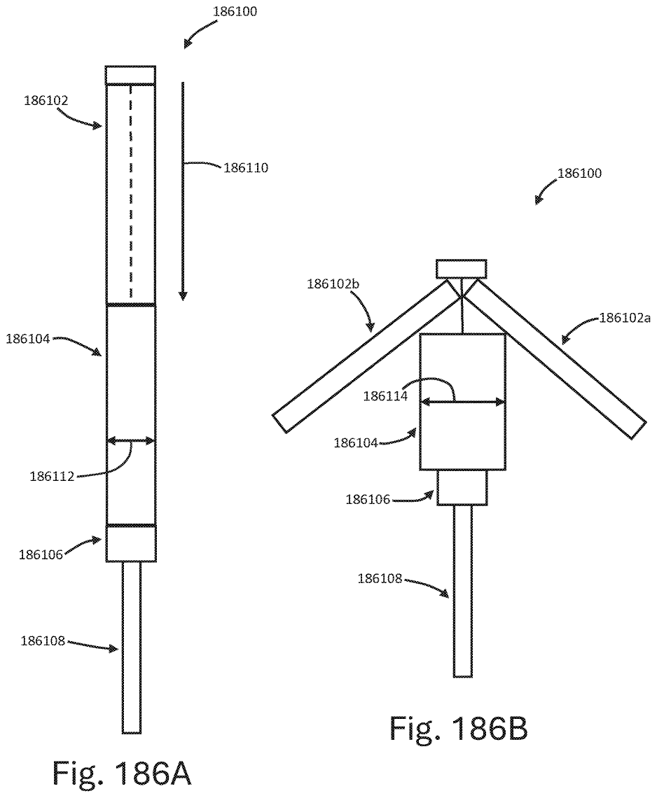

FIGS. 186A and 186B are schematic diagrams of another example of a device 186100 having an expandable coaptation element or spacer that can optionally provide a reduced echogenic profile. In some implementations, the spacer can be self-narrowing in that it is shape-set to a narrow configuration, which can then expand under an applied (spring) force. The spacer changes from a narrow collapsed state (FIG. 186A) to an expanded or repair state (FIG. 186B) via an actuation that applies a force, which can be compressive. Device 186100 includes a paddle portion 186102 (similar to any of the previous described paddle implementations herein), a spacer portion 186104, a retainer or holder portion 186106 and an actuation element 186108.

FIG. 186A shows the device 186100 and the spacer portion 186104 in the narrow or collapsed state. This state assists in allowing device 186100 to travel through a catheter (or similar device) to the repair location because the spacer portion 186104 has a small width 186112. And, as previously described, this narrow or collapsed state also provides the spacer with a narrower echogenic or image profile that gives increased visibility during the tissue or leaflet capture. As a compressive actuation force 186110 is applied, paddle portion 186102 and paddles 186102a and 186102b spread to the schematic position shown in FIG. 186B. Also, the compressive force 186110 causes spacer portion 186104 to move to an expanded or repair state having a width 186114 that is larger than width 186112 of the collapsed state. The retainer/holder portion 186106 holds the spacer portion 186104 in its position as the compressive force or actuation 186110 is applied. Hence, a compressive force or actuation can be applied to change the spacer portion from its narrow collapsed state to its expanded state. In some implementations, the paddle portion 186102 can be adjusted (e.g., further opened or closed) while the spacer is in the expanded state.

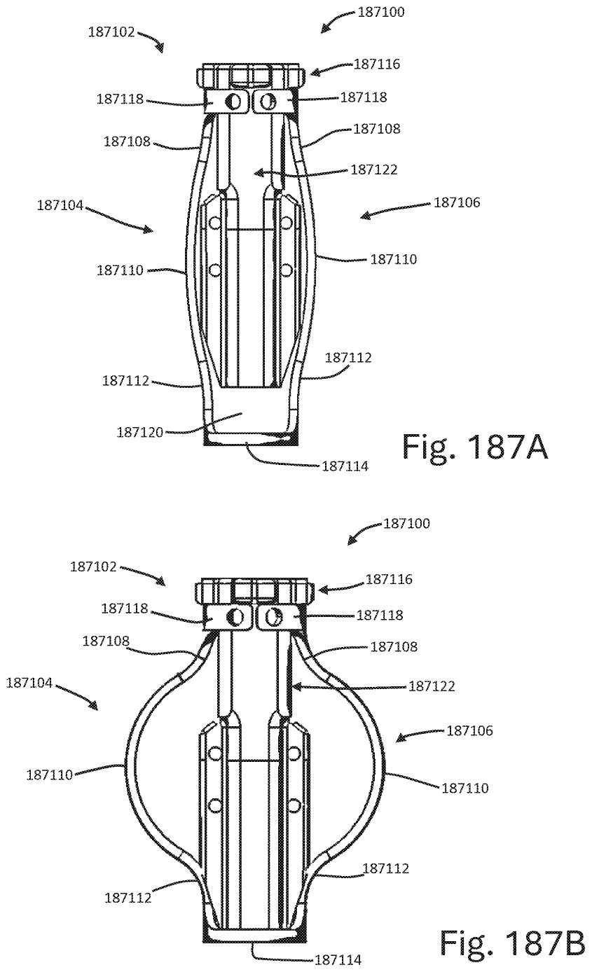

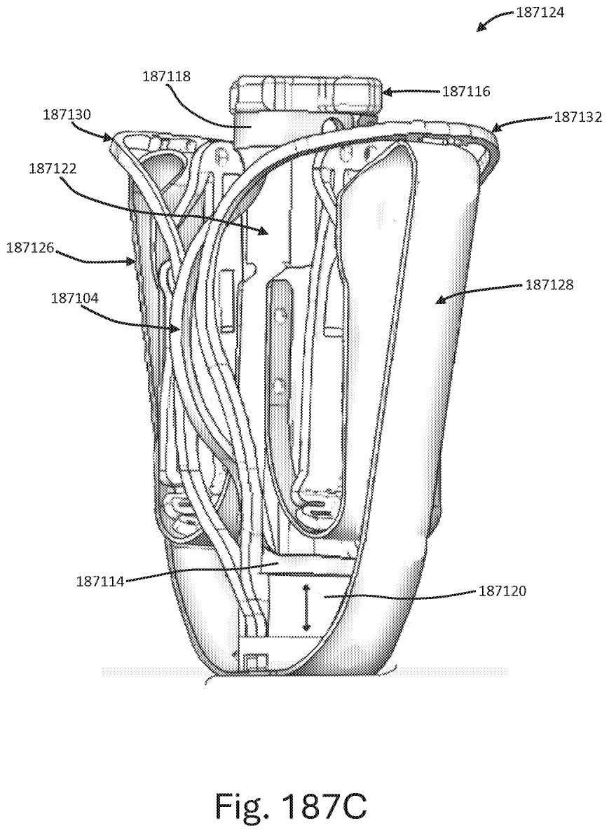

FIGS. 187A, 187B, and 187C illustrate an example of a repair device having a spacer 187100 that is self-narrowing and can optionally provide a reduced echogenic profile and that also narrows under actuation. The spacer 187100 includes a frame 187102 having first and second frame members 187104 and 187106. In this example, each frame member includes flex portions 187108 and 187112, and a middle portion 187110. A brace section 187114 and flanged mounting portions 187118 are also provided. The flanged mounting portions 187118 are positioned near or against a retaining flange, collar, or coupler section 187116 that can be part of a stem, support or bushing member 187122. A compression gap or space 187120 is provided between the end of the support or bushing member 187122 and brace section 187114. An actuating element passes through support or bushing member 187122, compressive gap/space 187120, and brace section 187114 and connects to an actuating cap 187040 and/or retainer according to any of the previously described implementations and examples. The cap 187040 can include an extension 187041 that can be integral with or a separate component from the cap. The cap 187040 and/or the extension 187041 engages the brace section when the device is in the closed position to force the spacer 187100 to the expanded condition shown in FIGS. 187B and 187C.

FIG. 187A shows the spacer 187100 in its self-narrowing collapsed state. This state occurs when the device is opened or elongated to move the cap 187040 and/or the extension 187041 away from the spacer 187100, allowing the spacer to elongate. Once the repair location is reached, the spacer 187100 can be placed in its expanded repair state as shown in FIGS. 187B and 187C. In operation, the actuating cap 187040 and/or extension 187041 applies a compressive actuation on brace section 187114. This pushes the brace section 187114 to move and reduce or eliminate the gap or space 187120. In some implementations, the brace section 187114 engages the bushing 187122 when the valve repair device is fully closed and the cap 187040 is at its most proximal position. The widening of the spacer 187100 is accomplished by the flexure of frame member flex portions 187108 and 187112 and of middle portion 187110 as shown in FIG. 187B to form an expanded sinuous, curved or arched shape. Other shapes are also possible. Spacer 187100 can flex because it is made of a flexible material such as nitinol wire or similar material. As shown in FIG. 187B, the expanded repair state or position of spacer 187100 is wider than that shown in the collapsed state of FIG. 187A.

FIG. 187C shows the spacer 187100 incorporated into an example heart valve repair device 187124. Repair device 187124 can be according to any of the previous described implementations and examples and includes paddles 187126 and 187128 and paddle frames 187130 and 187132. As described in previous implementations and examples, spacer 187100 is positioned in a space between the paddles and the paddle frame. In some implementations, when implanted in a native heart valve, the native valve leaflets wrap around the spacer 187100 and are secured or pinched between the paddle frames 187130, 187132 and the spacer 187100.

The spacer 187100 can be made in a variety of different ways and from a variety of different materials. In some implementations, the spacer can be cut from a tube and shape set in the expanded configuration. For example, the spacer 187100 can be cut from a tube and shape set in the narrow or collapsed configuration. The spacer 187100 can be made from a variety of different materials that can be shape set or molded in the narrow or collapsed configuration. For example, the spacer can be made from a metal, such as steel, nitinol, etc., plastic, etc.

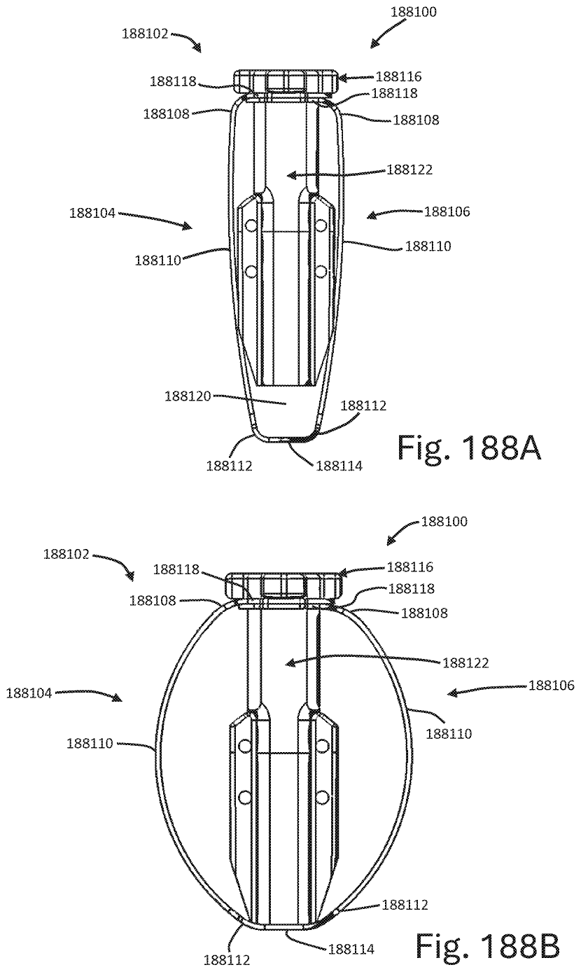

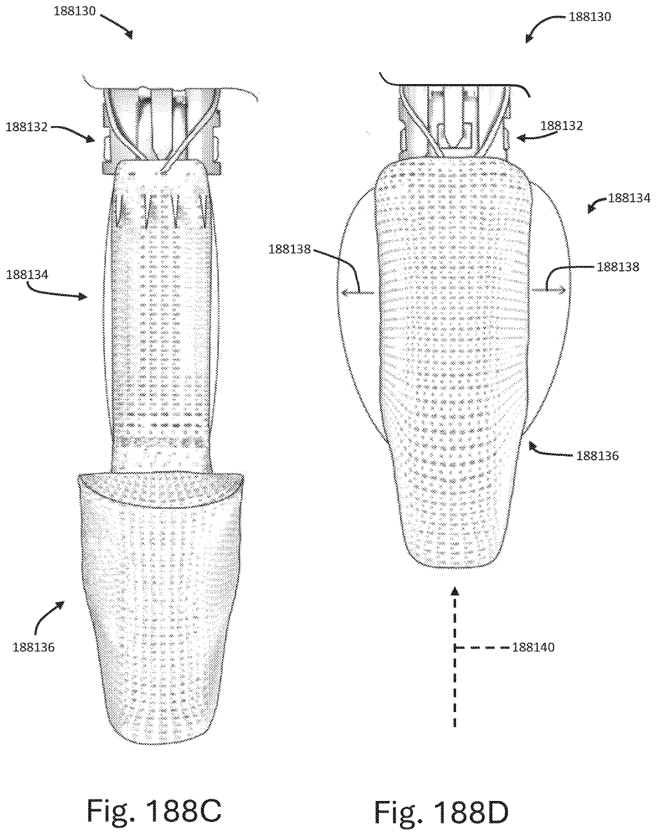

FIGS. 188A and 188B illustrate an example of a spacer 188100 that is made from sheet material, instead of being cut from a tube. The spacer 188100 can operate in the same or similar manner as the spacer 187100. In the example illustrated by FIGS. 188A-188B, the spacer can be cut from a sheet of metal and shape set in the narrow or collapsed configuration. The spacer can be made from a metal, such as steel, nitinol, etc., plastic, etc.

The example of FIGS. 188A and 188B can optionally provide a reduced echogenic profile. FIG. 188A shows the spacer 188100 in the self-narrowing collapsed state and FIG. 188B shows the spacer 188100 in the expanded repair state or position. Similar to the example of FIGS. 187A-187C, the spacer 188100 is a compressible spacer. The spacer 187100 includes a frame 188102 having first and second frame members 188104 and 188106 that can optionally be integrally formed (e.g., cut from a single sheet of material) as illustrated by FIGS. 188A and 188B. In this example, each frame member includes flex portions 188108 and 188112, and a middle portion 188110. A brace section 188114 and flanged mounting portions 188118 are also provided. Flanged mounting portions 187118 are positioned near or against a retaining flange, collar, or coupler section 188116 that can be part of a stem or support or bushing member 188122. In the example shown, the flanged mounting portions 187118 overlap with each other. In other examples, they do not need to overlap but can instead abut or face each other.

A compression gap or space 188120 is provided between the end of the support or bushing member 188122 and brace section 188114. An actuating element passes through support or bushing member 188122, compressive gap/space 188120, and brace section 188114 and connects to an actuating cap and/or retainer according to any of the previously described implementations and examples. The cap can include an extension that can be integral with or a separate component from the cap. The cap and/or the extension engages the brace section when the device is in the closed position to force the spacer to the expanded condition shown in FIG. 188B.

FIG. 188A shows the spacer 188100 in its self-narrowing collapsed state. Once the repair location is reached, the paddles of the device can be closed causing the spacer 188100 to be placed in its expanded repair state as shown in FIG. 188B. In operation, the actuating cap and/or extension a compressive actuation on brace section 188114. This causes the brace section 188114 to at least partially to fully take up the area of gap or space 188120. This is accomplished by the flexure of frame member flex portions 188108 and 188112 and of middle portion 188110 as shown in FIG. 188B to form a sinuous, curved or arched shape. Other shapes are also possible. Spacer 188100 can flex because it is made of a flexible material such as nitinol, steel, plastics or similar materials. As shown in FIG. 188B, the expanded repair state or position of spacer 188100 is wider than that shown in the self-narrowed collapsed state of FIG. 188A.

FIGS. 188C and 188D illustrate the examples of FIGS. 187A-187C and 188A-188B with covers thereon, and attached to a coupler of a delivery system, such as any of the couplers and delivery systems disclosed herein. FIG. 188C illustrates a device 188130 with paddles and clasps in an open condition and with the coaptation element or spacer automatically moved (i.e. simply by opening the paddles) to the self-narrowed collapsed position/state. The narrowed state of the spacer can allow the spacer to fit through a smaller catheter or guide sheath to a repair location. The narrowed state can also produce a reduced echogenic profile during tissue or leaflet capture.

FIG. 188D illustrates device 188130 with the paddles closed and the spacer in the expanded repair position/state suitable for use at the repair location and after application of an actuating or closing direction or force 188140. The actuation or closing force can be provided in a variety of different ways. In some implementations, the paddle frames are shape set to bias the paddles closed. In some implementations, a coupler is manually moved to open and close the paddles and a lock locks or sets the paddles at a user selected position.

In FIGS. 188C and 188D, the device 188130 includes, for example, an actuation portion 188132, spacer portion 188134, and paddle portion 188136. Each are shown having a bio-compatible cover as described herein above and below. FIGS. 188C and 188D illustrate the expansion direction 188138 of spacer portion 188134. FIG. 188D also illustrates, by way of one example, how much farther spacer portion 188134 in its expanded configuration/state can extend beyond the dimensions of paddle portion 188136. In other examples, the expansion can be more or less than that shown.

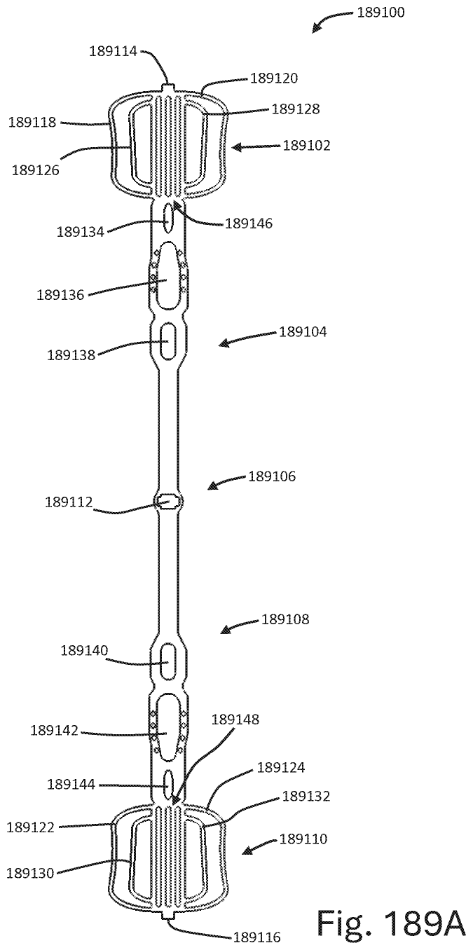

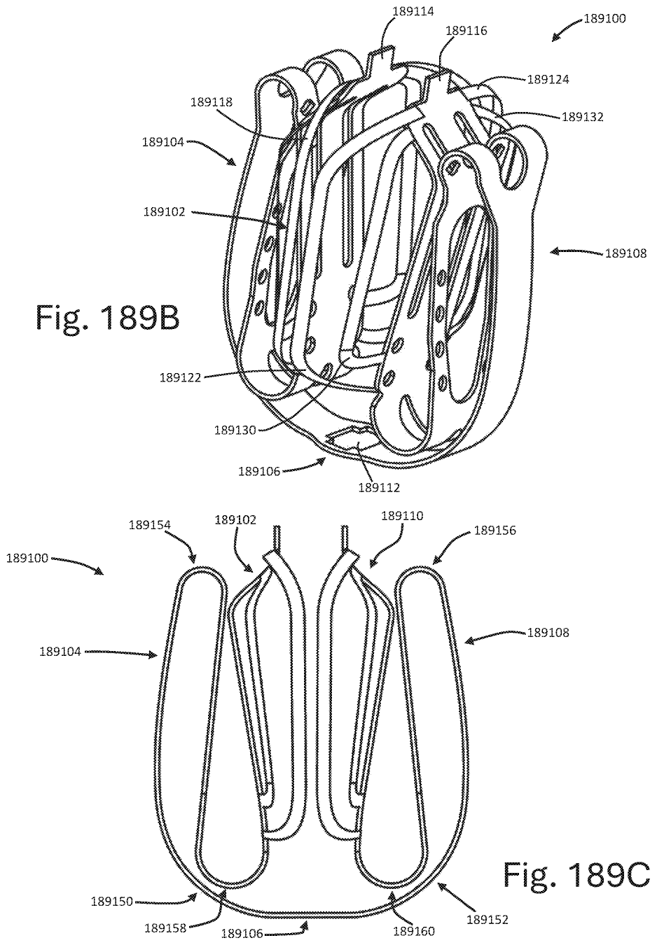

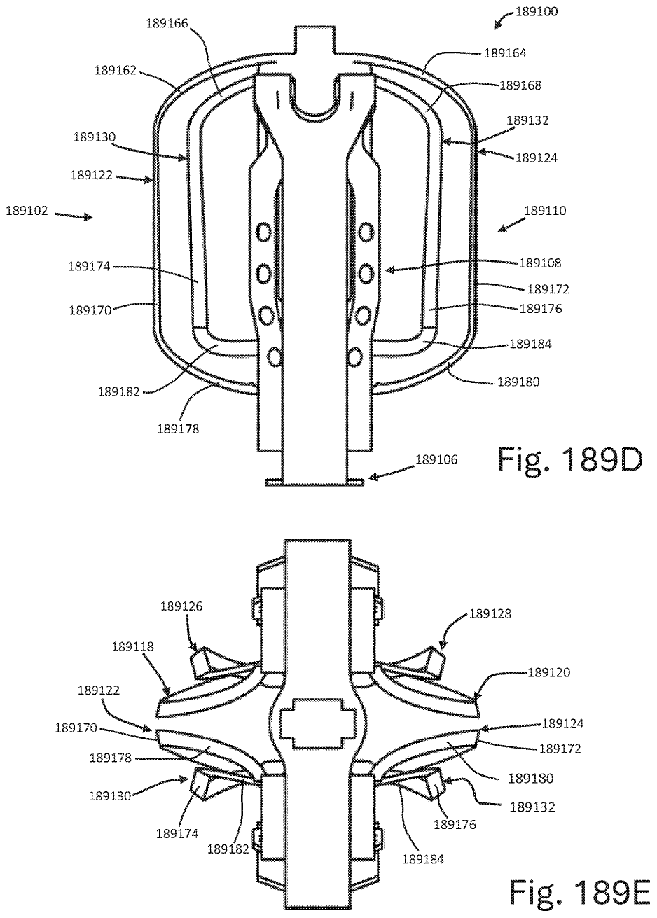

FIGS. 188C and 188D also illustrate example profile views of the device 188130. FIG. 188C shows an example profile of repair device 188130 with paddle portion 188136 in its opened or partially opened or deployed state (e.g., see FIG. 18) whereby tissue or leaflet capture can be initiated. In this profile example, spacer portion 189134 can still be in its self-narrowing collapsed state. This can optionally provide the repair device 188130 with a reduced echogenic imaging shadow and clearer view of the tissue capture area between the spacer portion 188134 and the paddle portion 188136. This is because in the collapsed position/state, the spacer portion 189134 is not in its expanded configuration/state thereby occupying regions of the tissue or leaflet capture area. Once tissue capture is accomplished, spacer portion 188134 can be changed to its expanded repair position/state as shown in FIG. 189D by closing the paddles. Hence, in this example, the collapsed state of the spacer portion 188134 can be used to provide an imaging profile having reduced shadowing in the tissue or leaflet capture area during the capture process and with expansion occurring after capture of the tissue or leaflet. This reduced imaging profile applies to any of the herein described implementations and examples having a coaptation or spacer with a collapsed state or position.

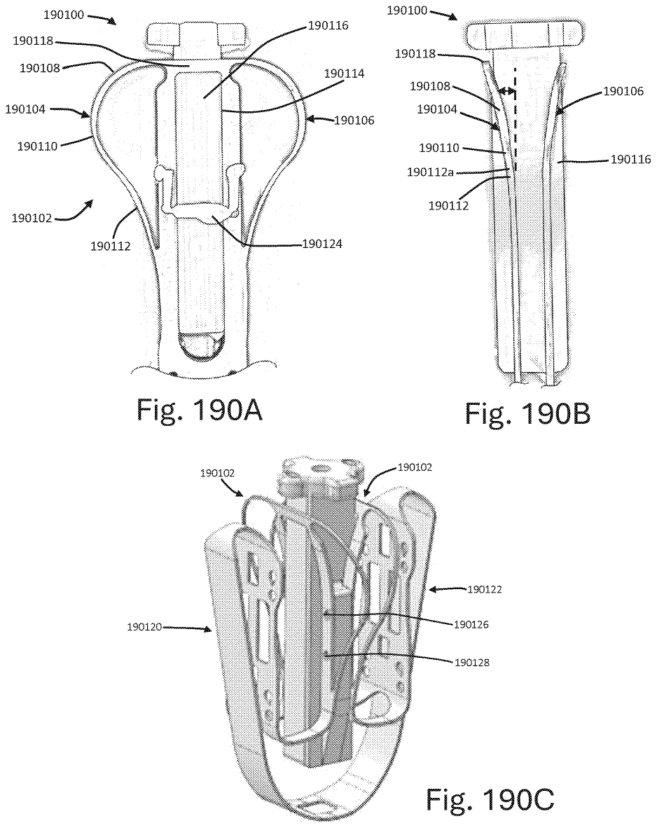

FIGS. 189A-189E illustrate an implementation of a repair device frame 189100 having an integrated coaptation element or spacer. In this example, the device frame 189100 can optionally be made of a single piece of material. This can simplify construction and production of the repair device as assembly of separate components is not necessary. Referring to FIG. 189A, a flat, cut out piece of material 189101 used to make a heart valve repair device frame 189100 is shown and can be made from a thin sheet of material, such as a sheet of plastic material or a sheet of metal material. Other similar materials can also be used. The device frame 189100 includes a plurality of shapes and cutouts made when the sheet material 189101 is flat. The sheet material can then be shaped with applied bends to portions thereof to form the repair device frame 189100 shown in FIGS. 189B-189E. Further examples are illustrated in FIGS. 190A, 190B, and 190C, and 191 and 192.

Referring now to FIG. 189A, the cutout sheet 189101 can include portions that are bent, folded, and/or shape set to form spacer frame portions 189102 and 189110, paddle portions 189104 and 189108, and a middle or actuator base portion 189106. In this example, actuator portion 189106 includes an aperture 189112 for interfacing with an actuation cap and/or retainer section. Extension flanges 189114 and 189116 are provided for interfacing or connecting with a proximal collar or catheter coupling member. Spacer frame portions 189102 and 189110 have a plurality of frame members including outer members 189118-189124 and inner frame members 189126-18932. Optional apertures or windows 189136-189144 are provided as well as cut-outs or openings 189146-189148.

The frame 189100 is formed by bending the flat cutout 189101 at bending portions 189150, 189152, 189154, 189156, and 189160 (FIG. 189C). In addition, the spacer portions 189102, 189110 are formed by bending or contouring the material. In some implementations, the spacer can be formed by contouring the spacer portions around a three-dimensional template or jig. The flat pattern shapes and features shown in FIG. 189A can be formed by, for example, laser cutting a nitinol sheet of material, die stamping or other process. The exact process of fabricating the device frame 189100 is not critical. So constructed, device frame 189100 includes within it the inner paddles, the outer paddles, and spacers in one structure, as compared to two or more separate structures.

Referring now to FIGS. 189B, 189C, 189D and 189E, the formed device frame 189100 shown can be produced by, for example, bending of the flat cutout sheet 189101 illustrated in FIG. 189A. The bending can be accomplished by any means including, for example, a shape-setting fixture or other device/process. The exact process by which the bending or shaping occurs is not critical.

Referring to FIG. 189C, bending portions 189150 and 189152 are located between actuator portion 189106 and paddle portions 189104 and 189108. Bending portions 189154 and 189156 are located within paddle portions 189104 and 189108. Bending portions 189158 and 189160 are located between paddle portions 189104 and 189108 and spacer portions 189102 and 189110. As shown in FIG. 189C, each of the bending portions includes a formation bend or curve from the actuator base portion 189106 through the paddle portions 189104 and 189108 and up to the spacer portions 189102 and 189110. Bending portions 189150 and 189152 can have a more gradual bend (e.g., larger bend radius) compared to the other bending portions. In this example, these bends provide the repair device frame with a single structure for the paddle and spacer formed from a unitary sinuous or curved structure.

FIG. 189D illustrates a view of the repair device frame 189100 showing, among other things, the outer spacer struts 189122 and 189124 and inner spacer struts 189130 and 189132. In this example, outer struts 189118 and 189120 and inner struts 189126 and 189128 are similarly constructed. The outer struts 189122 and 189124 include several portions including first end portions 189162 and 189164, second end portions 189178 and 189180, and central portions 189222, 189224. The inner spacer frame members 189130 and 189132 include portions including first end portions 189166 and 189168, second end portions 189182 and 189184, and central portions 189174 and 189176.

The outer and inner spacer members form a generally square or rectangular shape with rounded end sections. As previously described, other shapes are also included such as for example, polygonal, circular, elliptical, conical, frustoconical, blends of these shapes, etc. As shown in FIG. 189E, the outer spacer struts 189118, 189120, 189122, 189124 laterally extend out further than inner spacer struts 189126, 189128, 189130, 189132. As also shown, inner spacer struts 189126, 189128, 189130, 189132 longitudinally extend out further than outer spacer struts 189118, 189120, 189122, 189124. A bio-compatible cover material can be applied to the spacer frame portions 189102 and 189110 to provide a smoother and/or mor biocompatible spacer.

Furthermore, barbs or barb-like retention structures can be included on the outer struts, the inner struts, and/or the paddles. These barbs or barb-like structures can be formed by cutting the flat sheet 189101 to include barbs or barb-like retention structures and bending the barbs or barb-like structures to point in a direction appropriate for securing native valve leaflet tissue. A first set of barbs can be provided on the outer spacer struts 189118, 189120, 189122, 189124 that, for example, interlock leaflet tissue with the paddles so the leaflet would be held or pinned in place upon or during paddle closure. A second set of barbs can be included on inner spacer struts 189126, 189128, 189130, 189132 that are ventricular facing and prevent, inhibit or retain the leaflet from coming out of the repair device.

So constructed, the example device frame 189100 of FIGS. 189A-189E provides both paddles and a spacer from a unitary sheet material that simplifies construction and production of the repair device. This reduces the number of parts in the device and the assembly time. Further, by shaping and spacing the spacer frame members by length, width and spacing, the resistance to compression can be controlled or regulated, which can create a better seal between the paddles, leaflet, and spacer. Further yet, spacer geometries with a plurality of posterior and medial to lateral shapes that are self-expanding using the elastic properties of nitinol or other shape settable material are provided. And, barb or barb-like projections can be included in one or more of the spacer frame members to assist in tissue or leaflet capture and retention.

The single piece spacer and paddles or device frame 189100 of FIGS. 189A-189E can take a variety of different forms and the spacer can have a variety of different shapes and sizes. In some implementations, the spacer can be configured such that the spacer does not need to be shape set around a three-dimensional tool or die. For example, the single piece spacer and paddles can be bent and shape set to form the paddles and the transition, but the spacer portion is left in its flat state. In some implementations, assembling the flat spacer portions to a bushing bends or flexes the spacer portions to have a curved and/or three-dimensional shape.

FIGS. 190A, 190B, and 190C illustrate an example of a single piece spacer and paddles 190100 where the spacer portions are originally left in a flat configuration. When the flat spacer portions 190102 are attached to a bushing 190116, the spacer portions bend or flex to a curved and/or three-dimensional shape. Referring to FIGS. 190A-190C, the single piece spacer and paddles 190100 have a spacer portion 190102 that includes outer first and second frame members 190104 and 190106. The outer first and second frame members 190104 and 190106 can be connected by brace portion 190118. Each of the frame members are similarly constructed. In this regard, frame member 190104 includes portions 190108, 190110, and 190112. Each of these portions can be curved as shown due to the attachment to the bushing 190116 and interference between the brace portion 190118 and the bushing 190116.

Frame member portion 190112 can include a bend angle at or near portion 190112a as shown due to the attachment to the bushing 190116 and interference between the brace portion 190118 and the bushing 190116. In some implementations, the bend angle can occur at other locations of the frame member and the frame member can bend or contour at multiple locations. Additionally, a plurality of bend angles can also be provided in the frame member to accomplish the same or similar geometry. Further, the bend angle can be increased from that shown to allow the spacer frame portion to extend further away from the bushing 190116, while allowing for bending or spring action back towards the stem, sleeve, or bushing.

The spacer frame portion 190102 also includes an opening or cut-out 190114. As shown in FIG. 190B, opening or cut-out 190114 can be used to allow a portion of the bushing 190116 to protrude therethrough thereby allowing for a recessing of sections of the spacer frame portion 190102, with a resulting contour of the spacer portions. One or more sutures or fasteners 190124 can be used to connect the spacer frame portion 190102 to the actuator stem, sleeve, or bushing 190116. In this regard, one or more holes or apertures 190126 and 190128 can be provided for fastening to the actuator stem, sleeve or bushing 190116.

As previously described, the spacer portion 190102 can be made of a metal, such as nitinol, which can be shape-set (if desired) and/or is resilient (i.e., spring-like). FIG. 190C shows the spacer portion 190102 as part of the integrated spacer and paddles similar to that of FIG. 189A and bent or formed similar to that of FIGS. 189B-189E to have, among other things, paddle portions 190120 and 190122. More specifically, the spacer portion 190102 can be formed in place of the spacer portion 189102 and 189110 shown in FIGS. 189A-189E to provide an integrated spacer and paddles.

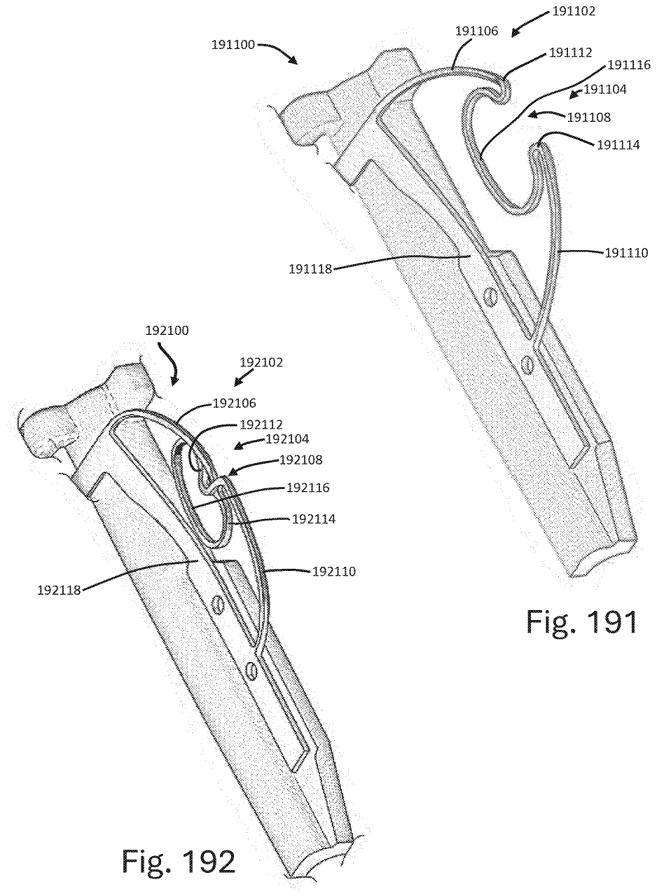

In some implementations, spacers can be configured to have a wide configuration and a narrow configuration by making a small adjustment to the spacer. FIGS. 191 and 192 illustrate an example of a spacer portion 191102 that can be adjusted to have a wide (FIG. 191) and narrow (FIG. 192) width profiles. In some implementations, the spacer portion 191102 can be used in a paddles and spacer that are made from a single piece of material, such as the examples illustrated by FIGS. 189A-189E and 190A-190C. In some implementations, the spacer portion 191102 can be used with paddles that are separate from the spacer, such as in any of the valve repair devices with spacers disclosed herein other than the examples illustrated by FIGS. 189A-189E and 190A-190C.

In FIG. 191, a heart valve repair device 191100 includes a spacer frame portion 191102 having a frame member 191104. The frame member 191104 has a plurality of bent or curved portions including side portions 191106 and 191110. The side portions 191106 and 191110 each include curved end portions 191112 and 191114 that are spaced apart from each other. The end portions 191112 and 191114 connect to a curved recessed portion 191116. A bend angle can be implemented at or near portion 191118 similar to that shown in FIG. 190B. So formed, the example of frame member 191104 provides a wide width spacer frame. As previously described, the spacer frame portion 191102 can be made of nitinol or similar material that can be non-shape set or alternatively shape-set.

FIG. 192 illustrates another example of a repair device 192100, where the spacer portion 191102 has been adjusted to a configuration that provides a narrower width profile compared to FIG. 191. The spacer portion 191112 can be adjusted in a variety of different ways to provide a narrower width. In some implementations, the side portions 191106 and 191110 are pressed together and attached to narrow the spacer portion 191102. The side portions 191106 and 191110 can be attached in a variety of different ways, such as with sutures, fasteners, etc. In some implementations, the end extensions 191112 and 191114 are pressed together and attached to form the narrower spacer portion 191102.

A bend angle can be implemented at or near portion 191118 similar to that shown in FIG. 190B. So formed, frame member 192104 provides narrower width profile spacer frame. As previously described, the spacer frame portion 192102 can be made of nitinol or similar material that can be non-shape set or alternatively shape-set. In some implementations, the frame member of FIGS. 191 and 192 can be part of a paddle and spacer structure instead of being separate components affixable to a bushing.

In any of the previous embodiments, the geometry or amount of bending or curvature can be used to regulate the amount of resiliency or spring force provided by the components or portions. Additionally, the width and/or thickness of the material (e.g., nitinol) can also be used to regulate the amount of resiliency or spring force provided. Further yet, shape-setting or non-shape-setting can also be used to regulate the amount of resiliency or spring force provided. Any one or more of these techniques can be used in combination to obtain the desired resiliency and/or spring force.

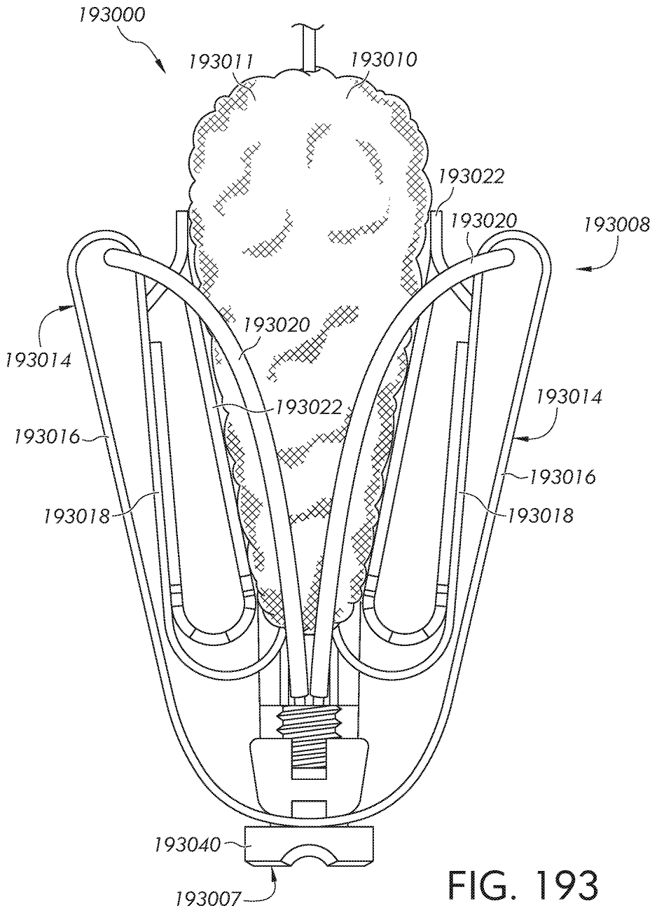

FIGS. 193-195 illustrate an example of a device 193000 (e.g., valve repair device, valve treatment device, implantable device, implant, etc.) having a coaptation element 193010 that is configured to reduce or compress against the leaflets when the device 193000 is attached to a native heart valve (e.g., the mitral valve or the tricuspid valve).

The coaptation element 193010 can be used in a variety of different devices or valve repair devices, including but not limited to, any of the devices and valve repair devices disclosed in the present application. The device 193000 can include any other features for a device or valve repair device discussed in the present application.

In some implementations, the coaptation element 193010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application). The device 193000 can be deployed from a delivery sheath, and/or an implant catheter. The device 193000 can be a prosthetic spacer device, valve repair device, valve treatment device, implant, or another type of device that attaches to leaflets of a native valve.

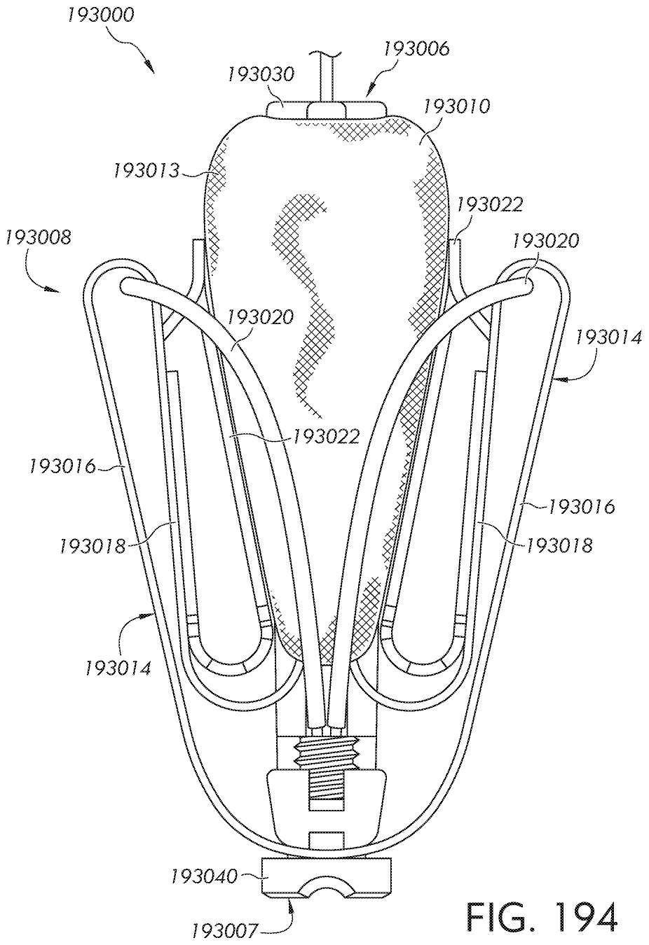

In some implementations, the device or valve repair device 193000 includes a proximal or attachment portion 193006 (shown in FIG. 194), an anchor portion 193008, a coaptation element 193010, and a distal portion 193007.

In some implementations, the anchor portion 193008 includes a plurality of anchors 193014. The anchors 193014 can be configured in a variety of ways. In some implementations, the anchors 26014 include outer paddles 193016, inner paddles 193018, paddle extension members or paddle frames 193020, and clasps 193022.

In some implementations, the attachment portion 193006 includes a first or proximal collar 193030 (or other attachment element) (shown in FIG. 194) for engaging with a capture mechanism or coupler of a delivery system.

A delivery system for the implant 193000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the outer and inner paddles 193016, 193018 are formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, an actuation element (e.g., actuation shaft, actuation rod, actuation tube, actuation wire, actuation line, etc.) can extend from a delivery system (not shown) to engage and enable actuation of the device 193000 (see actuation element 112 in FIGS. 8-14). In some implementations, the actuation element extends through the proximal collar 193030 and the coaptation element 193010 to engage a cap 193040 of the distal portion 193007. The actuation element can be configured to removably engage the cap 193040 with a threaded connection, or the like, so that the actuation element can be disengaged and removed from the implant 193000 after implantation.

In some implementations, the outer paddles 193016 are jointably attached to the cap 193040 of the distal portion 193007 and to the inner paddles 193018 by any suitable means, such as, for example, any means for connecting outer paddles to a distal cap and inner paddles described in the present application. In some implementations, the paddle frames 193020 are attached to the cap 193040 at the distal portion 193007 and extend to the transition portions (e.g., any transition portions of a paddle frame described in the present application) between the inner and outer paddles 193018, 193016.

In some implementations, the paddle frames 193020 are formed of a material that is more rigid and stiff than the material forming the paddles 193018, 193016 so that the paddle frames 193020 provide support for the paddles 193018, 193016.

In some implementations, the paddle frames 193020 can provide additional pinching force between the inner paddles 193018 and the coaptation element 193010. The connections between the paddle frames 193020, the outer and inner paddles 193016, 26018, and the cap 193040 can constrain each of these parts to the movements and positions of the devices or valve repair devices described herein.

In some implementations, the coaptation element 193010 includes a fabric material 193011 and an optional cover 193013 disposed over the fabric material 193011. In some implementations, the coaptation element 193010 does not include a metal material (e.g., Nitinol or other metal material) or other solid material that defines a shape of the coaptation element 193010, but rather the fabric material 193011 is positioned over a bushing (not shown) or other element of the device 193000 that is configured to receive an actuation element of the delivery device (such that the actuation element can extend through the coaptation element 193010 to engage the cap 193040 of the distal portion 193007). In such implementations, the fabric material 193011 defines the shape of the coaptation element 193010.

In some implementations, the fabric material 193011 can be made of, for example, a lofted cloth material (e.g., high loft polyester, high loft cotton, high loft wool, high loft polyethylene, high loft polyurethane, high loft polypropylene, a blend of any of these, etc.) The fabric material 193011 can be any fabric that has a thick and/or fluffy texture. In some implementations, the fabric material 193011 can be made from a biocompatible material that is configured to promote tissue ingrowth, such as, for example a woven, spun bond, air laid, etc. biocompatible fabric that is configured to promote tissue ingrowth.

In some implementations, the fabric material 193011 is configured to reduce the stress on the leaflets when the device 193000 is attached to a native heart valve. When the device 193000 is attached to a native heart valve, the leaflets are captured between the anchors 193014 and the coaptation element 193010. The fabric material 193011 of the coaptation element 193010 can cushion and/or reduce the stresses on the leaflets resulting from the engagement between the leaflets and the coaptation element 193010.

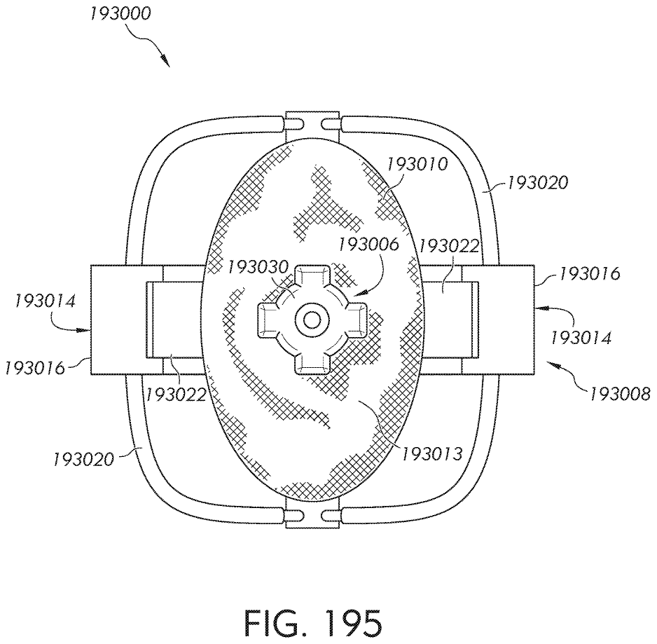

Referring to FIGS. 194 and 195, the optional cover 193013 is configured to be positioned over the fabric material 193011 such that the optional cover 193013 defines the shape of the coaptation element 193010. The optional cover 193013 can optionally be configured to protect the fabric material 193011 when the device 193000 is attached to a native heart valve. The cover 193013 can also remove issues regarding tight tolerances of the fabric material 193011 because the fabric material 193011 is covered by the cover 193013.

The cover 193013 can be configured in a variety of ways. In some implementations, the cover 193013 can include a sheet, material, fabric, layer or membrane. In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

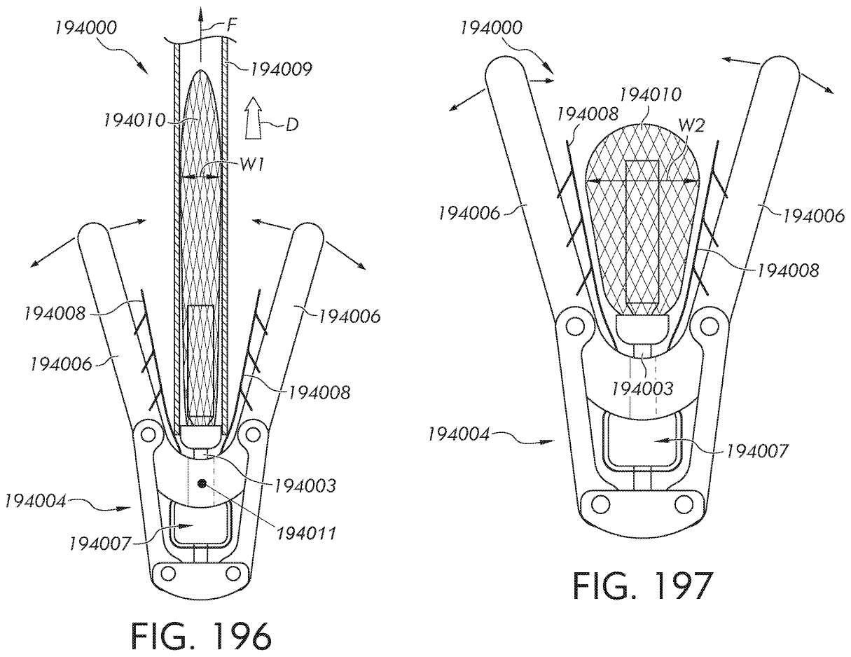

FIGS. 196-197 illustrate an example of a device 194000 (e.g., valve repair device, valve treatment device, implantable device, implant, etc.) having a coaptation element 194010 that is movable between an elongated configuration (as shown in FIG. 196) and an expanded configuration (as shown in FIG. 197).

The coaptation element 194010 can be used in a variety of different devices or valve repair devices, including but not limited to, any of the devices and valve repair devices disclosed in the present application. The device 194000 can include any other features for a device or valve repair device discussed in the present application, and the coaptation element 194010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application).

The device 194000 can be deployed from a delivery sheath, and/or an implant catheter. The device 194000 can be a prosthetic spacer device, valve repair device, valve treatment device, implant, or another type of device that attaches to leaflets of a native valve.

In some implementations, the device 194000 can take the form of the device 402 shown in FIG. 24, but the device 194000 further includes the coaptation element 194010. For instance, the device 194000 can include a base assembly 194004, a pair of paddles 194006, and a pair of gripping members 194008 (e.g., clasps, clasp arms, grippers, gripping arms, latches, etc.).

In some implementations, the paddles 194006 can form part of the base assembly 194004. For example, the paddles 194006 can be links of the base assembly. In the illustrated example, the base assembly 194004 has a shaft 194003, a coupler (not shown) configured to move along the shaft 194003, and a lock 194007 configured to lock the coupler in a stationary position on the shaft.

In some implementations, the coupler is mechanically connected to the paddles 194006, such that movement of the coupler along the shaft 194003 causes the paddles to move between an open position and a closed position. For example, each of the paddles 194006 can be pivotally connected to the coupler at a pivot point 194011. Distal ends of the paddles can overlie one another near the pivot point 194011 as indicated by the solid and dashed lines in the figures. The coupler can mechanically couple the paddles 194006 to the shaft 194003 and, when moving along the shaft 194003, can cause the paddles 194006 to move between their open and closed positions.

In some implementations, the gripping members 194008 are connected to the base assembly 194004 (e.g., the gripping members 194008 can be connected to the shaft 194003, or any other suitable member of the base assembly), such that the gripping members can be moved and/or flexed to adjust the width of the opening between the paddles 194006 and the gripping members 194008.

In some implementations, the gripping member 194008 can include an optional barbed portion for attaching the gripping members to valve tissue when the device 194000 is attached to the valve tissue. The gripping members 194008 can take any suitable form, such as, for example, any form of a gripping member or clasp disclosed in the present application.

In some implementations, when the paddles 194006 are in the closed position, the paddles engage the gripping members 194008, such that, when valve tissue is attached to the barbed portion of the gripping members, the paddles secure the device 194000 to the valve tissue.

In some implementations, the gripping members 194008 are configured to engage the paddles 194006 such that the barbed portion engages the valve tissue member and the paddles 194006 to secure the device 194000 to the valve tissue member. For example, in some instances, it can be advantageous to have the paddles 194006 maintain an open position and have the gripping members 194008 move outward toward the paddles 194006 to engage valve tissue and the paddles 194006.

While the device 194000 shown in FIGS. 196-197 illustrates a pair of paddles 194006 and a pair of gripping members 194008, it should be understood that the device 194000 can include any suitable number of paddles and gripping members.

In some implementations, the coaptation element 194010 can be configured to move between an elongated configuration (as shown in FIG. 196) and an expanded configuration (as shown in FIG. 197). The coaptation element 194010 can have a width W1 (FIG. 196) when in the elongated configuration and a width W2 (FIG. 197) when in the expanded configuration. In some implementations, the width W1 is less than the width W2.

When the device 194000 is being attached to a native heart valve, it can be advantageous to have the coaptation element 194010 be in the elongated configuration because the smaller width W1 of the coaptation element 194010 allows for additional space between the coaptation element 194010 and the paddles 194006. This can make it easier for a user to secure the device 194000 to one or more leaflets of the native heart valve with the paddles 194006 and gripping member 194008, e.g., because the opening between the paddle 194006 and the gripping member 194008 is larger.

After the device 194000 is secured to the native heart valve by the paddles 194006 and gripping members 194008, it can be advantageous to have the coaptation element 194010 be in the expanded configuration because the larger width W2 of the coaptation element 194010 reduces the space between the paddles 194006 and the coaptation element 194010 such that the paddles 194006 and coaptation element 194010 create a pinching force on a leaflet of the native heart valve to maintain the device 194000 in a secured connection with the one or more leaflets. In some implementations, the paddles 194006 and coaptation element 194010 exert a pinching force on a leaflet of the native heart valve such that expansion of the coaptation element 194010 increases and/or maintains the pinching force.

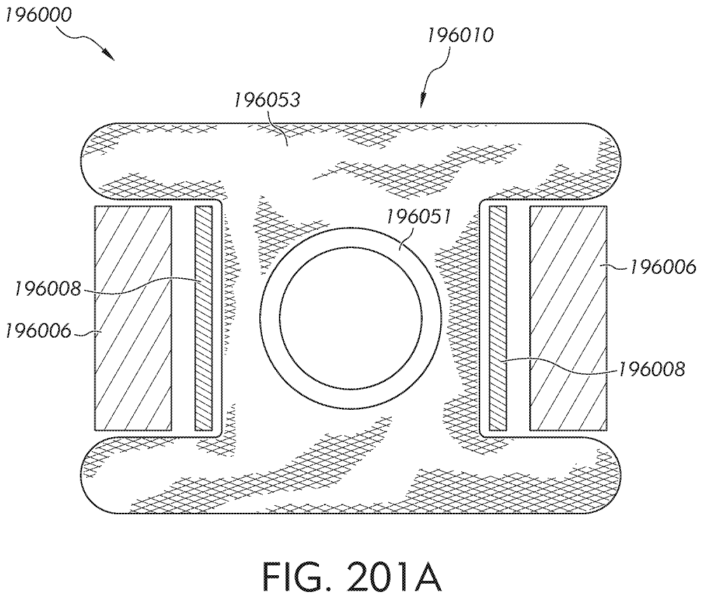

In some implementations, the coaptation element 194010 engages the paddles 194006 and/or the gripping members 194008 when in the expanded configuration such that the coaptation element 194010 forms around the paddles 194006 and/or the gripping members 194008 (e.g., similar to the coaptation element 196010 shown in FIG. 201A).

In some implementations, a ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 194010 can be shape set in the expanded configuration (as shown in FIG. 197), and an actuation element can be used to engage the coaptation element 194010 to cause the coaptation element 194010 to be in the elongated configuration (as shown in FIG. 196).

For example, referring to FIG. 196, in some implementations, the valve repair system can include an actuation tube 194009, and the coaptation element 194010 can be disposed within the actuation tube 194009 when the valve repair device 194000 is delivered to a native heart valve such that the coaptation element 194010 is maintained in the elongated configuration.

After the device 194000 is secured to the native heart valve (e.g., via the paddles 194006 and gripping members 194008), the actuation tube 194009 can be moved in the direction D such that the coaptation element 194010 is no longer disposed within the actuation tube 194009, which causes the coaptation element 194010 to move back to its normally expanded configuration (as shown in FIG. 197). While the coaptation element is shown being in a normally expanded configuration and engaged by an actuation element to be moved to the elongated configuration, it should be understood that the coaptation element 194010 can be moved between the elongated and expanded configurations by any suitable means.

In some implementations, the coaptation element 194010 can be attached to the shaft 194003 of the base assembly 194004. However, it should be understood that the coaptation element 194010 can be attached to any other portion of the device 194000 that allows for the coaptation element to be positioned such that one or more leaflets of the native heart valve can coapt against the coaptation element 194010 when the device 194000 is attached to the native heart valve.

In some implementations, the coaptation element 194010 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, the coaptation element 194010 can include an optional cover. The cover can be configured in a variety of ways. In some implementations, the cover can include a sheet, material, fabric, layer or membrane that is attached to the coaptation element 194010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 194003 of the base assembly 194004 of the device 194000. In some implementations, after the device 194000 is secured to valve tissue, the placement shaft can be removed from the shaft 194003 to remove the device 194000 from the remainder of the treatment and/or repair system, such that the device 194000 can remain attached to the valve tissue, and the delivery device can be removed from a patient's body.

In some implementations, the treatment and/or repair system can also include a paddle control mechanism, a gripper control mechanism, and/or a lock control mechanism.

In some implementations, the paddle control mechanism can be mechanically attached to the coupler of the base assembly 194004 to move the coupler along the shaft 194003, which causes the paddles 194006 to move between the open and closed positions. The paddle control mechanism can take any suitable form, such as, for example, any form for a paddle control mechanism described in the present application.

In some implementations, the gripper control mechanism can be configured to move the gripping members 194008 such that the width of the opening between the gripping members 194008 and the paddles 194006 can be altered. The gripper control mechanism can take any suitable form, such as, for example, any form for a gripper control mechanism described in the present application.

In some implementations, the lock control mechanism can be configured to lock and unlock the lock 194007. That is, the lock 194007 can be configured to lock the coupler in a stationary position with respect to the shaft 194003 and can take a wide variety of different forms and the type of lock control mechanism can be dictated by the type of lock used. The lock control mechanism can take any suitable form, such as, for example, any form described in the present application.

Additional features of the device 194000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

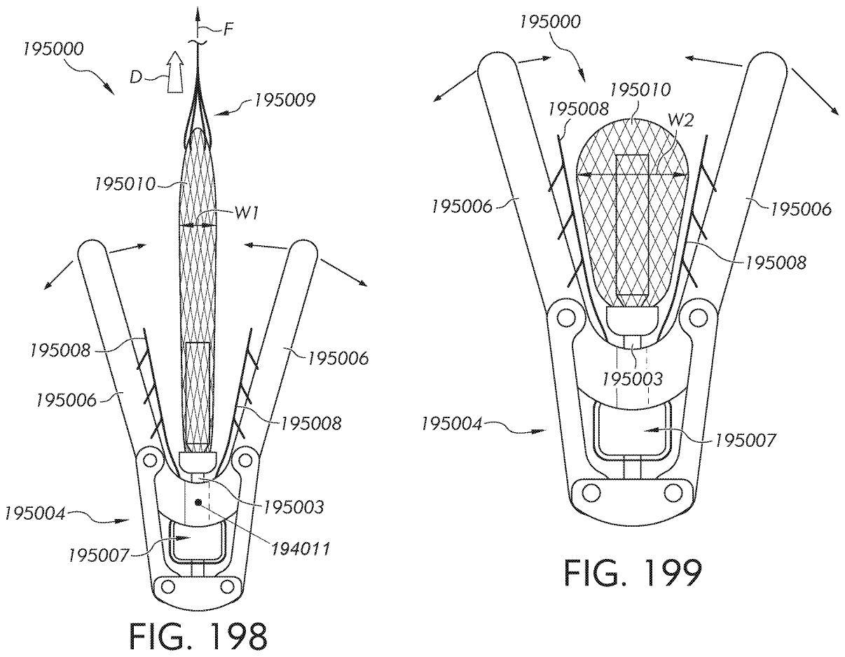

FIGS. 198-199 illustrate an example of a device 195000 (e.g., valve repair device, valve treatment device, implantable device, implant, etc.) having a coaptation element 195010 that is movable between an elongated configuration (as shown in FIG. 198) and an expanded configuration (as shown in FIG. 199).

The coaptation element 195010 can be used in a variety of different devices or valve repair devices, including but not limited to, any of the devices and valve repair devices disclosed in the present application. The device 195000 can include any other features for a device or valve repair device discussed in the present application, and the coaptation element 195010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application). The device 195000 can be deployed from a delivery sheath, and/or an implant catheter. The device 195000 can be a prosthetic spacer device, valve repair device, valve treatment device, implant, or another type of device that attaches to leaflets of a native valve.

In some implementations, the device 195000 can take the form of the device 402 shown in FIG. 24, but the device 195000 further includes the coaptation element 195010. For example, the device 195000 can include a base assembly 195004, a pair of paddles 195006, and a pair of gripping members 195008 (e.g., clasps, clasp arms, grippers, gripping arms, latches, etc.).

In some implementations, the paddles 195006 can be part of the base assembly 195004. For example, the paddles 195006 can be formed as extensions or parts of links of the base assembly. In the illustrated example, the base assembly 195004 has a shaft 195003, a coupler (not shown) configured to move along the shaft 195003, and a lock 195007 configured to lock the coupler in a stationary position on the shaft.

In some implementations, the coupler is mechanically connected to the paddles 195006, such that movement of the coupler along the shaft 195003 causes the paddles to move between an open position and a closed position. For example, distal end portions of the paddles can be coupled to the coupler by a pivot connection 194011. In this way, the coupler serves as a means for mechanically coupling the paddles 195006 to the shaft 195003 and, when moving along the shaft 195003, for causing the paddles 195006 to move between their open and closed positions.

In some implementations, the gripping members 195008 are connected to the base assembly 195004 (e.g., the gripping members 195008 can be connected to the shaft 195003, or any other suitable member of the base assembly), such that the gripping members can be moved and/or flexed to adjust the width of the opening between the paddles 195006 and the gripping members 195008.

In some implementations, the gripping member 195008 can include an optional barbed portion for attaching the gripping members to valve tissue when the device 195000 is attached to the valve tissue. The gripping members 195008 can take any suitable form, such as, for example, any form of a gripping member or clasp disclosed in the present application.

In some implementations, when the paddles 195006 are in the closed position, the paddles engage the gripping members 195008, such that, when valve tissue is attached to the barbed portion of the gripping members, the paddles secure the device 195000 to the valve tissue.

In some implementations, the gripping members 195008 are configured to engage the paddles 195006 such that the barbed portion engages the valve tissue member and the paddles 195006 to secure the device 195000 to the valve tissue member. For example, in certain situations, it can be advantageous to have the paddles 195006 maintain an open position and have the gripping members 195008 move outward toward the paddles 195006 to engage valve tissue and the paddles 195006.

While the device 195000 shown in FIGS. 198-199 illustrates a pair of paddles 195006 and a pair of gripping members 195008, it should be understood that the device 195000 can include any suitable number of paddles and gripping members.

In some implementations, the coaptation element 195010 can be configured to move between an elongated configuration (as shown in FIG. 198) and an expanded configuration (as shown in FIG. 199). The coaptation element 195010 can have a width W1 (FIG. 198) when in the elongated configuration and a width W2 (FIG. 199) when in the expanded configuration. In some implementations, the width W1 is less than the width W2.

When the device 195000 is being attached to a native heart valve, it can be advantageous to have the coaptation element 195010 be in the elongated configuration because the smaller width W1 of the coaptation element 195010 allows for additional space between the coaptation element 195010 and the paddles 195006. This can make it easier for a user to secure the device 195000 to one or more leaflets of the native heart valve with the paddles 195006 and gripping member 195008, e.g., because the opening between the paddle 195006 and the gripping member 195008 is larger.

After the device 195000 is secured to the native heart valve by the paddles 195006 and gripping members 195008, it can be advantageous to have the coaptation element 195010 be in the expanded configuration because the larger width W2 of the coaptation element 195010 reduces the space between the paddles 195006 and the coaptation element 195010 such that the paddles 195006 and coaptation element 195010 create a pinching force on a leaflet of the native heart valve to maintain the device 195000 in a secured connection with the one or more leaflets. In some implementations, the paddles 195006 and coaptation element 195010 exert a pinching force on a leaflet of the native heart valve such that expansion of the coaptation element 195010 increases and/or maintains the pinching force.

In some implementations, the coaptation element 195010 engages the paddles 195006 and/or the gripping members 195008 when in the expanded configuration such that the coaptation element 195010 forms around the paddles 195006 and/or the gripping members 195008 (e.g., similar to the coaptation element 196010 shown in FIG. 201A).

In some implementations, a ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 195010 can be shape set in the expanded configuration (as shown in FIG. 199), and an actuation element can be used to engage the coaptation element 195010 to cause the coaptation element 195010 to be in the elongated configuration (as shown in FIG. 198).

For example, referring to FIG. 198, in some implementations, the valve repair system can include one or more actuation elements 195009 (e.g., sutures, wires, etc.), and a user can pull on the actuation element(s) 195009 in the direction D to create a force F on the coaptation element 195010 that cause the coaptation element 195010 to be in the elongated configuration.

After the device 195000 is secured to the native heart valve (e.g., via the paddles 195006 and gripping members 195008), the actuation element(s) 195009 can be removed from the coaptation element 195010 or otherwise disengaged by a user such that force F is no longer applied to the coaptation element 195010, which causes the coaptation element 195010 to move back to its normally expanded configuration (as shown in FIG. 199). While the coaptation element 195010 is shown being in a normally expanded configuration and engaged by an actuation element to be moved to the elongated configuration, it should be understood that the coaptation element 195010 can be moved between the elongated and expanded configurations by any suitable means.

In some implementations, the coaptation element 195010 can be attached to the shaft 195003 of the base assembly 195004. However, it should be understood that the coaptation element 195010 can be attached to any other portion of the device 195000 that allows for the coaptation element to be positioned such that one or more leaflets of the native heart valve can coapt against the coaptation element 195010 when the device 195000 is attached to the native heart valve.

In some implementations, the coaptation element 195010 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, the coaptation element 195010 can include an optional cover. The cover can be configured in a variety of ways. In some implementations, the cover can include a sheet, material, fabric, layer or membrane that is attached to the coaptation element 195010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 195003 of the base assembly 195004 of the device 195000. In some implementations, after the device 195000 is secured to valve tissue, the placement shaft can be removed from the shaft 195003 to remove the device 195000 from the remainder of the treatment and/or repair system, such that the device 195000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 195004 to move the coupler along the shaft 195003, which causes the paddles 195006 to move between the open and closed positions. The paddle control mechanism can take any suitable form, such as, for example, any form for a paddle control mechanism described in the present application.

In some implementations, the gripper control mechanism can be configured to move the gripping members 195008 such that the width of the opening between the gripping members 195008 and the paddles 195006 can be altered. The gripper control mechanism can take any suitable form, such as, for example, any form for a gripper control mechanism described in the present application.

In some implementations, the lock control mechanism can be configured to lock and unlock the lock 195007. That is, the lock 195007 can be configured to lock the coupler in a stationary position with respect to the shaft 195003 and can take a wide variety of different forms and the type of lock control mechanism can be dictated by the type of lock used. The lock control mechanism can take any suitable form, such as, for example, any form described in the present application.

Additional features of the device 195000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

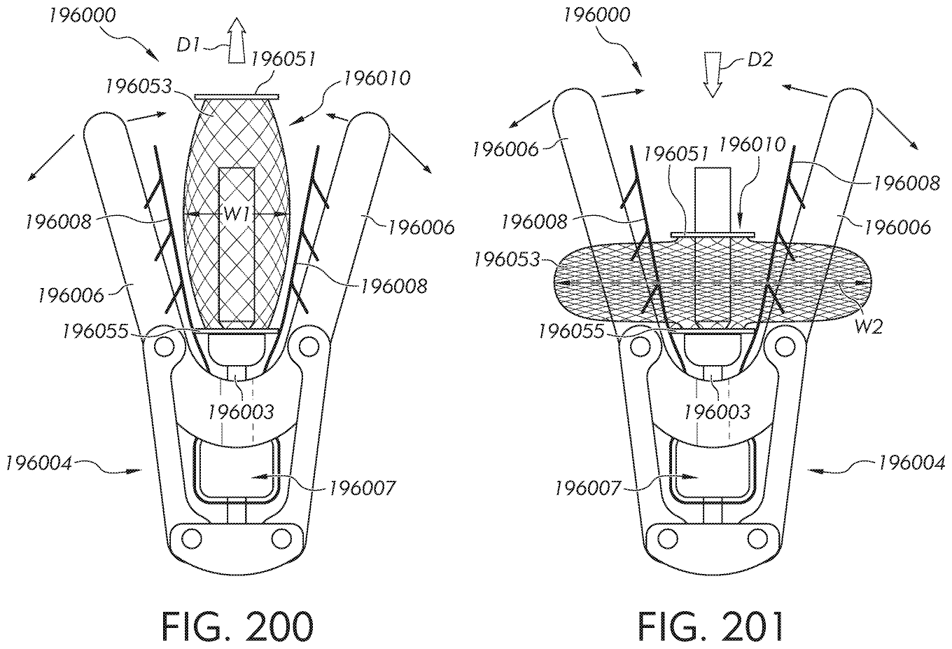

FIGS. 200-201 illustrate an example of a device 196000 (e.g., valve repair device, valve treatment device, implantable device, implant, etc.) having a coaptation element 196010 that is movable between an elongated configuration (as shown in FIG. 200) and an expanded configuration (as shown in FIG. 201).

The coaptation element 196010 can be used in a variety of different devices or valve repair devices, including but not limited to, any of the devices and valve repair devices disclosed in the present application. The device 196000 can include any other features for a device or valve repair device discussed in the present application, and the coaptation element 196010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application).

In some implementations, the device 196000 can be deployed from a delivery sheath, and/or an implant catheter. The device 196000 can be a prosthetic spacer device, valve repair device, valve treatment device, implant, or another type of device that attaches to leaflets of a native valve.

In some implementations, the device 196000 can take the form of the device 402 shown in FIG. 24, but the device 196000 further includes the coaptation element 196010. For example, the device 196000 can include a base assembly 196004, a pair of paddles 196006, and a pair of gripping members 196008 (e.g., clasps, clasp arms, grippers, gripping arms, latches, etc.).

In some implementations, the paddles 196006 can be part of the base assembly 196004. For example, the paddles 196006 can be formed as extensions of links of the base assembly. In the illustrated example, the base assembly 196004 has a shaft 196003, a coupler (not shown) configured to move along the shaft 196003, and a lock 196007 configured to lock the coupler in a stationary position on the shaft.

In some implementations, the coupler is mechanically connected to the paddles 196006, such that movement of the coupler along the shaft 196003 causes the paddles to move between an open position and a closed position. For example, the paddles 196006 can be pivotally coupled to the coupler at the pivot point 194011. In this way, the coupler can mechanically couple the paddles 196006 to the shaft 196003 and, when moving along the shaft 196003, can causing the paddles 196006 to move between their open and closed positions.

In some implementations, the gripping members 196008 are connected to the base assembly 196004 (e.g., the gripping members 196008 can be connected to the shaft 196003, or any other suitable member of the base assembly), such that the gripping members can be moved and/or flexed to adjust the width of the opening between the paddles 196006 and the gripping members 196008.

In some implementations, the gripping member 196008 can include an optional barbed portion for attaching the gripping members to valve tissue when the device 196000 is attached to the valve tissue. The gripping members 196008 can take any suitable form, such as, for example, any form of a gripping member or clasp disclosed in the present application.

In some implementations, when the paddles 196006 are in the closed position, the paddles engage the gripping members 196008, such that, when valve tissue is attached to the barbed portion of the gripping members, the paddles secure the device 196000 to the valve tissue.

In some implementations, the gripping members 196008 are configured to engage the paddles 196006 such that the barbed portion engages the valve tissue member and the paddles 196006 to secure the device 196000 to the valve tissue member. For example, in some instances, it can be advantageous to have the paddles 196006 maintain an open position and have the gripping members 196008 move outward toward the paddles 196006 to engage valve tissue and the paddles 196006.

While the device 196000 shown in FIGS. 200-201 illustrates a pair of paddles 196006 and a pair of gripping members 196008, it should be understood that the device 196000 can include any suitable number of paddles and gripping members.

In some implementations, the coaptation element 196010 can be configured to move between an elongated configuration (as shown in FIG. 200) and an expanded configuration (as shown in FIG. 201). In some implementations, the coaptation element 196010 can have a width W1 (FIG. 200) when in the elongated configuration and a width W2 (FIG. 201) when in the expanded configuration, where the width W1 is less than the width W2.

When the device 196000 is being attached to a native heart valve, it can be advantageous to have the coaptation element 196010 be in the elongated configuration because the smaller width W1 of the coaptation element 196010 allows for additional space between the coaptation element 196010 and the paddles 196006. This can which make it easier for a user to secure the device 196000 to one or more leaflets of the native heart valve with the paddles 196006 and gripping member 196008, e.g., because the opening between the paddle 196006 and the gripping member 196008 is larger.

After the device 196000 is secured to the native heart valve by the paddles 196006 and gripping members 196008, it can be advantageous to have the coaptation element 196010 be in the expanded configuration because the larger width W2 of the coaptation element 196010 reduces the space between the paddles 196006 and the coaptation element 196010 such that the paddles 196006 and coaptation element 196010 create a pinching force on a leaflet of the native heart valve to maintain the device 196000 in a secured connection with the one or more leaflets. In some implementations, the paddles 196006 and coaptation element 196010 exert a pinching force on a leaflet of the native heart valve such that expansion of the coaptation element 196010 increases and/or maintains the pinching force.

Referring to FIG. 201A, in some implementations, the coaptation element 196010 engages the paddles 196006 and/or the gripping members 196008 when in the expanded configuration such that the coaptation element 196010 forms around the paddles 196006 and/or the gripping members 196008. In some implementations, a ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 196010 can include a first end member 196051, a second end member 196055, and a flexible body portion 196053 attached to and extending between the first and second end members. One or both of the first end member 196051 and the second end member 196055 can be attached to a component of the device 196000 and movable in the directions D1, D2.

In some implementations, the flexible body portion 196053 can be made of a flexible material such that movement of the first end member 196051 and/or the second end member 196055 in the directions D1, D2 causes the flexible body portion 196053 to stretch when in coaptation element 196010 is in the elongated configuration and flex outward when the coaptation element 196010 is in the expanded configuration.

In some implementations, the first and/or second end members 196051, 196055 can be moved in the directions D1, D2 by an actuation element, such as, for example, any actuation element described in the present application.

In some implementations, one or both of the first end member 196051 and the second end member 196055 is attached to the shaft 196003 of the base assembly 196004 such that the first end member 196051 and/or the second end member 196055 is movable along the shaft 196003 to move the coaptation element 196010 between the elongated and expanded configurations.

For example, referring to FIG. 200, movement of the first end member 196051 in the direction D1 can cause the coaptation element 196010 to move to the elongated configuration. Referring to FIG. 201, movement of the first end member 196051 in the direction D2 can cause the coaptation element 196010 to move to the expanded configuration.

In some implementations, movement of the second end member 196055 in the direction D1 (FIG. 200) can cause the coaptation element 196010 to move to the expanded configuration, and movement of the second end member 196055 in the direction D2 (FIG. 201) can cause the coaptation element 196010 to move to the elongated configuration. In some implementations, only one of the first and second end members 196051, 196055 is movable along the shaft 196003 (or other component of the device 196000), and the other of the first and second end members is fixed relative to the shaft 196003 (or other component of the device 196000).

In some implementations, the first and/or second end members 196051, 196055 can be threadably attached to the shaft (or other component of the device 196000) such that rotation of the first and/or second end members allows for movement of such member(s) in the directions D1, D2. However, it should be understood that the first and/or second end member 196051, 196055 can be moved in the directions D1, D2 by any other suitable means.

While the first and/or second end members 196051, 196055 are described as being attached to and movable relative to the shaft 196003, it should be understood that the first and/or second end members 196051, 196055 can be attached to and movable relative to any other component of the device 196000 that allows for the coaptation element 196010 to be moved between the elongated and expanded configurations as a result of movement of the movable member 196051.

In some implementations, the first and/or second end members 196051, 196055 can be a flange or other component that is made of a material having a greater stiffness than that of the flexible body portion 196053 such that movement of the first and/or second end members 196051, 196055 causes the flexible body portion 196053 to stretch or flex outward.

In some implementations, the first and/or second end members 196051, 196055 can be made of any suitable plastic or metal material. In the illustrated example, the flexible body portion 196053 is made of a flexible, braided metal fabric material. The material can be cloth, shape-memory alloy wire—such as Nitinol—to provide shape-setting capability, or any other flexible material suitable for implantation in the human body.

While the coaptation element 196010 is described as having both a first end member 196051 and a second end member 196055, it should be understood that the coaptation element 196010 can have one of the first and second end members attached to one end of the flexible body portion 196053 and the other end of the flexible body portion 196053 can be attached to another component of the device 196000 that allows for the flexible body portion to stretch and flex outward to move the coaptation element between the expanded and elongated configurations when the first or second end member is moved in the directions D1, D2.

In some implementations, the coaptation element 196010 can include an optional cover. The cover can be configured in a variety of ways. In some implementations, the cover can include a sheet, material, fabric, layer or membrane that is attached to the coaptation element 196010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 196003 of the base assembly 196004 of the device 196000. In some implementations, after the device 196000 is secured to valve tissue, the placement shaft can be removed from the shaft 196003 to remove the device 196000 from the remainder of the treatment and/or repair system, such that the device 196000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 196004 to move the coupler along the shaft 196003, which causes the paddles 196006 to move between the open and closed positions. The paddle control mechanism can take any suitable form, such as, for example, any form for a paddle control mechanism described in the present application.

In some implementations, the gripper control mechanism can be configured to move the gripping members 196008 such that the width of the opening between the gripping members 196008 and the paddles 196006 can be altered. The gripper control mechanism can take any suitable form, such as, for example, any form for a gripper control mechanism described in the present application.

In some implementations, the lock control mechanism can be configured to lock and unlock the lock 196007. That is, the lock 196007 can be configured to lock the coupler in a stationary position with respect to the shaft 196003 and can take a wide variety of different forms and the type of lock control mechanism can be dictated by the type of lock used. The lock control mechanism can take any suitable form, such as, for example, any form described in the present application.

Additional features of the device 196000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

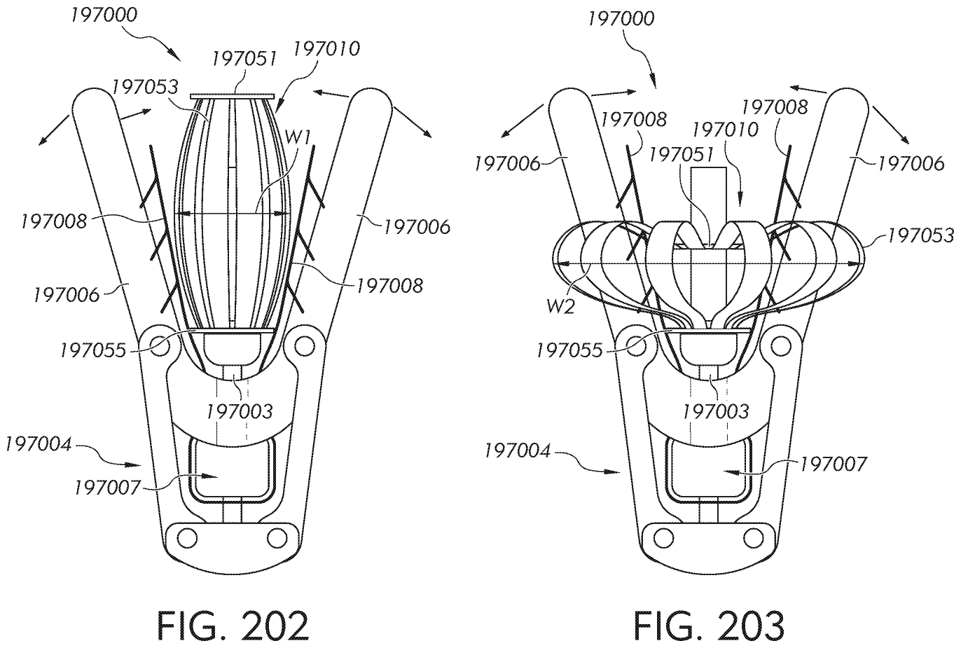

FIGS. 202-203 illustrate an example of a device 197000 (e.g., valve repair device, valve treatment device, implantable device, implant, etc.) having a coaptation element 197010 that is movable between an elongated configuration (as shown in FIG. 202) and an expanded configuration (as shown in FIG. 203).

The coaptation element 197010 can be used in a variety of different devices or valve repair devices, including but not limited to, any of the devices and valve repair devices disclosed in the present application. The device 197000 can include any other features for a device or valve repair device discussed in the present application, and the coaptation element 197010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application). The device 197000 can be deployed from a delivery sheath, and/or an implant catheter.

In some implementations, the device 197000 can be a prosthetic spacer device, valve repair device, valve treatment device, implant, or another type of device that attaches to leaflets of a native valve.

In some implementations, the device 197000 can take the form of the device 402 shown in FIG. 24, but the device 197000 further includes the coaptation element 197010. For example, the device 197000 can include a base assembly 197004, a pair of paddles 197006, and a pair of gripping members 197008 (e.g., clasps, clasp arms, grippers, gripping arms, latches, etc.).

In some implementations, the paddles 197006 can be part of the base assembly 197004. For example, the paddles 197006 can be formed as extensions of links of the base assembly. In the illustrated example, the base assembly 197004 has a shaft 197003, a coupler (not shown) configured to move along the shaft 197003, and a lock 197007 configured to lock the coupler in a stationary position on the shaft.

In some implementations, the coupler is mechanically connected to the paddles 197006, such that movement of the coupler along the shaft 197003 causes the paddles to move between an open position and a closed position. For example, the paddles 197006 can be pivotally coupled to the coupler at the pivot point or axis 194011. In this way, the coupler can mechanically couple the paddles 197006 to the shaft 197003 and, when moving along the shaft 197003, the coupler can cause the paddles 197006 to move between their open and closed positions.

In some implementations, the gripping members 197008 are connected to the base assembly 197004 (e.g., the gripping members 197008 can be connected to the shaft 197003, or any other suitable member of the base assembly), such that the gripping members can be moved and/or flexed to adjust the width of the opening between the paddles 197006 and the gripping members 197008.

In some implementations, the gripping member 197008 can include an optional barbed portion for attaching the gripping members to valve tissue when the device 197000 is attached to the valve tissue. The gripping members 197008 can take any suitable form, such as, for example, any form of a gripping member or clasp disclosed in the present application.

In some implementations, when the paddles 197006 are in the closed position, the paddles engage the gripping members 197008, such that, when valve tissue is attached to the barbed portion of the gripping members, the paddles secure the device 197000 to the valve tissue.

In some implementations, the gripping members 197008 are configured to engage the paddles 197006 such that the barbed portion engages the valve tissue member and the paddles 197006 to secure the device 197000 to the valve tissue member. For example, in in some instances, it can be advantageous to have the paddles 197006 maintain an open position and have the gripping members 197008 move outward toward the paddles 197006 to engage valve tissue and the paddles 197006.

While the device 197000 shown in FIGS. 202-203 illustrates a pair of paddles 197006 and a pair of gripping members 197008, it should be understood that the device 197000 can include any suitable number of paddles and gripping members.

In some implementations, the coaptation element 197010 can be configured to move between an elongated configuration (as shown in FIG. 202) and an expanded configuration (as shown in FIG. 203). In some implementations, the coaptation element 197010 can have a width W1 (FIG. 202) when in the elongated configuration and a width W2 (FIG. 203) when in the expanded configuration, where the width W1 is less than the width W2.

When the device 197000 is being attached to a native heart valve, it can be advantageous to have the coaptation element 197010 be in the elongated configuration because the smaller width W1 of the coaptation element 197010 allows for additional space between the coaptation element 197010 and the paddles 197006. This can make it easier for a user to secure the device 197000 to one or more leaflets of the native heart valve with the paddles 197006 and gripping member 197008, e.g., because the opening between the paddle 197006 and the gripping member 197008 is larger.

After the device 197000 is secured to the native heart valve by the paddles 197006 and gripping members 197008, it can be advantageous to have the coaptation element 197010 be in the expanded configuration because the larger width W2 of the coaptation element 197010 reduces the space between the paddles 197006 and the coaptation element 197010 such that the paddles 197006 and coaptation element 197010 create a pinching force on a leaflet of the native heart valve to maintain the device 197000 in a secured connection with the one or more leaflets. In some implementations, the paddles 197006 and coaptation element 197010 exert a pinching force on a leaflet of the native heart valve such that expansion of the coaptation element 197010 increases and/or maintains the pinching force.

In some implementations, the coaptation element 197010 engages the paddles 197006 and/or the gripping members 197008 when in the expanded configuration such that the coaptation element 197010 forms around the paddles 197006 and/or the gripping members 197008 (e.g., similar to the coaptation element 196010 shown in FIG. 201A). A ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 197010 can include a first end member 197051, a second end member 197055, and a flexible body portion 197053 attached to and extending between the first and second end members. One or both of the first end member 197051 and the second end member 197055 can be attached to a component of the device 197000 and movable in the directions D1, D2.

In some implementations, the flexible body portion 197053 can be made of a flexible material such that movement of the first end member 197051 and/or the second end member 197055 in the directions D1, D2 causes the flexible body portion 197053 to stretch when in coaptation element 197010 is in the elongated configuration and flex outward when the coaptation element 197010 is in the expanded configuration.

In some implementations, the first and/or second end members 197051, 197055 can be moved in the directions D1, D2 by an actuation element, such as, for example, any actuation element described in the present application.

In some implementations, one or both of the first end member 197051 and the second end member 197055 is attached to the shaft 197003 of the base assembly 197004 such that the first end member 197051 and/or the second end member 197055 is movable along the shaft 197003 to move the coaptation element 197010 between the elongated and expanded configurations.

For example, referring to FIG. 202, movement of the first end member 197051 in the direction D1 can cause the coaptation element 197010 to move to the elongated configuration. Referring to FIG. 203, movement of the first end member 197051 in the direction D2 can cause the coaptation element 197010 to move to the expanded configuration.

In some implementations, movement of the second end member 197055 in the direction D1 (FIG. 202) can cause the coaptation element 197010 to move to the expanded configuration, and movement of the second end member 197055 in the direction D2 (FIG. 203) can cause the coaptation element 197010 to move to the elongated configuration. In some implementations, only one of the first and second end members 197051, 197055 is movable along the shaft 197003 (or other component of the device 197000), and the other of the first and second end members is fixed relative to the shaft 197003 (or other component of the device 197000).

In some implementations, the first and/or second end members 197051, 197055 can be threadably attached to the shaft (or other component of the device 197000) such that rotation of the first and/or second end members allows for movement of such member(s) in the directions D1, D2. However, it should be understood that the first and/or second end member 197051, 197055 can be moved in the directions D1, D2 by any other suitable means.

While the first and/or second end members 197051, 197055 are described as being attached to and movable relative to the shaft 197003, it should be understood that the first and/or second end members 197051, 197055 can be attached to and movable relative to any other component of the device 197000 that allows for the coaptation element 197010 to be moved between the elongated and expanded configurations as a result of movement of the movable member 197051.

In some implementations, the first and/or second end members 197051, 197055 can be a flange or other component that is made of a material having a greater stiffness than that of the flexible body portion 197053 such that movement of the first and/or second end members 197051, 197055 causes the flexible body portion 197053 to stretch or flex outward.

In some implementations, the first and/or second end members 197051, 197055 can be made of any suitable plastic or metal material. In the illustrated example, the flexible body portion 197053 is made of a flexible metal fabric material that includes strips extending between the first and second end members 197051, 197055. The material can be cloth, shape-memory alloy wire—such as Nitinol—to provide shape-setting capability, or any other flexible material suitable for implantation in the human body.

While the coaptation element 197010 is described as having both a first end member 197051 and a second end member 197055, it should be understood that the coaptation element 197010 can have one of the first and second end members attached to one end of the flexible body portion 197053 and the other end of the flexible body portion 197053 can be attached to another component of the device 197000 that allows for the flexible body portion to stretch and flex outward to move the coaptation element between the expanded and elongated configurations when the first or second end member is moved in the directions D1, D2.

In some implementations, the coaptation element 197010 can include an optional cover. The cover can be configured in a variety of ways. In some implementations, the cover can include a sheet, material, fabric, layer or membrane that is attached to the coaptation element 197010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 197003 of the base assembly 197004 of the device 197000. In some implementations, after the device 197000 is secured to valve tissue, the placement shaft can be removed from the shaft 197003 to remove the device 197000 from the remainder of the treatment and/or repair system, such that the device 197000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 197004 to move the coupler along the shaft 197003, which causes the paddles 197006 to move between the open and closed positions. The paddle control mechanism can take any suitable form, such as, for example, any form for a paddle control mechanism described in the present application.

In some implementations, the gripper control mechanism can be configured to move the gripping members 197008 such that the width of the opening between the gripping members 197008 and the paddles 197006 can be altered. The gripper control mechanism can take any suitable form, such as, for example, any form for a gripper control mechanism described in the present application.

In some implementations, the lock control mechanism can be configured to lock and unlock the lock 197007. That is, the lock 197007 can be configured to lock the coupler in a stationary position with respect to the shaft 197003 and can take a wide variety of different forms and the type of lock control mechanism can be dictated by the type of lock used. The lock control mechanism can take any suitable form, such as, for example, any form described in the present application.

Additional features of the device 197000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

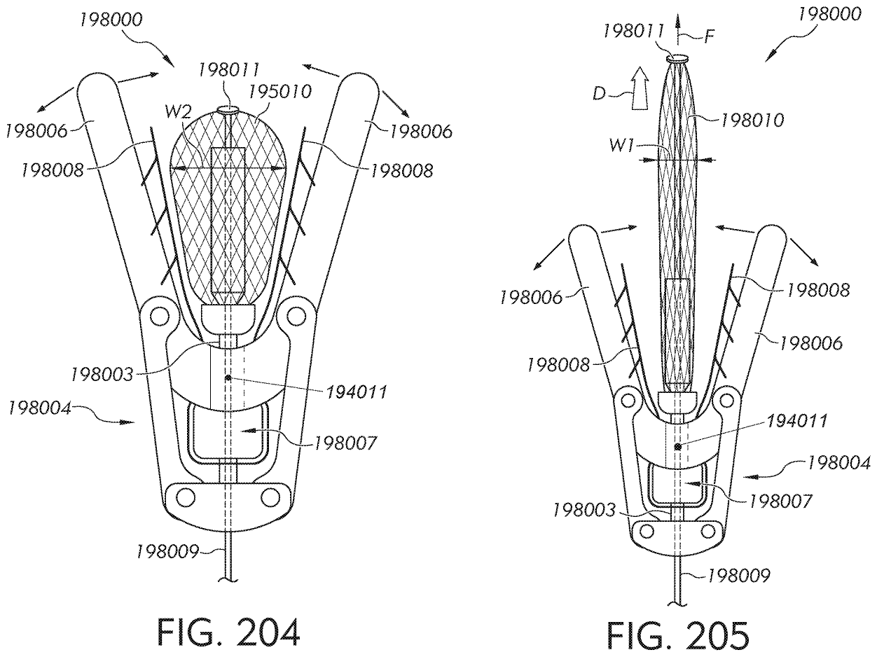

FIGS. 204-205 illustrate an example of a device 198000 (e.g., valve repair device, valve treatment device, implantable device, implant, etc.) having a coaptation element 198010 that is movable between an elongated configuration (as shown in FIG. 205) and an expanded configuration (as shown in FIG. 204).

In some implementations, the coaptation element 198010 can be used in a variety of different devices or valve repair devices, including but not limited to, any of the devices and valve repair devices disclosed in the present application. The device 198000 can include any other features for a device or valve repair device discussed in the present application, and the coaptation element 198010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application).

In some implementations, the device 198000 can be deployed from a delivery sheath, and/or an implant catheter. The device 198000 can be a prosthetic spacer device, valve repair device, valve treatment device, implant, or another type of device that attaches to leaflets of a native valve.

In some implementations, the device 198000 can take the form of the device 402 shown in FIG. 24, but the device 198000 further includes the coaptation element 198010. For example, the device 198000 can include a base assembly 198004, a pair of paddles 198006, and a pair of gripping members 198008 (e.g., clasps, clasp arms, grippers, gripping arms, latches, etc.).

In some implementations, the paddles 198006 can be part of the base assembly 198004. For example, the paddles 198006 can be formed as extensions of links of the base assembly. In the illustrated example, the base assembly 198004 has a shaft 198003, a coupler (not shown) configured to move along the shaft 198003, and a lock 198007 configured to lock the coupler in a stationary position on the shaft.

In some implementations, the coupler is mechanically connected to the paddles 198006, such that movement of the coupler along the shaft 198003 causes the paddles to move between an open position and a closed position. For example, the paddles 198006 can be pivotally coupled to the coupler at a pivot axis 194011. In this way, the coupler serves can mechanically couple the paddles 198006 to the shaft 198003 and, when moving along the shaft 198003, for the coupler can cause the paddles 198006 to move between their open and closed positions.

In some implementations, the gripping members 198008 are connected to the base assembly 198004 (e.g., the gripping members 198008 can be connected to the shaft 198003, or any other suitable member of the base assembly), such that the gripping members can be moved and/or flexed to adjust the width of the opening between the paddles 198006 and the gripping members 198008.

In some implementations, the gripping member 198008 can include an optional barbed portion for attaching the gripping members to valve tissue when the device 198000 is attached to the valve tissue. The gripping members 198008 can take any suitable form, such as, for example, any form of a gripping member or clasp disclosed in the present application.

In some implementations, when the paddles 198006 are in the closed position, the paddles engage the gripping members 198008, such that, when valve tissue is attached to the barbed portion of the gripping members, the paddles secure the device 198000 to the valve tissue.

In some implementations, the gripping members 198008 are configured to engage the paddles 198006 such that the barbed portion engages the valve tissue member and the paddles 198006 to secure the device 198000 to the valve tissue member. For example, in certain situations, it can be advantageous to have the paddles 198006 maintain an open position and have the gripping members 198008 move outward toward the paddles 198006 to engage valve tissue and the paddles 198006.

While the device 198000 shown in FIGS. 204-205 illustrates a pair of paddles 198006 and a pair of gripping members 198008, it should be understood that the device 198000 can include any suitable number of paddles and gripping members.

In some implementations, the coaptation element 198010 can be configured to move between an elongated configuration (as shown in FIG. 205) and an expanded configuration (as shown in FIG. 204). In some implementations, the coaptation element 198010 can have a width W1 (FIG. 205) when in the elongated configuration and a width W2 (FIG. 204) when in the expanded configuration, where the width W1 is less than the width W2.

When the device 198000 is being attached to a native heart valve, it can be advantageous to have the coaptation element 198010 be in the elongated configuration because the smaller width W1 of the coaptation element 198010 allows for additional space between the coaptation element 198010 and the paddles 198006. This can make it easier for a user to secure the device 198000 to one or more leaflets of the native heart valve with the paddles 198006 and gripping member 198008.

After the device 198000 is secured to the native heart valve by the paddles 198006 and gripping members 198008, it can be advantageous to have the coaptation element 198010 be in the expanded configuration because the larger width W2 of the coaptation element 198010 reduces the space between the paddles 198006 and the coaptation element 198010 such that the paddles 198006 and coaptation element 198010 create a pinching force on a leaflet of the native heart valve to maintain the device 198000 in a secured connection with the one or more leaflets. In some implementations, the paddles 198006 and coaptation element 198010 exert a pinching force on a leaflet of the native heart valve such that expansion of the coaptation element 198010 increases and/or maintains the pinching force.

In some implementations, the coaptation element 198010 engages the paddles 198006 and/or the gripping members 198008 when in the expanded configuration such that the coaptation element 198010 forms around the paddles 198006 and/or the gripping members 198008 (e.g., similar to the coaptation element 196010 shown in FIG. 201A). A ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 198010 can be shape set in the expanded configuration (as shown in FIG. 204), and an actuation element can be used to engage the coaptation element 198010 to cause the coaptation element 198010 to be in the elongated configuration (as shown in FIG. 205). To this end, the valve repair system can include an actuation element 198009 (e.g., a wire, tube, etc.), and coaptation element 198010 can include a cap 198011 that is configured to be engaged by the actuation element 198009 to move the coaptation element 198010 from the expanded configuration to the elongated configuration. In the illustrated example, movement of the actuation element 198009 in the direction D1 (FIG. 205) creates a force F on the coaptation element 198010 that causes the coaptation element 198010 to be in the elongated configuration.

After the device 198000 is secured to the native heart valve (e.g., via the paddles 198006 and gripping members 198008), the actuation element 198009 can be removed from the coaptation element 198010 or otherwise disengaged by a user such that force F is no longer applied to the coaptation element 198010, which causes the coaptation element 198010 to move back to its normally expanded configuration (as shown in FIG. 204). While the coaptation element 198010 is shown being in a normally expanded configuration and engaged by an actuation element to be moved to the elongated configuration, it should be understood that the coaptation element 198010 can be moved between the elongated and expanded configurations by any suitable means.

In some implementations, the coaptation element 198010 can be attached to the shaft 198003 of the base assembly 198004. However, it should be understood that the coaptation element 198010 can be attached to any other portion of the device 198000 that allows for the coaptation element to be positioned such that one or more leaflets of the native heart valve can coapt against the coaptation element 198010 when the device 198000 is attached to the native heart valve.

In some implementations, the coaptation element 198010 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, the coaptation element 198010 can include an optional cover. The cover can be configured in a variety of ways. In some implementations, the cover can include a sheet, material, fabric, layer or membrane that is attached to the coaptation element 198010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 198003 of the base assembly 198004 of the device 198000. In some implementations, after the device 198000 is secured to valve tissue, the placement shaft can be removed from the shaft 198003 to remove the device 198000 from the remainder of the treatment and/or repair system, such that the device 198000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 198004 to move the coupler along the shaft 198003, which causes the paddles 198006 to move between the open and closed positions. The paddle control mechanism can take any suitable form, such as, for example, any form for a paddle control mechanism described in the present application.

In some implementations, the gripper control mechanism can be configured to move the gripping members 198008 such that the width of the opening between the gripping members 198008 and the paddles 198006 can be altered. The gripper control mechanism can take any suitable form, such as, for example, any form for a gripper control mechanism described in the present application.

In some implementations, the lock control mechanism can be configured to lock and unlock the lock 198007. That is, the lock 198007 can be configured to lock the coupler in a stationary position with respect to the shaft 198003 and can take a wide variety of different forms and the type of lock control mechanism can be dictated by the type of lock used. The lock control mechanism can take any suitable form, such as, for example, any form described in the present application.

Additional features of the device 198000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

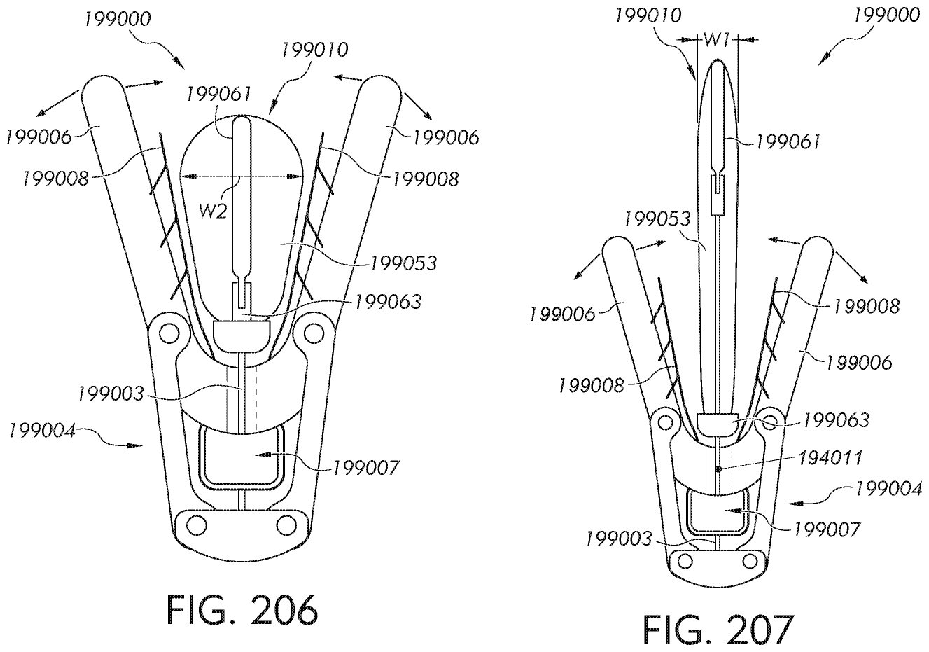

FIGS. 206-207 illustrate an example of a device 199000 (e.g., valve repair device, valve treatment device, implantable device, implant, etc.) having a coaptation element 199010 that is movable between an elongated configuration (as shown in FIG. 207) and an expanded configuration (as shown in FIG. 206).

The coaptation element 199010 can be used in a variety of different devices or valve repair devices, including but not limited to, any of the devices and valve repair devices disclosed in the present application. The device 199000 can include any other features for a device or valve repair device discussed in the present application, and the coaptation element 199010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application). The device 199000 can be deployed from a delivery sheath, and/or an implant catheter.

In some implementations, the device 199000 can be a prosthetic spacer device, valve repair device, valve treatment device, implant, or another type of device that attaches to leaflets of a native valve.

In some implementations, the device 199000 can take the form of the device 402 shown in FIG. 24, but the device 199000 further includes the coaptation element 199010. For example, the device 199000 can include a base assembly 199004, a pair of paddles 199006, and a pair of gripping members 199008 (e.g., clasps, clasp arms, grippers, gripping arms, latches, etc.).

In some implementations, the paddles 199006 can be part of the base assembly 199004. For example, the paddles 199006 can be formed as extensions of links of the base assembly. In the illustrated example, the base assembly 199004 has a shaft 199003, a coupler (not shown) configured to move along the shaft 199003, and a lock 199007 configured to lock the coupler in a stationary position on the shaft.

In some implementations, the coupler is mechanically connected to the paddles 199006, such that movement of the coupler along the shaft 199003 causes the paddles to move between an open position and a closed position. For example, the paddles can be pivotally connected to the coupler at a pivot axis 194011. In this way, the coupler can mechanically couple the paddles 199006 to the shaft 199003 and, when moving along the shaft 199003, the coupler can cause the paddles 199006 to move between their open and closed positions.

In some implementations, the gripping members 199008 are connected to the base assembly 199004 (e.g., the gripping members 199008 can be connected to the shaft 199003, or any other suitable member of the base assembly), such that the gripping members can be moved to adjust the width of the opening between the paddles 199006 and the gripping members 199008.

In some implementations, the gripping member 199008 can include an optional barbed portion for attaching the gripping members to valve tissue when the device 199000 is attached to the valve tissue. The gripping members 199008 can take any suitable form, such as, for example, any form of a gripping member or clasp disclosed in the present application.

In some implementations, when the paddles 199006 are in the closed position, the paddles engage the gripping members 199008, such that, when valve tissue is attached to the barbed portion of the gripping members, the paddles secure the device 199000 to the valve tissue.

In some implementations, the gripping members 199008 are configured to engage the paddles 199006 such that the barbed portion engages the valve tissue member and the paddles 199006 to secure the device 199000 to the valve tissue member. For example, in some instances, it can be advantageous to have the paddles 199006 maintain an open position and have the gripping members 199008 move outward toward the paddles 199006 to engage valve tissue and the paddles 199006.

While the device 199000 shown in FIGS. 206-207 illustrates a pair of paddles 199006 and a pair of gripping members 199008, it should be understood that the device 199000 can include any suitable number of paddles and gripping members.

In some implementations, the coaptation element 199010 can be configured to move between an elongated configuration (as shown in FIG. 207) and an expanded configuration (as shown in FIG. 206). The coaptation element 199010 can have a width W1 (FIG. 207) when in the elongated configuration and a width W2 (FIG. 206) when in the expanded configuration, where the width W1 is less than the width W2.

When the device 199000 is being attached to a native heart valve, it can be advantageous to have the coaptation element 199010 be in the elongated configuration because the smaller width W1 of the coaptation element 199010 allows for additional space between the coaptation element 199010 and the paddles 199006. This can which make it easier for a user to secure the device 199000 to one or more leaflets of the native heart valve with the paddles 199006 and gripping member 199008, e.g., because the opening between the paddle 199006 and the gripping member 199008 is larger.

After the device 199000 is secured to the native heart valve by the paddles 199006 and gripping members 199008, it can be advantageous to have the coaptation element 199010 be in the expanded configuration because the larger width W2 of the coaptation element 199010 reduces the space between the paddles 199006 and the coaptation element 199010 such that the paddles 199006 and coaptation element 199010 create a pinching force on a leaflet of the native heart valve to maintain the device 199000 in a secured connection with the one or more leaflets. In some implementations, the paddles 199006 and coaptation element 199010 exert a pinching force on a leaflet of the native heart valve such that expansion of the coaptation element 199010 increases and/or maintains the pinching force.

In some implementations, the coaptation element 199010 engages the paddles 199006 and/or the gripping members 199008 when in the expanded configuration such that the coaptation element 199010 forms around the paddles 199006 and/or the gripping members 199008 (e.g., similar to the coaptation element 196010 shown in FIG. 201A). A ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 199010 can include a flexible body member 199053, a proximal element 199061 attached to a proximal end of the flexible body member 199053, and a distal element 199063 attached to a distal end of the flexible body member, where the proximal element 199061 is movable between an extended position (as shown in FIG. 207) and a retracted position (as shown in FIG. 206) relative to the distal element 199063.

When the proximal element 199061 is in the extended position relative to the distal element 199063, the engagement between the proximal element 199061 and the flexible body member 199053 causes the coaptation element 199010 to be in the elongated configuration (as shown in FIG. 207). When the proximal element 199061 is in the retracted position relative to the distal element 199063, the engagement between the proximal element 199061 and the flexible body member 199053 causes the coaptation element 199010 to be in the expanded configuration (as shown in FIG. 206).

In some implementations, the proximal and distal elements 199061, 199063 take a form similar to a retractable mechanism for a retractable pen. For example, the proximal element 199061 can comprise a cam body member and the distal element 199063 can comprise a plunger member and one or more springs allow for the cam body member to move relative to the plunger member to cause the proximal element 199061 to move between the extended and retracted positions.

In some implementations, the distal element 199063 can be a cam body member and the proximal element 199061 can be a plunger member, and one or more springs can cause the distal element 199063 to move between extended and retracted positions relative to the proximal element 199061 to move the coaptation element 199010 between the elongated and expanded configurations.

While the proximal and distal elements 199061, 199063 are described as taking the form of a cam body/plunger mechanism, it should be understood that the proximal and distal elements 199061, 199063 can take any other suitable form that allows for one of the proximal and distal elements to move between extended and retracted positions relative to the other of the proximal and distal elements. In some implementations, an actuation element (e.g., any actuation element described in the present application) can be configured to engage the proximal and/or distal element to move the coaptation element 199010 between the elongated and expanded configurations.

In some implementations, the coaptation element 199010 can be shape set in the expanded configuration (as shown in FIG. 206), and movement of the proximal element 199061 to an extended position relative to the distal element 199063 causes the coaptation element 199010 to move to the elongated configuration (as shown in FIG. 207).

After the device 199000 is secured to the native heart valve (e.g., via the paddles 199006 and gripping members 199008), the proximal element 199061 can be moved to the retracted position relative to the distal element 199063, which causes the coaptation element 199010 to move back to the expanded configuration (as shown in FIG. 206).

In some implementations, the coaptation element 199010 can be attached to the shaft 199003 of the base assembly 199004. However, it should be understood that the coaptation element 199010 can be attached to any other portion of the device 199000 that allows for the coaptation element to be positioned such that one or more leaflets of the native heart valve can coapt against the coaptation element 199010 when the device 199000 is attached to the native heart valve.

In some implementations, the coaptation element 199010 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, the coaptation element 199010 can include an optional cover. The cover can be configured in a variety of ways. In some implementations, the cover can include a sheet, material, fabric, layer or membrane that is attached to the coaptation element 199010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 199003 of the base assembly 199004 of the device 199000. In some implementations, after the device 199000 is secured to valve tissue, the placement shaft can be removed from the shaft 199003 to remove the device 199000 from the remainder of the treatment and/or repair system, such that the device 199000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 199004 to move the coupler along the shaft 199003, which causes the paddles 199006 to move between the open and closed positions. The paddle control mechanism can take any suitable form, such as, for example, any form for a paddle control mechanism described in the present application.

In some implementations, the gripper control mechanism can be configured to move the gripping members 199008 such that the width of the opening between the gripping members 199008 and the paddles 199006 can be altered. The gripper control mechanism can take any suitable form, such as, for example, any form for a gripper control mechanism described in the present application.

In some implementations, the lock control mechanism can be configured to lock and unlock the lock 199007. That is, the lock 199007 can be configured to lock the coupler in a stationary position with respect to the shaft 199003 and can take a wide variety of different forms and the type of lock control mechanism can be dictated by the type of lock used. The lock control mechanism can take any suitable form, such as, for example, any form described in the present application.

Additional features of the device 199000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

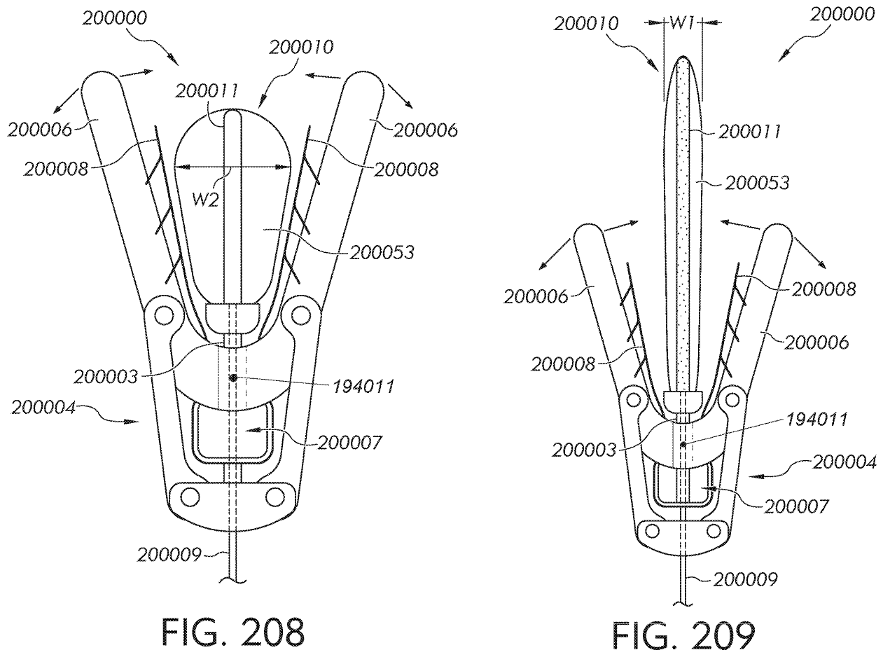

FIGS. 208-209 illustrate an example of a device 200000 (e.g., valve repair device, valve treatment device, implantable device, implant, etc.) having a coaptation element 200010 that is movable between an elongated configuration (as shown in FIG. 209) and an expanded configuration (as shown in FIG. 208).

In some implementations, the coaptation element 200010 can be used in a variety of different devices or valve repair devices, including but not limited to, any of the devices and valve repair devices disclosed in the present application. The device 200000 can include any other features for a device or valve repair device discussed in the present application, and the coaptation element 200010 can be positioned to engage leaflets 30, 32, 34 (see FIGS. 7 and 34) or leaflets 20, 22 (see FIGS. 6 and 36) as part of any suitable device (e.g., any device or valve repair device disclosed in the present application).

In some implementations, the device 200000 can be deployed from a delivery sheath, and/or an implant catheter. The device 200000 can be a prosthetic spacer device, valve repair device, valve treatment device, implant, or another type of device that attaches to leaflets of a native valve.

In some implementations, the device 200000 can take the form of the device 402 shown in FIG. 24, but the device 200000 further includes the coaptation element 200010. For example, the device 200000 can include a base assembly 200004, a pair of paddles 200006, and a pair of gripping members 200008 (e.g., clasps, clasp arms, grippers, gripping arms, latches, etc.).

In some implementations, the paddles 200006 can be part of with the base assembly 200004. For example, the paddles 200006 can be formed as extensions of links of the base assembly. In the illustrated example, the base assembly 200004 has a shaft 200003, a coupler (not shown) configured to move along the shaft 200003, and a lock 200007 configured to lock the coupler in a stationary position on the shaft.

In some implementations, the coupler is mechanically connected to the paddles 200006, such that movement of the coupler along the shaft 200003 causes the paddles to move between an open position and a closed position. For example, the paddles can be pivotally coupled to the coupler at a pivot axis 194011. In this way, the coupler can mechanically couple the paddles 200006 to the shaft 200003 and, when moving along the shaft 200003, for the coupler can cause the paddles 200006 to move between their open and closed positions.

In some implementations, the gripping members 200008 are connected to the base assembly 200004 (e.g., the gripping members 200008 can be connected to the shaft 200003, or any other suitable member of the base assembly), such that the gripping members can be moved and/or flexed to adjust the width of the opening between the paddles 200006 and the gripping members 200008.

In some implementations, the gripping member 200008 can include an optional barbed portion for attaching the gripping members to valve tissue when the device 200000 is attached to the valve tissue. The gripping members 200008 can take any suitable form, such as, for example, any form of a gripping member or clasp disclosed in the present application.

In some implementations, when the paddles 200006 are in the closed position, the paddles engage the gripping members 200008, such that, when valve tissue is attached to the barbed portion of the gripping members, the paddles secure the device 200000 to the valve tissue.

In some implementations, the gripping members 200008 are configured to engage the paddles 200006 such that the barbed portion engages the valve tissue member and the paddles 200006 to secure the device 200000 to the valve tissue member. For example, in some instances, it can be advantageous to have the paddles 200006 maintain an open position and have the gripping members 200008 move outward toward the paddles 200006 to engage valve tissue and the paddles 200006.

While the device 200000 shown in FIGS. 208-209 illustrates a pair of paddles 200006 and a pair of gripping members 200008, it should be understood that the device 200000 can include any suitable number of paddles and gripping members.

In some implementations, the coaptation element 200010 can be configured to move between an elongated configuration (as shown in FIG. 209) and an expanded configuration (as shown in FIG. 208). The coaptation element 200010 can have a width W1 (FIG. 207) when in the elongated configuration and a width W2 (FIG. 206) when in the expanded configuration, where the width W1 is less than the width W2.

When the device 200000 is being attached to a native heart valve, it can be advantageous to have the coaptation element 200010 be in the elongated configuration because the smaller width W1 of the coaptation element 200010 allows for additional space between the coaptation element 200010 and the paddles 200006. This can make it easier for a user to secure the device 200000 to one or more leaflets of the native heart valve with the paddles 200006 and gripping member 200008, e.g., because the opening between the paddle 200006 and the gripping member 200008 is larger.

After the device 200000 is secured to the native heart valve by the paddles 200006 and gripping members 200008, it can be advantageous to have the coaptation element 200010 be in the expanded configuration because the larger width W2 of the coaptation element 200010 reduces the space between the paddles 200006 and the coaptation element 200010 such that the paddles 200006 and coaptation element 200010 create a pinching force on a leaflet of the native heart valve to maintain the device 200000 in a secured connection with the one or more leaflets. In some implementations, the paddles 200006 and coaptation element 200010 exert a pinching force on a leaflet of the native heart valve such that expansion of the coaptation element 200010 increases and/or maintains the pinching force.

In some implementations, the coaptation element 200010 engages the paddles 200006 and/or the gripping members 200008 when in the expanded configuration such that the coaptation element 200010 forms around the paddles 200006 and/or the gripping members 200008 (e.g., similar to the coaptation element 196010 shown in FIG. 201A). A ratio of the width W2 to the width W1 can be between 10/1 and 4/3, between 8/1 and 3/2, between 5/1 and 2/1, between 3/1 and 2/1 or any subrange of these ranges.

In some implementations, the coaptation element 200010 can include a flexible body member 200053, a fluid actuator 200011 (e.g., a soft fluidic actuator) attached to the flexible body member 200053, and a fluid line 200009 in fluid communication with the fluid actuator 200011. The fluid line 200009 is configured to provide fluid to and remove fluid from the fluid actuator 200011 such that the fluid actuator 200011 can be moved between an extended position (as shown in FIG. 209) and a shortened position (as shown in FIG. 208).

When the fluid actuator 200011 is in the extended position, the engagement between the fluid actuator 200011 and the flexible body member 200053 causes the coaptation element 200010 to be in the elongated configuration (as shown in FIG. 209). When the fluid actuator 200011 is in the shortened position, the engagement between the fluid actuator 200011 and the flexible body member 200053 causes the coaptation element 200010 to be in the expanded configuration (as shown in FIG. 208). In some implementations, the fluid provided to the fluid actuator 200011 by the fluid line 200009 can be 10/1 and 4/3, between 8/1 and 3/2, between 5/1 and 2/1, between 3/1 and 2/1 or any subrange of these ranges.

In some implementations, the coaptation element 200010 can be shape set in the expanded configuration (as shown in FIG. 208), and movement of the fluid actuator 200011 to the extended position causes the coaptation element 200010 to move to the elongated configuration (as shown in FIG. 209).

After the device 200000 is secured to the native heart valve (e.g., via the paddles 200006 and gripping members 200008), fluid can be removed from the fluid actuator 200011 to cause the fluid actuator 200011 to move to the shortened position, which causes the coaptation element 200010 to move back to the expanded configuration (as shown in FIG. 208).

In some implementations, the coaptation element 200010 can be attached to the shaft 200003 of the base assembly 200004. However, it should be understood that the coaptation element 200010 can be attached to any other portion of the device 200000 that allows for the coaptation element to be positioned such that one or more leaflets of the native heart valve can coapt against the coaptation element 200010 when the device 200000 is attached to the native heart valve.

In some implementations, the coaptation element 200010 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, the coaptation element 200010 can include an optional cover. The cover can be configured in a variety of ways. In some implementations, the cover can include a sheet, material, fabric, layer or membrane that is attached to the coaptation element 200010 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

In some implementations, an example valve repair system can include a placement shaft (not shown) that is removably attached to the shaft 200003 of the base assembly 200004 of the device 200000. In some implementations, after the device 200000 is secured to valve tissue, the placement shaft can be removed from the shaft 200003 to remove the device 200000 from the remainder of the treatment and/or repair system, such that the device 200000 can remain attached to the valve tissue, and the delivery device can be removed from a patient's body.

In some implementations, the treatment and/or repair system can also include a paddle control mechanism, a gripper control mechanism, and a lock control mechanism.

In some implementations, the paddle control mechanism can be mechanically attached to the coupler of the base assembly 200004 to move the coupler along the shaft 200003, which causes the paddles 200006 to move between the open and closed positions. The paddle control mechanism can take any suitable form, such as, for example, any form for a paddle control mechanism described in the present application.

In some implementations, the gripper control mechanism can be configured to move the gripping members 200008 such that the width of the opening between the gripping members 200008 and the paddles 200006 can be altered. The gripper control mechanism can take any suitable form, such as, for example, any form for a gripper control mechanism described in the present application.

In some implementations, the lock control mechanism can be configured to lock and unlock the lock 200007. That is, the lock 200007 can be configured to lock the coupler in a stationary position with respect to the shaft 200003 and can take a wide variety of different forms and the type of lock control mechanism can be dictated by the type of lock used. The lock control mechanism can take any suitable form, such as, for example, any form described in the present application.

Additional features of the device 200000, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

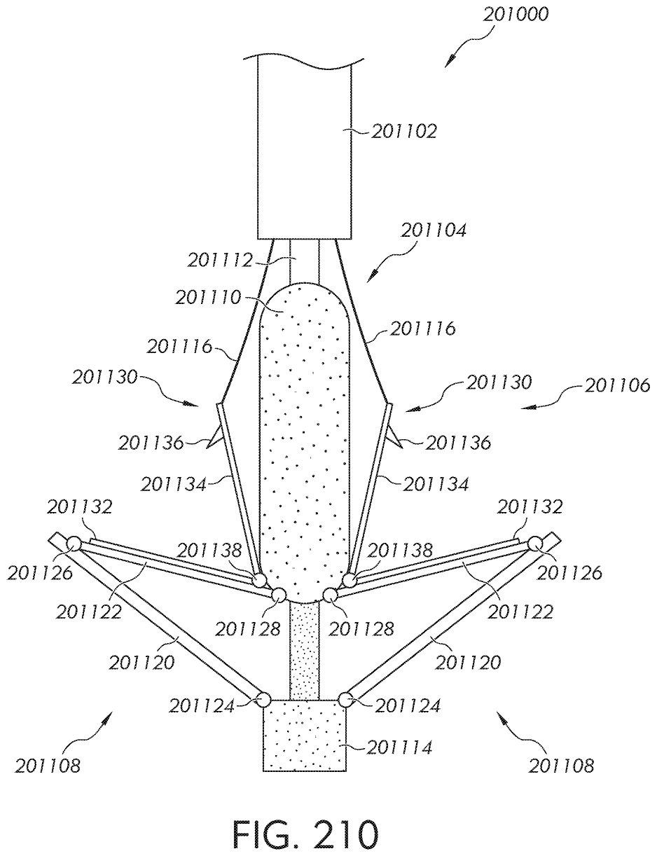

FIG. 210 illustrates an example of a device 201000 (e.g., valve repair device, valve treatment device, implantable device, implant, etc.) having one or more components configured to improve the visibility when using an imaging device (e.g., a commercial ultrasound imaging device) during implantation of the device 201000 on a native heart valve. In some implementations, one or more components of the device 201000 can be configured to have an acoustic impedance that is substantially similar to the acoustic impedance of blood, can improve the visibility of the device 201000 during implantation of the device 201000. In some implementations, the one or more components can have an acoustic impedance that is within 0.5 MRayl of blood, within 0.3 MRayl of blood, within 0.1 MRayl of blood, within 0.05 MRayl of blood, or within 0.02 MRayl of blood.

In some implementations, the device 201000 can include any features for a device or valve repair device discussed in the present application. The device 201000 can be deployed from a delivery sheath, and/or an implant catheter. The device 201000 can be a prosthetic spacer device, valve repair device, valve treatment device, implant, or another type of device that attaches to leaflets of a native valve.

In some implementations, the device 201000 can take the form of the device 100 shown in FIGS. 8-15, but the device 201000 further includes one or more components configured to improve the visibility when using an imaging device during implantation of the device 201000 on a native heart valve (e.g., by matching the acoustic impedance of one or more components to the acoustic impedance of blood).

In some implementations, the device 201000 can include a coaptation portion 201104 and an anchor portion 201106. In some implementations, the coaptation portion 201104 includes a coaptation element 201110 that is adapted to be deployed and/or implanted between leaflets of a native valve (e.g., a native mitral valve, native tricuspid valve, etc.) and is slidably attached to an actuation element 201112 (e.g., actuation wire, shaft, tube, hypotube, line, suture, braid, etc.) of a delivery system 201102.

In some implementations, the anchor portion 201106 can include one or more anchors 201108 that are actuatable between open and closed conditions and can take a wide variety of forms, such as, for example, paddles, gripping elements, or the like. Actuation of the actuation element 201112 opens and closes the anchor portion 201106 to grasp the native valve leaflets during deployment and/or implantation.

The actuation element 201112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element of the delivery system 201102 can be threaded such that rotation of the actuation element moves the anchor portion 201106 relative to the coaptation portion 201104. Or, the actuation element can be unthreaded, such that pushing or pulling the actuation element 201112 moves the anchor portion 201106 relative to the coaptation portion 201104.

In some implementations, the anchor portion 201106 and/or anchors 201108 of the device 201000 include outer paddles 201120 and inner paddles 201122 that are, in some implementations, connected between a cap 201114 and a coaptation element 201110 by portions 201124, 201126, 201128. The portions 201124, 201126, 201128 can be jointed and/or flexible to move between all of the positions described below. The interconnection of the outer paddles 201120, the inner paddles 201122, the coaptation element 201110, and the cap 201114 by the portions 201124, 201126, and 201128 can constrain the device to the positions and movements illustrated herein.

In some implementations, the anchor portion 201106 and/or anchors 201108 can include attachment portions or gripping members (e.g., gripping arms, clasp arms, etc.). The illustrated gripping members can comprise clasps 201130 that include a base or fixed arm 201132, a moveable arm 201134, optional friction-enhancing elements, other securing structures 201136 (e.g., barbs, protrusions, ridges, grooves, textured surfaces, adhesive, etc.), and a joint portion 201138. The fixed arms 201132 are attached to the inner paddles 201122.

In some implementations, the fixed arms 201132 are attached to the inner paddles 201122 with the joint portion 201138 disposed proximate the coaptation element 201110. The joint portion 201138 provides a spring force between the fixed and moveable arms 201132, 201134 of the clasp 201130. The joint portion 201138 can be any suitable joint, such as a flexible joint, a spring joint, a pivot joint, or the like. In some implementations, the joint portion 201138 is a flexible piece of material integrally formed with the fixed and moveable arms 201132, 201134.

In some implementations, the fixed arms 201132 are attached to the inner paddles 201122 and remain stationary or substantially stationary relative to the inner paddles 201122 when the moveable arms 201134 are opened to open the clasps 201130 and expose the optional barbs or other friction-enhancing elements 201136.

In some implementations, the clasps 201130 are opened by applying tension to actuation lines 201116 attached to the moveable arms 201134, thereby causing the moveable arms 201134 to articulate, flex, or pivot on the joint portions 201138. The actuation lines 201116 extend through the delivery system 201102 (e.g., through a steerable catheter, an implant catheter, etc.). Other actuation mechanisms are also possible.

In some implementations, the device 201000 is deployed from a delivery system 201102. The delivery system 201102 can comprise one or more of a catheter, a sheath, a guide catheter/sheath, a delivery catheter/sheath, a steerable catheter, an implant catheter, a tube, a channel, a pathway, combinations of these, etc. The actuation element 201112 can be configured to move through the delivery system 201102 to engage the device 201000 to move the anchor portion 201106 between open and closed positions.

The actuation line 201116 can take a wide variety of forms, such as, for example, a line, a suture, a wire, a rod, a catheter, or the like. The clasps 201130 can be spring loaded so that in the closed position the clasps 201130 continue to provide a pinching force on the grasped native leaflet. Optional barbs or other friction-enhancing elements 201136 of the clasps 201130 can grab, pinch, and/or pierce the native leaflets to further secure the native leaflets.

In some implementations, the delivery system 201102 includes a steerable catheter, implant catheter, and the actuation element 201112 (e.g., actuation wire, shaft, tube, hypotube, line, suture, braid, etc.). These can be configured to extend through a guide catheter/sheath (e.g., a transseptal sheath, etc.). In some implementations, the actuation element 201112 extends through a delivery catheter and the coaptation element 201110 to the distal end (e.g., a cap 201114 or other attachment portion at the distal connection of the anchor portion 201106).

Extending and retracting the actuation element 201112 increases and decreases the spacing between the coaptation element 201110 and the distal end of the device (e.g., the cap 201114 or other attachment portion), respectively. In some implementations, a collar or other attachment element (e.g., clamp, clip, lock, sutures, friction fit, buckle, snap fit, lasso, etc.) removably attaches the coaptation element 201110 to the delivery system 201102, either directly or indirectly, so that the actuation element 201112 slides through the collar or other attachment element and, in some implementations, through a coaptation element 201110 during actuation to open and close the paddles 201120, 201122 of the anchor portion 201106 and/or anchors 201108.

In some implementations, one or more components of the device 201000 and/or delivery device 201102 can be configured to improve the visibility when using an imaging device during implantation of the device 201000 on a native heart valve. For example, one or more components of the device 201000 can include material that causes the components to have an acoustic impedance that is substantially similar to the acoustic impedance of blood. That is, blood typically has an acoustic impedance of 1.68 MRayl, and the one or more components can have an acoustic impedance that is similar to this acoustic impedance of blood. In some implementations, the one or more components can have an acoustic impedance that is within 0.5 MRayl of blood, within 0.3 MRayl of blood, within 0.1 MRayl of blood, within 0.05 MRayl of blood, or within 0.02 MRayl of blood.

In the illustrated example, the coaptation element 201110, cap 201114, and actuation element 201112 include a material that allows for these components to have a desired acoustic impedance. While each of the coaptation element 201110, cap 20114, and actuation element 201112 are shown as including such a material, it should be understood that one or more of these components can include such a material, it should also be understood that any other component of the device 201000 and/or the delivery device 201102 can include a material for obtaining a desired acoustic impedance.

In some implementations, one or more components of the device 201000 and/or delivery device 201102 can be coated with a metal coating (e.g., a metal powder) and/or a ceramic coating (e.g., a ceramic powder) to cause the component(s) to have a desired acoustic impedance. For example, the one or more components can be coated with one or more of barium sulfate, titanium dioxide, zirconium dioxide, or any other suitable material that is capable of causing component(s) of the device 201000 and/or delivery device 201102 to have a desired acoustic impedance.

In some implementations, one or more of components can be made of a material that includes silicone rubber that is coated with one of the above-mentioned materials (or other suitable materials) to cause the component(s) to have a desired acoustic impedance. In some implementations, one or more of the components can be made of a combination of silicone rubber with one or more of the above-mentioned materials (or other suitable materials) to cause the component(s) to have a desired acoustic impedance. In some implementations, one or more components can be made of any other suitable material other than silicon rubber in combination with or coated with one or more of the above-mentioned materials (or other suitable materials) to cause the component(s) to have a desired acoustic impedance.

In some implementations, one or more components of the device 201000 and/or the delivery device can be made of a material that includes silicone rubber and about 15 vol % barium sulfate. In some examples, one or more components of the device 201000 and/or the delivery device can be made of a material that includes silicone rubber and about 17 vol % titanium dioxide. In some examples, one or more components of the device 201000 and/or the delivery device can be made of a material that includes silicone rubber and about 10 vol % zirconium dioxide.

In some implementations, one or more components of the device 201000 and/or delivery device 201102 can have an acoustic impedance of between about 1.5 MRayl and about 1.8 MRayl, such as between about 1.55 MRayl and about 1.75 MRayl, such as between about 1.60 MRayl and about 1.75 MRayl or any subrange of these ranges. In some implementations, one or more components of the device 201000 and/or delivery device 201102 can have an acoustic impedance of greater than or equal to 1.4 MRayl, such as greater than or equal to 1.5 MRayl, such as greater than or equal to 1.55 MRayl, such as greater than or equal to 1.60 MRayl.

During deployment and/or implantation, the paddles 201120, 201122 can be opened and closed, for example, to grasp the native leaflets (e.g., native mitral valve leaflets, etc.) between the paddles 201120, 201122 and/or between the paddles 201120, 201122 and a coaptation element 201110 (e.g., a spacer, plug, membrane, etc.).

The clasps 201130 can be used to grasp and/or further secure the native leaflets by engaging the leaflets with optional barbs or other friction-enhancing elements 201136 and pinching the leaflets between the moveable and fixed arms 201134, 201132. The optional barbs or other friction-enhancing elements 201136 (e.g., protrusions, ridges, grooves, textured surfaces, adhesive, etc.) of the clasps 201130 increase friction with the leaflets or can partially or completely puncture the leaflets.

In some implementations, the actuation lines 201116 can be actuated separately (or both separately and simultaneously) so that each clasp 201130 can be opened and closed separately. Separate operation allows one leaflet to be grasped at a time, or for the repositioning of a clasp 201130 on a leaflet that was insufficiently grasped, without altering a successful grasp on the other leaflet. The clasps 201130 can be opened and closed relative to the position of the inner paddle 201122 (as long as the inner paddle is in an open or at least partially open position), thereby allowing leaflets to be grasped in a variety of positions as the particular situation requires.

The device 20100 can be moved to various positioned during delivery and implantation. For example, the device 201000 can be moved to any of the positions shown for the device 100 in FIGS. 8-15.

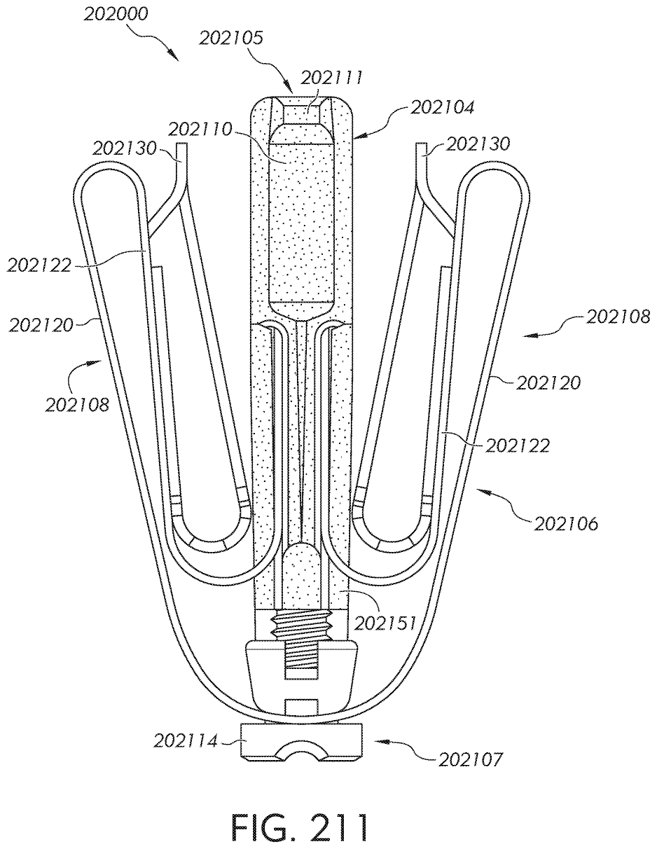

FIG. 211 illustrates an example of a device 202000 (e.g., valve repair device, valve treatment device, implantable device, implant, etc.) that includes a smaller coaptation element 202110, where one or more components of the device 202000 (including or not including the coaptation element 202110) are configured to improve the visibility when using an imaging device (e.g., a commercial ultrasound imaging device) during implantation of the device 202000 on a native heart valve.

In some implementations, one or more components of the device 202000 can be configured to have an acoustic impedance that is substantially similar to the acoustic impedance of blood, which improves the visibility during implantation of the device 202000. For example, the one or more components can have an acoustic impedance that is within 0.5 MRayl of blood, within 0.3 MRayl of blood, within 0.1 MRayl of blood, within 0.05 MRayl of blood, or within 0.02 MRayl of blood.

The device 202000 can include any features for a device or valve repair device discussed in the present application. The device 202000 can be deployed from a delivery sheath, and/or an implant catheter. The device 202000 can be a prosthetic spacer device, valve repair device, valve treatment device, implant, or another type of device that attaches to leaflets of a native valve.

In some implementations, the device 202000 can take the form of the device 102200 described in WO 2023/278663, which is incorporated herein by reference in its entirety for all purposes, except that one or more components of the device 102200 are configured to have an acoustic impedance that is substantially similar to the acoustic impedance of blood. For example, the one or more components can have an acoustic impedance that is within 0.5 MRayl of blood, within 0.3 MRayl of blood, within 0.1 MRayl of blood, within 0.05 MRayl of blood, or within 0.02 MRayl of blood.

In some implementations, the device 202000 can include a coaptation portion 202104, a proximal or attachment portion 202105, an anchor portion 202106, and a distal portion 202107. The anchor portion 102206 can include one or more anchors 202108. The anchors 202108 can be configured in a variety of ways, such as, for example, any of the ways described in the present application.

In the illustrated example, the anchor portion 202106 includes two anchors 202108 with each anchor 202108 having outer paddles 202120, inner paddles 202122, paddle extension members or paddle frames (not shown), and clasps 202130. The outer paddles 202120, inner paddles 202122, paddle frames, and clasps 202130 can take any suitable form, such as, for example, any form described in the present application.

In some implementations, the attachment portion 202105 includes a first or proximal collar 202111 (or other attachment element) for engaging with a coupler of a delivery system. In some implementations, the distal portion 202107 includes a cap 202114 that is operatively connected to the anchor portion 202106 of the device 202000 such that movement of the cap 202114 relative to a spacer or coaptation element 202110 of the coaptation portion 202104 causes the anchors 202108 to move between open and closed positions.

In some implementations, the coaptation portion 202104 of the device can include a coaptation element 202110 (e.g., a spacer, coaptation element, bushing, etc.) for implantation between leaflets of a native valve. The coaptation element 202110 extends from the proximal collar 202111 (or other attachment element) to a distal end 202151 and includes a lumen (not shown) for receiving one or more components of a delivery device, such as, for example, an actuation element (e.g., actuation shafts, actuation rods, actuation tubes, actuation wires, actuation lines, etc.), a width adjustment element (e.g. width adjustment wire, width adjustment shaft, width adjustment tube, width adjustment line, width adjustment cord, width adjustment suture, etc.), etc. In some implementations, the proximal collar 202111 of the attachment portion 202105 can be integral with the coaptation element 202110. In some implementations, the proximal collar 202111 and the coaptation element 202110 can be separate components.

In some implementations, the proximal end of the coaptation element 202110 has a proximal opening (not shown) that allows an actuation element and/or a width adjustment element to move relative to the lumen. The proximal opening allows the actuation element and/or the width adjustment element to engage one or more controllable components of the device or implant 202000.

In some implementations, the distal end 202151 of the coaptation element 202110 can include a distal opening (not shown). An actuation element and/or one or more components that couple the cap 202114 of the device 202000 to the actuation element can extend through the distal opening and attach to the cap 202114. In some implementations, an actuation element extends from the delivery system to engage and enable actuation of the device 202000 between the open and closed positions and/or a width adjustment element extends from the delivery system to adjust widths of the paddles.

Movement of the cap 202114 away from the coaptation element 202110 can cause the anchors 202108 to move to the open position, and movement of the cap 202114 toward the coaptation element 202110 can cause the anchors 202108 to move to the closed position. The actuation element, the cap, and any components that couple the cap to the actuation element can be configured to removably engage the cap 202114 in any suitable manner that allows the actuation element to be disengaged and removed from the device 202000 after implantation, such as, for example, any manner described in the present application or WO 2023/278663.

In some implementations, one or more width adjustment elements extend through the lumen of the coaptation element 202110 and engage the paddle frames of the device 202000 to move the paddle frames between a narrowed configuration and an expanded configuration. For example, the one or more width adjustment elements can engage the paddle frames of the device 202000 to move the paddle frames between narrowed and expanded configurations by any means described in WO 2023/278663.

In some implementations, when viewed from above, the coaptation element 202110 has a smaller size relative to the outer periphery of the device 202000 (e.g., similar to the coaptation element 102210 of the device 102200 shown in FIG. 171 of WO 2023/278663). For example, a ratio of an area defined by the outer periphery of the paddle frames when viewed from above relative to the outer periphery of the coaptation element 202110 when viewed from above can be greater than or equal to 2 to 1. In some implementations, this ration can be greater than or equal to 3 to 1, greater than or equal to 4 to 1, greater than equal to 5 to 1, greater than or equal to 6 to 1, greater than or equal to 10 to 1.

In implementations with a smaller coaptation element, an optional cover (e.g., the optional cover described in WO 2023/278663) can be attached to the paddle frames to cover any open portions between the paddle frames and the coaptation element 202110 to block regurgitative blood flow between the paddle frames and the coaptation element 202110.

The smaller size of the coaptation element 202110 can allow for easier movement of the various components of the anchors 202108 as the anchors 202108 are moved between the open and closed positions. In some implementations, the coaptation element 202110 can include planar surfaces for allowing the clasps 202130 to rest on when the device 202000 is in the closed position.

In some implementations, the coaptation element 202110 can have tapered walls that extend inward from the proximal end to the distal end of the coaptation element 202110 to create additional space for the anchors 202108 to move when the device is moved to the opened position. That is, as the device 202000 is moved to the opened position, one or more components of the anchors 202108 (e.g., the paddle frames) pivot downward, and the tapered walls provide for additional space for the components of the anchors to move.

In some implementations, the coaptation element 202110 has a proximal portion having a generally rectangular shape and a distal portion having a generally rounded shape. However, it should be understood that other configurations are also contemplated.

In some implementations, the coaptation element 202110 can be made by injection molding. However, it should be understood that other configurations are also contemplated. In some implementations, the coaptation element 202110 can be made from or comprise any known polymer material or other non-polymer material(s). In some implementations, the coaptation element 202110 can be made from or comprise a metal material or any other suitable material.

In some implementations, one or more components of the device 202000 and/or delivery device can be configured to improve the visibility when using an imaging device during implantation of the device 202000 on a native heart valve. For example, one or more components of the device 202000 can include material that causes the components to have an acoustic impedance that is substantially similar to the acoustic impedance of blood. That is, blood typically has an acoustic impedance of 1.68 MRayl, and the one or more components can have an acoustic impedance that is similar to this acoustic impedance of blood.

In some implementations, the one or more components can have an acoustic impedance that is within 0.5 MRayl of blood, within 0.3 MRayl of blood, within 0.1 MRayl of blood, within 0.05 MRayl of blood, or within 0.02 MRayl of blood. In the illustrated example, the coaptation element 202110 includes a material that allows for the coaptation element 202110 to have a desired acoustic impedance. However, it should be understood that any other component of the device 202000 and/or the delivery device can include a material for obtaining a desired acoustic impedance.

In some implementations, one or more components of the device 202000 and/or delivery device can be coated with a metal coating (e.g., a metal powder) and/or a ceramic coating (e.g., a ceramic powder) to cause the component(s) to have a desired acoustic impedance. For example, the one or more components can be coated with one or more of barium sulfate, titanium dioxide, zirconium dioxide, or any other suitable material that is capable of causing component(s) of the device 202000 and/or delivery device to have a desired acoustic impedance.

In some implementations, one or more of components can be made of a material that includes silicone rubber that is coated with one of the above-mentioned materials (or other suitable materials) to cause the component(s) to have a desired acoustic impedance. In some implementations, one or more of the components can be made of a combination of silicone rubber with one or more of the above-mentioned materials (or other suitable materials) to cause the component(s) to have a desired acoustic impedance.

In some implementations, one or more components can be made of any other suitable material other than silicon rubber in combination with or coated with one or more of the above-mentioned materials (or other suitable materials) to cause the component(s) to have a desired acoustic impedance.

In some examples, one or more components of the device 202000 and/or the delivery device can be made of a material that includes silicone rubber and about 15 vol % barium sulfate. In some examples, one or more components of the device 202000 and/or the delivery device can be made of a material that includes silicone rubber and about 17 vol % titanium dioxide. In some examples, one or more components of the device 202000 and/or the delivery device can be made of a material that includes silicone rubber and about 10 vol % zirconium dioxide.

In some implementations, one or more components of the device 202000 and/or delivery device can have an acoustic impedance of between about 1.5 MRayl and about 1.8 MRayl, such as between about 1.55 MRayl and about 1.75 MRayl, such as between about 1.60 MRayl and about 1.75 MRayl. In some implementations, one or more components of the device 202000 and/or delivery device can have an acoustic impedance of greater than or equal to 1.4 MRayl, such as greater than or equal to 1.5 MRayl, such as greater than or equal to 1.55 MRayl, such as greater than or equal to 1.60 MRayl.

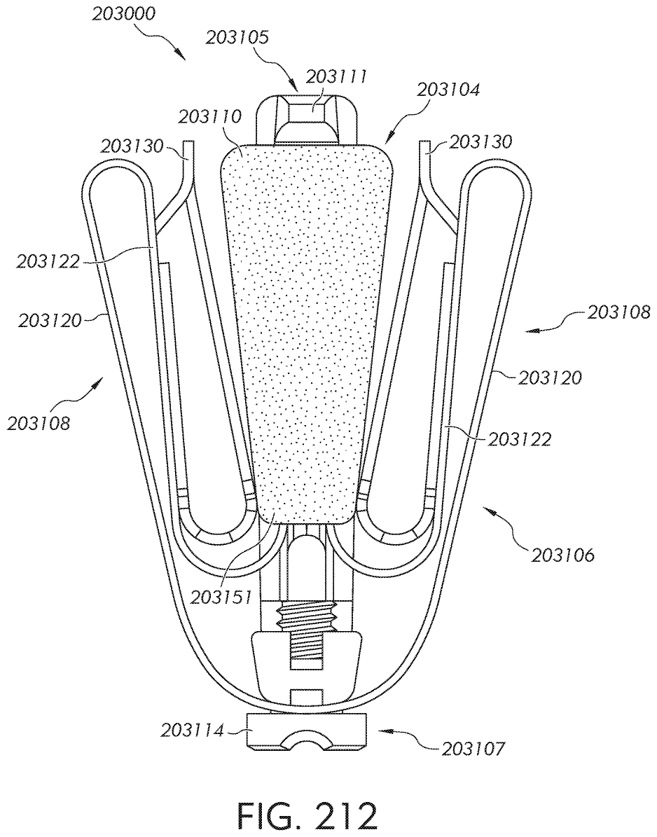

FIG. 212 illustrates an example of a device 203000 (e.g., valve repair device, valve treatment device, implantable device, implant, etc.) that includes a larger coaptation element 203110 relative to the coaptation element 202000 described with reference to FIG. 211, where one or more components of the device 203000 (including or not including the coaptation element 202110) are configured to improve the visibility when using an imaging device (e.g., a commercial ultrasound imaging device) during implantation of the device 203000 on a native heart valve.

In some implementations, one or more components of the device 203000 can be configured to have an acoustic impedance that is substantially similar to the acoustic impedance of blood, which improves the visibility during implantation of the device 203000. For example, the one or more components can have an acoustic impedance that is within 0.5 MRayl of blood, within 0.3 MRayl of blood, within 0.1 MRayl of blood, within 0.05 MRayl of blood, or within 0.02 MRayl of blood.

In some implementations, the device 203000 can include any features for a device or valve repair device discussed in the present application. In some implementations, the device 203000 can be deployed from a delivery sheath, and/or an implant catheter. In some implementations, the device 203000 can be a prosthetic spacer device, valve repair device, valve treatment device, implant, or another type of device that attaches to leaflets of a native valve.

In some implementations, the device 203000 can take the form of the device 102200 described in WO 2023/278663, which is incorporated herein by reference in its entirety for all purposes, except that the coaptation element 203110 of the device 203000 is larger than the coaptation element 102210 of the device 102200, and except that one or more components of the device 203000 are configured to have an acoustic impedance that is substantially similar to the acoustic impedance of blood.

In some implementations, the one or more components can have an acoustic impedance that is within 0.5 MRayl of blood, within 0.3 MRayl of blood, within 0.1 MRayl of blood, within 0.05 MRayl of blood, or within 0.02 MRayl of blood. In some implementations, the device 203000 takes the form of the device 102200 described in WO 2023/278663, but the coaptation element 203110 shown in FIG. 212 is positioned over the coaptation element 102210 of the device 102200.

In some implementations, the device 203000 can include a coaptation portion 203104, a proximal or attachment portion 203105, an anchor portion 203106, and a distal portion 203107. The anchor portion 203106 can include one or more anchors 203108. The anchors 202108 can be configured in a variety of ways, such as, for example, any of the ways described in the present application.

In the illustrated example, the anchor portion 203106 includes two anchors 203108 with each anchor 203108 having outer paddles 203120, inner paddles 203122, paddle extension members or paddle frames (not shown), and clasps 203130. The outer paddles 203120, inner paddles 203122, paddle frames, and clasps 203130 can take any suitable form, such as, for example, any form described in the present application.

In some implementations, the attachment portion 203105 includes a first or proximal collar 203111 (or other attachment element) for engaging with a coupler of a delivery system. In some implementations, the distal portion 203107 includes a cap 203114 that is operatively connected to the anchor portion 203106 of the device 203000 such that movement of the cap 203114 relative to a spacer or coaptation element 203110 of the coaptation portion 203104 causes the anchors 203108 to move between open and closed positions.

In some implementations, the coaptation portion 203104 of the device can include a coaptation element 203110 (e.g., a spacer, coaptation element, bushing, etc.) for implantation between leaflets of a native valve. In some implementations, the coaptation element 203110 extends from the proximal collar 203111 (or other attachment element) to a distal end 203151 and includes a lumen (not shown) for receiving one or more components of a delivery device, such as, for example, an actuation element (e.g., actuation shafts, actuation rods, actuation tubes, actuation wires, actuation lines, etc.), a width adjustment element (e.g. width adjustment wire, width adjustment shaft, width adjustment tube, width adjustment line, width adjustment cord, width adjustment suture, etc.), etc. In the illustrated example, the proximal collar 203111 of the attachment portion 203105 is integral with the coaptation element 203110.

In some implementations, the proximal end of the coaptation element 203110 has a proximal opening (not shown) that allows an actuation element and/or a width adjustment element to move relative to the lumen. The proximal opening allows the actuation element and/or the width adjustment element to engage one or more controllable components of the device or implant 203000.

In some implementations, the distal end 203151 of the coaptation element 203110 can include a distal opening (not shown). An actuation element and/or one or more components that couple the cap 203114 of the device 203000 to the actuation element can extend through the distal opening and attach to the cap 203114.

In some implementations, an actuation element extends from the delivery system to engage and enable actuation of the device 203000 between the open and closed positions and/or a width adjustment element extends from the delivery system to adjust widths of the paddles. Movement of the cap 203114 away from the coaptation element 203110 can cause the anchors 203108 to move to the open position, and movement of the cap 203114 toward the coaptation element 203110 can cause the anchors 203108 to move to the closed position.

In some implementations, the actuation element, the cap, and any components that couple the cap to the actuation element can be configured to removably engage the cap 203114 in any suitable manner that allows the actuation element to be disengaged and removed from the device 203000 after implantation, such as, for example, any manner described in the present application or WO 2023/278663.

In some implementations, one or more width adjustment elements extend through the lumen of the coaptation element 203110 and engage the paddle frames of the device 203000 to move the paddle frames between a narrowed configuration and an expanded configuration. For example, the one or more width adjustment elements can engage the paddle frames of the device 203000 to move the paddle frames between narrowed and expanded configurations by any means described in WO 2023/278663.

In some implementations, one or more components of the device 203000 and/or delivery device can be configured to improve the visibility when using an imaging device during implantation of the device 203000 on a native heart valve. For example, one or more components of the device 203000 can include material that causes the components to have an acoustic impedance that is substantially similar to the acoustic impedance of blood. That is, blood typically has an acoustic impedance of 1.68 MRayl, and the one or more components can have an acoustic impedance that is similar to this acoustic impedance of blood.

In some implementations, the one or more components can have an acoustic impedance that is within 0.5 MRayl of blood, within 0.3 MRayl of blood, within 0.1 MRayl of blood, within 0.05 MRayl of blood, or within 0.02 MRayl of blood. In the illustrated example, the coaptation element 203110 includes a material that allows for the coaptation element 202110 to have a desired acoustic impedance. However, it should be understood that any other component of the device 203000 and/or the delivery device can include a material for obtaining a desired acoustic impedance.

In some implementations, one or more components of the device 203000 and/or delivery device can be coated with a metal coating (e.g., a metal powder) and/or a ceramic coating (e.g., a ceramic powder) to cause the component(s) to have a desired acoustic impedance. For example, the one or more components can be coated with one or more of barium sulfate, titanium dioxide, zirconium dioxide, or any other suitable material that is capable of causing component(s) of the device 203000 and/or delivery device to have a desired acoustic impedance.

In some implementations, one or more of components can be made of a material that includes silicone rubber that is coated with one of the above-mentioned materials (or other suitable materials) to cause the component(s) to have a desired acoustic impedance. In some implementations, one or more of the components can be made of a combination of silicone rubber with one or more of the above-mentioned materials (or other suitable materials) to cause the component(s) to have a desired acoustic impedance.

In some implementations, one or more components can be made of any other suitable material other than silicon rubber in combination with or coated with one or more of the above-mentioned materials (or other suitable materials) to cause the component(s) to have a desired acoustic impedance.

In some examples, one or more components of the device 203000 and/or the delivery device can be made of a material that includes silicone rubber and about 15 vol % barium sulfate. In some examples, one or more components of the device 203000 and/or the delivery device can be made of a material that includes silicone rubber and about 17 vol % titanium dioxide. In some examples, one or more components of the device 203000 and/or the delivery device can be made of a material that includes silicone rubber and about 10 vol % zirconium dioxide.

In some implementations, one or more components of the device 203000 and/or delivery device can have an acoustic impedance of between about 1.5 MRayl and about 1.8 MRayl, such as between about 1.55 MRayl and about 1.75 MRayl, such as between about 1.60 MRayl and about 1.75 MRayl. In some implementations, one or more components of the device 203000 and/or delivery device can have an acoustic impedance of greater than or equal to 1.4 MRayl, such as greater than or equal to 1.5 MRayl, such as greater than or equal to 1.55 MRayl, such as greater than or equal to 1.60 MRayl.

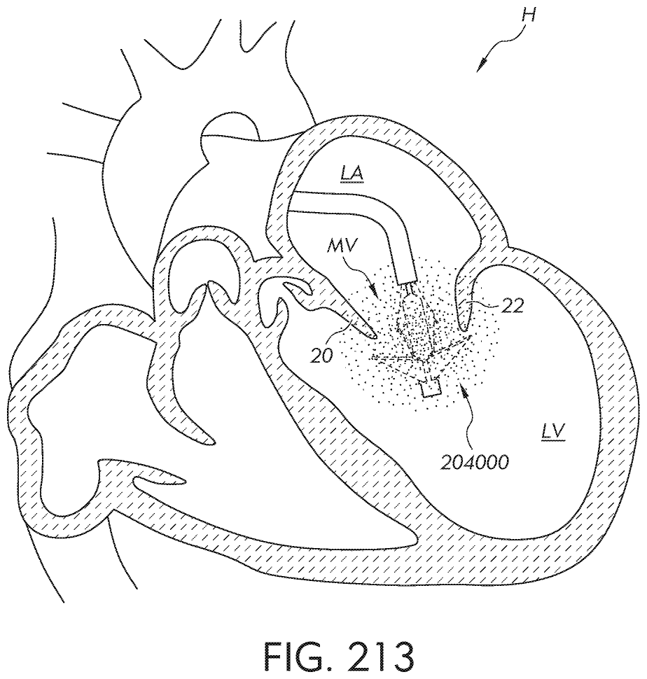

FIG. 213 shows an implantable device 204000 being delivered within a native mitral valve MV of the heart H. The device 204000 does not include components that are configured to have an acoustic impedance that is substantially similar to the acoustic impedance of blood. This mismatch between the acoustic impedance of components of the device 204000 and the acoustic impedance of blood may cause imaging artifacts when using an imaging device (e.g., a commercial ultrasound imaging device) to observe the device during implantation. The imaging artifacts can cause the visibility of the device 204000 to appear blurry to the individual and/or team implanting the device.

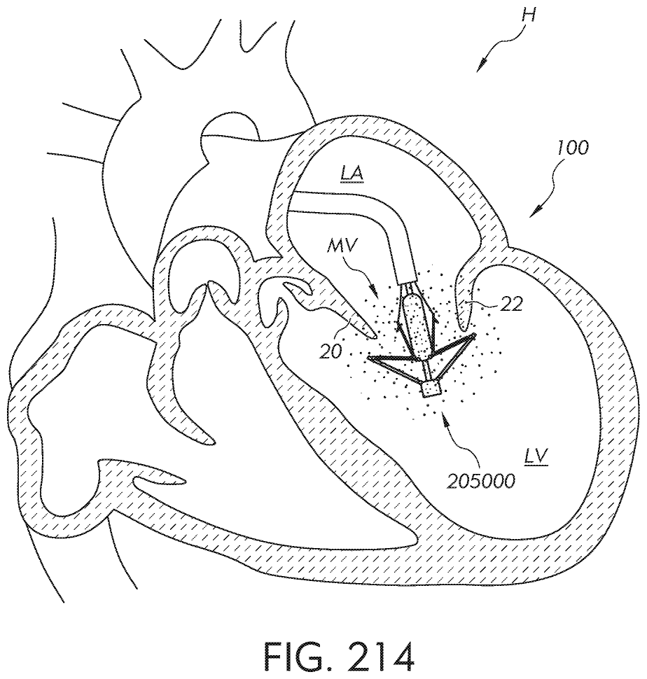

FIG. 214 shows another implantable device 205000 being delivered within a native mitral valve MV of the heart H. The device 205000 includes one or more components that are configured to have an acoustic impedance that is substantially similar to the acoustic impedance of blood which improves the visibility when using an imaging device (e.g., a commercial ultrasound imaging device) during implantation of the device 205000). For example, having one or more components with an acoustic impedance substantially similar to blood can reduce imaging artifacts when imaging the device during an implantation.

The device 20500 can take any suitable form, such as any form described for a device described with reference to FIGS. 210-212 of the present application. In some implementations, components of the device 205000 that are not opened and closed during leaflet capture (e.g., the spacer, cap, etc.) can have an acoustic impedance that is substantially similar to blood. In some implementations, these components can have an acoustic impedance that is within 0.5 MRayl of blood, within 0.3 MRayl of blood, within 0.1 MRayl of blood, within 0.05 MRayl of blood, or within 0.02 MRayl of blood. This can allow the imaging device to more clearly show the position of the components of device 205000 that are actively opened and closed for leaflet capture (e.g., gripping members, paddles, clasps, etc.).

The device 204000, 205000 can be delivered and deployed within the native mitral valve MV of the heart H by any suitable means, such as, for example, the means described for the device 100 shown in FIGS. 16-21 or any other means described in the present application.

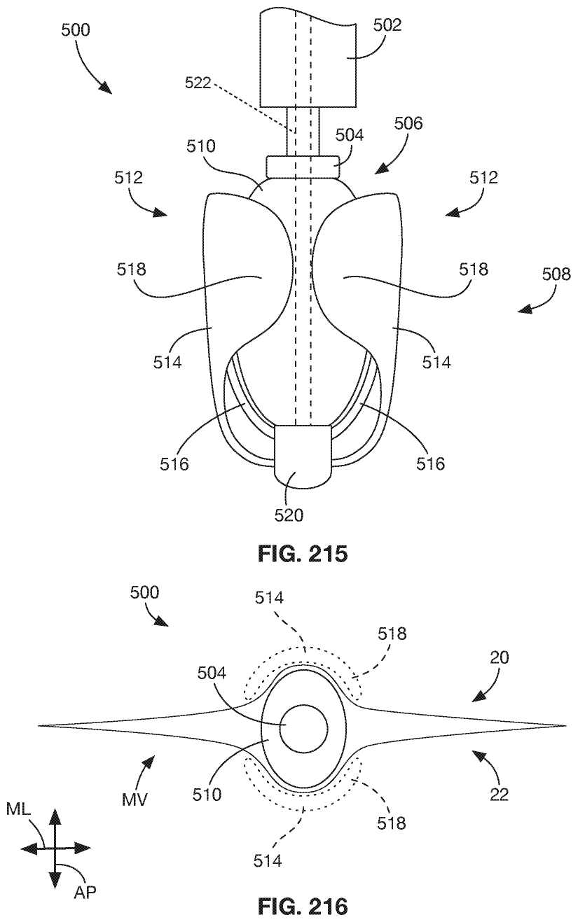

Referring now to FIGS. 215-218, an example of a device 500 (e.g., treatment device, repair device, implantable device, implant, etc.) is shown. The device 500 can be configured as an implantable device or implant or other valve treatment device (e.g., one that does not necessarily remain implanted). The device 500 is one of the many different configurations that the device 100 that is schematically illustrated in FIGS. 8-14 can take.

The device 500 can include any other features for a device or implant discussed in the present application, and the device 500 can be positioned to engage valve tissue 20, 22 as part of any suitable treatment and/or repair system (e.g., any treatment and/or repair system disclosed in the present application, or the applications cited herein). The device/implant 500 can be a prosthetic spacer device, valve repair device, treatment device, or another type of implant that attaches to leaflets of a native valve.

In some implementations, the device 500 is implanted in the heart of the patient via the use of a delivery system 502. The delivery system 502 for the device 500 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. In some implementations, the device 500 includes an attachment portion that includes a first or proximal collar 504 (or other attachment element) for engaging with a capture mechanism of a delivery system 502. The capture mechanism can be configured in a variety of ways and, in some implementations, can comprise one or more of a clamp, clip, pin, suture, line, lasso, noose, snare, buckle, lock, latch, etc.

In some implementations, the device 500 includes a coaptation portion 506, and an anchor portion 508. In some implementations, the coaptation portion 506 of the device optionally includes a coaptation element 510 (e.g., a spacer, coaptation element, plug, membrane, sheet, gap filler, plug, wedge, balloon, etc.) for deployment and/or implantation between leaflets of a native valve. In some implementations, the anchor portion 508 includes a plurality of anchors 512. The anchors 512 can be configured in a variety of ways. In some implementations, each anchor 512 includes outer paddles 514, inner paddles 516, paddle extension members or paddle frames 518, and clasps or gripping members (not shown).

In some implementations, the outer paddles 514 are jointably attached to a cap 520 and to the inner paddles 516. The inner paddles 516 are jointably attached to the coaptation element 510. In this manner, the anchors 512 are configured similar to legs in that the inner paddles 516 are like upper portions of the legs, the outer paddles 514 are like lower portions of the legs, and the connection between the inner paddles 516 and the outer paddles 514 are like knee portions of the legs. Similar to the device 100 shown in FIGS. 8-14, extending an actuation wire 522 in a distal direction also moves the cap 522 in the distal direction to cause the anchors 512 to open. Retracing the actuation wire 522 in the proximal direction causes the anchors 512 to close.

The paddle extension members 518 can provide additional pinching force between the inner paddles 516 and the coaptation element 510 and assist in wrapping the leaflets around the sides of the coaptation element 510. That is, the paddle extension members 518 can be configured with a round three-dimensional shape that follows the contour of the coaptation element 510. The wide configuration of the paddle extension members 518 provides increased surface area compared to the inner paddles 516 alone. The increased surface area can distribute the clamping force of the anchors 512 and the paddle extension members 518 against the native leaflets over a relatively larger surface of the native leaflets in order to further protect the native leaflet tissue.

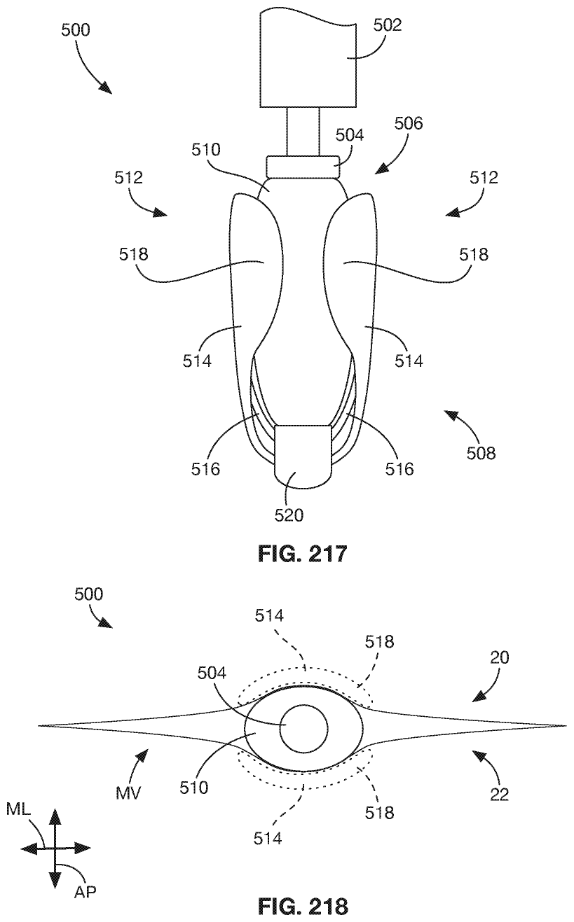

The anchors 512 can be moved from an open position to a first closed position (FIGS. 215-216). Further application of force in the closing direction moves the anchors 512 from the first closed position to a second closed position (FIGS. 217-218). Closing the anchors 512 from the first closed position to the second closed position presses the outer paddles 514, the inner paddles 516, and the paddle extension members 518 against the coaptation element 510. The coaptation element 510 comprises and/or is formed from a flexible material so that force applied to the coaptation element 510 by the anchors 512 deforms and changes the shape of the coaptation element 510. In some implementations, the closing force applied by the anchors 512 is aligned with the longest dimension—i.e., the anterior-posterior direction AP—of the coaptation element 510 so that the coaptation element 510 is shortened in the anterior-posterior direction AP. The flexible material forming the coaptation element 510 is flexible but sufficiently stiff so that the coaptation element 510 resists or pushes back against exterior forces. Thus, compressing or shortening the coaptation element 510 along one axis causes the coaptation element 510 to expand or lengthen along a transverse or orthogonal axis. For instance, compressing the coaptation element 510 along the anterior-posterior direction AP causes the coaptation element 510 to expand along the medial-lateral direction ML.

As the coaptation element 510 widens in the direction transverse to the application of force by the anchors 512—e.g., the medial-lateral direction ML—the paddle extension members 518 also spread out to correspond to the change in shape of the coaptation element 510. The changing shape of the coaptation element 510 and the paddle extension members 518 helps to maintain engagement with the leaflets 20, 22 as the anchor 512 is closed beyond the first closed position.

In some implementations, the paddle extension members 518 and the coaptation element 510 are shape set in the shape taken when the anchors 512 are in the first closed position—that is, the position where the outer paddles 516 and the paddle extension members 518 form a more rounded C-shape. Consequently, when the anchors 512 are moved from the second closed position back to the first closed position, or to any intermediate position between the first closed position and the second closed position, the paddle extension members 518 and the coaptation element 510 also change shape—e.g., the paddle extension members 518 move inward toward the coaptation element 510 and the coaptation element 510 lengthens in the anterior-posterior direction AP and shortens in the medial-lateral direction ML. In this way, the operator can adjust the shape of both the coaptation element 510 and the anchors 512 to compensate for the condition of the leaflets 20, 22 captured between the anchors 512 and the coaptation element 510. In some implementations, changing the shape of the coaptation element 510 and the paddle extension member 518 to a more curved shape can take up slack in the leaflet 20, 22 if the leaflet 20, 22 has stretched or there is excessive leaflet tissue caused by a degenerative condition.

It should also be noted that pairing the expansion of the paddle 516 and the coaptation element 510 to closure of the anchors 512 can provide additional benefits after implantation in the patient's heart. In some implementations, where the anchors 512 are closed to the second closed position or to a position between the first closed position and the second closed position, tension applied to the anchors 512 in an opening direction during diastole moves the anchors towards the first closed position so that the paddle extension members 518 and the coaptation element 510 become narrower in the medial-lateral direction ML. Because the shape of the coaptation element 510 is paired with the shape of the anchor 512, increasing the depth of the device 500 in the anterior-posterior direction AP does not result in the creation of a leak path through the device 500.

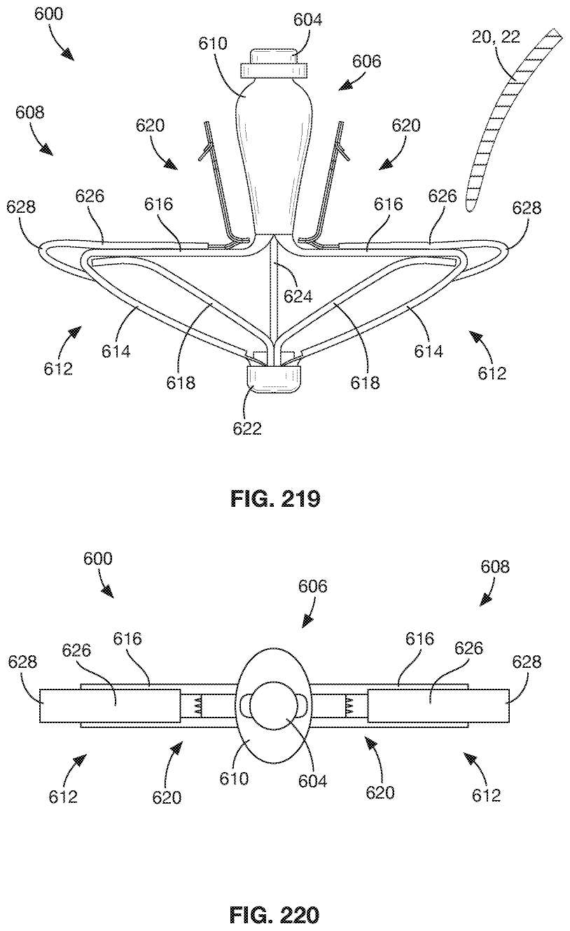

Referring now to FIGS. 219-220, an example of a device 600 (e.g., treatment device, repair device, implantable device, implant, etc.) is shown. The device 600 can be configured as an implantable device or implant or other valve treatment device (e.g., one that does not necessarily remain implanted). The device 600 is one of the many different configurations that the device 100 that is schematically illustrated in FIGS. 8-14 can take. The device 600 can include any other features for a device or implant discussed in the present application, and the device 600 can be positioned to engage valve tissue 20, 22 as part of any suitable treatment and/or repair system (e.g., any treatment and/or repair system disclosed in the present application, or the applications cited herein). The device/implant 600 can be a prosthetic spacer device, valve repair device, treatment device, or another type of implant that attaches to leaflets of a native valve.

In some implementations, the device 600 is implanted in the heart of the patient via the use of a delivery system. The delivery system for the device 600 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. In some implementations, the device 600 includes an attachment portion that includes a first or proximal collar 604 (or other attachment element) for engaging with a capture mechanism of the delivery system. The capture mechanism can be configured in a variety of ways and, in some implementations, can comprise one or more of a clamp, clip, pin, suture, line, lasso, noose, snare, buckle, lock, latch, etc.

In some implementations, the device 600 includes a coaptation portion 606, and an anchor portion 608. In some implementations, the coaptation portion 606 of the device optionally includes a coaptation element 610 (e.g., a spacer, coaptation element, plug, membrane, sheet, gap filler, plug, wedge, balloon, etc.) for deployment and/or implantation between leaflets of a native valve. In some implementations, the anchor portion 608 includes a plurality of anchors 612. The anchors 612 can be configured in a variety of ways. In some implementations, each anchor 612 includes outer paddles 614 and inner paddles 616. In some implementations, each anchor includes paddle extension members or paddle frames 618. In some implementations, each anchor includes clasps or gripping members 620.

In some implementations, the outer paddles 614 are jointably attached to a cap 622 and to the inner paddles 616. The inner paddles 616 are jointably attached to the coaptation element 610. In this manner, the anchors 612 are configured similar to legs in that the inner paddles 616 are like upper portions of the legs, the outer paddles 614 are like lower portions of the legs, and the connection between the inner paddles 616 and the outer paddles 614 are like knee portions of the legs. Similar to the device 100 shown in FIGS. 8-14, extending an actuation wire or line 624 in a distal direction also moves the cap 622 in the distal direction to cause the anchors 612 to open. Retracing the actuation wire or line 624 in the proximal direction causes the anchors 612 to close.

In some implementations, the anchors 612 each include a cover 626 formed from a sheet, material, fabric, layer or membrane that is attached to the device 600 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth. In some implementations, the cover 626 can be arranged on the inner paddle 616 and can cover a fixed arm of the gripping member 620.

In some implementations, the cover 626 on the inner paddle 616 and gripping member 620 can include an extension 628 that extends beyond the joint between the inner paddle 616 and the outer paddle 614. In some implementations, the extension 628 of the cover 626 loops back to attach to the outer paddle 614 and/or the paddle frame 618. The extension 628 of the cover 626 increases the width or wingspan of the anchor portion 608 of the device 600 when the anchor portion 608 is in a partially open or capture-ready position, as shown in FIG. 219. Increasing the width or wingspan of the anchor portion 608 enhances the ability of the operator to engage the leaflets 20, 22 with the inner paddles 616 prior to closure of the gripping members 620. That is, a leaflet 20, 22 can be engaged with the extensions 628 that would have otherwise been missed by or insufficiently engaged by the inner paddles 616. Consequently, the extensions 628 can enable the capture of leaflets 20, 22 that have a coaptation gap W (FIG. 6) that would be too large for devices without the extensions 628 to capture.

Because the extension 628 can be formed from the same flexible material that forms the cover 626, the extension 628 does not interfere with the movement of the leaflets 20, 22 when the anchor portion 608 of the device 600 is closed. It should also be noted that forming the extension 628 from the cover 626 enables the width or wingspan of the device 600 to be increased without modifying or replacing existing metal or plastic components of the device 600. In some implementations, the extensions 628 can be added to already-manufactured covers 626 for devices 600. In some implementations, the extensions 628 can be attached to an already-manufactured device 600.

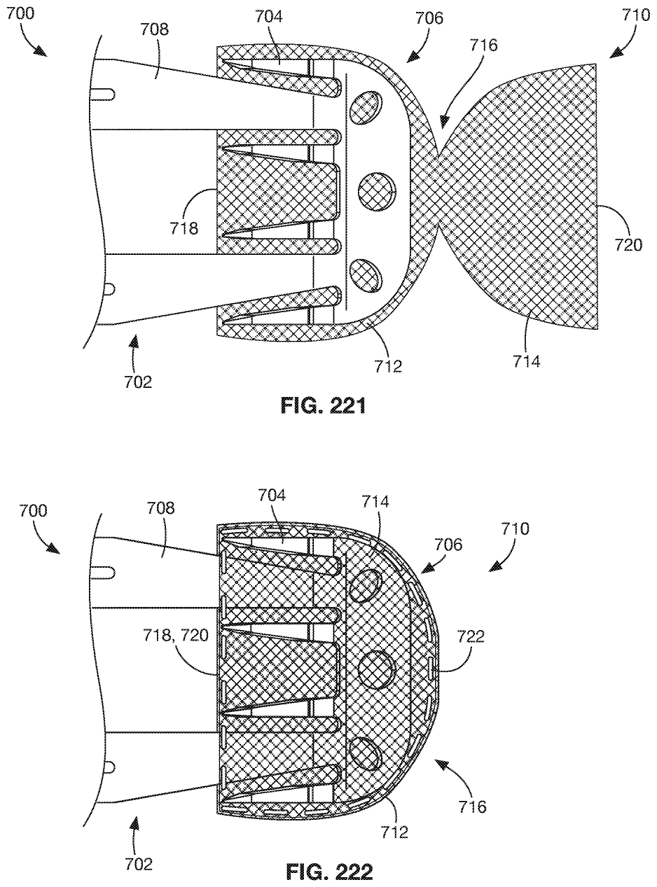

Referring now to FIGS. 221-228, a portion of a device 700 (e.g., treatment device, repair device, implantable device, implant, etc.) is shown. The device 700 can be configured as an implantable device or implant or other valve treatment device (e.g., one that does not necessarily remain implanted). The device 700 is one of the many different configurations that the device 100 that is schematically illustrated in FIGS. 8-14 can take. The device 700 can include any other features for a device or implant discussed in the present application, and the device 700 can be positioned to engage valve tissue 20, 22 as part of any suitable treatment and/or repair system (e.g., any treatment and/or repair system disclosed in the present application, or the applications cited herein). The device/implant 700 can be a prosthetic spacer device, valve repair device, treatment device, or another type of implant that attaches to leaflets of a native valve.

In FIGS. 221-222 and 225-228, an enlarged view of a portion of a clasp or a gripping member 702 of the device 700 is shown. The gripping member 702 (e.g., gripping arms, clasp arms, attachment portions, etc.) can be any of the clasps or gripping members described herein. The gripping member 702 can optionally include friction-enhancing elements or securing structures 704 (e.g., barbs, protrusions, ridges, grooves, textured surfaces, adhesive, etc.) to facilitate securing a leaflet captured with the gripping member 702. The portion of the gripping member 702 illustrated in FIGS. 221-222 and 225-228 is an end 706 of a moveable arm 708 of the gripping member 702 that includes a plurality of friction-enhancing elements or barbs 704.

Referring now to FIGS. 221-222, a cover 710 for the end 706 of the gripping member 702 is shown being attached to the gripping member 702. The cover 710 can be configured in a variety of ways. In some implementations, the cover can include a sheet, material, fabric, layer or membrane that is attached to the device 700 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth.

The cover 710 can take on a wide variety of forms. The example of the cover 710 shown in FIG. 221 can be cut from a flat sheet of material and has a first half 712 and a second half 714 that are joined together by a foldable region 716. Both the first half 712 and the second half 714 are shaped to approximately correspond to the shape of the end 706 of the gripping member 702. To attach the cover 710 to the gripping member 702, the first half 712 of the cover 710 is arranged below the end 706 of the gripping member 702 and the second half 714 is folded on top of the end 706 of the gripping member 702, as is shown in FIG. 222. The cover 710 is folded around the end 706 of the gripping member 702 so that a first edge 718 of the first half 712 is approximately aligned with a second edge 720 of the second half 714. When folding the cover 710, the friction-enhancing elements 704 are allowed to penetrate and protrude through the second half 714 of the cover 710. To secure the folded cover 710 to the end 706 of the gripping member 702, a plurality of stitches 722 (or other connectors as described herein) are formed around the perimeter of the overlaid first and second halves 712, 714 of the cover 710.

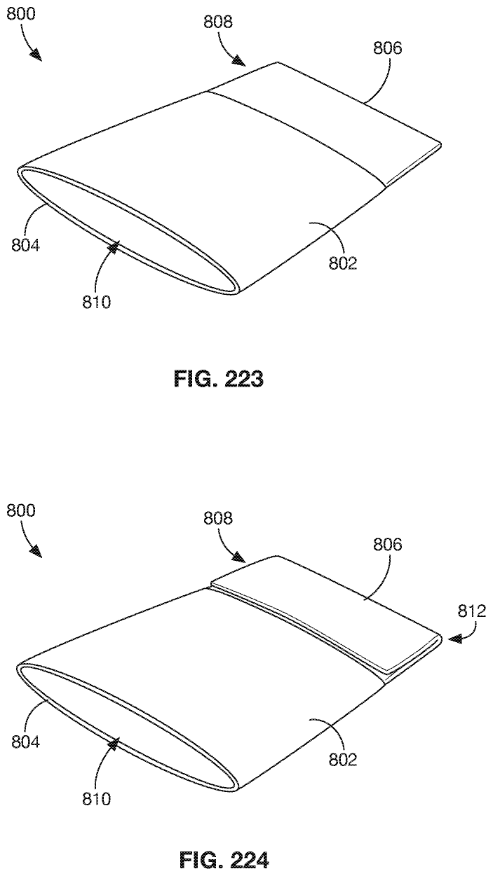

Referring now to FIGS. 223-224, an example of a cover 800 for the end 706 of the gripping member 702 comprises and/or is formed from a tube of material 802 that extends from a first end 804 to a second end 806. In some implementations, the tube, material, fabric, layer or membrane can be made of a flexible material, a porous material, and/or a material that is impermeable to blood flow. In some implementations, the tube, material, fabric, layer, or membrane is made from a biocompatible material, such as a woven biocompatible fabric that is configured to promote tissue ingrowth. To form the cover 800, the second end 806 of the tube of material 802 is flattened and fused together to form a closed portion 808 while an opening 810 remains in the first end 804. The tube of material 802 can be formed from a thermoplastic material so that the tube of material 802 can be flattened and then fused the tube of material 802 to itself to form the closed portion 808. The tube of material 802 can be fused to itself in any suitable way, such as, for example, via the application of heat from a heater, via ultrasonic welding (that generates heat from friction), via a laser, or the like. The tube of material 802 can optionally be folded along a fold 812 after being flattened and before being fused together, as is shown in FIG. 224. Before installing the cover 800 on the gripping member 702, the cover 800 can optionally be turned inside-out so that the closed portion 808 is moved to the interior of the tube of material 802.

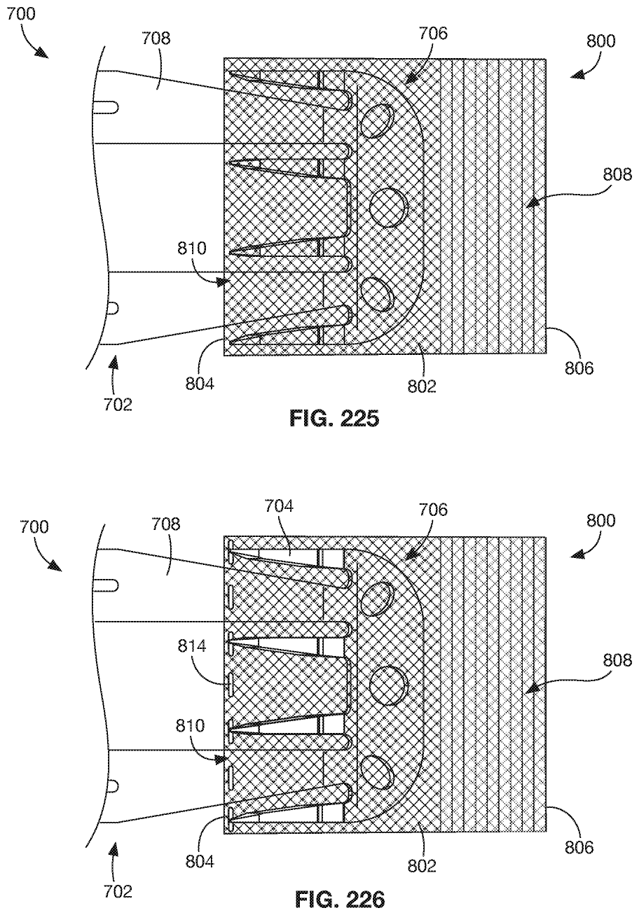

To attach the cover 800 to the gripping member 702, the end 706 of the moveable arm 708 of the gripping member 702 can be inserted into the opening 810 of the cover 800 until the end 706 engages the closed portion 808 of the second end 806 of the cover 800, as can be seen in FIGS. 225-226. In some implementations where the cover 800 is turned inside out, the end 706 of the gripping member 702 can overlap the closed portion 808 of the second end 806 of the cover 800. The friction-enhancing elements 704 are allowed to penetrate and protrude through the tube of material 802 by moving the tube of material 802 over the friction-enhancing elements 704 and/or by applying pressure to the tube of material 802 to push the friction-enhancing elements 704 through the tube of material 802. Once the cover 800 is in place, the opening 810 of the cover 800 can be closed by forming a plurality of stitches 814 (or other connectors as described herein) across the opening 810 of the tube of material 802 to attach and secure the cover 800 to the gripping member 702.

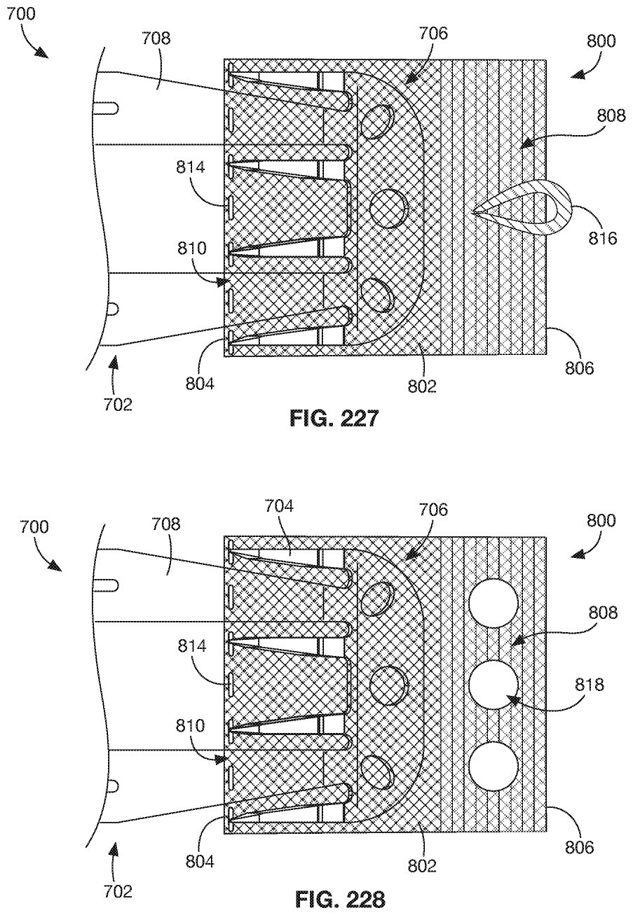

Referring now to FIGS. 227-228, the closed portion 808 of the cover 800 can include additional features for securing an actuation element (e.g., line, suture, wire, etc.) to the end 706 of the gripping member 702. The closed portion 808—and in particular the closed portion 808 formed from a folded portion of the tube of material 802—provides a reinforced portion of the cover 800 for attaching to an actuation element or suture (not shown). Referring now to FIG. 227, an attachment loop 816 is secured to the closed portion 808 of the cover 800. The attachment loop 816 can be attached to the cover 800 by stitching or any other suitable mechanical means, and/or by fusing the attachment loop 816 to the flattened tube of material 802 when the closed portion 808 is formed. In some implementations, one or more attachment holes 818 can be formed in the closed portion 808 of the cover 800 to which an actuation element or suture can be attached.

The small size of the gripping member 702 make assembly of the cover 710 to the end 706 of the gripping member 702 challenging and increase the potential for the attachment to take longer than expected or to result in an insufficient attachment. In particular, folding the cover 700 along the foldable region 716 and forming stitches 722 around the perimeter of the cover 700 present assembly challenges. Assembling the cover 800 to the gripping member 702 can take less time than the assembly of the cover 710 because the cover 800 can be attached by forming a single line of stitches 814 to close the opening 810 of the cover 800. Fusing the second end 806 of the cover 800 also increases the strength of the second end 806 of the cover 800.

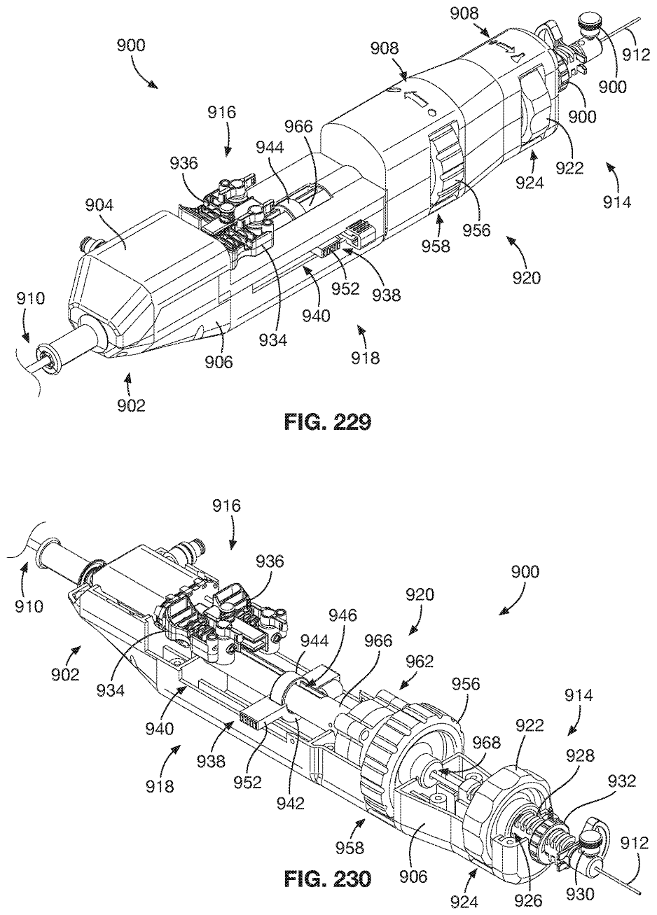

As was noted above, a delivery system for the devices described herein includes a control handle and a catheter. Referring now to FIGS. 229-233, an example of a control handle 900 is shown that facilitates the manipulation/actuation of a device, such as the devices 100, 200, 300, 400, 500, 600, 8200, 10000, 13110, 13210, 13310, 15000, 18000, 19000, 20500, 20600, 20700, and the like that are described herein. The control handle 900 includes various control mechanisms for interacting with the components of the valve repair devices described herein. The control handle 900 can incorporate any of the features of the control handles disclosed in U.S. Published Patent Application No. US20180325661 and Patent Cooperation Treaty International Application No. PCT/US2023/011452 (International Publication No. WO2023146859A1), which are incorporated herein by reference in their entirety.

The control handle 900 mechanisms are enclosed by a housing 902 having a top case 904 and a bottom case 906. The top case 904 and the bottom case 906 provide an ergonomic grip for the operator and can optionally include graphic indicators 908 of the function of the various control mechanisms of the control handle 900. A catheter assembly 910 extending from the control handle 900 provides a plurality of lumens through which a plurality of actuation elements 912 (e.g., rods, wires, tubes, shafts, lines, sutures, etc.) connect from the control handle 900 to an implantable device such as the devices described herein. The control handle 900 shown in FIGS. 229-230 can include a paddle actuation control 914. The control handle 900 can include a clasp actuation control 916. The control handle 900 can include a paddle width control 918. The control handle 900 can include a spacer width control 920.

In some implementations, the paddle actuation control 914 includes a control knob 922 that protrudes through a slot 924 in the housing 902 on both sides of the control handle 900. The control knob 922 includes a female threaded opening 926 through which a threaded lead screw 928 extends. One of the actuation elements 912 (e.g., rod, shaft, tube, line, suture, etc.) for actuating the paddles of the device extends through the lead screw 928 and is secured to the lead screw 928 with a set screw 930. Thus, rotating the control knob 922 in a clockwise or counter-clockwise direction causes the lead screw 928 and the connected actuation element 912 to translate in a proximal-distal direction. In some implementations, a stop nut 932 is arranged on the threads of the lead screw 928 so that the operator can establish a point at which further proximal translation of the lead screw 928 and the actuation element 912 is prohibited by the stop nut 932 engaging the housing 902 of the control handle 900.

In some implementations, the clasp actuation control 916 includes a first control member 934 (e.g., slider, etc.) and a second control member 936 (e.g., slider, etc.) that are each configured to move the clasps or gripping members of the valve repair device via actuation elements (e.g., the clasps 130 and actuation lines 116 of FIGS. 8-15). In some implementations, the catheter assembly 910 includes two clasp actuation lines that are each coupled to a corresponding clasp actuation member—i.e., to the first control slider 934 and the second control slider 936—at the proximal ends of the clasp actuation lines. Each of the first clasp control member 934 and the second control member 936 can be, for example, an axially movable control or slider coupled to a corresponding clasp actuation line to axially move the clasp actuation line. Each of the clasp control members 934, 936 can be operated independently of the other clasp control member 934, 936 such that each clasp actuation line is moved relative to the other clasp actuation line. The clasp control members 934, 936 can also be fixed with respect to one another (e.g., locked) such that the clasp actuation lines are axially moved together.

In some implementations, the paddle width control 918 includes a slider 938 that can be moved proximally and distally along a longitudinal slot 940 in the sides of the housing 902. The slider 938 can include a semi-circular grooved cradle 942 for supporting and engaging a support ring 944 of a drive nut 946. The drive nut 946 includes a main body 948 for engaging an actuation element 912 extending from the catheter assembly 910. A set screw 950 in the main body 948 can be tightened to secure the actuation element 912 to the main body 948 so that proximal and distal movement of the drive nut 946 also causes the actuation element 912 to translate in a similar manner. To enable proximal and distal movement by the operator, the slider 938 includes two actuation protrusions 952 that can be gripped by the operator to slide the slider 938 along the longitudinal slot 940 of the control handle 900. In some implementations, the slider 938 further includes a lock screw 954 that can be actuated to lock the longitudinal position of the slider 938 within the longitudinal slot 940, thereby fixing or locking the width of the paddles of the implantable valve repair device.

In some implementations, the actuation element 912 for the paddle width control 918 is a hypotube through which an actuation element 912 for the paddle actuation control 914 extends. Forming the actuation element 912 for the paddle width control 918 as a tube also enables the same actuation element 912 to be rotated by the drive nut 946 to engage other mechanisms of the implantable device, such as, for example, a screw and nut or other threaded member that can be rotated to cause a spacer or coaptation element of the implantable device to expand and retract. Rotation of the actuation element 912 for the paddle width control 918 to control the width of the spacer can be accomplished via the spacer width control 920.

In some implementations, the spacer width control 920 includes a control knob 956 that protrudes through a slot 958 in the housing 902 on both sides of the control handle 900. The control knob 956 connects to a drive shaft 960 that engages a multiplier gear box 962 so that each rotation of the control knob 956 results in multiple rotations of an output shaft 964 and an attached drive barrel 966. In some implementations, the gear box 962 has a gear ratio in a range of 1:2 to 1:10, or 1:3 to 1:8, or 1:4 to 1:6, or 1:5—that is, one turn of the control knob 956 and drive shaft 960 results in multiple turns of the output shaft 964 and drive barrel 966. Multiplying the rotations of the control knob 956 allows smaller movements of the control knob 956 to result in multiple rotations of the corresponding actuation element 912 that can be used, for example, to engage a screw or other threaded member of the connected valve repair device. Consequently, a finer thread pitch can be used for a screw and a nut or any other suitable threaded member of the valve repair device, thereby providing finer control of the width of the spacer. In some implementations, the spacer width control 920 includes a central opening 968 through which the actuation element 912 of the paddle actuation control 914 extends.

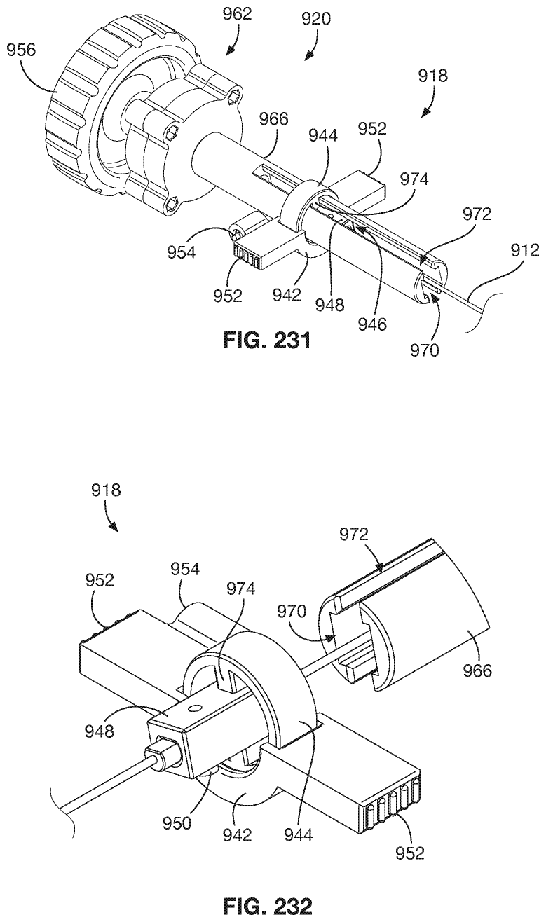

In some implementations, the drive barrel 966 includes a central drive slot 970 that is shaped to receive the main body 948 of the drive nut 946. In some implementations, as can be seen in FIGS. 231-232, the main body 948 is rectangular having a square cross-section and the drive slot 970 has a square shape for engaging the main body 948 with the drive slot 970. The drive slot 970 extends from a distal end of the drive barrel 966 and includes longitudinal slots 972 extending from the central drive slot 970 to the exterior of the drive barrel 966 to provide clearance for arms 974 that connect the main body 948 of the drive nut 946 to the support ring 944 of the drive nut 946. The drive slot 970 and longitudinal slots 972 allow the drive nut 946 to slide along the drive barrel 966 while also being rotatable by the drive barrel 966. Thus, the actuation element 912 can be translated and rotated simultaneously by the paddle width control 918 and the spacer width control 920, respectively.

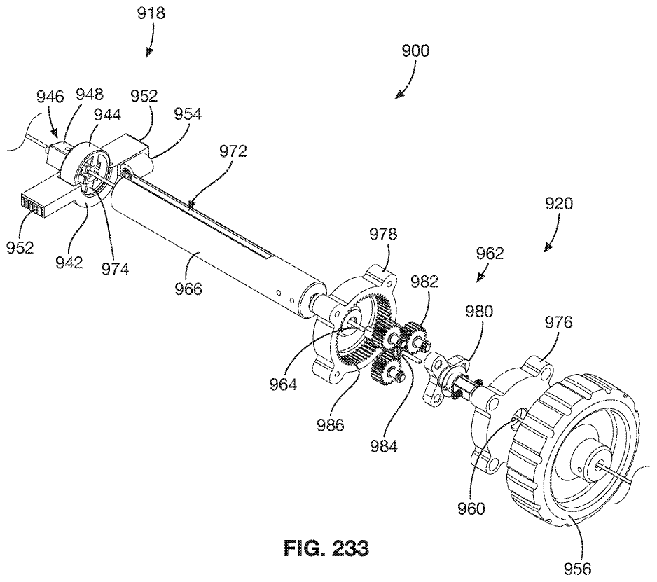

Referring now to FIG. 233, an exploded view of the spacer width control 920 is shown. As can be seen in FIG. 233, the multiplier gear box 962 includes a first housing half 976 and a second housing half 978. The drive shaft 960 extends through the cover of the first housing half 976 and is connected to a carrier 980 that rotatably engages a plurality of planetary gears 982 that surround a sun gear 984. The sun gear 984 is fixedly attached to the output shaft 964 that extends through the second housing half 978. The planetary gears 982 engage the sun gear 984 and also engage a ring gear 986 formed within the second housing half 978. Rotating the drive shaft 960 with the control knob 956 causes the carrier 980 to rotate, thereby moving the planetary gears 982 in a circular path within the second housing half 978. The planetary gears 982 engage and are rotated by the ring gear 986 as the carrier 980 moves the planetary gears 982. The rotating planetary gears 982 cause the sun gear 984 to rotate, thereby causing the rotation of the output shaft 964.

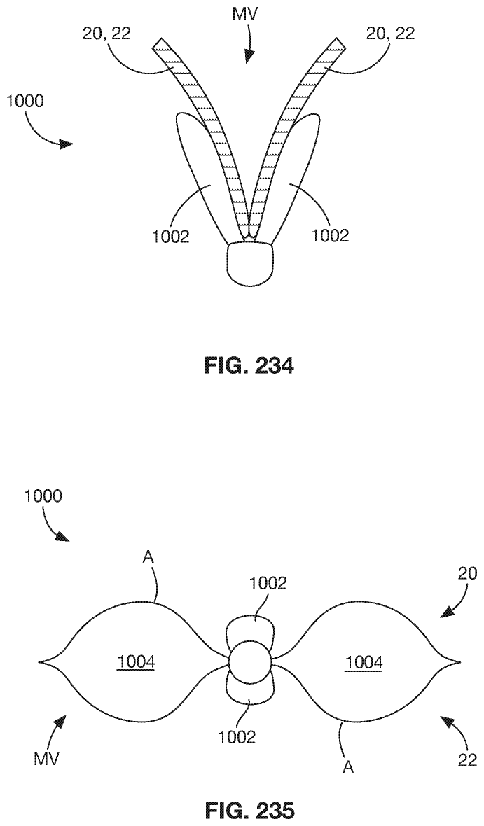

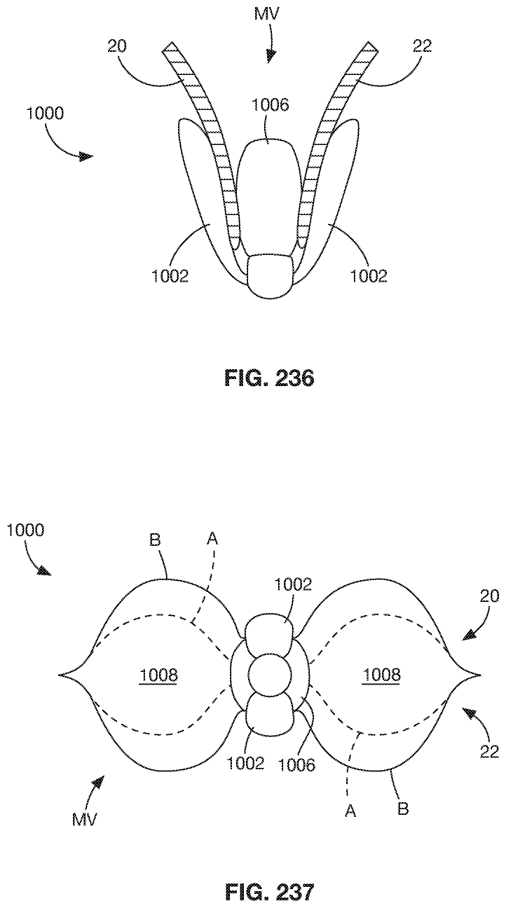

Referring now to FIGS. 234-237, an example of an implantable device 1000 is shown implanted in a mitral valve MV. The device 1000 includes a pair of anchors 1002 for capturing the native valve leaflets 20, 22. In FIGS. 234-235, the leaflets 20, 22 are pulled together by the anchors 1002 so that the leaflets 20, 22 coapt together. As can be seen in FIG. 235, pinching the leaflets 20, 22 together in the middle of the mitral valve MV results in the opening of the mitral valve MV being divided into two openings 1004 during diastole that are defined by a leaflet edge A. In FIGS. 236-237, the leaflets 20, 22 are pulled together by the anchors 1002 so that the leaflets 20, 22 coapt against a coaptation element or spacer 1006 that is arranged between the anchors 1002. As can be seen in FIG. 237, coapting the leaflets 20, 22 against the coaptation element 1006 allows the openings 1008 formed by the leaflets 20, 22 during diastole—defined by leaflet edge B—to be larger than the openings 1004 shown in FIG. 235. That is, the area of the openings 1008 defined by leaflet edge B is greater than the area of the openings 1004 defined by leaflet edge A. In some implementations, the openings 1008 have an area that is 10-100 percent larger, or 20-80 percent larger, or 30-70 percent larger, or 40-60 percent larger, or 50 percent larger than the area of the openings 1004. Consequently, the device 1000 that includes the coaptation element or spacer 1006 arranged between the leaflets 20, 22 provides greater blood flow through the mitral valve MV during diastole.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

### EXAMPLES

Example 1. A heart valve repair device comprising:
first and second paddle portions;
a spacer between the first and second paddle portions, the spacer comprising:
   a frame having at least first and second frame members;
   at least one pivot connection allowing one of the at least first and second frame members to pivot with respect to the other; and
wherein the at least first and second frame members are pivotable about the at least one pivot connection to move the spacer between a collapsed position and an expanded configuration.

Example 2. The heart valve repair device of example 1, wherein the collapsed position comprises the at least first and second frame members having a first distance between them.

Example 3. The heart valve repair device of example 2, wherein the expanded configuration comprises the at least first and second frame members having a second distance between them that is greater than the first distance.

Example 4. The heart valve repair device of any one of examples 1-3, wherein the at least one pivot connection comprises a first pivot connection at a first spacer frame location.

Example 5. The heart valve repair device of any one of examples 1-3, wherein the at least one pivot connection comprises a second pivot connection at a second spacer frame location.

Example 6. The heart valve repair device of any one of examples 1-5, wherein the at least first and second frame members each comprise first and second cross portions and a middle section therebetween.

Example 7. The heart valve repair device of any one of examples 1-6, wherein the at least one pivot connection comprises first and second pivots defining a rotational axis.

Example 8. The heart valve repair device of any one of examples 1-7, wherein the collapsed position comprises a first spacer volume.

Example 9. The heart valve repair device of example 8, wherein the expanded configuration comprises a second spacer volume greater than the first spacer volume.

Example 10. A heart valve repair device comprising:
first and second paddle portions that are movable between an open position and a closed position;
a spacer between the first and second paddle portions; and
wherein moving the first and second paddle portions from the closed position to the open position narrows the spacer.

Example 11. The heart valve repair device of example 10, wherein the spacer comprises:
a frame having at least first and second frame members and a cap engagement portion for engaging an actuation cap;
wherein the actuation cap moves away from the cap engagement portion when the first and second paddle portions move from the closed position toward the open position.

Example 12. The heart valve repair device of any one of examples 10-11, wherein the spacer is shape set in a narrow configuration.

Example 13. The heart valve repair device of example 10, wherein moving the first and second paddle portions from the open position to the closed position widens the spacer.

Example 14. The heart valve repair device of any one of examples 10-13, wherein moving the first and second paddle portions from the closed position to the open position automatically narrows the spacer.

Example 15. A heart valve repair device comprising:
first and second paddle portions that are movable between an open position and a closed position;
a spacer between the first and second paddle portions; and
wherein moving the first and second paddle portions from the open position to the closed position widens the spacer.

Example 16. The heart valve repair device of example 15, wherein the spacer comprises:
a frame having at least first and second frame members and a cap engagement portion for engaging an actuation cap;
wherein the actuation cap applies force to the cap engagement portion when the first and second paddle portions move from the open position to the closed position.

Example 17. The heart valve repair device of any one of examples 15-16, wherein the spacer is shape set in a narrow configuration.

Example 18. The heart valve repair device of example 15, wherein moving the first and second paddle portions from the open position to the closed position automatically widens the spacer.

Example 19. The heart valve repair device of any one of examples 15-18, wherein moving the first and second paddle portions from the closed position to the open position automatically narrows the spacer.

Example 20. A heart valve repair device comprising:
first and second paddle portions;
a spacer between the first and second paddle portions, the spacer comprising:
   a frame having at least first and second frame members;
   each frame member comprising:
      first and second flex portions and a middle portion therebetween;
      a flanged mounting portion;
   a brace portion connecting each frame member to the other frame member; and
wherein opening the first and second paddle portions moves the spacer from an expanded configuration to a collapsed position.

Example 21. The heart valve repair device of example 20, wherein the expanded configuration provides an expanded spacer width that is greater than a narrowed spacer width of the spacer in the collapsed position.

Example 22. The heart valve repair device of any one examples 20-21, wherein the expanded configuration comprises at least the first and second flex portions having a bent shape that is different from a bent shape of first and second flex portions in the collapsed position.

Example 23. The heart valve repair device of any one of examples 20-22, wherein the collapsed position comprises the spacer without an actuating force being applied.

Example 24. The heart valve repair device of any one of examples 20-22, wherein the expanded configuration comprises the spacer having an actuating force applied.

Example 25. The heart valve repair device of any one of examples 20-22, wherein the first and second flex portions flex outwardly from the device upon application of an actuating force.

Example 26. The heart valve repair device of any one of examples 20-25, wherein the spacer includes a space to allow movement of the brace portion as the spacer changes from the collapsed to the expanded configuration.

Example 27. The heart valve repair device of any one of examples 20-26, wherein the spacer is shape-set to the collapsed position.

Example 28. The heart valve repair device of any one of examples 20-27, further comprising a bushing to which the spacer is connected and the bushing comprising a retainer portion adjacent the spacer flanged mounting portion.

Example 29. The heart valve repair device of any one of examples 20-28, wherein the first and second flex portions and the middle portion comprise a sinuous shape in the expanded configuration.

Example 30. A heart valve repair device comprising:
a pair of paddles and a spacer formed from a unitary sheet of material, the unitary sheet of material having:
   a middle portion between first and second end portions of the unitary sheet of material;
   first and second spacer portions at the first and second end portions of the unitary sheet of material;
   first and second paddle portions between the first and second spacer portions;
   the middle portion between the first and second paddle portions; and
a plurality of first bend portions between the first and second spacer portions and the first and second paddle portions.

Example 31. The heart valve repair device of example 30, further comprising a plurality of second bend portions between the middle portion and the first and second paddle portions.

Example 32. The heart valve repair device of any one of examples 30-31, further comprising a plurality of third bend portions disposed within the paddles.

Example 33. The heart valve repair device of any one of examples 30-32, wherein the first and second spacer portions each comprise at least one spacer member.

Example 34. The heart valve repair device of any one of examples 30-33, wherein the first and second spacer portions each comprise a plurality of struts.

Example 35. The heart valve repair device of any one of examples 30-34, wherein the first and second spacer portions each comprise at least one outer strut and at least one inner strut.

Example 36. The heart valve repair device of any one of examples 30-35, wherein the middle portion comprises an aperture.

Example 37. The heart valve repair device of any one of examples 30-36, wherein the first and second paddle portions comprise one or more apertures.

Example 38. The heart valve repair device of any one of examples 30-37, wherein the unitary sheet of material further comprises a sinuous shape.

Example 39. The heart valve repair device of any one of examples 30-33, wherein the first and second spacer portions each comprise a sinuous middle region.

Example 40. The heart valve repair device of any one of examples 30-33, wherein the first and second spacer portions each comprise at least one spacer frame member having a looped middle region.

Example 41. The heart valve repair device of any one of examples 30-40, wherein a same spacer portion is manually sizable between a wide configuration and a narrow configuration.

Example 42. A heart valve repair device for attaching to a native heart valve, the heart valve repair device comprising:
first and second anchors that are movable between an open position and a closed position;
a coaptation element between the first and second anchors, wherein the coaptation element comprises a fabric material that defines shape of the coaptation element; and
wherein the fabric material of the coaptation element is configured to reduce stress on leaflets of the native heart valve when the leaflets are pressed against the coaptation element by the first and second anchors.

Example 43. The heart valve repair device of example 42, wherein the fabric material comprises a lofted cloth material.

Example 44. The heart valve repair device of any one of examples 42-43, further comprising a cover that is positioned over the fabric material.

Example 45. The heart valve repair device of example 44, wherein the cover comprises a woven biocompatible fabric that is configured to promote tissue ingrowth.

Example 46. The heart valve repair device of any one of examples 42-45, wherein the fabric material is disposed around a shaft.

Example 47. The heart valve repair device of any one of examples 42-46, wherein each of the first and second anchors comprises an inner paddle, an outer paddle, and a paddle frame.

Example 48. The heart valve repair device of any one of examples 42-46, further comprising a distal cap, wherein the first and second anchors are connected to the distal cap such that movement of the distal cap relative to the coaptation element causes the first and second anchors to move between the opened and closed positions.

Example 49. A heart valve repair device for attaching to a native heart valve, the heart valve repair device comprising:
first and second anchors that are movable between an open position and a closed position;
a coaptation element between the first and second anchors, wherein the coaptation element is movable between an elongated configuration and an expanded configuration;
wherein the coaptation element has a first width when in the elongated configuration and a second width when in the expanded configuration; and
wherein the second width is greater than the first width.

Example 50. The heart valve repair device of example 49, wherein a ratio of the second width to the first width is between 10/1 and 4/3, between 8/1 and 3/2, between 5/1 and 2/1, between 3/1 and 2/1 or any subrange of these ranges.

Example 51. The heart valve repair device of any one of examples 49-50, wherein a gap between the first anchor and the coaptation element when the first anchor is in the open position and the coaptation element is in the elongated configuration is larger than when the first anchor is in the open position and the coaptation element is in the expanded configuration.

Example 52. The heart valve repair device of any one of examples 49-51, wherein the coaptation element comprises Nitinol.

Example 53. The heart valve repair device of any one of examples 49-52, wherein the coaptation element is shape set in the expanded configuration.

Example 54. The heart valve repair device of example 53, wherein the coaptation element is configured to be moved to the elongated configuration by one or more actuation elements.

Example 55. The heart valve repair device of any one of examples 49-54, wherein the coaptation element comprises a first end member, a second end member, and a flexible body portion attached to and extending between the first and second end members, wherein at least one of the first and second end members is movable to move the flexible body portion such that the coaptation element moves between the elongated configuration and the expanded configuration.

Example 56. The heart valve repair device of example 55, wherein the first end member is disposed on a proximal end of the flexible body portion, and wherein the first end member is movable in a proximal direction to move the coaptation element to the elongated configuration and movable in a distal direction to move the coaptation element to the expanded configuration.

Example 57. The heart valve repair device of example 56, wherein the first end member is threadably attached to a shaft such that rotation of the first end member relative to the shaft causes the first end member to move and cause the coaptation element to move between the elongated and expanded configurations.

Example 58. The heart valve repair device of any one of examples 55-57, wherein at least one of the first and second end members is made of a material that has a greater stiffness than the flexible body portion.

Example 59. The heart valve repair device of any one of examples 55-58, wherein the flexible body portion comprises a braided metal fabric material.

Example 60. The heart valve repair device of any one of examples 55-58, wherein the flexible body portion comprises a flexible metal fabric material that includes strips extending between the first and second end members.

Example 61. The heart valve repair device of any one of examples 49-54, wherein the coaptation element comprises a cap that is configured to be engaged by an actuation element to move the coaptation element between the elongated and expanded configurations.

Example 62. The heart valve repair device of any one of examples 49-54, wherein the coaptation element comprises a flexible body member, a proximal element attached to a proximal end of the flexible body member, and a distal element attached to a distal end of the flexible body member, wherein the proximal element is movable between an extended position and a retracted position relative to the distal element, wherein the coaptation element is in the elongated configuration when the proximal element is in the extended position, and wherein the coaptation element is in the expanded configuration when the proximal element is in the retracted position.

Example 63. The heart valve repair device of example 62, wherein the proximal element comprises a cam body member and the distal element comprises a plunger member.

Example 64. The heart valve repair device of any one of examples 49-54, wherein the coaptation element comprises a flexible body member and a fluid actuator attached to the flexible body member, wherein the fluid actuator is movable between an extended position and a shortened position, wherein the coaptation element is in the elongated configuration when the fluid actuator is in the extended position, and wherein the coaptation element is in the expanded configuration when the fluid actuator is in the shortened position.

Example 65. The heart valve repair device of example 64, wherein the coaptation element further comprises a fluid line that is in fluid communication with the fluid actuator.

Example 66. The heart valve repair device of any one of examples 49-65, wherein the coaptation element comprises a coating that causes the coaptation element to have an acoustic impedance that is between about 1.5 MRayl and about 1.8 MRayl.

Example 67. The heart valve repair device of example 66, wherein the coaptation element comprises silicone rubber, and wherein the coating comprises at least one of barium sulfate, titanium dioxide, and zirconium dioxide.

Example 68. A heart valve repair system comprising:
a delivery device that includes an actuation tube;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration;
   wherein the coaptation element has a first width when in the elongated configuration and a second width when in the expanded configuration;
   wherein the second width is greater than the first width;
wherein the actuation tube of the delivery device is configured to engage and be extended over the coaptation element of the heart valve repair device to cause the coaptation element to be in the elongated configuration; and
wherein disengagement between the actuation tube and the coaptation element causes the coaptation element to be in the expanded configuration.

Example 69. The heart valve repair system of example 68, wherein a ratio of the second width to the first width is between 10/1 and 4/3, between 8/1 and 3/2, between 5/1 and 2/1, between 3/1 and 2/1 or any subrange of these ranges.

Example 70. The heart valve repair system of any one of examples 68-69, wherein a gap between the first anchor and the coaptation element when the first anchor is in the open position and the coaptation element is in the elongated configuration is larger than when the first anchor is in the open position and the coaptation element is in the expanded configuration.

Example 71. The heart valve repair system of any one of examples 68-70, wherein the coaptation element comprises Nitinol.

Example 72. The heart valve repair system of any one of examples 68-71, wherein the coaptation element is shape set in the expanded configuration.

Example 73. A heart valve repair system comprising:
a delivery device that includes one or more actuation lines;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration;
   wherein the coaptation element has a first width when in the elongated configuration and a second width when in the expanded configuration;
   wherein the second width is greater than the first width;
wherein, when the one or more actuation lines are attached to the coaptation element, providing a force to the one or more actuation lines causes the coaptation element to be in the elongated configuration;
wherein removal of the force from the one or more actuation lines causes the coaptation element to be in the expanded configuration.

Example 74. The heart valve repair system of example 73, wherein a ratio of the second width to the first width is between 10/1 and 4/3, between 8/1 and 3/2, between 5/1 and 2/1, between 3/1 and 2/1 or any subrange of these ranges.

Example 75. The heart valve repair system of any one of examples 73-74, wherein a gap between the first anchor and the coaptation element when the first anchor is in the open position and the coaptation element is in the elongated configuration is larger than when the first anchor is in the open position and the coaptation element is in the expanded configuration.

Example 76. The heart valve repair system of any one of examples 73-75, wherein the coaptation element comprises Nitinol.

Example 77. The heart valve repair system of any one of examples 73-76, wherein the coaptation element is shape set in the expanded configuration.

Example 78. A heart valve repair system comprising:
a delivery device that includes an actuation element;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration, the coaptation element comprising:
      a first end member;
      a second end member;
      a flexible body portion attached to and extending between the first and second end members, wherein the first end member is movable relative to the second end member to move the flexible body portion such that the coaptation element moves between the elongated configuration and the expanded configuration;
      wherein the coaptation element has a first width when in the elongated configuration and a second width when in the expanded configuration;
      wherein the second width is greater than the first width; and
wherein the actuation element of the delivery device is configured to engage the first end member of the coaptation element to move the first end member relative to the second end member.

Example 79. The heart valve repair system of example 78, wherein a ratio of the second width to the first width is between 10/1 and 4/3, between 8/1 and 3/2, between 5/1 and 2/1, between 3/1 and 2/1 or any subrange of these ranges.

Example 80. The heart valve repair system of any one of examples 78-79, wherein a gap between the first anchor and the coaptation element when the first anchor is in the open position and the coaptation element is in the elongated configuration is larger than when the first anchor is in the open position and the coaptation element is in the expanded configuration.

Example 81. The heart valve repair system of any one of examples 78-80, wherein the coaptation element comprises Nitinol.

Example 82. The heart valve repair system of any one of examples 78-81, wherein the coaptation element is shape set in the expanded configuration.

Example 83. The heart valve repair system of any one of examples 78-82, wherein the first end member is disposed on a proximal end of the flexible body portion, and wherein the first end member is movable in a proximal direction to move the coaptation element to the elongated configuration and movable in a distal direction to move the coaptation element to the expanded configuration.

Example 84. The heart valve repair system of any one of examples 78-83, wherein the first end member is threadably attached to a shaft, and wherein the actuation element is configured to engage the first end member to cause the first end member to rotate relative to the shaft to cause the first end member to move and cause the coaptation element to move between the elongated and expanded configurations.

Example 85. The heart valve repair system of any one of examples 78-84, wherein at least one of the first and second end members is made of a material that has a greater stiffness than the flexible body portion.

Example 86. The heart valve repair system of any one of examples 78-85, wherein the flexible body portion comprises a braided metal fabric material.

Example 87. The heart valve repair system of any one of examples 78-85, wherein the flexible body portion comprises a flexible metal fabric material that includes strips extending between the first and second end members.

Example 88. A heart valve repair system comprising:
a delivery device that includes an actuation element;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration, the coaptation element comprising a cap;
   wherein the coaptation element has a first width when in the elongated configuration and a second width when in the expanded configuration;
   wherein the second width is greater than the first width;
wherein the actuation element of the delivery device is configured to engage the cap of the coaptation element to move the cap and cause the coaptation element to be in the elongated configuration; and
wherein disengagement between the actuation element and the cap causes the coaptation element to be in the expanded configuration.

Example 89. The heart valve repair system of example 88, wherein a ratio of the second width to the first width is between 10/1 and 4/3, between 8/1 and 3/2, between 5/1 and 2/1, between 3/1 and 2/1 or any subrange of these ranges.

Example 90. The heart valve repair system of any one of examples 88-89, wherein a gap between the first anchor and the coaptation element when the first anchor is in the open position and the coaptation element is in the elongated configuration is larger than when the first anchor is in the open position and the coaptation element is in the expanded configuration.

Example 91. The heart valve repair system of any one of examples 88-90, wherein the coaptation element comprises Nitinol.

Example 92. The heart valve repair system of any one of examples 88-91, wherein the coaptation element is shape set in the expanded configuration.

Example 93. A heart valve repair system comprising:
a delivery device that includes an actuation element;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration, the coaptation element comprising:
      a flexible body member;
      a proximal element attached to a proximal end of the flexible body member;
      a distal element attached to a distal end of the flexible body member; and
      wherein the proximal element is movable between an extended position and a retracted position relative to the distal element;
      wherein the coaptation element is in the elongated configuration when the proximal element is in the extended position;
      wherein the coaptation element is in the expanded configuration when the proximal element is in the retracted position;
      wherein the coaptation element has a first width when in the elongated configuration and a second width when in the expanded configuration;
      wherein the second width is greater than the first width; and
wherein the actuation element of the delivery device is configured to engage the proximal element of the coaptation element to move the proximal element between the extended position and the retracted position.

Example 94. The heart valve repair system of example 93, wherein a ratio of the second width to the first width is between 10/1 and 4/3, between 8/1 and 3/2, between 5/1 and 2/1, between 3/1 and 2/1 or any subrange of these ranges.

Example 95. The heart valve repair system of any one of examples 93-94, wherein a gap between the first anchor and the coaptation element when the first anchor is in the open position and the coaptation element is in the elongated configuration is larger than when the first anchor is in the open position and the coaptation element is in the expanded configuration.

Example 96. The heart valve repair system of any one of examples 93-95, wherein the coaptation element comprises Nitinol.

Example 97. The heart valve repair system of any one of examples 93-96, wherein the coaptation element is shape set in the expanded configuration.

Example 98. The heart valve repair system of any one of examples 93-97, wherein the proximal element comprises a cam body member and the distal element comprises a plunger member.

Example 99. A heart valve repair system comprising:
a delivery device that includes a fluid line;
a heart valve repair device for attaching to a native heart valve, the heart valve repair device comprising:
   first and second anchors that are movable between an open position and a closed position;
   a coaptation element between the first and second anchors, wherein the coaptation element is movable between an elongated configuration and an expanded configuration, the coaptation element comprising a fluid actuator that is movable between an extended position and a shortened position;
   wherein the coaptation element is in the elongated configuration when the fluid actuator is in the extended position;
   wherein the coaptation element is in the expanded configuration when the fluid actuator is in the shortened position;
   wherein the coaptation element has a first width when in the elongated configuration and a second width when in the expanded configuration;
   wherein the second width is greater than the first width; and
wherein the fluid line of the delivery device is configured to move the fluid actuator between the extended position and the shortened position.

Example 100. The heart valve repair system of example 99, wherein a ratio of the second width to the first width is between 10/1 and 4/3, between 8/1 and 3/2, between 5/1 and 2/1, between 3/1 and 2/1 or any subrange of these ranges.

Example 101. The heart valve repair system of any one of examples 99-100, wherein a gap between the first anchor and the coaptation element when the first anchor is in the open position and the coaptation element is in the elongated configuration is larger than when the first anchor is in the open position and the coaptation element is in the expanded configuration.

Example 102. The heart valve repair system of any one of examples 99-101, wherein the coaptation element comprises Nitinol.

Example 103. The heart valve repair system of any one of examples 99-102, wherein the coaptation element is shape set in the expanded configuration.

Example 104. A heart valve repair device for attaching to a native heart valve, the heart valve repair device comprising:
first and second anchors that are movable between an open position and a closed position; and
a coaptation element between the first and second anchors, wherein the coaptation element comprises a material that causes the coaptation element to have an acoustic impedance that is between about 1.5 MRayl and about 1.8 MRayl.

Example 105. The heart valve repair device of example 104, wherein the coaptation element comprises silicone rubber.

Example 106. The heart valve repair device of any one of examples 104-105, wherein the material comprises at least one of barium sulfate, titanium dioxide, and zirconium dioxide.

Example 107. The heart valve repair device of example 106, wherein the material comprises a coating that includes the at least one of barium sulfate, titanium dioxide, and zirconium dioxide.

Example 108. The heart valve repair device of any one of examples 106-107, wherein the material comprises silicone rubber and 15 vol % barium sulfate.

Example 109. The heart valve repair device of any one of examples 106-107, wherein the material comprises silicone rubber and 17 vol % titanium dioxide.

Example 110. The heart valve repair device of any one of examples 106-107, wherein the material comprises silicone rubber and 10 vol % zirconium dioxide.

Example 111. The heart valve repair device of any one of examples 104-110, wherein each of the first and second anchors comprises an inner paddle, and outer paddle, and a paddle frame.

Example 112. The heart valve repair device of example 104, further comprising a distal cap, wherein the first and second anchors are connected to the distal cap such that movement of the distal cap relative to the coaptation element causes the first and second anchors to move between the opened and closed positions.

Example 113. The heart valve repair device of example 112, wherein the distal cap comprises a material that causes the distal cap to have an acoustic impedance that is between about 1.5 MRayl and about 1.8 MRayl.

Example 114. The heart valve repair device of example 113, wherein the material comprises at least one of barium sulfate, titanium dioxide, and zirconium dioxide.

Example 115. The heart valve repair device of example 114, wherein the material comprises a coating that includes the at least one of barium sulfate, titanium dioxide, and zirconium dioxide.

Example 116. The heart valve repair device of any one of examples 114-115, wherein the material comprises silicone rubber and 15 vol % barium sulfate.

Example 117. The heart valve repair device of any one of examples 114-115, wherein the material comprises silicone rubber and 17 vol % titanium dioxide.

Example 118. The heart valve repair device of any one of examples 114-115, wherein the material comprises silicone rubber and 10 vol % zirconium dioxide.

Example 119. A heart valve repair device for attaching to a native heart valve, the heart valve repair device comprising:
first and second anchors that are movable between an open position, a first closed position, and a second closed position;
a coaptation element between the first and second anchors, wherein the coaptation element is movable between an elongated configuration and an expanded configuration;
wherein the coaptation element has a first width when in the elongated configuration and a second width when in the expanded configuration; and
wherein moving the anchors from the first closed position to the second closed position moves the coaptation element from the elongated configuration to the expanded configuration.

Example 120. The heart valve repair device of example 119, wherein the second width is greater than the first width.

Example 121. The heart valve repair device of any of examples 119-120, wherein each of the first and second anchors comprises an inner paddle, an outer paddle, and a paddle frame.

Example 122. The heart valve repair device of any of examples 119-121, further comprising a distal cap, wherein the first and second anchors are connected to the distal cap such that movement of the distal cap relative to the coaptation element causes the first and second anchors to move between the opened and closed positions.

Example 123. The heart valve repair device of any of examples 121-122, wherein the outer paddles comprise paddle extension members.

Example 124. The heart valve repair device of example 123, wherein when the paddle extension members are narrower when the anchors are in the first closed position than when the anchors are in the second closed position.

Example 125. The heart valve repair device of example 123, wherein the paddle extension members are shape set when the anchors are in the first closed position.

Example 126. The heart valve repair device of example 125, wherein the coaptation element and the outer paddles are shape set when the anchors are in the first closed position.

Example 127. The heart valve repair device of any of examples 119-126, wherein tension applied to the anchors by captured leaflets of the native heart valve causes the coaptation element to decrease in width.

Example 128. A heart valve repair device for attaching to a native heart valve, the heart valve repair device comprising:
first and second anchors that are movable between an open position, a partially open position, and a closed position, wherein each of the first and second anchors comprises an inner paddle, an outer paddle, and a paddle frame;
a cover covering at least one of the inner paddle and the outer paddle of the first and second anchors; and
an extension of the cover extending from the anchors when the anchors are in the partially open position so that the width of the anchors including the extension is wider than the width of the anchors without the extension.

Example 129. The heart valve repair device of example 128, wherein the extension is attached to the cover covering the inner paddle.

Example 130. The heart valve repair device of example 128, wherein the extension is attached to the outer paddle.

Example 131. The heart valve repair device of example 128, wherein the extension is attached to the paddle frame.

Example 132. The heart valve repair device of example 128, wherein the extension is attached to the cover covering the outer paddle.

Example 133. The heart valve repair device of example 132, wherein the extension is attached to the inner paddle.

Example 134. The heart valve repair device of example 128, wherein the extension is attached to both the inner paddle and the outer paddle.

Example 135. The heart valve repair device of any of examples 128-134, wherein the extension is integrally formed with the cover.

Example 136. The heart valve repair device of any of examples 128-134, wherein the extension comprises and/or is formed from a loop of material attached to the cover.

Example 137. A heart valve repair device for attaching to a native heart valve, the heart valve repair device comprising:
a plurality of anchors that are movable between an open position and a closed position;
a gripping member attached to each of the plurality of anchors;
a cover covering an end of each gripping member, the cover comprising a tube of material extending from a first end to a second end, wherein the first end comprises an opening and the second end comprises a closed portion; and
wherein the gripping member is inserted into the opening of the cover.

Example 138. The heart valve repair device of example 137, wherein the gripping member comprises a plurality of friction enhancing-elements that protrude through the cover.

Example 139. The heart valve repair device of any of examples 137-138, wherein a single line of stitching is formed across the first end of the cover to close the opening of the cover.

Example 140. The heart valve repair device of any of examples 137-139, wherein the closed portion of the cover is formed by flattening and fusing the second end of the tube of material.

Example 141. The heart valve repair device of example 140, wherein the second end of the tube of material is folded before being fused.

Example 142. The heart valve repair device of any of examples 137-141, wherein the closed portion of the cover comprises an attachment loop.

Example 143. The heart valve repair device of any of examples 137-141, wherein the closed portion of the cover comprises an attachment hole.

Example 144. The heart valve repair device of any of examples 137-143, wherein:
a coaptation element is arranged between the plurality of anchors in the closed position so that leaflets of the native heart valve are captured between the coaptation element and the plurality of anchors when the anchors are closed; and
openings formed between the leaflets during diastole are larger than openings formed between the leaflets when pinched together by the anchors absent the coaptation element.

Example 145. A heart valve repair device for attaching to a native heart valve, the heart valve repair device comprising:
first and second anchors that are movable between an open position, a first closed position wherein the anchors have a first width, and a second closed position wherein the anchors have a second width;
a coaptation element between the first and second anchors, wherein the coaptation element is expandable between an elongated configuration and an expanded configuration;
wherein the coaptation element has a first width when in the elongated configuration and a second width when in the expanded configuration;
wherein when the first and second anchors are in the first closed position the coaptation element is in the elongated configuration; and
wherein when the first and second anchors are in the second closed position the coaptation element is in the expanded configuration.

Example 146. The heart valve repair device of example 145, wherein the second width of the anchors is greater than the first width of the anchors.

Example 147. The heart valve repair device of any of examples 145-146, wherein the second width of the coaptation element is greater than the first width of the coaptation element.

Example 148. The heart valve repair device of any of examples 145-147, wherein moving the anchors from the first closed position to the second closed position moves the coaptation element from the elongated configuration to the expanded configuration.

Example 149. The heart valve repair device of any of examples 145-148, wherein moving the coaptation element from the expanded configuration to the elongated configuration moves the anchors from the second closed position to the first closed position.

Example 150. The heart valve repair device of any of examples 145-148, wherein moving the anchors in a closing direction causes the coaptation element to expand.

Example 151. The heart valve repair device of any of examples 145-150, wherein moving the anchors in an opening direction causes the coaptation element to elongate.

Example 152. The heart valve repair device of any of examples 145-151, wherein each of the first and second anchors comprises an inner paddle, an outer paddle, and a paddle frame.

Example 153. The heart valve repair device of any of examples 145-152, further comprising a distal cap, wherein the first and second anchors are connected to the distal cap such that movement of the distal cap relative to the coaptation element causes the first and second anchors to move between the opened and closed positions.

Example 154. The heart valve repair device of any of examples 145-153, wherein the outer paddles comprise paddle extension members.

Example 155. The heart valve repair device of example 154, wherein when the paddle extension members are narrower when the anchors are in the first closed position than when the anchors are in the second closed position.

Example 156. The heart valve repair device of example 155, wherein the paddle extension members are shape set when the anchors are in the first closed position.

Example 157. The heart valve repair device of example 156, wherein the coaptation element and the outer paddles are shape set when the anchors are in the first closed position.

Example 158. The heart valve repair device of any of examples 145-157, wherein tension applied to the anchors by captured leaflets of the native valve causes the coaptation element to decrease in width.

Example 159. A heart valve repair system comprising:
a valve repair device of any of examples 119-158;
a delivery system comprising:
   a catheter assembly;
   a control handle, the control handle comprising:
      a control knob connected to a drive shaft;
a multiplier gearbox that is engaged by the drive shaft;
an actuation element that is operatively coupled to an output shaft of the multiplier gearbox, wherein the actuation element extends through the catheter assembly to the valve repair device; and
wherein rotating the control knob causes the actuation element to rotate via the multiplier gearbox so that a width of the coaptation element of the valve repair device is changed.

Example 160. The heart valve repair system of example 159, wherein:
the output shaft of the multiplier gearbox is connected to a drive barrel, the drive barrel comprises a central drive slot; and
the actuation element is attached to a drive nut that slideably engages the central drive slot of the drive barrel.

Example 161. The heart valve repair system of example 160, wherein:
the drive nut comprises a support ring that is connected to a main body of the drive nut via arms; and
the drive barrel comprises longitudinal slots connected to the central drive slot for providing clearance for the arms of the drive nut.

Example 162. The heart valve repair system of example 161, further comprising:
a slider comprising a semi-circular grooved cradled for supporting and engaging the support ring of the drive nut;
wherein moving the slider in a proximal and a distal direction engages and moves the drive nut and actuation element in the proximal and the distal direction.

Example 163. The heart valve repair system of any of examples 159-162, wherein the multiplier gearbox is a planetary gearbox, the drive shaft is connected to a carrier of the planetary gear box and the output shaft is connected to a sun gear of the planetary gearbox.

Example 164. The heart valve repair system of any of the examples 159-163, wherein the multiplier gearbox has a gear ratio between the drive shaft and the output shaft in a range of 1:2 to 1:10, or in a range of 1:3 to 1:8, or in a range of 1:4 to 1:6, or 1:5.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A heart valve repair device comprising:
first and second paddle portions that are movable between an open position and a closed position;
a spacer between the first and second paddle portions; and
wherein moving the first and second paddle portions from the closed position to the open position narrows the spacer.

2. The heart valve repair device of claim 1, wherein the spacer comprises a stent and a polymer based cover disposed over the stent.

3. The heart valve repair device of claim 1, wherein the spacer is configured to be expanded to act as a dock for a prosthetic valve.

4. The heart valve repair device of claim 1, wherein the first and second paddle portions are connected to the spacer by a torsional spring hinge portion.

5. The heart valve repair device of claim 1, wherein the first and second paddle portions and the spacer are integrally formed from a single piece of sheet material.

6. The heart valve repair device of claim 1, wherein the spacer comprises a frame that includes first and second cross portions that are connected by first and second pivots.

7. The heart valve repair device of claim 1, further comprising:
a bushing that supports the spacer; and
a brace section of the first and second paddle portions,
wherein a compression gap is defined between the bushing the brace section of the first and second paddle portions, and
wherein, while the paddles are in the closed position, the spacer expands by reducing the compression gap.

8. The heart valve repair device of claim 1, wherein the spacer is made of one or more of silicone rubber and barium sulfate, silicone rubber and titanium dioxide, and silicone rubber and zirconium dioxide.

9. The heart valve repair device of claim 1, wherein each of the first and second paddle portions comprise an inner paddle, an outer paddle, and a joint between the inner paddle and the outer paddle, wherein the valve repair device comprises fabric extensions that extends from each of the joints.

10. The heart valve repair device of claim 1, further comprising a pair of gripping members between the spacer and the first and second paddles and a cover covering an end of each gripping member, the cover comprising a tube of material extending from a first end to a second end, wherein the first end comprises an opening and the second end that is closed by an ultrasonic seal.

11. The valve repair device of claim 1 wherein the spacer is configured to increase an area between native heart valve leaflets when the heart valve is in the diastolic phase.

12. The valve repair device of claim 11 wherein the spacer is configured to increase an area between the native heart valve leaflets when the heart valve is in the diastolic phase by 20 percent to 80 percent.

13. A heart valve repair system comprising:
a delivery device that includes an actuation element;
a heart valve repair device for attaching to a native heart valve, the heart valve repair device comprising:
first and second anchors that are movable between an open position and a closed position;
a coaptation element between the first and second anchors, wherein the coaptation element is movable between an elongated configuration and an expanded configuration, the coaptation element comprising:
a flexible body member;
a proximal element attached to a proximal end of the flexible body member;
a distal element attached to a distal end of the flexible body member;
wherein the proximal element is movable between an extended position and a retracted position relative to the distal element;
wherein the coaptation element is in the elongated configuration when the proximal element is in the extended position;
wherein the coaptation element is in the expanded configuration when the proximal element is in the retracted position;
wherein the coaptation element has a first width when in the elongated configuration and a second width when in the expanded configuration;
wherein the second width is greater than the first width; and
wherein the actuation element of the delivery device is configured to engage the proximal element of the coaptation element to move the proximal element between the extended position and the retracted position.

14. The heart valve repair system of claim 13 wherein the delivery device comprises:
a catheter assembly;
a control handle, the control handle comprising:
a control knob connected to a drive shaft;
a multiplier gearbox that is engaged by the drive shaft;
an actuation element that is operatively coupled to an output shaft of the multiplier gearbox, wherein the actuation element extends through the catheter assembly to the valve repair device; and
wherein rotating the control knob causes the actuation element to rotate via the multiplier gearbox so that a width of the coaptation element of the valve repair device is changed.

15. The heart valve repair system of claim 14, wherein:
the output shaft of the multiplier gearbox is connected to a drive barrel, the drive barrel comprises a central drive slot; and
the actuation element is attached to a drive nut that slideably engages the central drive slot of the drive barrel.

16. A heart valve repair device for attaching to a native heart valve, the heart valve repair device comprising:
first and second anchors that are movable between an open position and a closed position;
a coaptation element between the first and second anchors, wherein the coaptation element comprises a fabric material that defines shape of the coaptation element; and
wherein the fabric material of the coaptation element is configured to reduce stress on leaflets of the native heart valve when the leaflets are pressed against the coaptation element by the first and second anchors.

17. The heart valve repair device of claim 16, wherein the first and second anchors comprise a torsional spring hinge portion.

18. The heart valve repair device of claim 16, wherein each of the first and second paddle portions comprise an inner paddle, an outer paddle, and a joint between the inner paddle and the outer paddle, wherein the valve repair device comprises fabric extensions that extends from each of the joints.

19. The heart valve repair device of claim 16, wherein the first and second anchors comprise a pair of gripping members and a cover covering an end of each gripping member, the cover comprising a tube of material extending from a first end to a second end, wherein the first end comprises an opening and the second end that is closed by an ultrasonic seal.

20. The heart valve repair device of claim 16, further comprising a cover over the fabric material of the coaptation element.
